(19) United States
(12) Patent Application Publication
(10) Pub. No.: US 2026/0007680 A1
(43) Pub. Date: Jan. 8, 2026

(54) Compounds and Their Use as PDE4 Activators

(71) Applicant: Mironid Limited, Glasgow (GB)

(72) Inventors: Julia Mary Adam, Glasgow (GB); David Roger Adams, Glasgow (GB)

(73) Assignee: Mironid Limited, Glasgow (GB)

(21) Appl. No.: 19/103,768

(22) PCT Filed: Aug. 17, 2023
(86) PCT No.: PCT/EP2023/072657

§ 371 Date: Feb. 13, 2025

(30) Foreign Application Priority Data
Aug. 17, 2022 (GB) 2211999.4

(51) Int. Cl. A61K 31/5377; A61K 31/423; A61K 31/4439; A61K 31/444; A61K 31/454; A61K 31/4545; A61K 31/496; A61K 31/4995; A61K 31/519; A61P 5/18; A61P 13/12; A61P 35/00; C07D 401/04; C07D 405/14; C07D 413/04; C07D 413/14; C07D 471/04; C07D 487/04; C07D 513/04; C07D 519/00

(52) U.S. Cl. CPC A61K 31/5377; A61K 31/423; A61K 31/4439; A61K 31/444; A61K 31/454; A61K 31/4545; A61K 31/496; A61K 31/4995; A61K 31/519; A61P 5/18; A61P 13/12; A61P 35/00; C07D 401/04; C07D 405/14; C07D 413/04; C07D 413/14; C07D 471/04; C07D 487/04; C07D 513/04; C07D 519/00

(57) ABSTRACT

The present invention relates to compounds of Formulas I to IV, their use as activators of long form cyclic nucleotide phosphodiesterase-4 (PDE4) enzymes (isoforms) and to these compounds for use in a method for the treatment or prevention of disorders requiring a reduction of second messenger responses mediated by cyclic 3′,5′-adenosine monophosphate (cAMP).

### FIELD OF THE INVENTION

The present invention relates to compounds as defined herein, their use as activators of long form cyclic nucleotide phosphodiesterase-4 (PDE4) enzymes (isoforms) and to therapies using these compounds. In particular, the invention relates to these compounds for use in a method for the treatment or prevention of disorders requiring a reduction of second messenger responses mediated by cyclic 3′,5′-adenosine monophosphate (cAMP).

### BACKGROUND TO THE INVENTION

Cyclic 3′,5′-adenosine monophosphate—“cAMP”—is a critical intracellular biochemical messenger that is involved in the transduction of the cellular effects of a variety of hormones, neurotransmitters, and other extracellular biological factors in most animal and human cells.

The intracellular concentration of cAMP is controlled by the relative balance between its rate of production and degradation. cAMP is generated by biosynthetic enzymes of the adenylyl cyclase superfamily and degraded by members of the cyclic nucleotide phosphodiesterase (PDE) superfamily. Certain members of the PDE superfamily, such as PDE4, specifically degrade cAMP, while others either specifically degrade cyclic guanosine monophosphate (cGMP) or degrade both cAMP and cGMP. PDE4 enzymes inactivate cAMP, thereby terminating its signalling, by hydrolysing cAMP to 5′-AMP (Lugnier, C. Pharmacol Ther. 109: 366-398, 2006).

Four PDE4 genes (PDE4A, PDE4B, PDE4C and PDE4D) have been identified, each of which encodes a number of different enzyme isoforms through the use of alternative promoters and mRNA splicing. On the basis of their primary structures, the catalytically active PDE4 splice variants can be classified as “long”, “short” or “super-short” forms (Houslay, M. D. Prog Nucleic Acid Res Mol Biol. 69: 249-315, 2001). A “dead short” form also exists, which is not catalytically active (Houslay, M. D., Baillie, G. S. and Maurice, D. H. Circ Res. 100: 950-66, 2007). PDE4 long forms have two regulatory regions, called upstream conserved regions 1 and 2 (UCR1 and UCR2), located between their isoform-specific N-terminal portion and the catalytic domain. The UCR1 domain is absent in short forms, whereas the super-short forms not only lack UCR1, but also have a truncated UCR2 domain (Houslay, M. D., Schafer, P. and Zhang, K. Drug Discovery Today 10: 1503-1519, 2005).

PDE4 long forms, but not short forms, associate into dimers within cells (Richter, W and Conti, M. J. Biol. Chem. 277: 40212-40221, 2002; Bolger, G. B. et al., Cell. Signal. 27: 756-769, 2015). A proposed negative allosteric modulation of PDE4 long forms by small molecules has been reported (Burgin A. B. et al., Nat. Biotechnol. 28: 63-70, 2010; Gurney M. E. et al., Handb. Exp. Pharmacol. 204: 167-192, 2011).

It is known in the art that PDE4 long forms may be activated by endogenous cellular mechanisms, such as phosphorylation (MacKenzie, S. J. et al., Br. J. Pharmacol. 136: 421-433, 2002) and phosphatidic acid (Grange et al., J. Biol. Chem. 275: 33379-33387, 2000). Activation of PDE4 long forms by ectopic expression of a 57 amino acid protein (called ‘UCR1C’) whose precise sequence reflects part of that of the upstream conserved region 1 of PDE4D (‘UCR1C’ sequence reflects that of amino acids 80-136 while UCR is amino acids 17-136: numbering based on the PDE4D3 long isoform) has been reported (Wang, L. et al., Cell. Signal. 27: 908-922, 2015: “UCR1C is a novel activator of phosphodiesterase 4 (PDE4) long isoforms and attenuates cardiomyocyte hypertrophy”). The authors hypothesised that PDE4 activation might be used as a potential therapeutic strategy for preventing cardiac hypertrophy.

The first small molecules that act as activators of PDE4 long forms were recently disclosed in WO2016151300, WO2018060704 and WO2019193342. A small molecule activator of PDE4 long forms was recently evaluated in cell-based models of Autosomal Dominant Polycystic Kidney Disease (ADPKD) (Omar et al., PNAS 116: 13320-13329, 2019). No small molecule activators of PDE4 long forms have yet been reported in clinical development. There remains a need for further, structurally distinct small molecule activators of PDE4 long forms for potential development as therapeutic agents.

It is amongst the objects of the present invention to provide new small molecule activators of at least one of the long forms of PDE4 for use in a method of therapy, as well as specific disease treatment or prevention.

### SUMMARY OF THE INVENTION

In a first aspect of the present invention, there is provided a compound of Formula I:

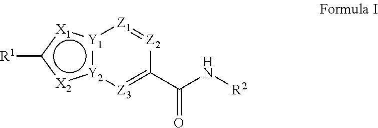

or a pharmaceutically acceptable salt or derivative thereof, wherein:
   one of X₁ and X₂ is N and the other is N or CR^{3a}, one of Y₁ and Y₂ is N and the other is C, and one of Z₁, Z₂ and Z₃ is N or CR^{3b} and the others are each CR^{3b}; or
   one of X₁ and X₂ is N and the other is NR^{3c} or O, Y₁ and Y₂ are each C, and one of Z₁, Z₂ and Z₃ is N or CR^{3b} and the others are each CR^{3b}; or
   one of X₁ and X₂ is S and the other is N or CR^{3a}, Y₁ and Y₂ are each C, and one of Z₁, Z₂ and Z₃ is N and the others are each CR^{3b};
   R¹ is a 4- to 10-membered monocyclic, bridged or bicyclic ring containing at least 1 ring N heteroatom and optionally a ring O heteroatom, and wherein R¹ is optionally substituted with 1 or more R⁴;
   R² is
   (i) (C5-7)cycloalkyl, fused to a 6-membered aromatic or heteroaromatic ring that contains 0, 1 or 2 ring N atoms;
   (ii) a 5- to 7-membered non-aromatic heterocycle containing one ring O heteroatom, optionally fused to a 6-membered aromatic or heteroaromatic ring that contains 0, 1 or 2 ring N atoms;
   (iii) CH₂Ar, where Ar is a 6-membered aromatic or heteroaromatic ring that contains 0, 1 or 2 ring N atoms; or
   (iv) a (C3-8)alkyl group that may be straight chain, branched or cyclic, or a combination thereof;
   and wherein R² is optionally substituted with 1 or more R⁵;
   each R^{3a} is independently H or (C1-6)alkyl, the (C1-6)alkyl being optionally substituted by 1 or more halogen;
   each R^{3b} is independently H, (C1-6)alkyl, (C1-6)alkoxy, CN or halogen, the (C1-6)alkyl and (C1-6)alkoxy being optionally substituted by 1 or more halogen;
   each R^{3c} is independently H or (C1-6)alkyl, the (C1-6)alkyl being optionally substituted by 1 or more halogen;
   each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy; and
   each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH;
for use in the treatment or prevention of a disease or disorder that can be ameliorated by activation of long isoforms of PDE4 or a disease or disorder mediated by excessive intracellular cyclic AMP signalling.

In a second aspect of the present invention, there is provided a compound of Formula II

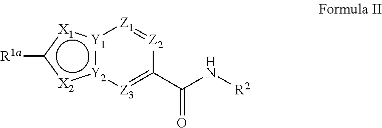

or a pharmaceutically acceptable salt or derivative thereof, wherein:
   one of X₁ and X₂ is N and the other is N or CR^{3a}, one of Y₁ and Y₂ is N and the other is C, and one of Z₁, Z₂ and Z₃ is N or CR^{3b} and the others are each CR^{3b}; or
   one of X₁ and X₂ is S and the other is N or CR^{3a}, Y₁ and Y₂ are each C, and one of Z₁, Z₂ and Z₃ is N and the others are each CR^{3b};
   R^{1a} is a 4- to 10-membered non-aromatic ring that can be monocyclic, bridged or bicyclic containing at least 1 ring N heteroatom and optionally a ring O heteroatom, wherein at least 1 ring N heteroatom is not at the point of attachment of R^{1a}, and wherein R^{1a} is optionally substituted with 1 or more R⁴;
   R² is
   (i) (C5-7)cycloalkyl, fused to a 6-membered aromatic or heteroaromatic ring that contains 0, 1 or 2 ring N atoms;
   (ii) a 5- to 7-membered non-aromatic heterocycle containing one ring O heteroatom, optionally fused to a 6-membered aromatic or heteroaromatic ring that contains 0, 1 or 2 ring N atoms;
   (iii) CH₂Ar, where Ar is a 6-membered aromatic or heteroaromatic ring that contains 0, 1 or 2 ring N atoms; or
   (iv) a (C3-8)alkyl group that may be straight chain, branched or cyclic, or a combination thereof;
   and wherein R² is optionally substituted with 1 or more R⁵;
   each R^{3a} is independently H or (C1-6)alkyl, the (C1-6)alkyl being optionally substituted by 1 or more halogen;
   each R^{3b} is independently H, (C1-6)alkyl, (C1-6)alkoxy, CN or halogen, the (C1-6)alkyl and (C1-6)alkoxy being optionally substituted by 1 or more halogen;
   each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy; and
   each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH.

In a third aspect of the present invention, there is provided a compound of Formula III

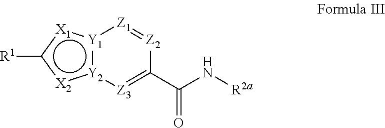

or a pharmaceutically acceptable salt or derivative thereof, wherein:
   one of X₁ and X₂ is N and the other is N or CR^{3a}, one of Y₁ and Y₂ is N and the other is C, and one of Z₁, Z₂ and Z₃ is N or CR^{3b} and the others are each CR^{3b}; or
   one of X₁ and X₂ is S and the other is N or CR^{3a}, Y₁ and Y₂ are each C, and one of Z₁, Z₂ and Z₃ is N and the others are each CR^{3b};
   R¹ is a 4- to 10-membered monocyclic, bridged or bicyclic ring containing at least 1 ring N heteroatom and optionally a ring O heteroatom, and wherein R¹ is optionally substituted with 1 or more R⁴;
   R^{2a} is
   (i) (C5-7)cycloalkyl fused to a 6-membered aromatic or heteroaromatic ring that contains 0, 1 or 2 ring N atoms;
   (ii) a 5- to 7-membered non-aromatic heterocycle containing one ring O heteroatom, optionally fused to a 6-membered aromatic or heteroaromatic ring that contains 0, 1 or 2 ring N atoms; or
   (35) (iv) a (C5-6)cycloalkyl group;
   and wherein R^{2a} is optionally substituted with 1 or more R⁵;
   each R^{3a} is independently H or (C1-6)alkyl, the (C1-6)alkyl being optionally substituted by 1 or more halogen;
   each R^{3b} is independently —H, (C1-6)alkyl, (C1-6)alkoxy, CN or halogen, the (C1-6)alkyl and (C1-6)alkoxy being optionally substituted by 1 or more halogen;
   each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy; and
   each R⁵ is independently halogen, OH, CN, (C1-6)alkyl, (C1-6)alkoxy or —(C1-6)alkylene-(C1-6)alkoxy the (C1-6)alkyl and (C1-6)alkoxy being optionally substituted by 1 or more halogen or OH; and wherein when one of X₁ and X₂ is S and the other is N, Y₁ and Y₂ are each C, one of Z₁, Z₂ and Z₃ is N and R^{2a} is (iv) a (C5-6)cycloalkyl group, R^{2a} is substituted by at least 2 R⁵.

In a fourth aspect of the present invention, there is provided a compound of Formula IV

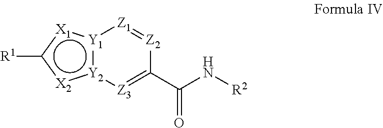

or a pharmaceutically acceptable salt or derivative thereof, wherein:
   one of X₁ and X₂ is N and the other is N or CR^{3a}, one of Y₁ and Y₂ is N and the other is C, and one of Z₁, Z₂ and Z₃ is N or CR^{3b} and the others are each CR^{3b}; or
   one of X₁ and X₂ is S and the other is N or CR^{3a}, Y₁ and Y₂ are each C, and one of Z₁, Z₂ and Z₃ is N and the others are each CR^{3b};
   R¹ is a 4- to 10-membered monocyclic, bridged or bicyclic ring containing at least 1 ring N heteroatom and optionally a ring O heteroatom, and wherein R¹ is optionally substituted with 1 or more R⁴;
   R² is
   (i) (C5-7)cycloalkyl, fused to a 6-membered aromatic or heteroaromatic ring that contains 0, 1 or 2 ring N atoms;
   (ii) a 5- to 7-membered non-aromatic heterocycle containing one ring O heteroatom, optionally fused to a 6-membered aromatic or heteroaromatic ring that contains 0, 1 or 2 ring N atoms;
   (iii) CH₂Ar, where Ar is a 6-membered aromatic or heteroaromatic ring that contains 0, 1 or 2 ring N atoms; or
   (iv) a (C3-8)alkyl group that may be straight chain, branched or cyclic, or a combination thereof;
   and wherein R² is optionally substituted with 1 or more R⁵;
   each R^{3a} is independently H or (C1-6)alkyl, the (C1-6)alkyl being optionally substituted by 1 or more halogen;
   each R^{3b} is independently H, (C1-6)alkyl, (C1-6)alkoxy, CN or halogen, the (C1-6)alkyl and (C1-6)alkoxy being optionally substituted by 1 or more halogen, wherein at least 1 R^{3b} is other than H;
   each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and (C1-6)alkylene-(C1-6)alkoxy being optionally substituted with 1 or more substituents independently selected from halogen, OH and (C1-6)alkoxy; and
each R⁵ is independently halogen, OH, CN, (C1-6)alkyl, (C1-6)alkoxy or —(C1-6)alkylene-(C1-6)alkoxy the (C1-6)alkyl and (C1-6)alkoxy being optionally substituted by 1 or more halogen or OH.

Compounds described herein are shown in the Examples to activate PDE4 long form enzymes.

In a further aspect, the present invention provides a pharmaceutical composition comprising a compound or a pharmaceutically acceptable salt or derivative as described herein, and a pharmaceutically acceptable excipient.

In a further aspect, the present invention provides a compound or pharmaceutical composition described herein for use in therapy. The therapy may be the treatment or prevention of any disease or disorder as described herein. The therapy may be the treatment or prevention of a disease or disorder that can be ameliorated by activation of long isoforms of PDE4. The therapy may be the treatment or prevention of a disease or disorder mediated by excessive intracellular cAMP signalling. In these diseases, a reduction of second messenger responses mediated by cyclic 3′,5′-adenosine monophosphate (cAMP) should provide a therapeutic benefit.

Also provided is a method of treating or preventing a disease or disorder that can be ameliorated by activation of long isoforms of PDE4, comprising the step of administering an effective amount of a compound or pharmaceutical composition described herein to a patient in need thereof. Also provided is a method of treating or preventing a disease or disorder mediated by excessive intracellular cAMP signalling, comprising the step of administering an effective amount of a compound or pharmaceutical composition described herein to a patient in need thereof.

Also provided is the use of a compound or pharmaceutical composition described herein in the manufacture of a medicament for treating or preventing a disease or disorder that can be ameliorated by activation of long isoforms of PDE4. Also provided is the use of a compound or pharmaceutical composition described herein in the manufacture of a medicament for treating or preventing a disease or disorder mediated by excessive intracellular cAMP signalling.

In certain embodiments of the foregoing aspects, the compounds of the invention are provided for the treatment or prevention of cancer. In certain embodiments of the foregoing aspects, the compounds of the invention are provided for the treatment or prevention of a disease or disorder selected from hyperthyroidism, Jansens's metaphyseal chondrodysplasia, hyperparathyroidism, familial male-limited precocious puberty, pituitary adenomas, Cushing's disease, polycystic kidney disease, polycystic liver disease, McCune-Albright syndrome, cholera, whooping cough, anthrax, tuberculosis, HIV, AIDS, Common Variable Immunodeficiency (CVID), melanoma, pancreatic cancer, leukaemia, prostate cancer, adrenocortical tumours, testicular cancer, primary pigmented nodular adrenocortical diseases (PPNAD), Carney Complex, autosomal dominant polycystic kidney disease (ADPKD), autosomal recessive polycystic kidney disease (ARPKD), maturity onset diabetes of young type 5 (MODY5), or cardiac hypertrophy.

### BRIEF DESCRIPTION OF THE FIGURES

FIG. 1 shows concentration-dependent activation of a PDE4 long form, PDE4D5, by Example 4 using the method described in Experiment 1.

FIG. 2 shows inhibition of cyst formation in a 3D culture of m-IMCD3 mouse kidney cells treated with Example 51, using the method described in Experiment 4.

### DETAILED DESCRIPTION

The invention is based on the surprising identification of new compounds that are able to activate long isoforms of PDE4 enzymes. The compounds are small molecules and so are expected to be easier and cheaper to make and formulate into pharmaceuticals than large biological molecules such as polypeptides, proteins or antibodies. The compounds can be chemically synthesized, as demonstrated in the Examples.

The Examples demonstrate that a number of compounds of Formula I to IV are able to activate long isoforms of PDE4. The Examples go on to demonstrate that certain tested compounds of the invention do not activate a short form of PDE4, thereby demonstrating selectivity for activation of PDE4 long forms over PDE4 short forms. The Examples further demonstrate that PDE4 long form activators of the present invention reduce cAMP-driven cyst formation in an in vitro model of ADPKD.

Various aspects and embodiments are disclosed herein. It will be recognised that features specified in each embodiment may be combined with other specified features to provide further embodiments.

Described herein are compounds of Formula I to IV, or pharmaceutically acceptable salts or derivatives thereof, as set out above. Formula I to IV are illustrated herein. Compounds of Formula I to IV, or pharmaceutically acceptable salts or derivatives thereof, may be provided for use in the treatment or prevention of a disease or disorder that can be ameliorated by activation of long isoforms of PDE4. Compounds of Formula I to IV, or pharmaceutically acceptable salts or derivatives thereof, may be provided for use in the treatment or prevention of a disease or disorder mediated by excessive intracellular cAMP signalling.

In the compounds of Formula I, R¹ is a 4- to 10-membered monocyclic, bridged or bicyclic ring containing at least 1 ring N heteroatom and optionally a ring O heteroatom, and wherein R¹ is optionally substituted with 1 or more R⁴. The monocyclic, bridged or bicyclic ring may be saturated, partially saturated or aromatic, or in the case of a bicyclic ring, a combination thereof. It will be appreciated that the ring N atom in a saturated or partially saturated ring, when unsubstituted, may be NH (as valency allows). It will also be appreciated that no further ring heteroatoms are present other than the “at least 1 ring N heteroatom” (i.e. 1 or more ring N heteroatoms) and the optional “ring O heteroatom”.

In embodiment (1) of Formula I, R¹ comprises at least 1 ring N heteroatom not at the point of attachment of R¹ (i.e. a ring N atom must be present at a position that is not the point of attachment of R¹ to the ring containing X₁, X₂, Y₁ and Y₂). The remaining moieties may be as defined for Formula I or any of embodiments (5)-(19) of Formula I described herein, mutatis mutandis.

In embodiment (2) of Formula I, R¹ is a 4- to 10-membered monocyclic, bridged or bicyclic ring containing 1 ring N heteroatom, 2 ring N heteroatoms or 1 ring N heteroatom and 1 ring O heteroatom, and wherein R¹ is optionally substituted with 1 or more R⁴. R¹ may comprise at least 1 ring N heteroatom not at the point of attachment of R¹. The remaining moieties may be as defined for Formula I or any of embodiments (5)-(19) of Formula I described herein, mutatis mutandis.

In embodiment (3) of Formula I, R¹ is a 5- to 6-membered saturated, monocyclic ring containing at least 1 ring N heteroatom and optionally a ring O heteroatom (for example, 1 ring N heteroatom, 2 ring N heteroatoms or 1 ring N heteroatom and 1 ring O heteroatom); a 5- to 6-membered aromatic, monocyclic ring containing 1 or 2 ring N heteroatoms; or a 7- to 8-membered saturated, bridged ring containing 1 or 2 ring N heteroatoms; a 9-membered saturated, bridged ring system containing 2 ring N heteroatoms and a ring O-heteroatom; or a 7- to 10-membered saturated, fused or spiro ring system system containing 1 or 2 ring N heteroatoms, optionally 2 ring N heteroatoms; and R¹ is optionally substituted with 1 or more R⁴, optionally wherein R¹ is optionally substituted with 1, 2 or 3 R⁴. R¹ may comprise at least 1 ring N heteroatom not at the point of attachment of R¹. The remaining moieties may be as defined for Formula I or any of embodiments (5)-(19) of Formula I described herein, mutatis mutandis.

In Formula I or any of the options for embodiments (1), (2) or (3), R¹ may be a 4- to 10-membered monocyclic, bridged or bicyclic ring containing at least 1 ring N heteroatom (i.e. with no ring O heteroatom). R¹ may be a 6-membered saturated or aromatic monocyclic ring containing 1 or 2 ring N heteroatoms, or a 7- to 8-membered saturated, bridged ring system containing 1 or 2 ring N heteroatoms, and R¹ is optionally substituted with 1 or more R⁴. R¹ may be a 6-membered saturated or aromatic monocyclic ring containing 1 or 2 ring N heteroatoms, or a 7- to 8-membered saturated, bridged ring system containing 1 or 2 ring N heteroatoms, wherein at least 1 ring N heteroatom is not at the point of attachment of R¹, and R¹ is optionally substituted with 1 or more R⁴. R¹ may be a 6-membered saturated or aromatic monocyclic ring containing 1 or 2 ring N heteroatoms, or a 7- to 8-membered saturated, bridged ring system containing 1 or 2 ring N heteroatoms, wherein at least 1 ring N heteroatom is not at the point of attachment of R¹, and wherein R¹ is optionally substituted with 1 R⁴. R¹ may be a 6-membered saturated or aromatic monocyclic ring containing 2 ring N heteroatoms; or a 7- to 8-membered saturated, bridged ring system containing 2 ring N heteroatoms, wherein R¹ is optionally substituted with 1 R⁴. R¹ may be a 7- to 8-membered saturated, bridged ring system containing 2 ring N heteroatoms, wherein R¹ is optionally substituted with 1 R⁴. R¹ may be a 7- to 8-membered saturated, bridged ring system containing 2 ring N heteroatoms, for example a bridged piperazine, such as 3,8-diazabicyclo[3.2.1]octanyl, wherein R¹ is optionally substituted with 1 R⁴. The remaining moieties may be as defined for Formula I or any of embodiments (5)-(19) of Formula I described herein, mutatis mutandis.

In Formula I or any of the options for embodiments (1), (2) or (3), R¹ may be piperidinyl, piperazinyl, morpholinyl, pyrrolidinyl, pyrazolyl, imidazolyl, pyridinyl, azetidinyl, 2,5-diazabicyclo[2.2.1]heptanyl, 2,5-diazabicyclo[2.2.2]octanyl or 3,8-diazabicyclo[3.2.1]octanyl, 3,6-diazabicyclo[3.1.1]heptanyl, 4,7-diazaspiro[2.5]octanyl, 2,6-diazaspiro[3.3]heptanyl, 2,6-diazaspiro[3.4]octanyl, 2,7-diazaspiro[3.5]nonanyl, octahydro-4H-pyrrolo[3,2-b]pyridinyl, octahydro-5H-pyrrolo[3,2-c]pyridinyl or hexahydropyrrolo[3,4-c]pyrrol-(1)-yl, each of which is optionally substituted with 1 or more R⁴, preferably optionally substituted with 1-3 R⁴, preferably optionally substituted with 1 R⁴. R¹ may be a group of structure:

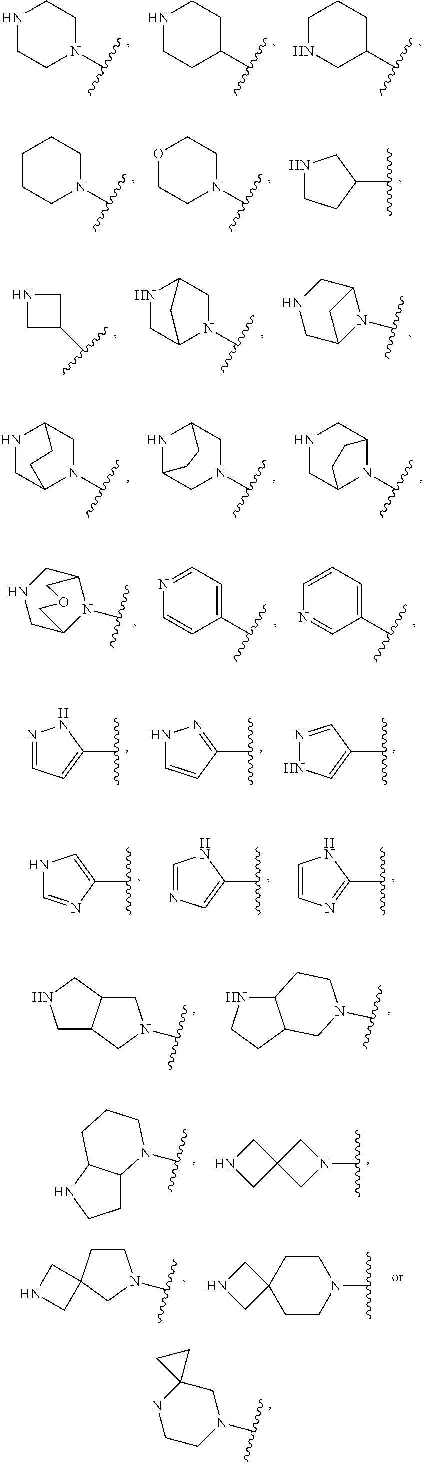

and wherein R¹ is optionally substituted with 1 or more R⁴, optionally wherein R¹ is optionally substituted with 1-3 R⁴. R¹ may be piperidinyl, piperazinyl, pyrrolidinyl, pyrazolyl, imidazolyl, pyridinyl, 2,5-diazabicyclo[2.2.1]heptanyl, 2,5-diazabicyclo[2.2.2]octanyl or 3,8-diazabicyclo[3.2.1]octanyl, each of which is optionally substituted with 1 or more R⁴, preferably optionally substituted with 1-3 R⁴, preferably optionally substituted with 1 R⁴. R¹ may be piperidinyl, piperazinyl or pyridinyl, each of which is optionally substituted with 1 or more R⁴, preferably optionally substituted with 1-3 R⁴, preferably optionally substituted with 1 R⁴. R¹ may be: a group of structure

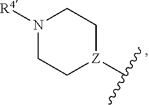

wherein Z is CH or N and R^{4′} is H or R⁴; or pyridyl (optionally 3-pyridyl) optionally substituted with 1 R⁴. R¹ may be a 7- to 8-membered saturated, bridged ring system containing 2 ring N heteroatoms, for example a bridged piperazine such as

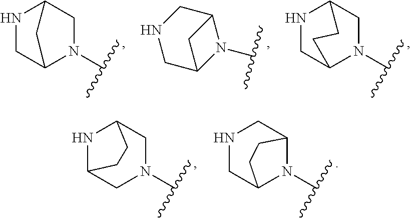 [SMILES: *N1CC2CC1CN2 ; *N1C2CNCC1C2 ; *N1CC2CCC1CN2 ; *N1CC2CCC(C1)N2 ; *N1C2CCC1CNC2]

The remaining moieties may be as defined for Formula I or any of embodiments (5)-(19) of Formula I described herein, mutatis mutandis.

In embodiment (4) of Formula I, R¹ is a 5- to 6-membered saturated, monocyclic ring containing at least 1 ring N heteroatom and optionally a ring O heteroatom; or a 7- to 8-membered saturated, bridged ring containing 1 or 2 ring N heteroatoms, and wherein R¹ is optionally substituted with 1, 2 or 3 R⁴. In any of the options for embodiment (4), R¹ may comprise at least 1 ring N heteroatom not at the point of attachment of R¹. R¹ may be a 6-membered saturated monocyclic ring containing 1 or 2 ring N heteroatoms, or a 7- to 8-membered saturated, bridged ring system containing 1 or 2 ring N heteroatoms. R¹ may be a 6-membered saturated monocyclic ring containing 1 or 2 ring N heteroatoms, or a 7- to 8-membered saturated, bridged ring system containing 1 or 2 ring N heteroatoms, wherein R¹ is optionally substituted with 1 R⁴. The remaining moieties may be as defined for Formula I or any of embodiments (5)-(19) of Formula I described herein, mutatis mutandis.

In Formula I or any of the options for embodiments (1)-(4), R¹ may be substituted with 1 or more R⁴. Where R¹ contains a substitutable ring N atom, R¹ may be substituted on a substitutable ring N atom. In embodiments where R¹ is saturated ring, R¹ may be substituted by 1 R⁴, preferably on a ring N atom. In embodiments where R¹ is an aromatic ring, R¹ may be substituted by 1, 2 or 3 R⁴. In embodiments where R¹ is a 6-membered ring, R¹ may be substituted by 1 R⁴. In embodiments where R¹ is a 5-membered ring, R¹ may be substituted by 1,2 or3 R⁴.

In the compounds of Formula I, each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy. Each R⁴ may, independently, represent a substituent on a carbon atom or a substitutable N atom.

In embodiment (5) of Formula I, each R⁴ is independently halogen, OH, CN, (C1-4)alkyl, (C1-3)alkoxy, (C3-6)cycloalkyl or —(C1-3)alkylene-(C1-3)alkoxy, the (C1-3)alkyl, (C1-3)alkoxy, (C3-6)cycloalkyl and —(C1-3)alkylene-(C1-3)alkoxy being optionally substituted with 1 or more substituents independently selected from halogen, OH and (C1-3)alkoxy. Each R⁴ may independently be F, Cl, OH, CN, (C1-4)alkyl, methoxy, ethoxy, cyclopropyl or —(CH₂)₂—O—(CH₂)₂O—CH₃, the (C1-4)alkyl being optionally substituted with 1 or more substituents independently selected from halogen and OH. The remaining moieties may be as defined for Formula I or any of embodiments (1)-(4) or (8)-(19) of Formula I described herein, mutatis mutandis.

In embodiment (6) of Formula I, each R⁴ is independently halogen, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy. Each R⁴ may independently be halogen, OH, (C1-4)alkyl, (C1-3)alkoxy, (C3-6)cycloalkyl or —(C1-3)alkylene-(C1-3)alkoxy, the (C1-3)alkyl, (C1-3)alkoxy, (C3-6)cycloalkyl and —(C1-3)alkylene-(C1-3)alkoxy being optionally substituted with 1 or more substituents independently selected from halogen, OH and (C1-3)alkoxy. Each R⁴ may independently be F, Cl, OH, (C1-4)alkyl, methoxy, ethoxy, cyclopropyl or —(CH₂)₂—O—(CH₂)₂—O—CH₃, the (C1-4)alkyl being optionally substituted with 1 or more substituents independently selected from halogen and OH. The remaining moieties may be as defined for Formula I or any of embodiments (1)-(4) or (8)-(19) of Formula I described herein, mutatis mutandis.

In embodiment (7) of Formula I, each R⁴ is independently halogen, CN, OH, (C1-2)alkyl, (C1-6)alkoxy, or —(C1-6)alkylene-(C1-6)alkoxy, the (C1-2)alkyl, (C1-6)alkoxy and —(C1-6)alkylene-(C1-6)alkoxy being optionally substituted with 1 or more substituents independently selected from halogen, OH and (C1-6)alkoxy. Each R⁴ may independently be F, Cl, OH, (C1-2)alkyl, methoxy, ethoxy or —(CH₂)₂—O—(CH₂)₂O—CH₃, the (C1-2)alkyl being optionally substituted with 1 or more substituents independently selected from halogen and OH. The remaining moieties may be as defined for Formula I or any of embodiments (1)-(4) or (8)-(19) of Formula I described herein, mutatis mutandis.

In Formula I or any of the options for embodiments (5)-(7), when one of X₁ and X₂ is S and the other is N or CR^{3a}, Y₁ and Y₂ are each C, and one of Z₁, Z₂ and Z₃ is N and the others are each CR^{3b}, and R¹ is a 6-membered saturated monocyclic ring containing 1 or 2 ring N heteroatoms substituted with 1 R⁴, R⁴ is halogen, CN, OH, (C1-6)alkyl that is straight-chain or branched, (C1-6)alkoxy or —(C1-6)alkylene-(C1-6)alkoxy, the (C1-6)alkyl, (C1-6)alkoxy and —(C1-6)alkylene-(C1-6)alkoxy being optionally substituted with 1 or more substituents independently selected from halogen, OH and (C1-6)alkoxy.

In Formula I or any of the options for embodiments (5)-(7), when attached to a ring N atom, R⁴ may independently be any of the options identified herein for R⁴, except for halogen, CN, OH, and —(C1-6)alkoxy.

In the compounds of Formula I, R² is (C5-7)cycloalkyl, fused to a 6-membered aromatic or heteroaromatic ring that contains 0, 1 or 2 ring N atoms; a 5- to 7-membered non-aromatic heterocycle containing one ring 0 heteroatom, optionally fused to a 6-membered aromatic or heteroaromatic ring that contains 0, 1 or 2 ring N atoms; CH₂Ar, where Ar is a 6-membered aromatic or heteroaromatic ring that contains 0, 1 or 2 ring N atoms; or (C3-8)alkyl group that may be straight chain, branched or cyclic, or a combination thereof; wherein R² is optionally substituted with 1 or more R⁵.

In the compounds of Formula I, each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH.

In embodiment (8) of Formula I, each R⁵ is independently halogen, OH, CN, (C1-4)alkyl, or (C1-4)alkoxy, the (C1-4)alkyl and (C1-4)alkoxy group being optionally substituted with 1 or more halogen or OH, preferably optionally substituted with 1 or more fluoro or 1 OH. The remaining moieties may be as defined for Formula I or any of embodiments (1)-(7) or (9)-(19) of Formula I described herein, mutatis mutandis.

In embodiment (9) of Formula I, R² is (C5-7)cycloalkyl, fused to a 6-membered aromatic or heteroaromatic ring that contains 0, 1 or 2 ring N atoms, wherein R² is optionally substituted with 1 or more R⁵. R² may be (C5-7)cycloalkyl, fused to a 6-membered aromatic or heteroaromatic ring that contains 0, 1 or 2 ring N atoms, wherein the (C5-7)cycloalkyl is optionally substituted with 1 to 3 substituents independently selected from OH, halogen, (C1-4)alkyl and (C1-4)alkoxy, the (C1-4)alkyl and (C1-4)alkoxy groups being optionally substituted with one or more fluoro, and the 6-membered aromatic or heteroaromatic ring is optionally substituted with 1 to 3 substituents independently selected from (C1-4)alkyl, (C1-4)alkoxy, CN and halogen, the (C1-4)alkyl and (C1-4)alkoxy groups being optionally substituted with one or more fluoro. R² may be indane optionally substituted with 1 to 3 R⁵, preferably 1 R⁵. The remaining moieties may be as defined for Formula I or any of embodiments (1)-(8) or (15)-(19) of Formula I described herein, mutatis mutandis. In any of the options for embodiment (9), R² may be optionally substituted with 1 instance of halogen, OH, CN, (C1-4)alkyl or (C1-4)alkoxy.

In embodiment (10) of Formula I, R² is a 5- to 7-membered non-aromatic heterocycle containing one ring O heteroatom, optionally fused to a 6-membered aromatic or heteroaromatic ring that contains 0, 1 or 2 ring N atoms, wherein R² is optionally substituted with 1 or more R⁵. R² may be a 5- to 7-membered non-aromatic heterocycle containing one ring O heteroatom, optionally fused to a 6-membered aromatic or heteroaromatic ring that contains 0, 1 or 2 ring N atoms, wherein the 5- to 7-membered non-aromatic heterocycle is optionally substituted with 1 to 3 substituents on one or more ring carbon atoms independently selected from OH, halogen, (C1-4)alkyl and (C1-4)alkoxy, the (C1-4)alkyl and (C1-4)alkoxy groups being optionally substituted with one or more fluoro, and the 6-membered aromatic or heteroaromatic ring is optionally substituted with 1 to 3 substituents independently selected from (C1-4)alkyl, (C1-4)alkoxy, CN and halogen, the (C1-4)alkyl and (C1-4)alkoxy groups being optionally substituted with one or more fluoro. R² may be chromane or tetrahydropyran optionally substituted with 1 to 3 R⁵, preferably 1 R⁵. The remaining moieties may be as defined for Formula I or any of embodiments (1)-(8) or (15)-(19) of Formula I described herein, mutatis mutandis. In any of the options for embodiment (10), R² may be optionally substituted with 1 instance of halogen, OH, CN, (C1-4)alkyl or (C1-4)alkoxy. The remaining moieties may be as defined for any aspect or embodiment of Formula I described herein, mutatis mutandis.

In embodiment (11) of Formula I, R² is CH₂Ar, where Ar is a 6-membered aromatic or heteroaromatic ring that contains 0, 1 or 2 ring N atoms, wherein R² is optionally substituted with 1 or more R⁵. It will be appreciated that substitution by R⁵ is possible on the —CH₂— linker or Ar moiety of R². R² may be CH₂Ar, wherein the Ar is optionally substituted with 1 to 3 substituents selected from halogen, CN, (C1-4)alkyl, (C1-4)alkoxy and the CH₂ is optionally substituted with (C1-4)alkyl or —(C1-6)alkylene-(C1-6)alkoxy, the (C1-4)alkyl group being optionally substituted with OH. R² may be benzyl optionally substituted with 1 to 3 R⁵, preferably 1 R⁵. The remaining moieties may be as defined for Formula I or any of embodiments (1)-(8) or (15)-(19) of Formula I described herein, mutatis mutandis. In any of the options for embodiment (11), R² may be optionally substituted with 1 instance of halogen, OH, CN, (C1-4)alkyl or (C1-4)alkoxy, the (C1-4)alkyl group being optionally substituted with OH.

In embodiment (12) of Formula I, R² is a (C3-8)alkyl group that may be straight chain, branched or cyclic, or a combination thereof, wherein R² is optionally substituted with 1 or more R⁵. R² may be a (C4-8)alkyl group that may be straight chain, branched or cyclic, or a combination thereof, wherein R² is optionally substituted with 1 or more R⁵. R² may be an optionally substituted (C3-6)alkyl group that may be branched or cyclic. R² may be an optionally substituted (C4-6)alkyl group that may be branched or cyclic, preferably an optionally substituted (C5-6)cycloalkyl group. R² may be an optionally substituted (C4-6)cycloalkyl group, preferably an optionally substituted (C5-6)cycloalkyl group. R² may be cyclohexyl, cyclopentyl, cyclobutyl or isopropyl optionally substituted with 1 to 3 R⁵, preferably 1 R⁵. R² may be cyclohexyl, cyclopentyl or cyclobutyl optionally substituted with 1 to 3 R⁵. R² may be (C5-6)cycloalkyl substituted with 2 or more R⁵. The remaining moieties may be as defined for Formula I or any of embodiments (1)-(8) or (15)-(19) of Formula I described herein, mutatis mutandis. In any of the options for embodiment (12), R² may be optionally substituted with 1 or more halogen, (C1-4)alkoxy or OH. R² may be optionally substituted with 1 or 2 instances of halogen or OH. R² may be optionally substituted with 1 OH. R² may be optionally substituted with 2 or 3 instances of fluoro, preferably 2 instances of fluoro on the same carbon atom. In any of the options for embodiment (12), R² may be substituted by 2 or 3 substituents on one or more ring carbon atoms independently selected from OH, halogen, (C1-4)alkyl, (C1-4)alkoxy, the (C1-4)alkyl and (C1-4)alkoxy groups being optionally substituted with one or more fluoro. R² may be a (C5-6)cycloalkyl group substituted by 2 halogen substituents (optionally on a single ring carbon atom).

In embodiment (13) of Formula I, R² is as defined in embodiment (9), embodiment (10) or embodiment (12) of Formula 1. R² may be (C5-6)cycloalkyl fused to a phenyl ring; a 5- to 6-membered non-aromatic heterocycle containing one ring O heteroatom, optionally fused to a phenyl ring; or (C5-6)cycloalkyl; wherein R² is optionally substituted with 1 or more R⁵. R² may be a group of formula

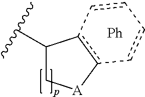

wherein A is O or CH₂; p is 1 or 2; Ph is an optionally present, fused phenyl ring, and wherein R² is optionally substituted with 1 or more R^{S}(for example, 1 or 2 R⁵); optionally wherein when A is O, p is 2 or when A is CH₂, p is 1 or 2. A may be O or C(R⁵)₂ (i.e CH₂, with two R⁵ substituents, for example, CF₂). Ph may be absent. The remaining moieties may be as defined for Formula I or any of embodiments (1)-(8) or (15)-(19) of Formula I described herein, mutatis mutandis.

In embodiment (14) of Formula I, R² is as defined in embodiment (9), embodiment (10) or embodiment (11) of Formula 1. The remaining moieties may be as defined for Formula I or any of embodiments (1)-(8) or (15)-(19) of Formula I described herein, mutatis mutandis.

In Formula I or any of the options for embodiments (9)-(14), R² may be substituted with 1 or more R⁵, preferably 1, 2 or 3 R⁵. R² may be substituted with 1 R⁵. R² may be substituted with 2 R⁵.

In the compounds of Formula I, each R^{3a} is independently H or (C1-6)alkyl, the (C1-6)alkyl being optionally substituted by 1 or more halogen, each R^{3b} is independently H, (C1-6)alkyl, (C1-6)alkoxy, CN or halogen, the (C1-6)alkyl and (C1-6)alkoxy being optionally substituted by 1 or more halogen, and each R^{3c} is independently H or (C1-6)alkyl, the (C1-6)alkyl being optionally substituted by 1 or more halogen.

In embodiment (15) of Formula I, each R^{3a} is independently H or (C1-3)alkyl, the (C1-3)alkyl being optionally substituted by 1 or more halogen, each R^{3b} is independently H, (C1-3)alkyl, (C1-3)alkoxy, CN or halogen, the (C1-3)alkyl and (C1-3)alkoxy being optionally substituted by 1 or more halogen, and/or each R^{3c} is independently H or (C1-3)alkyl, the (C1-3)alkyl being optionally substituted by 1 or more halogen. Each R^{3a} may independently be —H or CH₃. Each R^{3b} may independently be —H, —CH₃, —OCH₃, halo, CN or cyclopropyl. Each R^{3c} may independently be —H or CH₃. The remaining moieties may be as defined for Formula I or any of embodiments (1)-(14) or (18)-(19) of Formula I described herein, mutatis mutandis.

In embodiment (16) of Formula I, each of R^{3a}, R^{3b} and R^{3c} where present are H or methyl. In some embodiments, 0 or 1 of R^{3a}, R^{3b} and R^{3c} where present are methyl and the others where present are H. Preferably, each of R^{3a}, R^{3b} and R^{3c} where present are H. The remaining moieties may be as defined for Formula I or any of embodiments (1)-(14) or (18)-(19) of Formula I described herein, mutatis mutandis.

In embodiment (17) of Formula I, 1 or 2 of R^{3a}, R^{3b} and R^{3c} is a group other than H as defined for Formula I or embodiment (15) of Formula I described herein, and the others where present are each H. Preferably, 1 of R^{3a}, R^{3b} and R^{3c} is a group other than H as defined for Formula I or embodiment (15) of Formula I described herein, and the others where present are each H. The compound may be a compound of formula:

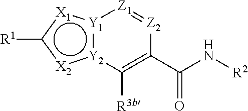

or a pharmaceutically acceptable salt or derivative thereof, wherein R^{3b′} is (C1-6)alkyl, (C1-6)alkoxy, CN or halogen, the (C1-6)alkyl and (C1-6)alkoxy being optionally substituted by 1 or more halogen; wherein R^{3a}, R^{3b} and R^{3c}, where present, are all H. The remaining moieties may be as defined for Formula I or any of embodiments (1)-(14) or (18)-(19) of Formula I described herein, mutatis mutandis.

In the compounds of Formula I, one of X₁ and X₂ is N and the other is N or CR^{3a}, one of Y₁ and Y₂ is N and the other is C, and one of Z₁, Z₂ and Z₃ is N or CR^{3b} and the others are each CR^{3b}; or one of X₁ and X₂ is N and the other is NR^{3c} or O, Y₁ and Y₂ are each C, and one of Z₁, Z₂ and Z₃ is N or CR^{3b} and the others are each CR^{3b}; or one of X₁ and X₂ is S and the other is N or CR^{3a}, Y₁ and Y₂ are each C, and one of Z₁, Z₂ and Z₃ is N and the others are each CR^{3b}.

In embodiment (18) of Formula I, the bicyclic heteroaromatic ring system containing X₁, X₂, Y₁, Y₂, Z₁, Z₂ and Z₃ is selected from:

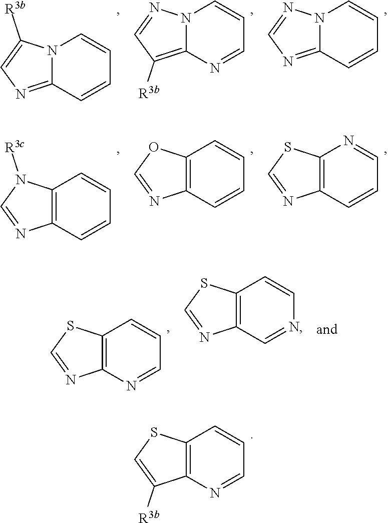

The bicyclic heteroaromatic ring system containing X₁, X₂, Y₁, Y₂, Z₁, Z₂ and Z₃ may be selected from:

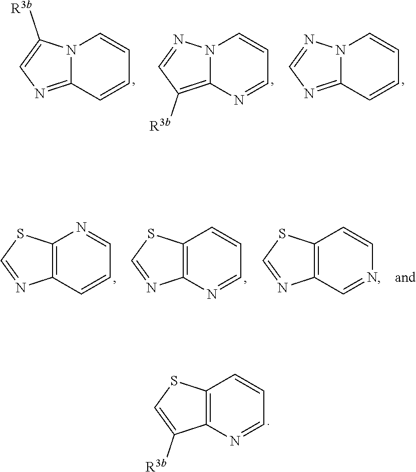

The remaining moieties may be as defined for Formula I or any of embodiments (1)-(17) of Formula I described herein, mutatis mutandis.

In embodiment (19) of Formula I, the compound is selected from the formula:

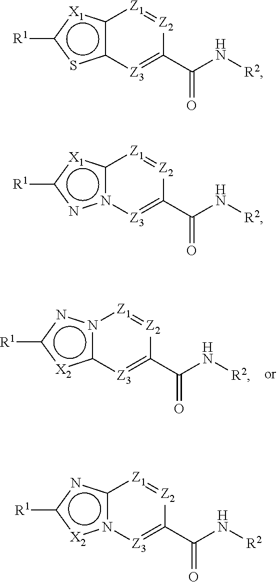

The remaining moieties may be as defined for Formula I or any of embodiments (1)-(17) of Formula I described herein, mutatis mutandis.

In Formula I or any of the options for embodiment (18) or (19) of Formula I, one of X₁ and X₂ may be N and the other may be N or CR^{3a}, one of Y₁ and Y₂ may be N and the other may be C, and one of Z₁, Z₂ and Z₃ may be N or CR^{3b} and the others may each be CR^{3b}.

In Formula I or any of the options for embodiment (18) or (19) of Formula I, one of X₁ and X₂ may be S and the other may be N or CR^{3a}, Y₁ and Y₂ may each be C, and one of Z₁, Z₂ and Z₃ may be N and the others may each be CR^{3b}.

In embodiment (20) of Formula I,
R¹ is a 6-membered saturated monocyclic ring containing 1 or 2 (optionally 2) ring N heteroatoms, or a 7- to 8-membered saturated, bridged ring system containing 1 or 2 (optionally 2) ring N heteroatoms, wherein at least 1 ring N heteroatom is not at the point of attachment of R¹, and wherein R¹ is optionally substituted with 1 R⁴;
R² is
(i) (C5-6)cycloalkyl, optionally fused to a phenyl ring; or
(ii) a 5- to 6-membered non-aromatic heterocycle containing one O heteroatom, optionally fused to a phenyl ring;
and wherein R² is optionally substituted with 1 or more (optionally 1 or 2) R⁵;
R^{3a}, R^{3b} and R^{3c}, where present, are each independently H or methyl (optionally wherein 0 or 1 of R^{3a}, R^{3b} and R^{3c} is methyl and the others are H);
R⁴, where present, is (C1-6)alkyl optionally substituted with OH, optionally (C1-2)alkyl optionally substituted with OH;
R⁵, where present, is OH or halo.

In embodiment (20) of Formula I, R² may be a group of formula

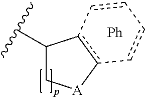

wherein A is O or CH₂; p is 1 or 2 or 3; Ph is an optionally present, fused phenyl ring, and wherein R² is optionally substituted with 1 or 2 R⁵; optionally wherein when A is O, p is 2 or when A is CH₂, p is 1 or 2. A may be O or C(R⁵)₂ (i.e CH₂, with two R⁵ substituents, for example, CF₂). Ph may be absent.

Embodiments (1) to (7) and (15) to (19) of Formula I may apply to any of the options for embodiment (20) of Formula I, mutatis mutandis.

In embodiment (21) of Formula I, when one of X₁ and X₂ is N and the other is O, Y₁ and Y₂ are each C, and each of Z₁, Z₂ and Z₃ are CR^{3b}, each R^{3b} is H, and R¹ is 4-cyclopentylpiperazin-1-yl, 4-cyclopropylpiperazin-1-yl or 4-isopropylpiperazin-1-yl, R² is not unsubstituted, straight chain or branched (C3-6)alkyl or unsubstituted (C3-8)cycloalkyl. The remaining moieties may be as defined for Formula I or any of embodiments (1)-(19) of Formula I described herein, mutatis mutandis.

Compounds of Formula I include compounds of Formulas II-IV. Embodiments (1)-(21) of Formula I may apply mutatis mutandis to each of Formulas II-IV.

Described herein is a compound of Formula II

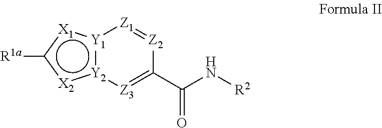

or a pharmaceutically acceptable salt or derivative thereof, wherein:
R^{1a} is a 4- to 10-membered non-aromatic ring that may be monocyclic, bridged or bicyclic containing at least 1 ring N heteroatom and optionally a ring O heteroatom, wherein at least 1 ring N heteroatom is not at the point of attachment of R^{1a}, and wherein R^{1a} is optionally substituted with 1 or more R⁴,
and X₁, X₂, Y₁, Y₂, Z₁, Z₂, Z₃, R², R^{3a}, R^{3b}, R^{3c}, R⁴ and R⁵ are as defined for Formula I or any of embodiments (5)-(19) of Formula I above.

In a compound of Formula II, R^{1a} comprises at least one ring N heteroatom not at the point of attachment to R^{1a}, i.e. a ring N atom must be present at a position that is not the point of attachment of R^{1a} to the ring containing X₁, X₂, Y₁ and Y₂.

In embodiment (1) of Formula II, when one of X₁ and X₂ is N and the other is O, Y₁ and Y₂ are each C, and each of Z₁, Z₂ and Z₃ are CR^{3b}, each R^{3b} is H, and R^{1a} is 4-cyclopentylpiperazin-1-yl, 4-cyclopropylpiperazin-1-yl or 4-isopropylpiperazin-1-yl, R² is not unsubstituted, straight chain or branched (C3-6)alkyl or unsubstituted (C3-8)cycloalkyl.

In embodiment (2) of Formula II:
(i) each R⁴ is independently halogen, CN, OH, (C1-2)alkyl, (C1-6)alkoxy or —(C1-6)alkylene-(C1-6)alkoxy, the (C1-2)alkyl, (C1-6)alkoxy and —(C1-6)alkylene-(C1-6)alkoxy being optionally substituted with 1 or more substituents independently selected from halogen, OH and (C1-6)alkoxy;
(ii) R² is (C5-7)cycloalkyl, fused to a 6-membered aromatic or heteroaromatic ring that contains 0, 1 or 2 ring N atoms; a 5- to 7-membered non-aromatic heterocycle containing one ring O heteroatom, optionally fused to a 6-membered aromatic or heteroaromatic ring that contains 0, 1 or 2 ring N atoms; or CH₂Ar, where Ar is a 6-membered aromatic or heteroaromatic ring that contains 0, 1 or 2 ring N atoms; and wherein R² is optionally substituted with 1 or more R⁵.

In embodiment (3) of Formula II, R^{1a} is a 4- to 10-membered non-aromatic, monocyclic, bridged or bicyclic ring containing 1 ring N heteroatom, 2 ring N heteroatoms or 1 ring N heteroatom and 1 ring O heteroatom, and wherein R¹ is optionally substituted with 1 or more R⁴. R^{1a} may be a 5- to 6-membered saturated, monocyclic ring containing at least 1 ring N heteroatom and optionally a ring O heteroatom (for example, 1 ring N heteroatom, 2 ring N heteroatoms or 1 ring N heteroatom and 1 ring O heteroatom); a 7- to 8-membered saturated, bridged ring system containing 1 or 2 ring N heteroatoms; or a 9-membered saturated, bridged ring system containing 2 ring N heteroatoms and a ring O-heteroatom; or a 7- to 10-membered saturated, fused or spiro ring system containing 1 or 2 ring N heteroatoms, and wherein R^{1a} is optionally substituted with 1 or more R⁴, optionally 1, 2 or 3 R⁴.

In embodiment (4) of Formula II, R^{1a} is a 5- to 6-membered saturated, monocyclic ring containing at least 1 ring N heteroatom and optionally a ring O heteroatom (for example, 1 ring N heteroatom, 2 ring N heteroatoms or 1 ring N heteroatom and 1 ring O heteroatom); or a 7- to 8-membered saturated, bridged ring containing 1 or 2 ring N heteroatoms, and wherein R^{1a} is optionally substituted with 1 or more R⁴, optionally 1, 2 or 3 R⁴. R^{1a} may be a 6-membered saturated monocyclic ring containing 1 or 2 ring N heteroatoms, optionally wherein at least 1 ring N heteroatom is not at the point of attachment of R^{1a}. R^{1a} may be a 6-membered saturated monocyclic ring containing 1 or 2 ring N heteroatoms, wherein R^{1a} is optionally substituted with 1 R⁴. R^{1a} may be a 7- to 8-membered saturated, bridged ring system containing 1 or 2 ring N heteroatoms, and wherein R^{1a} is optionally substituted with 1 or more R⁴, optionally 1, 2 or 3 R⁴. R^{1a} may be a 7- to 8-membered saturated, bridged ring system containing 2 ring N heteroatoms, for example a bridged piperazine, such as 3,8-diazabicyclo[3.2.1]octanyl, wherein R^{1a} is optionally substituted with 1 R⁴.

In Formula II or any of the options for embodiments (3) or (4), R^{1a} may be piperidinyl, piperazinyl, morpholinyl, pyrrolidinyl, 2,5-diazabicyclo[2.2.1]heptanyl, 2,5-diazabicyclo[2.2.2]octanyl or 3,8-diazabicyclo[3.2.1]octanyl, each of which is optionally substituted with 1 or more R⁴, preferably optionally substituted with 1-3 R⁴, preferably optionally substituted with 1 R⁴. R^{1a} may be group of structure:

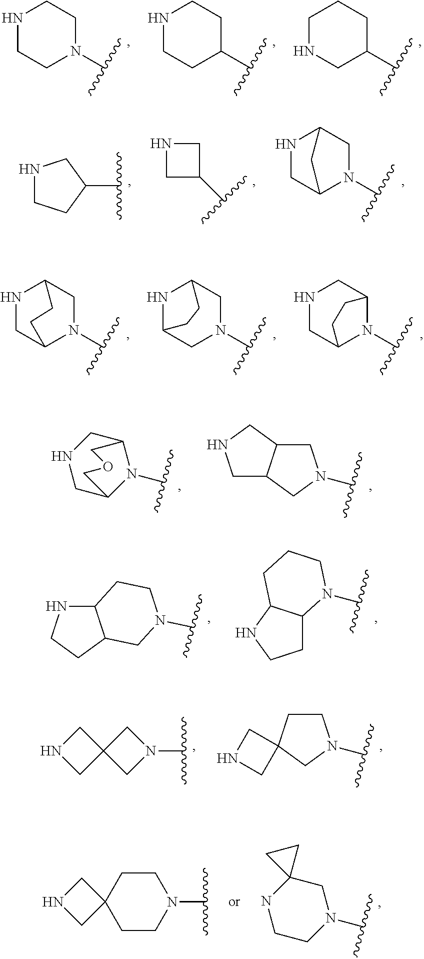

and wherein R^{1a} is optionally substituted with 1 or more R⁴, optionally wherein R^{1a} is optionally substituted with 1-3 R⁴, preferably optionally substituted with 1 R⁴. R^{1a} may be piperidinyl, piperazinyl, pyrrolidinyl, 2,5-diazabicyclo[2.2.1]heptanyl, 2,5-diazabicyclo[2.2.2]octanyl or 3,8-diazabicyclo[3.2.1]octanyl, each of which is optionally substituted with 1 or more R⁴, preferably optionally substituted with 1-3 R⁴, preferably optionally substituted with 1 R⁴. R^{1a} may be piperidinyl or piperazinyl, each of which is optionally substituted with 1 or more R⁴, preferably optionally substituted with 1-3 R⁴, preferably optionally substituted with 1 R⁴. R^{1a} may be a group of structure

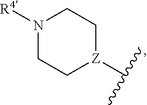

wherein Z is CH or N and R^{4′} is H or R⁴. R^{1a} may be a 7- to 8-membered saturated, bridged ring system containing 2 ring N heteroatoms, for example a bridged piperazine such as

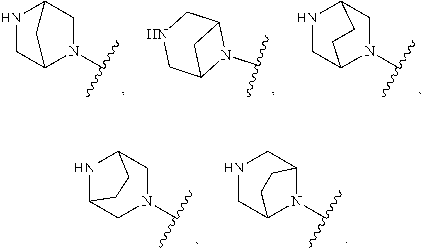 [SMILES: *N1CC2CC1CN2 ; *N1C2CNCC1C2 ; *N1CC2CCC1CN2 ; *N1CC2CCC(C1)N2 ; *N1C2CCC1CNC2]

In Formula II or any of the options for embodiment (3) or (4) of Formula II, R^{1a} may be optionally substituted with 1 or more R⁴. Where R^{1a} contains a substitutable ring N atom R^{1a} may preferably be substituted on a substitutable ring N atom. R^{1a} may be substituted by 1 R⁴, preferably on a ring N atom.

In Formula II or any of the options for embodiments (3) or (4) of Formula II, R^{1a} may be a 4- to 10-membered non-aromatic, monocyclic, bridged or bicyclic ring containing at least 1 ring N heteroatom (i.e. with no ring O heteroatom). R^{1a} may be a 6-membered saturated monocyclic ring containing 1 or 2 ring N heteroatoms, or a 7- to 8-membered saturated, bridged ring system containing 1 or 2 ring N heteroatoms, and R^{1a} is optionally substituted with 1 or more R⁴.

In embodiment (5) of Formula II, 1 or 2 of R^{3a}, R^{3b} and R^{3c} is a group other than H as defined for Formula I or embodiment (15) of Formula I described herein, and the others where present are each H. Preferably, 1 of R^{3a}, R^{3b} and R^{3c} is a group other than H as defined for Formula I or embodiment (15) of Formula I described herein, and the others where present are each H. The compound may be a compound of formula:

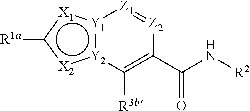

or a pharmaceutically acceptable salt or derivative thereof, wherein R^{3b′} is (C1-6)alkyl, (C1-6)alkoxy, CN or halogen, the (C1-6)alkyl and (C1-6)alkoxy being optionally substituted by 1 or more halogen; wherein R^{3a}, R^{3b} and R^{3c}, where present, are all H. R^{1a} may be as defined for Formula II or embodiment (3) or embodiment (4) of Formula II.

Embodiments (1) and (2) of Formula II may apply to any of embodiments (3)-(5) of Formula II, mutatis mutandis.

In embodiment (6) of Formula II,
R^{1a} is a 6-membered saturated monocyclic ring containing 1 or 2 (optionally 2) ring N heteroatoms or a 7- to 8-membered saturated, bridged ring system containing 1 or 2 (optionally 2) ring N heteroatoms, wherein at least 1 ring N heteroatom is not at the point of attachment of R^{1a}, and wherein R^{1a} is optionally substituted with 1 R⁴;
R² is
(i) (C5-6)cycloalkyl, optionally fused to a phenyl ring; or
(ii) a 5- to 6-membered non-aromatic heterocycle containing one O heteroatom, optionally fused to a phenyl ring;
and wherein R² is optionally substituted with 1 or 2 R⁵;
R^{3a}, R^{3b} and R^{3c}, where present, are each independently H or methyl (optionally wherein 0 or 1 of R^{3a}, R^{3b} and R^{3c} is methyl and the others are H); R⁴, where present, is (C1-6)alkyl optionally substituted with OH, optionally (C1-2)alkyl optionally substituted with OH; R⁵, where present, is OH or halo.

In embodiment (6) of Formula II, R² may be a group of formula

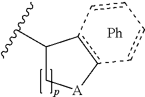

wherein A is O or CH₂; p is 1 or 2; Ph is an optionally present, fused phenyl ring, and wherein R² is optionally substituted with 1 or 2 R⁵; optionally wherein when A is O, p is 2 or when A is CH₂, p is 1 or 2. A may be O or C(R⁵)₂ (i.e CH₂, with two R₅ substituents, for example, CF₂). Ph may be absent.

Embodiment (1) of Formula II may apply to embodiment (6) of Formula II, mutatis mutandis.

In Formula II or any of the embodiments of Formula II, one of X₁ and X₂ may be N and the other may be N or CR^{3a}, one of Y₁ and Y₂ may be N and the other may be C, and one of Z₁, Z₂ and Z₃ may be N or CR^{3b} and the others may each be CR^{3b}; or one of X₁ and X₂ may be S and the other may be N or CR^{3a}, Y₁ and Y₂ may each be C, and one of Z₁, Z₂ and Z₃ may be N and the others may each be CR^{3b}.

In Formula II or any of the embodiments of Formula II, the compound may selected from the formula:

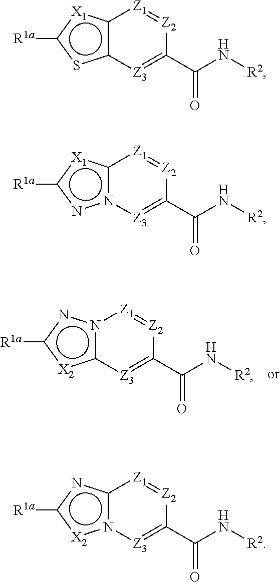

Described herein is a compound of Formula III

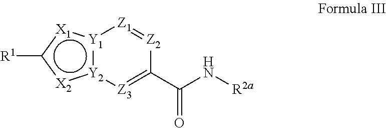

or a pharmaceutically acceptable salt or derivative thereof, wherein:
R^{2a} is
(i) (C5-7)cycloalkyl fused to a 6-membered aromatic or heteroaromatic ring that contains 0, 1 or 2 ring N atoms;
(ii) a 5- to 7-membered non-aromatic heterocycle containing one ring O heteroatom, optionally fused to a 6-membered aromatic or heteroaromatic ring that contains 0, 1 or 2 ring N atoms; or
(iv) a (C5-6)cycloalkyl group
and wherein R^{2a} is optionally substituted with 1 or more R⁵;
and X₁, X₂, Y₁, Y₂, Z₁, Z₂, Z₃, R¹, R^{3a}, R^{3b}, R^{3c}, R⁴ and R⁵ are as defined for Formula I or any of embodiments (1)-(8) or (15)-(19) of Formula I above.

In embodiment (1) of Formula III, R^{2a} is (C5-7)cycloalkyl, fused to a 6-membered aromatic or heteroaromatic ring that contains 0, 1 or 2 ring N atoms, wherein R^{2a} is optionally substituted with 1 or more R⁵. R^{2a} may be a (C5-7)cycloalkyl fused to a 6-membered aromatic or heteroaromatic ring that contains 0, 1 or 2 ring N atoms, wherein the (C5-7)cycloalkyl is optionally substituted with 1 to 3 substituents independently selected from OH, halogen, (C1-4)alkyl and (C1-4)alkoxy, the (C1-4)alkyl and (C1-4)alkoxy groups being optionally substituted with one or more fluoro, and the 6-membered aromatic or heteroaromatic ring is optionally substituted with 1 to 3 substituents independently selected from (C1-4)alkyl, (C1-4)alkoxy, CN and halogen, the (C1-4)alkyl and (C1-4)alkoxy groups being optionally substituted with 1 or more fluoro. R^{2a} may be indane optionally substituted with 1 to 3 R⁵, preferably 1 R⁵. In any ofthe options for embodiment (1), R^{2a} may be optionally substituted with 1 instance of halogen, OH, CN, (C1-4)alkyl or (C1-4)alkoxy.

In embodiment (2) of Formula III, R^{2a} is a 5- to 7-membered non-aromatic heterocycle containing one ring O heteroatom, optionally fused to a 6-membered aromatic or heteroaromatic ring that contains 0, 1 or 2 ring N atoms, wherein R^{2a} is optionally substituted with 1 or more R⁵. R^{2a} may be a 5- to 7-membered non-aromatic heterocycle containing one ring O heteroatom, optionally fused to a 6-membered aromatic or heteroaromatic ring that contains 0, 1 or 2 ring N atoms, wherein the 5- to 7-membered non-aromatic heterocycle is optionally substituted with 1 to 3 substituents on one or more ring carbon atoms independently selected from OH, halogen, (C1-4)alkyl and (C1-4)alkoxy, the (C1-4)alkyl and (C1-4)alkoxy groups being optionally substituted with one or more fluoro, and the 6-membered aromatic or heteroaromatic ring is optionally substituted with 1 to 3 substituents independently selected from (C1-4)alkyl, (C1-4)alkoxy, CN and halogen, the (C1-4)alkyl and (C1-4)alkoxy groups being optionally substituted with one or more fluoro. R^{2a} may be chromane or tetrahydropyran optionally substituted optionally substituted with 1 to 3 R⁵, preferably 1 R⁵. In any of the options for embodiment (2), R^{2a} may be optionally substituted with 1 instance of halogen, OH, CN, (C1-4)alkyl or (C1-4)alkoxy.

According to embodiment (1) or (2) of Formula III, R^{2a} may be (C5-6)cycloalkyl fused to a phenyl ring; or a 5- to 6-membered heterocycle containing one ring O heteroatom, optionally fused to a phenyl ring; wherein R^{2a} is optionally substituted.

In embodiment (3) of Formula III, R^{2a} is a (C5-6)cycloalkyl group substituted by at least 2 R⁵. R^{2a} may be cyclohexyl or cyclopentyl, for example substituted with 2 R⁵. R^{2a} may be optionally substituted with 2 or more halogen, (C1-4)alkoxy or OH. In any of the options for embodiment (3), R^{2a} may be optionally substituted with 2 or more substituents on one or more ring carbon atoms independently selected from OH, halogen, (C1-4)alkyl, (C1-4)alkoxy, the (C1-4)alkyl and (C1-4)alkoxy groups being optionally substituted with one or more fluoro. R^{2a} may be optionally substituted with 2 or 3 instances of halogen or OH. R^{2a} may be optionally substituted with 2 or 3 instances of halogen, preferably 2 instances of halogen, preferably on the same carbon atom. R^{2a} may be a (C5-6)cycloalkyl group substituted by 2 halogen substituents (optionally on a single ring carbon atom).

In embodiment (4) of Formula III,
R¹ is a 6-membered saturated monocyclic ring containing 1 or 2 (optionally 2) ring N heteroatoms or a 7- to 8-membered saturated, bridged ring system containing 1 or 2 (optionally 2) ring N heteroatoms, wherein at least 1 ring N heteroatom is not at the point of attachment of R¹, and wherein R¹ is optionally substituted with 1 R⁴;
R^{2a} is
(i) (C5-6)cycloalkyl fused to a phenyl ring; or
(ii) a 5- to 6-membered non-aromatic heterocycle containing one ring O heteroatom, optionally fused to a phenyl ring; or
(iv) a (C5-6)cycloalkyl group;
and wherein R^{2a} is optionally substituted with 1 or 2 R⁵, wherein when R^{2a} is (iv) a (C5-6)cycloalkyl group it is substituted by 2 R⁵;
R^{3a}, R^{3b} and R^{3c}, where present, are each independently H or methyl (optionally wherein 0 or 1 of R^{3a}, R^{3b} and R^{3c} is methyl and the others are H);
R⁴, where present, is (C1-6)alkyl optionally substituted with OH, optionally (C1-2)alkyl optionally substituted with OH;
R⁵, where present, is OH or halo.

According to any of embodiments (1)-(4) of Formula III, R^{2a} may be a group of formula

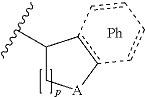

wherein A is O or CH₂; p is 1 or 2; Ph is an optionally present, fused phenyl ring, and wherein R^{2a} is optionally substituted with 1 or 2 R⁵ and wherein when A is CH₂, Ph is present or A is C(R⁵)₂ (i.e CH₂, with two R⁵ substituents, for example, CF₂); optionally wherein when A is O, p is 2 or when A is CH₂, p is 1 or 2. Ph may be absent.

In embodiment (5) of Formula III, 1 or 2 of R^{3a}, R^{3b} and R^{3c} is a group other than H as defined for Formula I or embodiment (15) of Formula I described herein, and the others where present are each H. Preferably, 1 of R^{3a}, R^{3b} and R^{3c} is a group other than H as defined for Formula I or embodiment (15) of Formula I described herein, and the others where present are each H.

The compound may be a compound of formula:

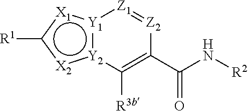

or a pharmaceutically acceptable salt or derivative thereof, wherein R^{3b′} is (C1-6)alkyl, (C1-6)alkoxy, CN or halogen, the (C1-6)alkyl and (C1-6)alkoxy being optionally substituted by 1 or more halogen; wherein R^{3a}, R^{3b} and R^{3c}, where present, are all H. R^{2a} may be as defined in relation to Formula III or any of embodiments (1)-(4) of Formula III.

In embodiment (6) of Formula III, R^{2a} is according to embodiment (2) or embodiment (3) of Formula III.

In embodiment (7) of Formula III, R¹ is according to embodiment (4) of Formula I and R^{2a} is according to embodiment (3) of Formula III. The remaining moieties may be as defined for Formula III or any of embodiments of Formula III described herein, mutatis mutandis. R¹ may be a 7- to 8-membered saturated, bridged ring system containing 1 or 2 ring N heteroatoms, wherein R¹ is optionally substituted with 1 R⁴; and R^{2a} may be according to embodiment (3) of Formula III. R¹ may be a 7- to 8-membered saturated, bridged ring system containing 1 or 2 ring N heteroatoms, wherein R¹ is optionally substituted with 1 R⁴; and R^{2a} may be (C5-6)cycloalkyl substituted with 2 or more R⁵. R¹ may be a 7- to 8-membered saturated, bridged ring system containing 1 or 2 ring N heteroatoms, wherein R¹ is optionally substituted with 1 R⁴; and R^{2a} may be (C5-6)cycloalkyl substituted with 2 or more R⁵; optionally wherein R⁵ may be halogen; and n may be 0 or 1.

In embodiment (8) of Formula III, when one of X₁ and X₂ is S and the other is N, Y₁ and Y₂ are each C, one of Z₁, Z₂ and Z₃ is N and R^{2a} is (iv) a (C5-6)cycloalkyl group, R^{2a} is substituted by at least 2 R⁵. The remaining moieties may be as defined for Formula III or embodiments of Formula III described herein, mutatis mutandis.

In Formula III or any of the embodiments of Formula III, one of X₁ and X₂ may be N and the other may be N or CR^{3a}, one of Y₁ and Y₂ may be N and the other may be C, and one of Z₁, Z₂ and Z₃ may be N or CR^{3b} and the others may each be CR^{3b}; or one of X₁ and X₂ may be S and the other may be N or CR^{3a}, Y₁ and Y₂ may each be C, and one of Z₁, Z₂ and Z₃ may be N and the others may each be CR^{3b}.

In Formula III or any of the embodiments of Formula III, the compound may selected from the formula:

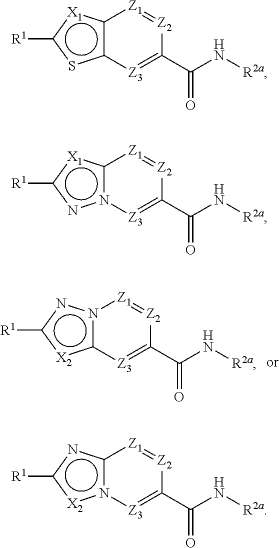

Described herein is a compound of Formula IV

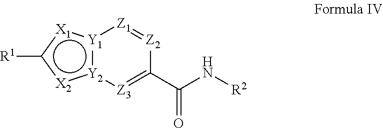

or a pharmaceutically acceptable salt or derivative thereof, wherein
each R^{3a} is independently H or (C1-6)alkyl, the (C1-6)alkyl being optionally substituted by 1 or more halogen;
each R^{3b} is independently H, (C1-6)alkyl, (C1-6)alkoxy, CN or halogen, the (C1-6)alkyl and (C1-6)alkoxy being optionally substituted by 1 or more halogen, wherein at least 1 R^{3b} is other than H;
each R^{3c} is independently H or (C1-6)alkyl, the (C1-6)alkyl being optionally substituted by 1 or more halogen; and
X₁, X₂, Y₁, Y₂, Z₁, Z₂, Z₃, R¹, R², R⁴ and R⁵ are as defined for Formula I or any of embodiments (1)-(15) or (18)-(19) of Formula I above.

In embodiment (1) of Formula IV, 1 or 2 of R^{3a}, R^{3b} and R^{3c} is a group other than H as defined for Formula I or embodiment (15) of Formula I described herein, and the others where present are each H. Preferably, 1 R^{3b} is a group other than H as defined for Formula I or embodiment (15) of Formula I described herein, and the others where present are each H. The compound may be a compound of formula:

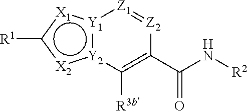

or a pharmaceutically acceptable salt or derivative thereof, wherein R^{3b′} is (C1-6)alkyl, (C1-6)alkoxy, CN or halogen, the (C1-6)alkyl and (C1-6)alkoxy being optionally substituted by 1 or more halogen; wherein R^{3a}, R^{3b} and R^{3c}, where present, are all H.

In Formula IV or any of the embodiments of Formula IV, one of X₁ and X₂ may be N and the other may be N or CR^{3a}, one of Y₁ and Y₂ may be N and the other may be C, and one of Z₁, Z₂ and Z₃ may be N or CR^{3b} and the others may each be CR^{3b}; or one of X₁ and X₂ may be S and the other may be N or CR^{3a}, Y₁ and Y₂ may each be C, and one of Z₁, Z₂ and Z₃ may be N and the others may each be CR^{3b}.

In a further embodiment of a compound of Formula I-IV or a pharmaceutically acceptable salt thereof, including any of the embodiments thereof described above, one or more hydrogen atoms are replaced by ²H. The remaining moieties may be as defined for any aspect or embodiment of Formula I-IV described herein, mutatis mutandis.

In an embodiment, the compound of Formula I is selected from:
(S)—N-(chroman-4-yl)-2-(4-methylpyridin-3-yl)-1H-benzo[d]imidazole-5-carboxamide;
(S)—N-(chroman-4-yl)-2-(6-methylpyridin-3-yl)-1H-benzo[d]imidazole-5-carboxamide;
(S)—N-(chroman-4-yl)-2-(2-methylpyridin-3-yl)-1H-benzo[d]imidazole-5-carboxamide;
(S)—N-(chroman-4-yl)-2-(5-methylpyridin-3-yl)-1H-benzo[d]imidazole-5-carboxamide;
(R)-2-(pyridin-3-yl)-N-(1,2,3,4-tetrahydronaphthalen-1-yl)-1H-benzo[d]imidazole-5-carboxamide;
(S)-2-(pyridin-3-yl)-N-(1,2,3,4-tetrahydronaphthalen-1-yl)-1H-benzo[d]imidazole-5-carboxamide;
(R)—N-(chroman-4-yl)-2-(pyridin-3-yl)-1H-benzo[d]imidazole-5-carboxamide;
(S)—N-(chroman-4-yl)-2-(pyridin-3-yl)-1H-benzo[d]imidazole-5-carboxamide;
(R)—N-(2,3-dihydro-1H-inden-1-yl)-2-(pyridin-3-yl)-1H-benzo[d]imidazole-5-carboxamide;
(S)—N-(2,3-dihydro-1H-inden-1-yl)-2-(pyridin-3-yl)-1H-benzo[d]imidazole-5-carboxamide;
(R)—N-(6-chlorochroman-4-yl)-2-(pyridin-3-yl)-1H-benzo[d]imidazole-5-carboxamide;
(S)—N-(6-chlorochroman-4-yl)-2-(pyridin-3-yl)-1H-benzo[d]imidazole-5-carboxamide;
(S)—N-(2,3-dihydro-1H-inden-1-yl)-2-(1-methylpiperidin-4-yl)-1H-benzo[d]imidazole-5-carboxamide;
(R)-2-(1-methylpiperidin-4-yl)-N-(1,2,3,4-tetrahydronaphthalen-1-yl)-1H-benzo[d]imidazole-5-carboxamide;
(S)-2-(1-methylpiperidin-4-yl)-N-(1,2,3,4-tetrahydronaphthalen-1-yl)-1H-benzo[d]imidazole-5-carboxamide;
(R)—N-(6-chlorochroman-4-yl)-2-(1-methylpiperidin-4-yl)-1H-benzo[d]imidazole-5-carboxamide;
(S)—N-(6-chlorochroman-4-yl)-2-(1-methylpiperidin-4-yl)-1H-benzo[d]imidazole-5-carboxamide;
(S)—N-(chroman-4-yl)-2-(1-methylpiperidin-4-yl)-1H-benzo[d]imidazole-5-carboxamide;
(S)—N-(chroman-4-yl)-2-(4-ethylpiperazin-1-yl)-1H-benzo[d]imidazole-5-carboxamide;
(S)—N-(chroman-4-yl)-1-methyl-2-(4-methylpyridin-3-yl)-1H-benzo[d]imidazole-5-carboxamide;
(S)—N-(chroman-4-yl)-1-methyl-2-(6-methylpyridin-3-yl)-1H-benzo[d]imidazole-5-carboxamide;
(S)—N-(chroman-4-yl)-1-methyl-2-(2-methylpyridin-3-yl)-1H-benzo[d]imidazole-5-carboxamide;
(S)—N-(chroman-4-yl)-1-methyl-2-(5-methylpyridin-3-yl)-1H-benzo[d]imidazole-5-carboxamide;
(S)—N-(chroman-4-yl)-1-methyl-2-(pyridin-3-yl)-1H-benzo[d]imidazole-5-carboxamide;
(S)—N-(chroman-4-yl)-1-methyl-2-(4-methylpyridin-3-yl)-1H-benzo[d]imidazole-6-carboxamide;
(S)—N-(chroman-4-yl)-1-methyl-2-(6-methylpyridin-3-yl)-1H-benzo[d]imidazole-6-carboxamide;
(S)—N-(chroman-4-yl)-1-methyl-2-(2-methylpyridin-3-yl)-1H-benzo[d]imidazole-6-carboxamide;
(S)—N-(chroman-4-yl)-1-methyl-2-(5-methylpyridin-3-yl)-1H-benzo[d]imidazole-6-carboxamide;
(S)—N-(chroman-4-yl)-1-methyl-2-(pyridin-3-yl)-1H-benzo[d]imidazole-6-carboxamide;
(S)—N-(chroman-4-yl)-2-(4-ethylpiperazin-1-yl)-1-methyl-1H-benzo[d]imidazole-6-carboxamide;
(S)—N-(chroman-4-yl)-1-methyl-2-(piperazin-1-yl)-1H-benzo[d]imidazole-6-carboxamide;
(S)—N-(chroman-4-yl)-2-(4-ethylpiperazin-1-yl)-1-methyl-1H-benzo[d]imidazole-5-carboxamide;
(S)—N-(chroman-4-yl)-1-methyl-2-(piperazin-1-yl)-1H-benzo[d]imidazole-5-carboxamide;
(S)—N-(chroman-4-yl)-2-(pyridin-3-yl)-[1,2,4]triazolo[1,5-a]pyridine-6-carboxamide;
(S)—N-(chroman-4-yl)-2-(6-methylpyridin-3-yl)-[1,2,4]triazolo[1,5-a]pyridine-6-carboxamide;
(S)—N-(chroman-4-yl)-2-(5-methylpyridin-3-yl)-[1,2,4]triazolo[1,5-a]pyridine-6-carboxamide;
(S)—N-(chroman-4-yl)-2-(4-methylpyridin-3-yl)-[1,2,4]triazolo[1,5-a]pyridine-6-carboxamide;
(S)—N-(chroman-4-yl)-2-(2-methylpyridin-3-yl)-[1,2,4]triazolo[1,5-a]pyridine-6-carboxamide;
(S)—N-(chroman-4-yl)-2-(2,6-dimethylpyridin-3-yl)-[1,2,4]triazolo[1,5-a]pyridine-6-carboxamide;
(S)—N-(chroman-4-yl)-2-(piperazin-1-yl)-[1,2,4]triazolo[1,5-a]pyridine-6-carboxamide;
(S)—N-(chroman-4-yl)-2-(4-methylpiperazin-1-yl)-[1,2,4]triazolo[1,5-a]pyridine-6-carboxamide;
(S)—N-(chroman-4-yl)-2-morpholino-[1,2,4]triazolo[1,5-a]pyridine-6-carboxamide;
(S)-2-(4-(tert-butyl)piperazin-1-yl)-N-(chroman-4-yl)-[1,2,4]triazolo[1,5-a]pyridine-6-carboxamide;
(S)—N-(chroman-4-yl)-2-(piperidin-1-yl)-[1,2,4]triazolo[1,5-a]pyridine-6-carboxamide;
(S)—N-(chroman-4-yl)-2-(4-ethylpiperazin-1-yl)-[1,2,4]triazolo[1,5-a]pyridine-6-carboxamide;
(S)—N-(chroman-4-yl)-2-(4-isopropylpiperazin-1-yl)-[1,2,4]triazolo[1,5-a]pyridine-6-carboxamide;
(R)—N-(2,3-dihydro-1H-inden-1-yl)-2-(piperazin-1-yl)-[1,2,4]triazolo[1,5-a]pyridine-6-carboxamide;
(S)—N-(2,3-dihydro-1H-inden-1-yl)-2-(piperazin-1-yl)-[1,2,4]triazolo[1,5-a]pyridine-6-carboxamide;
(S)—N-(chroman-4-yl)-2-(piperidin-4-yl)-[1,2,4]triazolo[1,5-a]pyridine-6-carboxamide;
(S)—N-(chroman-4-yl)-2-(1-methylpiperidin-4-yl)-[1,2,4]triazolo[1,5-a]pyridine-6-carboxamide;
(S)—N-(chroman-4-yl)-2-(1-ethylpiperidin-4-yl)-[1,2,4]triazolo[1,5-a]pyridine-6-carboxamide;
(S)—N-(chroman-4-yl)-2-(2-methylpyridin-3-yl)-[1,2,4]triazolo[1,5-a]pyridine-7-carboxamide;
(S)—N-(chroman-4-yl)-2-(4-methylpyridin-3-yl)-[1,2,4]triazolo[1,5-a]pyridine-7-carboxamide;
(S)—N-(chroman-4-yl)-2-(4-hydroxypiperidin-1-yl)-[1,2,4]triazolo[1,5-a]pyridine-7-carboxamide;
(S)—N-(chroman-4-yl)-2-(piperazin-1-yl)-[1,2,4]triazolo[1,5-a]pyridine-7-carboxamide;
(S)—N-(chroman-4-yl)-2-(4-methylpiperazin-1-yl)-[1,2,4]triazolo[1,5-a]pyridine-7-carboxamide;
(S)—N-(chroman-4-yl)-2-morpholino-[1,2,4]triazolo[1,5-a]pyridine-7-carboxamide;
(S)-2-(4-(tert-butyl)piperazin-1-yl)-N-(chroman-4-yl)-[1,2,4]triazolo[1,5-a]pyridine-7-carboxamide;
(S)—N-(chroman-4-yl)-2-(piperidin-1-yl)-[1,2,4]triazolo[1,5-a]pyridine-7-carboxamide;
(S)—N-(chroman-4-yl)-2-(4-ethylpiperazin-1-yl)-[1,2,4]triazolo[1,5-a]pyridine-7-carboxamide;
(S)—N-(chroman-4-yl)-2-(4-isopropylpiperazin-1-yl)-[1,2,4]triazolo[1,5-a]pyridine-7-carboxamide;
(S)—N-(2,3-dihydro-1H-inden-1-yl)-2-(piperazin-1-yl)-[1,2,4]triazolo[1,5-a]pyridine-7-carboxamide;
(R)—N-(2,3-dihydro-1H-inden-1-yl)-2-(piperazin-1-yl)-[1,2,4]triazolo[1,5-a]pyridine-7-carboxamide;
(S)—N-(2,3-dihydro-1H-inden-1-yl)-2-(4-methylpiperazin-1-yl)-[1,2,4]triazolo[1,5-a]pyridine-7-carboxamide;
(S)—N-(2,3-dihydro-1H-inden-1-yl)-2-(4-ethylpiperazin-1-yl)-[1,2,4]triazolo[1,5-a]pyridine-7-carboxamide;
2-(3,8-diazabicyclo[3.2.1]octan-8-yl)-N-(4,4-difluorocyclohexyl)imidazo[1,2-a]pyridine-6-carboxamide;
2-(3,8-diazabicyclo[3.2.1]octan-8-yl)-N-cyclopentylimidazo[1,2-a]pyridine-6-carboxamide;
(S)—N-(chroman-4-yl)-2-(piperazin-1-yl)pyrazolo[1,5-a]pyrimidine-6-carboxamide;
(S)—N-(chroman-4-yl)-2-(4-methylpiperazin-1-yl)pyrazolo[1,5-a]pyrimidine-6-carboxamide;
(S)—N-(chroman-4-yl)-2-(4-ethylpiperazin-1-yl)pyrazolo[1,5-a]pyrimidine-6-carboxamide;
(S)—N-(2,3-dihydro-1H-inden-1-yl)-2-(piperazin-1-yl)pyrazolo[1,5-a]pyrimidine-6-carboxamide;
N-cyclopentyl-2-(4-ethylpiperazin-1-yl)pyrazolo[1,5-a]pyrimidine-6-carboxamide;
N-cyclohexyl-2-(4-ethylpiperazin-1-yl)pyrazolo[1,5-a]pyrimidine-6-carboxamide;
N-(4,4-difluorocyclohexyl)-2-(4-ethylpiperazin-1-yl)pyrazolo[1,5-a]pyrimidine-6-carboxamide;
(S)—N-(2,3-dihydro-1H-inden-1-yl)-2-(4-ethylpiperazin-1-yl)pyrazolo[1,5-a]pyrimidine-6-carboxamide;
N-cyclopentyl-2-(4-methylpiperazin-1-yl)pyrazolo[1,5-a]pyrimidine-6-carboxamide;
N-cyclohexyl-2-(4-methylpiperazin-1-yl)pyrazolo[1,5-a]pyrimidine-6-carboxamide;
N-(4,4-difluorocyclohexyl)-2-(4-methylpiperazin-1-yl)pyrazolo[1,5-a]pyrimidine-6-carboxamide;
(S)—N-(2,3-dihydro-1H-inden-1-yl)-2-(4-methylpiperazin-1-yl)pyrazolo[1,5-a]pyrimidine-6-carboxamide;
2-(3,8-diazabicyclo[3.2.1]octan-8-yl)-N-cyclopentylpyrazolo[1,5-a]pyrimidine-6-carboxamide;
N-cyclopentyl-2-(3-methyl-3,8-diazabicyclo[3.2.1]octan-8-yl)pyrazolo[1,5-a]pyrimidine-6-carboxamide;
(S)—N-(chroman-4-yl)-2-(piperidin-4-yl)pyrazolo[1,5-a]pyrimidine-6-carboxamide;
(S)—N-(2,3-dihydro-1H-inden-1-yl)-2-(piperidin-4-yl)pyrazolo[1,5-a]pyrimidine-6-carboxamide;
(S)—N-(chroman-4-yl)-2-(1-ethylpiperidin-4-yl)pyrazolo[1,5-a]pyrimidine-6-carboxamide;
N-cyclopentyl-2-(1-methylpiperidin-4-yl)pyrazolo[1,5-a]pyrimidine-6-carboxamide;
N-cyclohexyl-2-(1-methylpiperidin-4-yl)pyrazolo[1,5-a]pyrimidine-6-carboxamide;
N-(4,4-difluorocyclohexyl)-2-(1-methylpiperidin-4-yl)pyrazolo[1,5-a]pyrimidine-6-carboxamide;
(S)—N-(chroman-4-yl)-2-(1-methylpiperidin-4-yl)pyrazolo[1,5-a]pyrimidine-6-carboxamide;
N-(4-fluorobenzyl)-2-(piperidin-4-yl)pyrazolo[1,5-a]pyrimidine-6-carboxamide;
(S)—N-(chroman-4-yl)-2-(pyridin-3-yl)pyrazolo[1,5-a]pyrimidine-6-carboxamide;
N-(4,4-difluorocyclohexyl)-7-methyl-2-(piperidin-4-yl)pyrazolo[1,5-a]pyrimidine-6-carboxamide;
(S)—N-(chroman-4-yl)-7-methyl-2-(piperidin-4-yl)pyrazolo[1,5-a]pyrimidine-6-carboxamide;
(S)—N-(chroman-4-yl)-2-(1-ethylpiperidin-4-yl)-7-methylpyrazolo[1,5-a]pyrimidine-6-carboxamide;
N-(4,4-difluorocyclohexyl)-2-(1-ethylpiperidin-4-yl)-7-methylpyrazolo[1,5-a]pyrimidine-6-carboxamide;
(S)—N-(chroman-4-yl)-7-methyl-2-(1-methylpiperidin-4-yl)pyrazolo[1,5-a]pyrimidine-6-carboxamide;
N-(4,4-difluorocyclohexyl)-7-methyl-2-(1-methylpiperidin-4-yl)pyrazolo[1,5-a]pyrimidine-6-carboxamide;
N-cyclopentyl-2-(piperidin-4-yl)benzo[d]oxazole-5-carboxamide;
N-cyclopentyl-2-(1-methylpiperidin-4-yl)benzo[d]oxazole-5-carboxamide;
N-cyclopentyl-2-(1-ethylpiperidin-4-yl)benzo[d]oxazole-5-carboxamide;
(S)—N-(chroman-4-yl)-2-(piperazin-1-yl)benzo[d]oxazole-5-carboxamide;
N-cyclopentyl-2-(piperazin-1-yl)benzo[d]oxazole-5-carboxamide;
(S)—N-(chroman-4-yl)-2-(4-ethylpiperazin-1-yl)benzo[d]oxazole-5-carboxamide;
N-cyclopentyl-2-(4-ethylpiperazin-1-yl)benzo[d]oxazole-5-carboxamide;
(S)—N-(chroman-4-yl)-2-(4-methylpiperazin-1-yl)benzo[d]oxazole-5-carboxamide;
N-cyclopentyl-2-(4-methylpiperazin-1-yl)benzo[d]oxazole-5-carboxamide;
(S)—N-(chroman-4-yl)-2-(4-(2-hydroxyethyl)piperazin-1-yl)benzo[d]oxazole-5-carboxamide;
N-cyclopentyl-2-(4-(2-hydroxyethyl)piperazin-1-yl)benzo[d]oxazole-5-carboxamide;
N-cyclopentyl-2-(4-ethylpiperazin-1-yl)benzo[d]oxazole-6-carboxamide;
N-cyclopentyl-2-(piperazin-1-yl)benzo[d]oxazole-6-carboxamide;
(S)—N-(chroman-4-yl)-2-(1-methyl-1H-pyrazol-4-yl)benzo[d]oxazole-6-carboxamide;
N-cyclopentyl-2-(piperidin-4-yl)thiazolo[5,4-b]pyridine-5-carboxamide;
N-(4,4-difluorocyclohexyl)-2-(piperidin-4-yl)thiazolo[5,4-b]pyridine-5-carboxamide;
N-cyclopentyl-2-(1-methylpiperidin-4-yl)thiazolo[5,4-b]pyridine-5-carboxamide;
N-(4,4-difluorocyclohexyl)-2-(1-methylpiperidin-4-yl)thiazolo[5,4-b]pyridine-5-carboxamide;
2-(3,8-diazabicyclo[3.2.1]octan-8-yl)-N-cyclopentylthiazolo[5,4-b]pyridine-5-carboxamide;
2-(3,8-diazabicyclo[3.2.1]octan-8-yl)-N-(4,4-difluorocyclohexyl)thiazolo[5,4-b]pyridine-5-carboxamide;
2-(3,8-diazabicyclo[3.2.1]octan-8-yl)-N-(4,4-difluorocyclohexyl)thiazolo[4,5-b]pyridine-6-carboxamide;
N-cyclopentyl-2-(piperidin-4-yl)thiazolo[4,5-b]pyridine-6-carboxamide;
2-(3,8-diazabicyclo[3.2.1]octan-8-yl)-N-cyclopentylthiazolo[4,5-c]pyridine-6-carboxamide;
2-(3,8-diazabicyclo[3.2.1]octan-8-yl)-N-(4,4-difluorocyclohexyl)thiazolo[4,5-c]pyridine-6-carboxamide;
2-(3,8-diazabicyclo[3.2.1]octan-8-yl)-N-cyclopentylthieno[3,2-b]pyridine-6-carboxamide;
2-(3,8-diazabicyclo[3.2.1]octan-8-yl)-N-(4,4-difluorocyclohexyl)thieno[3,2-b]pyridine-6-carboxamide;
and pharmaceutically acceptable salts thereof.

In a further embodiment of a compound of Formula I, R¹ may be as defined in any of the compounds of Formula I, above. In a further embodiment of a compound of Formula I, R² may be as defined in any of the compounds of Formula I, above.

### Definitions

The term “aromatic ring” refers to an aromatic carbocyclic ring system. The term “heteroaromatic ring” refers to an aromatic ring system wherein one or more of the ring-forming atoms is a heteroatom such as 0, S or N. An aromatic ring may be a 6-membered aromatic ring, i.e. a phenyl ring. A heteroaromatic ring may be a 6-membered heteroaromatic ring that contains one to three N atoms or a 5-membered heteroaromatic ring that contains one to three heteroatoms selected from O, S and N. Examples of such 6- or 5-membered heteroaromatic rings include pyridine, pyridazine, pyrazine, pyrimidine, thiophene, furan, thiazole, thiadiazole, oxazole, oxadiazole, imidazole, triazole and their isomers including isothiazole, isothiadiazole, isoxazole and isoxadiazole. In all instances described above, an aromatic ring may be optionally substituted as defined herein.

The term “carbocyclic ring” refers to a ring system with may be saturated, partially unsaturated or aromatic and wherein all ring forming atoms are carbon. The term “heterocyclic ring” refers to a ring system with may be saturated, partially unsaturated or aromatic and wherein one or more of the ring-forming atoms is a heteroatom such as O, S or N. A “non-aromatic carbocyclic or heterocyclic ring” may be saturated or partially unsaturated. Carbocyclic and heterocyclic rings may be bicyclic or multicyclic ring systems, for example bicyclic or multicyclic fused ring systems or bicyclic or multicyclic spiro ring systems or a combination thereof. Each ring within a fused ring system may independently be saturated, partially unsaturated or aromatic. Examples of such fused bicyclic ring systems include indane and chromane. A non-aromatic carbocyclic or heterocyclic ring may include fused ring systems, where for example two rings share two adjacent atoms, bridged ring systems, where for example two rings share three or more adjacent atoms, or spiro ring systems, where for example two rings share one adjacent atom. Examples of fused ring systems include octahydropyrrolo[1,2-a]pyrazine and octahydro-2H-pyrido[1,2-a]pyrazine. Bridged rings may comprise three or more rings. Examples of such bridged ring systems include 2,5-diazabicyclo[2.2.1]heptane, 2,5-diazabicyclo[2.2.2]octane and 3,8-diazabicyclo[3.2.1]octane. Examples of spiro ring systems include spiro[4.3]octane and 2,6-diazaspiro[3.4]octane. In all instances described above, a carbocyclic or heterocyclic ring may be optionally substituted as defined herein.

Where a ring is referred to herein as containing specified ring heteroatoms, it will be appreciated that no further ring heteroatoms are present beyond those specified.

A “monocyclic, bridged or bicyclic ring” includes monocyclic rings, bridged ring systems and bicyclic ring systems. A “monocyclic, bridged or bicyclic ring”, unless otherwise defined, may be saturated, partially unsaturated or aromatic. These may be aromatic, heteroaromatic, carbocyclic or heterocyclic rings or combinations thereof. Bicyclic ring systems may include fused and spiro rings.

Unless otherwise defined, the term “alkyl” as used herein refers to a saturated hydrocarbon which may be straight-chain, branched, cyclic or a combination thereof. Alkyl groups include linear, branched or cyclic alkyl groups and hybrids thereof, such as (cycloalkyl)alkyl. The term “(C1-6)alkyl” as used herein means an alkyl group having 1-6 carbon atoms, which may be branched or unbranched and optionally contains a ring. Examples of (C1-6)alkyl include hexyl, cyclohexyl, pentyl, cyclopentyl, butyl, isobutyl, cyclobutyl, tertiary butyl, propyl, isopropyl, cyclopropyl, cyclopropylmethyl, ethyl and methyl. The term “(C1-4)alkyl” as used herein means a branched or unbranched alkyl group having 1-4 carbon atoms, optionally containing a ring. Examples of (C1-4)alkyl include butyl, isobutyl, cyclobutyl, tertiary butyl, propyl, isopropyl, cyclopropyl, cyclopropylmethyl, ethyl and methyl. A (C1-4)alkyl as referenced herein may preferably be a (C1-2)alkyl. Where specified in the formulae above, (C1-4)alkyl may be substituted, for example with 1 to 3 fluoros. A particularly preferred example of a substituted (C1-4)alkyl is trifluoromethyl. Alternatively (C1-4)alkyl may be unsubstituted.

The term “alkylene” as used herein refers to a divalent alkyl group.

The term “cycloalkyl” refers to a cyclic alkyl group, for example cycloheptyl, cyclohexyl, cyclopentyl, cyclobutyl or cyclopropyl. Cycloalkyl may be substituted as defined herein.

The term “alkoxy” means —O-alkyl wherein alkyl has the meaning as defined above. Examples of (C1-4)alkoxy include methoxy, ethoxy, propoxy, isopropoxy, butoxy, isobutoxy and tertiary butoxy. A (C1-4)alkoxy as referenced herein may preferably be a (C1-2)alkoxy. Where specified in the formulae above, (C1-4)alkoxy may be substituted, for example with 1 to 3 fluoros. A particularly preferred example of a substituted (C1-4)alkoxy is trifluoromethoxy. Alternatively, (C1-4)alkoxy may be unsubstituted. In the present invention, alkoxy is attached to the rest of the molecule by the “oxy” moiety.

A group that is referred to herein as being “substituted”, whether preceded by the term “optionally” or not, means that at least one hydrogen present on a group (e.g. a C or N atom) is replaced with a permissible substituent, for example a substituent which upon substitution results in a stable compound, e.g. a compound which does not spontaneously undergo transformation such as by rearrangement, cyclization, elimination or other reaction. Unless otherwise indicated, when more than one substituent is present, the substituent is either the same or different at each occurrence. Unless otherwise indicated, a “substituted” group has one or more substituents at one or more substitutable positions of the group, and when more than one position in any given structure is substituted, the substituent is either the same or different at each position.

The term “halogen” means F, Cl, Br or I. F and Cl are particularly preferred, with F the most preferred.

### Activation of Long PDE4 Isoforms

PDE4 long isoforms have two regulatory regions, upstream conserved region 1 (UCR1) and upstream conserved region 2 (UCR2). These are between the isoform-specific N-terminal portion and the catalytic domain. The UCR1 domain is missing in the short forms, whereas the super-short forms not only lack UCR1, but also have a N-terminal truncated UCR2 domain (Houslay, M. D., Schafer, P. and Zhang, K. Drug Discovery Today 10: 1503-1519, 2005).

There are four PDE4 families, PDE4A, PDE4B, PDE4C and PDE4D. The present invention concerns compounds that are capable of activating one or more of the long isoforms from one or more of these four families. The long isoform PDE4 may therefore be long isoform PDE4A, long isoform PDE4B, long isoform PDE4C or long isoform PDE4D. For the avoidance of doubt, a long isoform PDE4 contains a UCR1 region. In some embodiments, a long isoform PDE4 as referred to herein is human. UCR1 is conserved within mammalian species (Houslay, MD, Sullivan, M and Bolger G B Adv. Pharmacol. 44: 225-342, 1998), so in other embodiments, the long isoform PDE4 can be from a non-human mammal.

Without wishing to be bound by theory, the compounds described herein may act as PDE4 long form activators. The compounds described herein are small molecules that are believed to bind directly to PDE4 long forms and induce structural changes that increase, stabilise, uncover and/or maintain the catalytic activity of these enzymes. Without wishing to be bound by theory, the activation of PDE4 long forms by PDE4 long form activators may be sensitive to the regulatory status of the enzyme, including post-translational modifications (such as phosphorylation) or the adoption of protein-protein complexes associated with a particular physiological localisation or with a cellular or biochemical assay context. PDE4 long form activators may manifest activation of the enzyme in one or more states but not necessarily all states.

In the field of pharmacology, and as used herein, a small molecule is defined as a low molecular weight organic compound that may serve as a regulator of biological processes. Preferred small molecule activators according to the present invention have a molecular weight of less than or equal to 700 Daltons. This allows for the possibility to rapidly diffuse across cell membranes and reach intracellular sites of action (Veber, D. F. et al., J. Med. Chem. 45: 2615-2623, 2002). Especially preferred small molecule activators according to the present invention have molecular weights of greater than or equal to 250 Daltons and less than or equal to 500 Daltons (Lipinski, C. A. Drug Discovery Today: Technologies 1: 337-341, 2004).

One suitable method of detecting whether or not a compound is capable of serving as an activator of a PDE4 long form is using a two-step radio-assay procedure described in Experiment 1. In summary, the method involves incubating a PDE4 long form with a test small molecule activator, together with [³H]-labelled cAMP to assess alterations in the breakdown of cAMP to the 5′-adenosine monophosphate (5′-AMP) product. A sample of the reaction mixture from such an incubation is subsequently treated with snake venom 5′-nucleotidase to allow conversion of the nucleotide [³H]-labelled 5′-AMP to the uncharged nucleoside [³H]-labelled adenosine, which can be separated and quantified to assess PDE4 activity and the effect of the test compound (Thompson, W. J. and Appleman, M. M. Biochemistry 10: 311-316, 1971, with some modifications as described in: Marchmont, R. J. and Houslay, M. D. Biochem J. 187: 381-92, 1980).

Using the above assay procedure, as described in detail in Experiment 1, preferred compounds described herein may produce an increase in the background activity of one or more PDE4 long forms of more than 20% or more than 30% at a test compound concentration of 100 micromolar or less. Especially preferred compunds described herein may produce an increase in the background activity of one or more PDE4 long forms of more than 20% or more than 30% at a test compound concentration of 10 micromolar, or less, for example 3 micromolar.

The compounds described herein may be selective for the long form of the PDE4 enzyme and, as such, do not act or act to a lesser extent as activators of the short or super-short isoforms of the PDE4 enzyme. The short or super-short isoform PDE4 may be short or super-short isoform PDE4A, short or super-short isoform PDE4B, short or super-short isoform PDE4C, or short or super-short isoform PDE4D. For the avoidance of doubt, short and super-short isoforms of PDE4 lack a UCR1 domain. Super-short isoforms are characterised by a truncated UCR2 domain and lack of a UCR1 domain. The short or super-short isoform PDE4 is, for example, human, but may also be from other mammalian species (where UCR2 is conserved, see Houslay, MD, Sullivan, M and Bolger G B Adv. Pharmacol. 44: 225-342, 1998).

Under the same assay conditions, as described in Experiment 1, the compounds described herein may produce a less than 30% or less than 20% increase in the background activity of the short or super-short forms of the PDE4A, PDE4B, PDE4C or PDE4D enzymes at a test compound concentration of 100 micromolar, or less.

Compounds described herein may therefore provide a positive result in an assay for activation of a long form PDE4 and a negative result in an assay for activation of a short form (or super-short form) of PDE4.

PDE4 long isoforms include those now known as PDE4A4, PDE4A4/5, PDE4A5, PDE4A8, PDE4A10, PDE4A11, PDE4B1, PDE4B3, PDE4B4, PDE4C1, PDE4C2, PDE4C3, PDE4C4, PDE4D3, PDE4D4, PDE4D5, PDE4D7, PDE4D8, PDE4D9 and PDE4D11. Further long isoforms may be or have already been identified or called by different nomenclature from any of the four PDE4 sub-families.

PDE4 short and super-short isoforms include PDE4A1, PDE4B2, PDE4B5, PDE4D1, PDE4D2, PDE4D6 and PDE4D10. Further short and super-short isoforms may be or have already been identified or called by different nomenclature from any of the four PDE4 sub-families.

The Examples below exemplify activity of compounds described herein in an assay for activation of the human PDE4D5 long isoforms and a lack of activity in an assay for activation of the human PDE4B2 short isoform. Details of these isoforms and a number of the other known isoforms, including GenBank accession numbers, are provided in Tables A to D immediately below.

TABLE A
Examples of known PDE4A Isoforms

| | | | Calculated | |
|---|---|---|---|---|
| | | | molecular | |
| Isoform | Species | Accession | weight (kDa) | Type |
| PDE4A1 | Human | NM_006202 | 73 | Short |
| PDE4A1 | Rodent | L27062 | 68 | Short |
| PDE4A4* | Human | L20965 | 98 | Long |
| PDE4A5 | Rodent | L27057 | 93 | Long |
| PDE4A7** | Human | U18088 | 37 | Dead-Short |
| PDE4A8 | Human | AY593872 | 96 | Long |
| PDE4A8 | Rodent | L36467 | 85 | Long |
| PDE4A10 | Human | AF073745 | 91 | Long |
| PDE4A11 | Human | AY618547 | 95 | Long |

*Note that the PDE4A4B clone is correct while PDE4A4A has a cloning artefact and PDE4A4C is a truncation artefact.
**Note that this species is C- as well as N-terminally truncated

TABLE B
Examples of known PDE4B Isoforms

| | | | Calculated | |
|---|---|---|---|---|
| | | | molecular | |
| Isoform | Species | Accession | weight (kDa) | Type |
| PDE4B1 | Human | NM_001037341.1 | 83 | Long |
| PDE4B2 | Human | NM_001037339.1 | 64 | Short |
| PDE4B3 | Human | NM_001037340 | 83 | Long |
| PDE4B4 | Rodent | AF202733.1 | 75 | Long |
| PDE4B5 | Human | EF595686.1 | 57 | Super-short |

TABLE C
Examples of known PDE4C Isoforms

| | | | Calculated | |
|---|---|---|---|---|
| | | | molecular | |
| Isoform | Species | Accession | weight (kDa) | Type |
| PDE4C1 | Human | NM_000923 | 79 | Long |
| PDE4C2 | Human | NM_001098819 | 67 | Long |
| PDE4C3 | Human | NM_001098818 | 76 | Long |
| PDE4C4 | Human | U66346 | 88 | Long |
| PDE4C5 | Human | U66347 | 47 | Partial |
| PDE4C6 | Human | U66348 | 58 | Partial |
| PDE4C7 | Human | U66349 | 48 | Partial |

TABLE D
Examples of known PDE4D Isoforms

| | | | Calculated | |
|---|---|---|---|---|
| | | | molecular | |
| Isoform | Species | Accession | weight (kDa) | Type |
| PDE4D1 | Human | NM_001197222 | 66 | Short |
| PDE4D2 | Human | NM_001197221 | 58 | Super-short |
| PDE4D3 | Human | NM_006203 | 76 | Long |
| PDE4D4 | Human | NM_001104631 | 91 | Long |
| PDE4D5 | Human | NM_001197218 | 84 | Long |
| PDE4D6 | Human | NM_001197223 | 59 | Super-short |
| PDE4D7 | Human | NM_001165899 | 85 | Long |
| PDE4D8 | Human | NM_001197219 | 78 | Long |
| PDE4D9 | Human | NM_001197220 | 77 | Long |
| PDE4D10 | Rodent | DQ665896.1 | 58 | Super-short |
| PDE4D11 | Rodent | EU489880.1 | 79 | Long |

* PDE4D8 was originally called PDE4D6 in the literature

Reduction of cAMP Levels

Without wishing to be bound by theory, the compounds described herein may function by reducing cAMP levels in one or more intracellular compartments. The PDE4 long form activators described herein may thus provide a means to regulate certain cellular processes that are dependent upon cAMP. Excessive intracellular cAMP signalling mediates a number of diseases and disorders. Therefore, the compounds described herein are expected to be of utility for the treatment of diseases associated with abnormally elevated cAMP levels, increased cAMP-mediated signalling and/or reduced cAMP elimination, enzymatic or otherwise (e.g. via efflux). The treatment is typically of a human, but may also be of a non-human animal, such as a non-human mammal (e.g. veterinary treatment).

In one aspect, the present invention provides a compound described here (i.e. a small molecule activator of a PDE4 long form), for use in a method for the treatment or prevention of disorders where a reduction of second messenger responses mediated by cyclic 3′,5′-adenosine monophosphate (cAMP) is required.

For example, gain-of-function gene mutations in proteins involved in driving cAMP signalling upstream of adenylyl cyclase, such as GPCRs and Gsa, can lead to abnormal excessive cAMP activity with pathological consequences (Lania A, Mantovani G, Spada A. Ann Endocrinol (Paris). 73: 73-75, 2012.; Thompson, M. D. et al., Methods Mol. Biol. 448: 109-137, 2008; Weinstein L S, Liu J, Sakamoto A, Xie T, Chen M. Endocrinology. 145: 5459-5464, 2004; Lania A, Mantovani G, Spada A. Eur J Endocrinol. 145: 543-559, 2001). PDE4 long form activators described herein, possessing the ability to accelerate the termination of cAMP action, would therefore be expected to be effective in the treatment, prevention or partial control of diseases characterised by undesirably high cAMP levels, or activity, as detailed below.

The treatment or prevention described herein may be treatment or prevention of a disease or disorder that can be ameliorated by activation of long isoforms of PDE4. The treatment or prevention described herein may be treatment or prevention of a disease or disorder mediated by excessive intracellular cAMP signalling. In these diseases, a reduction of second messenger responses mediated by cyclic 3′,5′-adenosine monophosphate (cAMP) should provide a therapeutic benefit.

Diseases Ameliorated by Activation of Long Isoforms of PDE4 or Characterised by Elevated cAMP Levels

### Hyperthyroidism

Stimulation of the thyroid-stimulating hormone (TSH) receptor (TSHR) leads to increased generation and release of thyroid hormones, thyroxine and triiodothyronine, through a cAMP-dependent signalling mechanism involving Gsa-mediated activation of adenylyl cyclase. Gain-of-function mutations in the TSHR have been reported to be involved in the development of hyperthyroidism (Duprez, L. et al., Nat. Genet. 7: 396-401, 1994; Biebermann, H. et al., J. Clin. Endocrinol. Metab. 86: 4429-4433, 2001; Karges, B. et al., J. Endocrinol. 186: 377-385, 2005). Activating mutations of both TSHR and Gsa have also been found in goitre and thyroid adenomas (Arturi, F. et al., Exp. Clin. Endocrinol. Diabetes 106: 234-236, 1998). The increased cAMP activity in thyroid adenomas, as a result of the activating TSHR or Gsa mutations, has been reported to produce a protective adaptive increase in PDE4 activity to counteract abnormal rise in cAMP levels and signal transduction (Persani, L. et al., J. Clin. Endocrinol. Metab. 85: 2872-2878, 2000).

The most common cause of hyperthyroidism is Graves' disease, an autoimmune disorder in which antibodies mimic TSH action at the TSHR, leading to excessive cAMP activity in thyroid follicle cells and consequently a state of hyperthyroidism.

PDE4 long form activators described herein are therefore expected to be effective in the treatment, prevention or partial control of hyperthyroidism. In one embodiment, the hyperthyroidism is associated with Graves' disease.

### Jansens's Metaphyseal Chondrodysplasia

Jansens's Metaphyseal Chondrodysplasia (JMC) is a very rare disease resulting from gain-of-function mutations of the parathyroid hormone (PTH) receptor 1 (PTHR1) (Thompson, M. D. et al., Methods Mol. Biol. 448: 109-137, 2008). The constitutive activation of the PTHR1 which couples to adenylyl cyclase as effector is associated with excessive cAMP signalling primarily in bone and kidney, leading to dysregulation of ion homeostasis characterised by hypercalcemia and hypophosphatemia (Calvi, L. M. and Schipani, E. J. Endocrinol. Invest. 23: 545-554, 2000) and developmental (e.g. short stature) and physical (e.g. protruding eyes) abnormalities. PDE4 long form activators described herein are therefore expected to be effective in the treatment, prevention or partial control of JMC.

### Hyperparathyroidism

Hyperparathyroidism (HPT) is characterized by excessive secretion from the parathyroid gland of PTH, which regulates plasma calcium and phosphate concentrations via PTHR1 receptors in the kidney, bone and GI tract. The resulting excessive stimulation of these receptors causes disruption of plasma ion homeostasis with patients showing hypercalcemia and hypophosphatemia. Primary HPT is driven by parathyroid gland hyperplasia or dysfunction, whereas secondary HPT is associated with underlying medical conditions, predominantly chronic renal disease. Left untreated, HPT causes a variety of debilitating symptoms and can become life-threatening.

By acting to down-regulate excessive cAMP generated by sustained PTH signalling, PDE4 long form activators described herein are expected to be effective in the treatment, prevention or partial control of hyperparathyroidism.

### Familial Male Precocious Puberty (Testotoxicosis)

Familial male-limited precocious puberty (FMPP), also known as familial sexual precocity or gonadotropin-independent testotoxicosis, is a disorder in which boys generally develop signs of precocious puberty in early childhood.

The spinal length in boys may be short due to a rapid advance in epiphyseal maturation. FMPP is an autosomal dominant condition with constitutively activating mutations in the luteinizing hormone (LH) receptor, which leads to increased cAMP production, associated with Leydig cell hyperplasia and low sperm cell count (Latronico, A. C. et al., J Clin. Endocrinol. Metab. 80: 2490-2494, 1995; Kosugi, S. et al., Hum. Mol. Genet. 4: 183-188, 1995). PDE4 long form activators described herein are therefore expected to be effective in the treatment, prevention or partial control of FMPP.

### Pituitary Adenomas and Cushing's Disease

Non-cancerous tumours of the pituitary gland are collectively referred to as pituitary adenomas and can lead to hypersecretion of adenohypophyseal hormones (e.g. growth hormone, thyroid stimulating hormone, luteinizing hormone, follicle stimulating hormone and adrenocorticotrophic hormone), which exert their action through GPCRs coupled to Gs and cAMP generation. Thus pituitary adenomas can lead to a state of enhanced cAMP mediated signalling in a variety of endocrine tissues which can precipitate a number of hormonal disorders such as acromegly (mainly due to excess growth hormone secretion), Cushing's disease (due to overproduction of adrenocorticotrophic hormone (ACTH) and the subsequent hypercortisolemia) and/or general hyperpituitarism (associated with excess release of multiple anterior pituitary hormones). Current treatment options for pituitary adenomas include treatment with dopamine receptor agonists, which reduce tumour size and lower pituitary hormonal output through a mechanism involving lowering of intracellular cAMP levels. PDE4 long form activators described herein may also be expected to attenuate the pathological effects of pituitary hormones in their target tissues, such as the adrenal glands.

In Cushing's disease, pituitary adenoma related overproduction of ACTH can lead to hypercortisolemia through an overactivation of melanocortin 2 receptor (MC2) and subsequent cAMP mediated stimulation of steroidogenesis and release of cortisol from the adrenal cortex (Tritos, N. A. and Biller, B. M. Discov. Med. 13: 171-179, 2012). PDE4 long form activators described herein are therefore expected to be effective in the treatment, prevention or partial control of Cushing's disease.

### Polycystic Kidney Disease

Polycystic kidney disease (PKD) is a genetic disorder of the kidneys characterised by development of pathological cysts, which damage renal structure and compromise kidney function (Takiar, V. and Caplan, M. J. Biochim. Biophys. Acta. 1812: 1337-1343, 2011; Masoumi, A. et al., Drugs 67: 2495-2510, 2007). There are two types of PKD: autosomal dominant polycystic kidney disease (ADPKD) and autosomal recessive polycystic kidney disease (ARPKD). ADPKD affects between 0.1% and 0.2% of the population worldwide and is characterized by progressive cyst development and enlarged kidneys. Approximately 50% of people with this disease will develop end stage kidney disease, usually between 40 and 70 years of age and require dialysis or kidney transplantation. ARPKD affects around 1:20,000 live births and is typically identified in the first few weeks after birth. Pulmonary hypoplasia results in a 30-50% death rate in neonates with ARPKD.

Defects in two genes are thought to be responsible for ADPKD. In around 85% of patients, development of ADPKD can be linked to mutations in the gene PKD1, encoding polycystin-1 (PC-1); in around 15% of patients mutations in PKD2, encoding polycystin-2 (PC-2) are implicated. Cyclic AMP has been identified as an important stimulus for proliferation and cyst expansion in polycystic kidney cells but not in normal human kidney cells (Yamaguchi, T. et al., Kidney Int. 57: 1460-1471, 2000). A considerable body of evidence has now developed to implicate cAMP as an important facilitator of renal cystogenesis (Masoumi, A. et al., Drugs 67: 2495-2510, 2007; Wallace, D. P. Biochim. Biophys. Acta. 1812:1291-1300, 2011). Consistent with the role of cAMP in cyst formation, agents that lower cAMP levels (e.g. vasopressin V2 receptor antagonists and the somatostatin receptor agonist octreotide) showed efficacy in rodent models of PKD (Torres, V. E. et al., Nat. Med. 10: 363-364, 2004; Gattone, V. H. 2^{nd} et al., Nat. Med. 9: 1323-1326, 2003; Belibi, F. A. and Edelstein, C. L. Expert Opin. Investig. Drugs. 19: 315-328, 2010). In zebrafish embryos, depletion of a cAMP-hydrolysing PDE enzyme subtype, PDE1A, resulted in development of a cystic phenotype, while PDE1A over-expression partially rescued cystic phenotypes resulting from PC2 depletion (Sussman, C. R., Ward, C. J., Leightner, A. C., Smith, J. L., Agarwal, R., Harris, P. C., Torres, V. E. J. Am. Soc. Nephrol. 25: 2222-2230, 2014). Phosphodiesterase activation has been suggested as a therapeutic strategy for PKD treatment (Sun, Y., Zhou, H. and Yang, B-X. Acta Pharmacologica Sinica 32: 805-816, 2011).

PDE4 long form activators described herein are therefore expected to be effective in the treatment, prevention or partial control of polycystic kidney disease.

### Polycystic Liver Disease

Polycystic liver disease (PLD) is a rare inherited condition associated with hepatic cystogenesis (usually defined when number of cysts exceeds 20), which often occurs in association with ADPKD (Strazzabosco, M. and Somlo, S. Gastroenterology 140: 1855-1859, 2011; Gevers, T. J. and Drenth, J. P. Curr. Opin. Gastroenterol. 27: 294-300, 2010). PLD may have a different genetic pathology when compared to ADPKD, driven by mutated proteins associated with the endoplasmic reticulum and the cilium. Increased cholangiocyte proliferation, neovascularisation and elevated fluid secretion act to drive liver cyst formation through dysregulation of multiple signal transduction pathways, including cAMP-mediated signalling. Elevation of hepatic cAMP levels stimulates cAMP-dependent chloride and fluid secretion in biliary epithelial cells and increases cholangiocyte proliferation (Janssen, M. J. et al., J. Hepatol. 52: 432-440, 2010). Somatostatin, which acts through a Gi-coupled mechanism to lower cAMP levels, reduced cholangiocyte proliferation and fluid secretion (Gong, A. Y. et al., Am. J. Physiol. Cell. Physiol. 284: C1205-1214, 2003). Furthermore, the synthetic somatostatin analogue, octreotide, showed efficacy in an animal model of PLD through a mechanism involving reduction in cAMP signalling (Masyuk, T. V. et al., Gastroenterology 132: 1104-1116, 2007). PDE4 long form activators described herein may therefore be effective in the treatment, prevention or partial control of polycystic liver disease due at least in part to cAMP.

### Maturity Onset Diabetes of Young Type 5 (MODY5)

MODY5 is a form of non-insulin-dependent diabetes mellitus associated with renal cysts. It is an autosomal dominant disorder caused by mutations in the gene encoding hepatocyte nuclear factor-1β (HNF-1β). The predominant clinical feature of patients affected by MODY5 is renal dysfunction, frequently diagnosed before the onset of diabetes. In some patients, HNF-1β mutations can result in additional phenotypic features, such as pancreatic atrophy, abnormal liver function and genital tract abnormalities. Studies in mice suggest that the mechanism responsible for renal cyst formation, associated with mutations of HNF-1β, involves a severe defect of the transcriptional activation of PKD2, in addition to effects on uromodulin (UMOD) and PKD1 genes. Down-regulation of PKD1 and PKD2 is associated with cAMP-driven formation of renal cysts (Mancusi, S. et al., J. Nephrol. 26: 207-12, 2013). HNF-1p also binds to the PDE4C promoter and regulates the expression of PDE4C (Ma et al., PNAS 104: 20386, 2007).

PDE4 long form activators described herein are therefore expected to be effective in the treatment, prevention or partial control of the symptoms of MODY5.

### Cardiac Hypertrophy, Heart Failure and Arrhythmia

Localized regulation and integration of cAMP signalling are important for proper cardiac function and perturbation of this signalling can lead to heart failure. Upon chronic β-adrenergic receptor stimulation, cardiomyocyte hypertrophy is induced via elevated cAMP and activation of its downstream effectors, including PKA and Epac (Wang, L. et al., Cell. Signal. 27: 908-922, 2015 and references therein). Cardiomyocyte hypertrophy increases the risk of heart failure and arrhythmia.

PDE4 long form activators described herein may therefore be effective in the treatment, prevention or partial control of cardiac hypertrophy, heart failure and/or arrhythmia.

Diseases Associated with Increased cAMP-Mediated Signalling
Disorders Associated with Activating Mutations of the Alpha Subunit of the G Protein (GNAS1)

The G-protein Gs acts as a transducer for GPCRs that stimulate adenylyl cyclase activity and exert their biological effects by increasing intracellular cAMP levels. Gs is a heterotrimeric protein composed of α, β and γ subunits. Activating mutations in the gene, GNAS1, for the α-subunit have been identified which lead to exaggerated abnormal cAMP signalling in a variety of tissues and give rise to a range of disorders.

### McCune-Albright Syndrome

McCune-Albright syndrome (MAS) is a rare genetic disorder typically characterised by three dominating features of precocious puberty, fibrous dysplasia of bone and café au lait lesions. The underlying molecular pathology for MAS involves an activating mutation of the GNAS1 gene (Diaz, A. Danon, M. and Crawford, J. J. Pediatr. Endocrinol. Metab. 20: 853-880, 2007). PDE4 long form activators described herein would therefore be expected to be effective in the treatment, prevention or partial control of disorders associated with activating mutations of GNAS1, including McCune-Albright syndrome.

### Amelioration of Toxin-Induced Increases in Adenylyl Cyclase Activity in Infectious Diseases.

Adenylyl cyclase, the enzyme responsible for production of cAMP, is a key biological target thought to be involved in mediating the effects of many bacterial toxins (Ahuja et al., Critical Reviews in Microbiology, 30: 187-196, 2004). These toxins produce their effects by raising cAMP levels through enhancement of host immune cell and/or pathogen related adenylyl cyclase activity. PDE4 long form activators described herein, by reducing cAMP levels, would therefore be expected to be of utility in the treatment or partial control of symptoms of infectious diseases that are associated with elevated cAMP activity. The following are some examples of such infectious diseases:

### Cholera

Vibrio cholerae produces cholera toxin, which through adenosine disphosphate ribosylation of the α subunit of Gs leads to host cell adenylyl cyclase activation and cAMP production. Diarrhoea caused by cholera toxin is believed to be a result of excessive cAMP accumulation in the cells of the gastrointestinal tract.

### Whooping Cough

Bordetella pertussis is the pathogen responsible for the childhood disease whooping cough. Bordetella pertussis toxin stimulates adenosine disphosphate ribosylation of the α subunit of Gi and indirectly augments cAMP levels in target cells. The bacterium also secretes an invasive adenylyl cyclase, which produces toxic cAMP levels and impairs host immune defence.

### Anthrax

Anthrax is caused by Bacillus anthracis and whilst it is primarily an animal disease it can be transmitted to humans through contact. Anthrax infections are associated with widespread oedema, the development of which is thought to be driven by oedema toxin. The latter is an adenylyl cyclase and is activated by host calmodulin to produce abnormally high levels of cAMP that have a toxic effect on host immune cells.

### Tuberculosis

Mycobactrium tuberculosis expresses a large and diverse range of adenylyl cyclases, which may play a role in virulence and generation of disease pathology. One adenylyl cyclase subtype, RV0386, has been demonstrated to enter host macrophages and elevate intracellular cAMP to cause toxicity (Agarwal et al., Nature, 460: 98-102, 2009).

PDE4 long form activators described herein may therefore be effective in the treatment, prevention or partial control of infectious diseases such as cholera, whooping cough, anthrax and tuberculosis.

Diseases Dependent Upon Activation of PKA by Elevated cAMP.

In eukaryotes, cAMP activates protein kinase A (PKA), which is also known as cAMP-dependent protein kinase. PKA is normally inactive as a tetrameric holoenzyme, consisting of two catalytic and two regulatory units, with the regulatory units blocking the catalytic centres of the catalytic units. cAMP binds to specific locations on the regulatory units of PKA and causes dissociation between the regulatory and catalytic units, thus activating the catalytic units. The active catalytic units catalyse the transfer of phosphate from ATP to specific residues of protein substrates, which may modulate the function of those protein substrates.

PDE4 long form activation reduces cAMP levels and cAMP mediated activation of PKA. PDE4 long form activators described herein would therefore be expected to be of utility in the treatment or partial control of disorders where inhibitors of PKA show evidence of therapeutic effects.

Disorders that are dependent upon activation of PKA by cAMP may be identified by their response to PKA inhibitors such as Rp-8-Br-cAMPS. Rp-8-Br-cAMPS is an analogue of cAMP that occupies the cAMP binding sites of PKA, preventing its dissociation and activation.

### HIV Infection and AIDS

T cells from HIV-infected patients have increased levels of cAMP and are more sensitive to inhibition by Rp-8-Br-cAMPS than are normal T cells. Excessive activation of PKA by cAMP has been associated with the progressive T cell dysfunction in HIV infection (Aandahl, E. M. et al., FASEB J. 12: 855-862, 1998). Furthermore, in vivo administration of Rp-8-Br-cAMPS has been shown to restore T cell responses in retrovirus-infected mice (Nayjib, B. et al., The Open Immunology Journal, 1: 20-24, 2008). PDE4 long form activators described herein are therefore expected to be of utility in the treatment, prevention or partial control of HIV infection and AIDS.

### Common Variable Immunodeficiency (CVID)

In vitro administration of Rp-8-Br-cAMPS has been shown to correct impaired secretion of the cytokine IL-10 by T cells from patients with Common Variable Immunodeficiency (CVID) (Holm, A. M. et al., J. Immunol. 170: 5772-5777, 2003). PDE4 long form activators described herein are therefore expected to be of utility in the treatment, prevention or partial control of CVID.

Diseases Dependent Upon Activation of Either or Both of Epacl and Epac2 by Elevated cAMP.

In addition to PKA, cAMP activates another intracellular receptor, known as exchange protein directly activated by cAMP (Epac). There are two isoforms of Epac, Epac1 and Epac2, both consisting of a regulatory region that binds cAMP and a catalytic region that promotes the exchange of GDP for GTP on the small G proteins, Rap1 and Rap2 of the Ras family. In addition, Epac proteins exert their functions through interactions with a number of other cellular partners at specific cellular loci. Pathophysiological changes in Epac signalling have been associated with a wide range of diseases (Breckler, M. et al., Cell. Signal. 23: 1257-1266, 2011).

Relevant disorders that are dependent upon activation of Epac proteins by cAMP may be identified by their response to Epac inhibitors, such as ESI-09, a novel non-cyclic nucleotide Epac1 and Epac2 antagonist that is capable of specifically blocking intracellular Epac-mediated Rap1 activation and Akt phosphorylation, as well as Epac-mediated insulin secretion in pancreatic beta cells (Almahariq, M. et al., Mol. Pharmacol. 83: 122-128, 2013).

### Melanoma

Epac1 has been implicated in promoting migration and metastasis in melanoma (Baljinnyam, E. et al., Pigment Cell Melanoma Res. 24: 680-687, 2011 and references cited therein). PDE4 long form activators described herein are therefore expected to be of utility in the treatment, prevention or partial control of melanoma.

### Pancreatic Cancer

It has recently been shown that Epac1 is markedly elevated in human pancreatic cancer cells as compared with normal pancreas or surrounding tissue (Lorenz, R. et al., Pancreas 37: 102-103, 2008).

Pancreatic cancer is often resistant to treatments that are usually effective for other types of cancer. Using the Epac inhibitor ESI-09, a functional role of Epac1 overexpression in pancreatic cancer cell migration and invasion was demonstrated (Almahariq, M. et al., Mol. Pharmacol. 83: 122-128, 2013). These findings are consistent with results based on RNAi silencing techniques and suggest that inhibition of Epac1 signalling could be an effective therapeutic strategy for pancreatic cancer.

PDE4 long form activators described herein would therefore be expected to be of utility in the treatment, prevention or partial control of pancreatic cancer.

Diseases Dependent Upon Modulation of cAMP-Gated Ion Channels by Elevated cAMP.

In addition to activation of PKA and Epac, another effector pathway for elevated cAMP is the activation of cAMP-gated ion channels. PDE4 long form activators described herein would therefore be expected to be of utility in the treatment of disorders where inhibitors of cAMP-gated ion channels show evidence of therapeutic effects.

Diseases Associated with Increased Activity of cAMP Response Element Binding Protein.

The cAMP response element binding protein (CREB) is an important transcription factor involved in the regulation of a variety of cellular functions such as cell proliferation, differentiation, survival, and apoptosis (Cho et al., Crit Rev Oncog, 16: 37-46, 2011). CREB activity is regulated by kinase dependant phosphorylation through a range of extracellular signals, such as stress, growth factors and neurotransmitters. Phosphorylation leads to dimerisation of CREB, and together with other co-activator partner proteins, enables it to bind to promoter regions of target genes containing the cAMP response element (CRE sites) and initiate transcriptional activity. The cAMP pathway (e.g. through cAMP-dependant protein kinase mediated phosphorylation) is an important positive modulator of CREB mediated biological activity. PDE4 long form activators described herein are therefore expected to be of utility in the treatment, prevention or partial control of disorders associated with elevated CREB activity.

### Leukaemia

Bone marrow cells from acute lymphoid and myeloid leukaemia patients have been reported to overexpress CREB protein and mRNA (Crans-Vargas et al., Blood, 99: 2617-9, 2002; Cho et al., Crit Rev Oncog, 16: 37-46, 2011). Furthermore, the increased CREB level correlates with poor clinical response in subjects with acute myeloid leukaemia (Crans-Vargas et al., Blood, 99: 2617-9, 2002; Shankar et al., Cancer Cell, 7:351-62, 2005). Upregulation of CREB is associated with stimulation of human leukaemia cell growth whilst downregulation inhibits myeloid cell proliferation and survival. PDE4 long form activators described herein would be expected to reduce CREB activity and function through attenuation of cAMP mediated stimulation of CREB and therefore expected to have utility in the treatment, prevention or partial control of acute lymphoid and myeloid leukaemia.

### Prostate Cancer

Abnormal excessive androgen activity is an important driver in the development of prostate cancer as it stimulates the development of intraepithelial neoplasias (Merkle et al., Cellular Signalling, 23: 507-515, 2011). This is strongly supported by the use of androgen ablation approaches, involving chemical or surgical castration, in the treatment of prostate cancer. Cyclic AMP elevating agents such as forskolin can enhance androgen receptor activity through multiple intracellular mechanisms including androgen receptor activation through phosphorylation and/or interaction with CREB. Epac1 activation has also been implicated in promoting cellular proliferation in prostate cancer (Misra, U. K. and Pizzo, S. V. J. Cell. Biochem. 108: 998-1011, 2009; Misra, U. K. and Pizzo, S. V. J. Cell. Biochem. 113: 1488-1500, 2012). PDE4 long form activators described herein are therefore expected to have utility in the treatment, prevention or partial control of prostate cancer.

Diseases Associated with Reduced Activity of cAMP-Hydrolysinq PDE Enzymes

Loss-of-function mutations in gene(s) for cAMP-hydrolysing PDE isoforms other than PDE4, such as PDE8 and PDE11, have been detected in a number of diseases (Vezzosi, D. and Bertherat, J., Eur. J. Endocrinol. 165: 177-188, 2011; Levy, I. et al., Curr. Opin. Pharmacol. 11: 689-697, 2011; Azevedo, M. F. and Stratakis, C. A. Endocr. Pract. 17 Suppl 3: 2-7, 2011). These mutations can lead to abnormally high cAMP levels and/or duration of cAMP action with pathological consequences as detailed below. PDE4 long form activators described herein are therefore expected to be of utility in the treatment, prevention or partial control of these diseases, such as adrenocortical tumours, testicular cancer, PPNAD and Carney Complex.

### Adrenocortical Tumours

Adrenocortical tumours associated with an inactivating point mutation in the gene encoding PDE11A4 have decreased expression of PDE11A4 and increased cAMP levels (Horvath, A. et al., Nat Genet. 38: 794-800, 2006; Horvath, A. et al., Cancer Res. 66: 11571-11575, 2006; Libe, R., et al., Clin. Cancer Res. 14: 4016-4024, 2008).

### Testicular Cancer

Mutations that reduce PDE11A activity and increase cAMP levels have been observed in some forms of testicular cancer (Horvath. A. et al., Cancer Res. 69: 5301-5306, 2009).

### Primary Pigmented Nodular Adrenocortical Diseases (PPNAD)

Mutations in the PDE8B gene have also been identified as a predisposing factor for PPNAD and the mutant protein shows reduced ability to degrade cAMP (Horvath, A., Mericq, V. and Stratakis, C. A. N. Engl. J. Med. 358: 750-752, 2008; Horvath, A. et al., Eur. J. Hum. Genet. 16: 1245-1253, 2008).

### Carney Complex

In Carney Complex (CNC) caused by PRKAR1A mutations, some patients also have defects in PDE11A that may exert a synergistic effect to enhance abnormal activation of the cAMP signal transduction pathway, leading to adrenal and testicular cancer (Libe, R. et al., J. Clin. Endocrinol. Metab. 96: E208-214, 2011).

### Treatment and Posology

By “treatment” herein is meant the treatment by therapy, whether of a human or a non-human animal (e.g., in veterinary applications) typically a non-human mammal, in which some desired therapeutic effect on the condition is achieved; for example, the inhibition of the progress of the disorder, including a reduction in the rate of progress, a halt in the rate of progress, amelioration of the disorder or cure of the condition. Treatment as a prophylactic measure is also included. References herein to prevention or prophylaxis do not indicate or require complete prevention of a condition; its manifestation may instead be reduced or delayed via prophylaxis or prevention according to the present invention.

Compounds or compositions as described herein, when used for preventing or treating a disorder, may be administered in an “effective amount”, which may also be referred to as a “therapeutically effective amount”. By a “therapeutically effective amount” herein is meant an amount of the one or more compounds described herein or a pharmaceutical formulation comprising such one or more compounds, which is effective for producing such a therapeutic effect, commensurate with a reasonable benefit/risk ratio.

It will be appreciated that appropriate dosages of the compounds described herein may vary from patient to patient. Determining the optimal dosage will generally involve the balancing of the level of therapeutic benefit against any risk or deleterious side effects of the treatments of the present invention. The selected dosage level will depend on a variety of factors including the activity of the particular compound, the route of administration, the time of administration, the rate of excretion of the compound, the duration of the treatment, other drugs, compounds or materials used in combination and the age, sex, weight, condition, general health and prior medical history of the patient. The amount of compound(s) and route of administration will ultimately be at the discretion of the physician, although generally the dosage will be to achieve local concentrations at the site of action so as to achieve the desired effect. Administration in vivo can be effected in one dose, continuously or intermittently throughout the course of treatment. Methods of determining the most effective means and dosage of administration are well known to a person skilled in the art and will vary with the formulation used for therapy, the purpose of the therapy, the target cell being treated, and the subject being treated. Single or multiple administrations can be carried out with the dose level and pattern being selected by the treating physician.

In general, a suitable dose of the one or more compounds described herein may be in the range of about 0.001 to 50 mg/kg body weight of the subject per day, preferably in a dosage of 0.01-25 mg per kg body weight per day, e.g., 0.01, 0.05, 0.10, 0.25, 0.50, 1.0, 2.5, 10 or 25 mg/kg per day. Where the compound(s) is a salt, solvate, prodrug or the like, the amount administered may be calculated on the basis of the parent compound and so the actual weight to be used may be increased proportionately.

### Combination Therapies

The compounds described herein may also find application in mimicking or enhancing the effects of drugs known to produce their therapeutic effect through lowering of intracellular cAMP levels.

A number of therapeutically beneficial drugs have a primary mode of action involving lowering intracellular cAMP levels and/or cAMP-mediated activity, as summarised below. Since PDE4 long form activators described herein will also act to lower cAMP levels it is expected that these agents will mimic and/or augment the pharmacological properties and therapeutic utility of drugs operating through a down-regulation of cAMP-mediated signalling. In certain embodiments, a compound described herein is therefore provided as part of a combination therapy with another agent that lowers intracellular cAMP levels and/or cAMP-mediated activity. The combination therapy may be administered simultaneously, contemporaneously, sequentially or separately. In one embodiment, the compound described herein and the separate cAMP lowering agent are provided in a single composition, as described in more detail below. The combination therapy may comprise a described herein and one or more of:
(i) a presynaptic α-2 adrenergic receptor agonist, optionally clonidine, dexmedetomidine, or guanfacine;
(ii) a β-1 Adrenergic receptor antagonist (“beta-blocker”), optionally Atenolol, Metoprolol, Bisoprolol, Acebutolol, or Betaxolol.
   Combination with α-2 Adrenergic Receptor Agonist α-2 Adrenergic receptor stimulation is known to reduce cAMP levels through a Gᵢ protein-mediated inhibition of adenylyl cyclase activity in a broad range of tissues. In noradrenergic neurones in the brain and peripheral sympathetic nervous system, presynaptic α-2 adrenergic receptor activation inhibits noradrenaline release and noradrenergic activity. Drugs (e.g. clonidine, dexmedetomidine, guanfacine) that act as agonists at these receptors are effective in the treatment of a variety of clinical conditions. Clonidine, the prototypic agent, has shown therapeutic utility in the treatment of hypertension, neuropathic pain, opioid detoxification, insomnia, ADHD, Tourette syndrome, sleep hyperhidrosis, addiction (narcotic, alcohol and nicotine withdrawal symptoms), migraine, hyperarousal, anxiety and also as a veterinary anaesthetic. Lowering of cAMP levels by PDE4 long form activation may be expected to yield similar effects to drugs acting through α-2 adrenergic receptor stimulation. Furthermore, PDE4 long form activators described herein may be expected to potentiate the pharmacodynamic effects of α-2 adrenergic receptor agonists when used in combination.
   Combination with β-1 Adrenergic receptor antagonist

β-1 Adrenergic receptor antagonists are used in the treatment a range of cardiovascular indications including hypertension, cardiac arrhythmias and cardioprotection following myocardial infarction. Their primary mechanism of action involves reducing the effects of excessive circulating adrenaline and sympathetic activity, mediated by noradrenaline, particularly at cardiac β-1 adrenergic receptors. Endogenous and synthetic β-1 adrenergic receptor agonists stimulate adenylyl cyclase activity through Gₛ activation and raise intracellular cAMP levels in a variety of tissues such as heart and kidney. Consequently, drugs that block β-1 adrenergic receptor mediated activity exert their pharmacological effects by attenuating the increase in cAMP mediated signalling. Given that PDE4 long form activation will also lower cAMP concentration and transduction in cardiac tissue, PDE4 long form activators described herein may be expected to find utility in the treatment or partial control of hypertension, cardiac arrhythmias, congestive heart failure and cardioprotection. Additional non-cardiovascular therapeutic utility may be expected in disorders such as post-traumatic stress related disorder, anxiety, essential tremor and glaucoma, which also respond to β-1 adrenergic antagonist treatment. Furthermore, PDE4 long form activators described herein may be expected to potentiate the pharmacodynamic effects of β-1 adrenergic receptor antagonists when used in combination.

### Methods of Treatment

Compounds as described here may be used for treating or preventing a disease or disorder that can be ameliorated by activation of long isoforms of PDE4. Compounds as described here may be used for treating or preventing a disease or disorder mediated by excessive intracellular cyclic AMP signalling. Compounds as described here may be used for treating or preventing a disease or disorder that can be ameliorated by activation of long isoforms of PDE4, wherein the disease or disorder that can be ameliorated by activation of long isoforms of PDE4 is a disease or disorder mediated by excessive intracellular cyclic AMP signalling. In a further aspect, the present invention provides a small molecule activator of a PDE4 long form described herein for use in a method for the treatment or prevention of a disease or disorder in a patient in need of such therapy. The invention also provides a method of treating or preventing a disease or disorder in a patient in need thereof, comprising administering to a patient in need thereof an effective amount of a compound described herein. The invention provides a method of treating or preventing a disease or disorder that can be ameliorated by activation of long isoforms of PDE4, comprising administering to a patient in need thereof a therapeutically effective amount of any compound or a pharmaceutically acceptable salt or derivative as described herein. The invention provides a method of treating or preventing a disease or disorder mediated by excessive intracellular cyclic AMP signalling, comprising administering to a patient in need thereof a therapeutically effective amount of any compound or a pharmaceutically acceptable salt or derivative as described herein. The disease or disorder may be any disease of disorder described herein, including: a disease associated with increased cAMP production and signalling (such as hyperthyroidism, Jansens's metaphyseal chondrodysplasia, hyperparathyroidism, familial male-limited precocious puberty, pituitary adenomas, Cushing's disease, polycystic kidney disease, polycystic liver disease, MODY5 and cardiac hypertrophy); diseases known to be associated with increased cAMP-mediated signalling, including disorders associated with activating mutations of the alpha subunit of the G protein (GNAS1) (such as McCune-Albright syndrome); amelioration of toxin-induced increases in adenylyl cyclase activity in infectious diseases (such as cholera, whooping cough, anthrax, and tuberculosis); treatment of diseases known to be dependent upon activation of PKA by elevated cAMP (such as HIV infection and AIDS, and Common Variable Immunodeficiency (CVID)); treatment of diseases known to be dependent upon activation of either or both of Epac1 and Epac2 by elevated cAMP (such as melanoma and pancreatic cancer); treatment of diseases dependent upon modulation of cAMP-gated ion channels by elevated cAMP; treatment of diseases known to be associated with increased activity of cAMP response element binding protein (such as leukaemia and prostate cancer); treatment of diseases known to be associated with reduced activity of cAMP-hydrolysing PDE enzymes (such as adrenocortical tumours, testicular cancer, primary pigmented nodular adrenocortical diseases (PPNAD) and Carney Complex); and mimicking or enhancing the effects of drugs known to produce their therapeutic effect through lowering of intracellular cAMP levels.

As used herein, the terms “compound of the invention”, “compound of the disclosure” “compound described herein” and “compound of Formula I”, etc, include pharmaceutically acceptable salts and derivatives thereof and polymorphs, isomers (e.g. stereoisomers and tautomers) and isotopically labelled variants thereof. For example, reference to compounds of Formula I includes pharmaceutically acceptable salts thereof. Furthermore, these terms include all the sub-embodiments of those compounds disclosed herein, including compounds of Formula II to IV, and all embodiments thereof.

A compound described herein may be provided as a solvate, for example a hydrate.

Pharmaceutically acceptable derivatives of a compound described herein include pharmaceutically acceptable esters, amides, prodrugs (e.g. a pyridine N-oxide) or isotopically labelled variants thereof. The present invention further provides pharmaceutical compositions comprising a compound described herein, including a pharmaceutically acceptable salt, solvate, ester, hydrate or amide thereof, in admixture with a pharmaceutically acceptable excipient(s), and optionally other therapeutic agents. The term “acceptable” means being compatible with the other ingredients of the composition and not deleterious to the recipient thereof. Compositions include e.g. those suitable for oral, sublingual, subcutaneous, intravenous, epidural, intrathecal, intramuscular, transdermal, intranasal, pulmonary, topical, local, or rectal administration, and the like, typically in unit dosage forms for administration.

The term “pharmaceutically acceptable salt” includes a salt prepared from pharmaceutically acceptable non-toxic acids or bases including inorganic or organic acids and bases.

Compounds which contain basic, e.g. amino, groups are capable of forming pharmaceutically acceptable salts with acids. Examples of pharmaceutically acceptable acid addition salts of the compounds described herein include acid addition salts formed with organic carboxylic acids such as acetic, lactic, tartaric, maleic, citric, pyruvic, oxalic, fumaric, oxaloacetic, isethionic, lactobionic and succinic acids; organic sulfonic acids such as methanesulfonic, ethanesulfonic, benzenesulfonic and p-toluenesulfonic acids and inorganic acids such as hydrochloric, sulfuric, phosphoric and sulfamic acids.

Compounds which contain acidic, e.g. carboxyl, groups are capable of forming pharmaceutically acceptable salts with bases. Pharmaceutically acceptable basic salts of the compounds described herein include, but are not limited to, metal salts such as alkali metal or alkaline earth metal salts (e.g. sodium, potassium, magnesium or calcium salts) and zinc or aluminium salts and salts formed with ammonia or pharmaceutically acceptable organic amines or heterocyclic bases such as ethanolamines (e.g. diethanolamine), benzylamines, N-methyl-glucamine, amino acids (e.g. lysine) or pyridine.

Hemisalts of acids and bases may also be formed, e.g. hemisulphate salts.

Pharmaceutically acceptable salts of compounds described herein may be prepared by methods well-known in the art. For a review of pharmaceutically acceptable salts, see Stahl and Wermuth, Handbook of Pharmaceutical Salts: Properties, Selection and Use (Wiley-VCH, Weinheim, Germany, 2002).

### Prodrugs

Compounds described herein may be provided as a prodrug. Prodrugs are derivatives of compounds described herein (which may have little or no pharmacological activity themselves), which can, when administered in vivo, be converted into compounds described herein.

Prodrugs can, for example, be produced by replacing functionalities present in the compounds described herein with appropriate moieties which are metabolised in vivo to form a compound described herein. The design of prodrugs is well-known in the art, as discussed in Bundgaard, Design of Prodrugs 1985 (Elsevier), The Practice of Medicinal Chemistry 2003, 2^{nd} Ed, 561-585 and Leinweber, Drug Metab. Res. 1987, 18: 379.

In vivo metabolism of prodrugs of compounds described herein may for example involve hydrolysis, oxidative metabolism or reductive metabolism of the prodrug. Examples of prodrugs of compounds described herein are amides and esters of those compounds that may be hydrolysed in vivo. For example, where the compound described herein contains a carboxylic acid group (—COOH), the hydrogen atom of the carboxylic acid group may be replaced in order to form an ester (e.g. the hydrogen atom may be replaced by C₁₋₆alkyl). Where a compound contains an alcohol group (—OH), the hydrogen atom of the alcohol group may be replaced in order to form an ester (e.g. the hydrogen atom may be replaced by —C(O)C₁₋₆alkyl). Further examples of prodrugs of compounds described herein include pyridine N-oxides that may be reductively metabolised in vivo to form compounds described herein containing a pyridine ring.

### Solvates

It may be convenient or desirable to prepare, purify, and/or handle a corresponding solvate of the compounds described herein, which may be used in the any one of the uses/methods described. The term solvate is used herein to refer to a complex of solute, such as a compound or salt of the compound, and a solvent. If the solvent is water, the solvate may be termed a hydrate, for example a mono-hydrate, di-hydrate, tri-hydrate etc, depending on the number of water molecules present per molecule of substrate.

### Isomers

It will be appreciated that the compounds described herein may exist in various isomeric forms and the compounds described herein include all stereoisomeric forms and mixtures thereof, including enantiomers and racemic mixtures. The present invention includes within its scope the use of any such stereoisomeric form or mixture of stereoisomers, including the individual enantiomers of the compounds described herein as well as wholly or partially racemic mixtures of such enantiomers. Where appropriate, isomers can be separated from their mixtures by the application or adaptation of known methods (e.g. chromatographic techniques and recrystallisation techniques). Where appropriate, isomers can be prepared by the application or adaptation of known methods (e.g. asymmetric synthesis). In addition, it will be appreciated that in some instances, compounds described herein may exist in tautomeric forms and the compounds described herein include all tautomers and mixtures thereof.

### Isotopes

The compounds described herein include pharmaceutically acceptable isotopically-labelled compounds wherein one or more atoms are replaced by atoms having the same atomic number, but an atomic mass or mass number different from the atomic mass or mass number usually found in nature.

Examples of isotopes suitable for inclusion in the compounds described herein include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, and sulphur, such as ³⁵S. Certain isotopically-labelled compounds, for example, those incorporating a radioactive isotope, are useful in drug and/or substrate tissue distribution studies. The radioactive isotopes ³H and ¹⁴C are particularly useful for this purpose in view of their ease of incorporation and ready means of detection. Substitution with positron emitting isotopes, such as ¹¹C, ¹⁸F, ¹⁵O and ¹³N, can be useful in Positron Emission Topography (PET) studies for examining substrate receptor occupancy. It is well known in the art that isotope substitution of a hydrogen atom that is bonded to carbon with deuterium [²H] may positively influence the ADME properties of drug candidates by slowing CYP-mediated metabolism [for a review see Nat. Rev. Drug Discov. 15(4): 219-21 (2016)].

Isotopically-labelled compounds can generally be prepared by conventional techniques known to those skilled in the art or by processes analogous to those described herein using an appropriate isotopically-labelled reagent in place of the non-labelled reagent previously employed.

### Pharmaceutical Compositions

A pharmaceutical composition may comprise any compound or a pharmaceutically acceptable salt or derivative as described herein, and a pharmaceutically acceptable excipient. A pharmaceutical composition as described herein may comprise one or more pharmaceutically acceptable excipients, for example pharmaceutically acceptable carriers, diluents, preserving agents, solubilising agents, stabilising agents, disintegrating agents, binding agents, lubricating agents, wetting agents, emulsifiers, sweeteners, colourants, odourants, salts, buffers, coating agents and antioxidants. Suitable excipients and techniques for formulating pharmaceutical compositions are well known in the art (see, e.g. Remington: The Science and ^{Pr}actice of Pharmacy, 20th Ed., ed. A. Gennaro, Lippincott Williams & Wilkins, 2000). Suitable excipients include, without limitation, pharmaceutical grade starch, mannitol, lactose, corn starch, magnesium stearate, stearic acid, alginic acid, sodium saccharin, talcum, cellulose, cellulose derivatives (e.g. hydroxypropylmethylcellulose, carboxymethylcellulose) glucose, sucrose (or other sugar), sodium carbonate, calcium carbonate, magnesium carbonate, sodium phosphate, calcium phosphate, gelatin, agar, pectin, liquid paraffin oil, olive oil, alcohol, detergents, emulsifiers or water (preferably sterile).

A pharmaceutical composition may further comprise an adjuvant and/or one or more additional therapeutically active agent(s).

A pharmaceutical composition may be provided in unit dosage form, will generally be provided in a sealed container and may be provided as part of a kit. Such a kit would normally (although not necessarily) include instructions for use. It may include a plurality of said unit dosage forms.

A pharmaceutical composition may be adapted for administration by any appropriate route, for example by oral, buccal or sublingual routes or parenteral routes, including subcutaneous, intramuscular, intravenous, intraperitoneal, and intradermal, rectal and topical administration, and inhalation. Such compositions may be prepared by any method known in the art of pharmacy, for example by admixing the active ingredient with a excipient(s) under sterile conditions.

For oral administration, the active ingredient may be presented as discrete units, such as tablets, capsules, powders, granulates, solutions, suspensions, and the like.

Formulations suitable for oral administration may also be designed to deliver the compounds described herein in an immediate release manner or in a rate-sustaining manner, wherein the release profile can be delayed, pulsed, controlled, sustained, or delayed and sustained or modified in such a manner which optimises the therapeutic efficacy of the said compounds. Means to deliver compounds in a rate-sustaining manner are known in the art and include slow release polymers that can be formulated with the said compounds to control their release.

Examples of rate-sustaining polymers include degradable and non-degradable polymers that can be used to release the said compounds by diffusion or a combination of diffusion and polymer erosion. Examples of rate-sustaining polymers include hydroxypropyl methylcellulose, hydroxypropyl cellulose, methyl cellulose, ethyl cellulose, sodium carboxymethyl cellulose, polyvinyl alcohol, polyvinyl pyrrolidone, xanthum gum, polymethacrylates, polyethylene oxide and polyethylene glycol.

Liquid (including multiple phases and dispersed systems) formulations include emulsions, suspensions, solutions, syrups and elixirs. Such formulations may be presented as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds described herein may also be used in fast-dissolving, fast-disintegrating dosage forms such as those described in Liang and Chen, Expert Opinion in Therapeutic Patents 2001, 11(6): 981-986.

The formulation of tablets is discussed in H. Lieberman and L. Lachman, Pharmaceutical Dosage Forms: Tablets 1980, vol. 1 (Marcel Dekker, New York).

For administration intranasally or by inhalation, the active ingredient may be presented in the form of a dry powder from a dry powder inhaler or in the form of an aerosol spray of a solution or suspension from a pressurised container, pump, spray, atomiser or nebuliser.

For parenteral administration, the pharmaceutical composition of the invention may be presented in unit-dose or multi-dose containers, e.g. injection liquids in predetermined amounts, for example in sealed vials and ampoules, and may also be stored in a freeze dried (lyophilized) condition requiring only the addition of sterile liquid carrier, e.g. water, prior to use.

For parenteral administration, the compounds described herein may be administered directly into the blood stream, into subcutaneous tissue, into muscle, or into an internal organ. Suitable means for administration include intravenous, intraarterial, intrathecal, intraventricular, intraurethral, intrasternal, intracranial, intramuscular, intrasynovial and subcutaneous. Suitable devices for administration include needle (including microneedle) injectors, needle-free injectors and infusion techniques.

Parenteral formulations are typically aqueous or oily solutions. Where the solution is aqueous, excipients such as sugars (including but not restricted to glucose, mannitol, sorbitol, etc.) salts, carbohydrates and buffering agents (preferably to a pH of from 3 to 9) may be used. For some applications, the compounds described herein may be more suitably formulated as a sterile non-aqueous solution or as a dried form to be used in conjunction with a suitable vehicle such as sterile, pyrogen-free water (WFI).

Parenteral formulations may include implants derived from degradable polymers such as polyesters (e.g. polylactic acid, polylactide, polylactide-co-glycolide, polycapro-lactone, polyhydroxybutyrate), polyorthoesters and polyanhydrides. These formulations may be administered via surgical incision into the subcutaneous tissue, muscular tissue or directly into specific organs.

The preparation of parenteral formulations under sterile conditions, for example, by lyophilisation, may readily be accomplished using standard pharmaceutical techniques well known to those skilled in the art.

The solubility of compounds described herein used in the preparation of parenteral solutions may be increased by the use of appropriate formulation techniques, such as the incorporation of co-solvents and/or solubility-enhancing agents such as surfactants, micelle structures and cyclodextrins.

Mixed with such pharmaceutically acceptable excipients, e.g. as described in the standard reference, Gennaro, A. R. et al, Remington: The Science and ^{Pr}actice of Pharmacy (21st Edition, Lippincott Williams & Wilkins, 2005, see especially Part 5: Pharmaceutical Manufacturing), the active agent may be compressed into solid dosage units, such as pills, tablets, or be processed into capsules, suppositories or patches. By means of pharmaceutically acceptable liquids the active agent can be applied as a fluid composition, e.g. as an injection preparation or as an aerosol spray, in the form of a solution, suspension, or emulsion.

For making solid dosage units, the use of conventional additives such as fillers, colorants, polymeric binders and the like is contemplated. In general any pharmaceutically acceptable additive that does not interfere with the function of the active compounds can be used. Suitable carriers with which the active agent described herein can be administered as solid compositions include lactose, starch, cellulose derivatives and the like, or mixtures thereof, used in suitable amounts. For parenteral administration, aqueous suspensions, isotonic saline solutions and sterile injectable solutions may be used, containing pharmaceutically acceptable dispersing agents and/or wetting agents, such as propylene glycol or butylene glycol.

The invention further includes a pharmaceutical composition, as hereinbefore described, in combination with packaging material suitable for said composition, said packaging material including instructions for the use of the composition for the use as hereinbefore described. In some embodiments, the one or more compounds described herein may be used in combination therapies for the treatment of the described conditions i.e., in conjunction with other therapeutic agents. For the case of active compounds combined with other therapies the two or more treatments may be given in individually varying dose schedules and via different routes.

The combination of the agents listed above with a compound described herein would be at the discretion of the physician who would select dosages using his common general knowledge and dosing regimens known to a skilled practitioner.

Where a compound described herein is administered in combination therapy with one, two, three, four or more, preferably one or two, preferably one other therapeutic agents, the compounds can be administered simultaneously or sequentially. When administered sequentially, they can be administered at closely spaced intervals (for example over a period of 5-10 minutes) or at longer intervals (for example 1, 2, 3, 4 or more hours apart, or even longer period apart where required), the precise dosage regimen being commensurate with the properties of the therapeutic agent(s).

In one embodiment, the invention provides a product comprising a compound described herein and another therapeutic agent as a combined preparation for simultaneous, separate or sequential use in therapy. In one embodiment, the therapy is the treatment or prevention of disorders where a reduction of second messenger responses mediated by cyclic 3′,5′-adenosine monophosphate (cAMP) is required. Products provided as a combined preparation include a composition comprising a compound described herein and the other therapeutic agent together in the same pharmaceutical composition, or the compound described herein and the other therapeutic agent in separate form, e.g. in the form of a kit.

In one embodiment, the invention provides a pharmaceutical composition comprising a compound of the invention and another therapeutic agent. Optionally, the pharmaceutical composition may comprise a pharmaceutically acceptable excipient, as described above.

In one embodiment, the invention provides a kit comprising two or more separate pharmaceutical compositions, at least one of which contains a compound described herein. In one embodiment, the kit comprises means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

In the combination therapies of the invention, the compound described herein and the other therapeutic agent may be manufactured and/or formulated by the same or different manufacturers. Moreover, the compound described herein and the other therapeutic may be brought together into a combination therapy: (i) prior to release of the combination product to physicians (e.g. in the case of a kit comprising the compound described herein and the other therapeutic agent); (ii) by the physician themselves (or under the guidance of the physician) shortly before administration; (iii) in the patient themselves, e.g. during sequential administration of the compound described herein and the other therapeutic agent.

### Method of Manufacture & Method of Treatment

The invention also provides the use of a compound described herein in the manufacture of a medicament for the treatment or prevention of disorders where a reduction of second messenger responses mediated by cyclic 3′,5′-adenosine monophosphate (cAMP) is required, wherein the medicament is prepared for administration with another therapeutic agent. The invention also provides the use of another therapeutic agent in the manufacture of medicament for treating a disease or condition mediated by cAMP for the treatment or prevention of disorders where a reduction of second messenger responses mediated by cAMP is required, wherein the medicament is prepared for administration with a compound described herein.

The invention also provides a compound described herein for use in the treatment or prevention of disorders where a reduction of second messenger responses mediated by cAMP is required, wherein the compound described herein is prepared for administration with another therapeutic agent. The invention also provides another therapeutic agent for use in the treatment or prevention of disorders where a reduction of second messenger responses mediated by cAMP is required, wherein the other therapeutic agent is prepared for administration with a compound described herein. The invention also provides a compound described herein for use in for the treatment or prevention of disorders where a reduction of second messenger responses mediated by cAMP is required, wherein the compound described herein is administered with another therapeutic agent. The invention also provides another therapeutic agent for use in the treatment or prevention of disorders where a reduction of second messenger responses mediated by cAMP is required, wherein the other therapeutic agent is administered with a compound described herein.

The invention also provides the use of a compound described herein in the manufacture of a medicament for the treatment or prevention of disorders where a reduction of second messenger responses mediated by cAMP is required, wherein the patient has previously (e.g. within 24 hours) been treated with another therapeutic agent. The invention also provides the use of another therapeutic agent in the manufacture of a medicament for the treatment or prevention of disorders where a reduction of second messenger responses mediated by cAMP is required, wherein the patient has previously (e.g. within 24 hours) been treated with a compound described herein.

In one embodiment, the other therapeutic agent is:
(i) a presynaptic α-2 adrenergic receptor agonist, optionally clonidine, dexmedetomidine, or guanfacine;
(ii) a β-1 Adrenergic receptor antagonist (“beta-blocker”), optionally Atenolol, Metoprolol, Bisoprolol, Acebutolol, or Betaxolol.

### EXAMPLES

The present invention will now be further described by way of the following non-limiting examples and with reference to the Tables and Figures:

Table 1 shows the structures of small molecule PDE4 long form activators, Examples 1 to 122, according to the present invention.

Table 2 shows enzyme assay data for PDE4D5, a long form of PDE4.

Table 3 shows enzyme assay data for PDE4B2, a short form of PDE4.

Table 4 shows inhibition of PGE2-stimulated cyst formation in a 3D culture of m-IMCD3 kidney cells treated with compounds of the present invention.

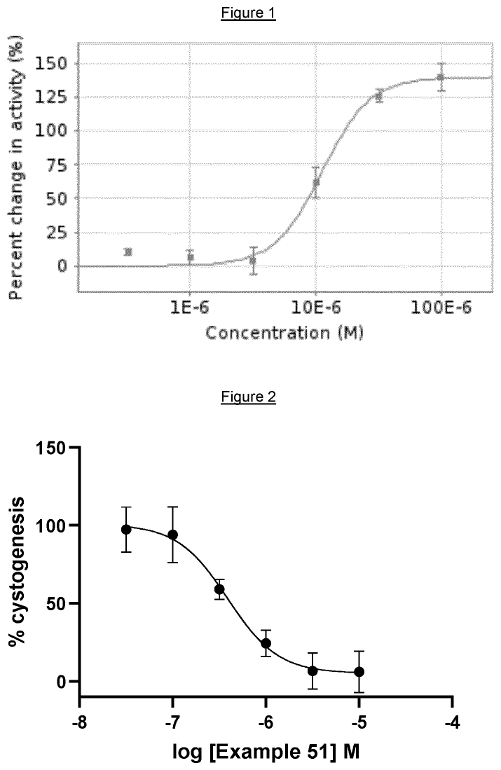

FIG. 1 shows concentration-dependent activation of a PDE4 long form, PDE4D5, by Example 4.

FIG. 2 shows concentration-dependent inhibition of PGE2-stimulated cyst formation in a 3D culture of m-IMCD3 cells treated with Example 51.

### EXPERIMENTAL DETAILS

### Preparation of Examples 1 to 122

Reactions were monitored by thin layer chromatography (Merck Millipore TLC Silica Gel 60 F₂₅₄). Flash column chromatography was performed on Biotage Isolera® or Buchi Reveleris® X2 Flash Chromatography systems using pre-packed silica gel columns. NMR spectra were recorded using Bruker 300 or 400 MHz spectrometers, using residual signal of deuterated solvent as internal reference at 25° C. Exchangeable NH and OH residues were not identifiable in the ¹H NMR spectra in some cases.

Typical preparative HPLC methods are as follows:

Method A (preparative HPLC with formic acid as buffer): MS instrument type: ACQ-SQD2; HPLC instrument type: Waters Modular Preparative HPLC system; column: Waters XSelect (C18, 100×30 mm, 10 μm); flow: 55 ml/min prep pump; column temp: RT; eluent A: 0.1% formic acid in water; eluent B: 100% acetonitrile; lin. gradient: t=0 min 2% B, t=4 min 2% B, t=13 min 30% B, t=14.5 100% B, t=17 min 100% B; detection: DAD (220-320 nm); detection: MSD (ESI pos/neg) mass range: 100-800; fraction collection based on MS and DAD

Method B (preparative HPLC with ammonium bicarbonate as buffer): MS instrument type: Agilent Technologies G6130B Quadrupole; HPLC instrument type: Agilent Technologies 1290 preparative LC; Column: Waters XSelect CSH (C18, 150×19 mm, 10p); Flow: 25 ml/min; Column temp: RT; Eluent A: 10 mM ammonium bicarbonate in water pH=9.5; Eluent B: 100% acetonitrile; lin. gradient: t=0 min 10% B, t=2.5 min 10% B, t=11 min 50% B, t=13 min 100% B, t=17 min 100% B; Detection: DAD (220-320 nm); Detection: MSD (ESI pos/neg) mass range: 100-1000; Fraction collection based on MS and DAD.

The following abbreviations are used in the experimental details: CDI (1,1′-carbonyldiimidazole), DCM (dichloromethane), DIPEA (N,N-diisopropylethylamine), DMF (dimethylformamide), EDC (N-ethyl-N′-(3-dimethylaminopropyl)carbodiimide), h (hours), HOBt (hydroxybenzotriazole), MW (microwave), r.t. (room temperature), SEM [2-(trimethylsilyl)ethoxymethyl], SFC (supercritical fluid chromatography), TBDPS (tert-butyldiphenylsilyl), THF (tetrahydrofuran). The following abbreviations are used in the assignment of NMR signals: s (singlet), d (doublet), t (triplet), q (quartet), m (multiplet), app. (approximate), br. (broad), dd (double doublet), dt (double triplet), td (triple doublet).

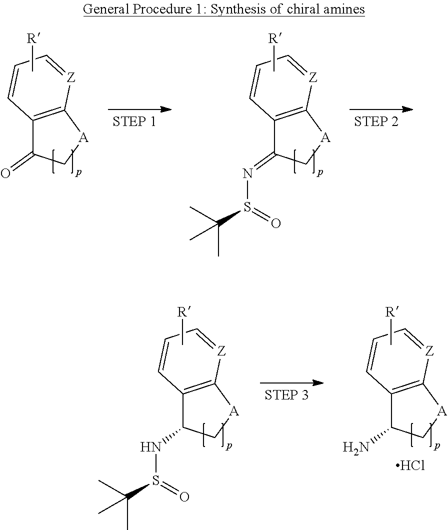

(In the above scheme, when utilised to synthesise a compound of Formula I, it will be appreciated that Z may be C or N, A may be C or a heteroatom (e.g. O), p is 1 or 2 and R′ is absent or represents one or more substituents suitable to provide a compound of Formula I.)

### Step 1:

To a stirred solution of ketone (1.0 equiv.) and (S)-2-methylpropane-2-sulfinamide (3.0 equiv.) in dry THF (0.25 M in substrate) was added titanium(IV) ethoxide (5 equiv.). The resulting mixture was stirred at 70° C. for 16 h. The reaction mixture was cooled to ambient temperature and then diluted with brine and EtOAc. The resulting suspension was filtered through Celite® and the filter cake was washed with EtOAc. The organic layer from the filtrate was separated, dried (Na₂SO₄) and concentrated under reduced pressure. The crude product was purified by flash column chromatography to afford the desired product:
(S)—N-(chroman-4-ylidene)-2-methylpropane-2-sulfinamide;
(S)—N-(6-chlorochroman-4-ylidene)-2-methylpropane-2-sulfinamide.

Similarly, the corresponding (R)-configured sulfinamides may be made using (R)-2-methylpropane-2-sulfinamide instead of (S)-2-methylpropane-2-sulfinamide.

### Step 2:

The N-sulfinyl imine (1.0 equiv.) was dissolved in wet THF (2-3% water; 0.31 M in substrate) and cooled to 0° C. Sodium borohydride (3.0 equiv.) was added in a single portion. The mixture was then stirred for 30 min at 0° C., after which time the bath temperature was allowed to gradually rise to ambient temperature. The reaction mixture was stirred at ambient temperature for 16 h (monitored by TLC). The mixture was then concentrated under reduced pressure to remove THF and diluted with DCM. The mixture was washed with water followed by brine, dried (Na₂SO₄) and concentrated under reduced pressure. The crude product was processed by flash column chromatography or/and SFC to separate the mixture of diastereoisomers and afford the required major diastereoisomer with good purity:
(S)—N—[(S)-6-chroman-4-yl]-2-methylpropane-2-sulfinamide;
(S)—N—[(S)-6-chlorochroman-4-yl]-2-methylpropane-2-sulfinamide.

Similarly, the corresponding (R,R)-configured sulfinamides may be made using the enantiomeric (R)-sulfinamide starting materials.

### Step 3:

To an ice-cold solution of sulfinamide (1 equiv.) in DCM (0.33 M in substrate) was added 4 N HCl in 1,4-dioxane (10 equiv. HCl). The resulting mixture was stirred at ambient temperature for 16 h (monitored by TLC). The mixture was then concentrated under reduced pressure to give a residue that was triturated with hexanes and dried to afford the amine hydrochloride salt as a solid:
(S)-chroman-4-amine.HCl;
(S)-6-chlorochroman-4-amine.HCl.

Similarly, the corresponding (R)-configured amines may be made using the enantiomeric (R,R)-configured sulfinamide starting materials. The amine hydrochlorides may be used in salt form without further purification for preparation of compounds in the present invention or alternatively desalted by partition between DCM and aqueous base, drying the separated organic phase (Na₂SO₄) and then recovering the free base amine by evaporation.

### Examples 1 to 4

Examples 1 to 4 may be prepared according to the route shown in Scheme 1

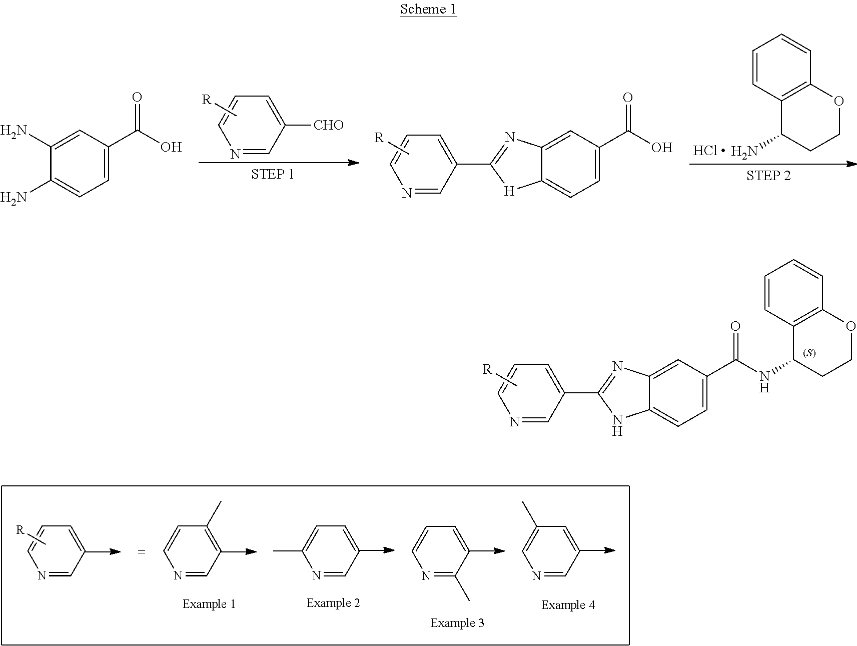

### General Procedure for Step 1 (Scheme 1):

To the solution of 3,4-diaminobenzoic acid (1.0 eq.) and Na₂S20₅ (1.3 eq.) in DMF (12 vol.), was added the nicotinaldehyde derivative (1.0 eq.). The reaction was stirred at 100° C. for 16 h, then cooled to r.t., quenched with ice water (15 vol.) and stirred for 15 min. The solid precipitate was filtered off and dried under vacuum to obtain 2-(pyridin-3-yl)-1H-benzo[d]imidazole-5-carboxylic acid derivatives as brown solids.

### General Procedure for Step 2 (Scheme 1):

To a solution of the 2-(pyridin-3-yl)-1H-benzo[d]imidazole-5-carboxylic acid derivative (1.0 eq.) in DMF (12 vol.) were added DIPEA (3.0 eq.), HOBt (2.0 eq.), and EDC.HCl (2.0 eq.) and the mixture was stirred at r.t. for 15 min. (S)-chroman-4-amine hydrochloride (1.1 eq.) was then added. The reaction mixture was stirred at r.t for 16 h, then quenched with ice water (15 vol.) and stirred for 10 min. The solid precipitate was filtered off and dried under vacuum to afford a crude product, which was purified by flash chromatography using 5% to 15% MeOH in DCM as eluent to afford the required product as an off-white solid.

### Example 1: (S)—N-(chroman-4-yl)-2-(4-methylpyridin-3-yl)-1H-benzo[d]imidazole-5-carboxamide

Prepared according to Scheme 1 using 4-methylnicotinaldehyde as the nicotinaldehyde derivative in Step 1.

¹H NMR: δ_{H} (400 MHz, DMSO-d₆) 8.94 (1H, d, J 1.6), 8.86 (1H, d, J 8.0), 8.57-8.55 (1H, m), 8.37 (1H, s), 8.16 (1H, s), 7.91 (1H, dd, J 8.4, 1.6), 7.62 (1H, d, J 8.4), 7.47-7.45 (1H, m), 7.23-7.15 (2H, m), 6.89 (1H, t, J 7.2), 6.81 (1H, d, J 8.0), 5.38-5.31 (1H, m), 4.37-4.34 (1H, m), 4.28-4.24 (1H, m), 2.66 (3H, s), 2.14-2.08 (2H, m).

### Example 2: (S)—N-(chroman-4-yl)-2-(6-methylpyridin-3-yl)-1H-benzo[d]imidazole-5-carboxamide

Prepared according to Scheme 1 using 6-methylnicotinaldehyde as the nicotinaldehyde derivative in Step 1.

¹H NMR: δ_{H} (400 MHz, DMSO-d₆) 13.23 (1H, s), 9.24 (1H, s), 8.92-8.84 (1H, m), 8.41 (1H, d, J 8.0), 8.31 (1H, s), 7.89-7.82 (1H, m), 7.73-7.59 (1H, m), 7.48 (1H, d, J 8.0), 7.22 (1H, d, J 7.6), 7.17 (1H, t, J 8.4), 6.89 (1H, t, J 7.2), 6.82 (1H, d, J 8.0), 5.35 (1H, d, J 6.4), 4.37-4.33 (1H, m), 4.29-4.24 (1H, m), 2.57 (3H, s), 2.16-2.13 (2H, m).

### Example 3: (S)—N-(chroman-4-yl)-2-(2-methylpyridin-3-yl)-1H-benzo[d]imidazole-5-carboxamide

Prepared according to Scheme 1 using 2-methylnicotinaldehyde as the nicotinaldehyde derivative in Step 1.

¹H NMR: δ_{H} (400 MHz, DMSO-d₆) 13.03 (1H, s), 8.93-8.85 (1H, m), 8.61-8.59 (1H, m), 8.37 (1H, s), 8.16 (1H, d, J 8.0), 7.91-7.85 (1H, m), 7.76 (1H, d, J 8.4), 7.47-7.43 (1H, m), 7.22 (1H, d, J 7.6), 7.17 (1H, t, J 7.6), 6.89 (1H, t, J 7.6), 6.81 (1H, d, J 8.4), 5.38-5.31 (1H, m), 4.38-4.33 (1H, m), 4.29-4.24 (1H, m), 2.83 (3H, s), 2.14-2.08 (2H, m).

### Example 4: (S)—N-(chroman-4-yl)-2-(5-methylpyridin-3-yl)-1H-benzo[d]imidazole-5-carboxamide

Prepared according to Scheme 1 using 5-methylnicotinaldehyde as the nicotinaldehyde derivative in Step 1.

¹H NMR: δ_{H} (400 MHz, DMSO-d₆) 13.28 (1H, s), 9.17 (1H, d, J 1.6), 8.85 (1H, dd, J 8.0, 2.4), 8.55 (1H, d, J 8.0), 8.35 (1H, dd, J 8.0, 2.4), 8.13 (1H, s), 7.88 (1H, dd, J 8.6, 1.2), 7.72 (1H, d, J 8.4), 7.21 (2H, d, J 7.6), 6.91 (2H, d, J 7.6), 5.35 (1H, q, J 6.8), 4.38-4.24 (2H, m), 2.49 (3H, s), 2.14-2.13 (2H, m).

### Examples 5 to 12

Examples 5 to 12 may be prepared according to the route shown in Scheme 2

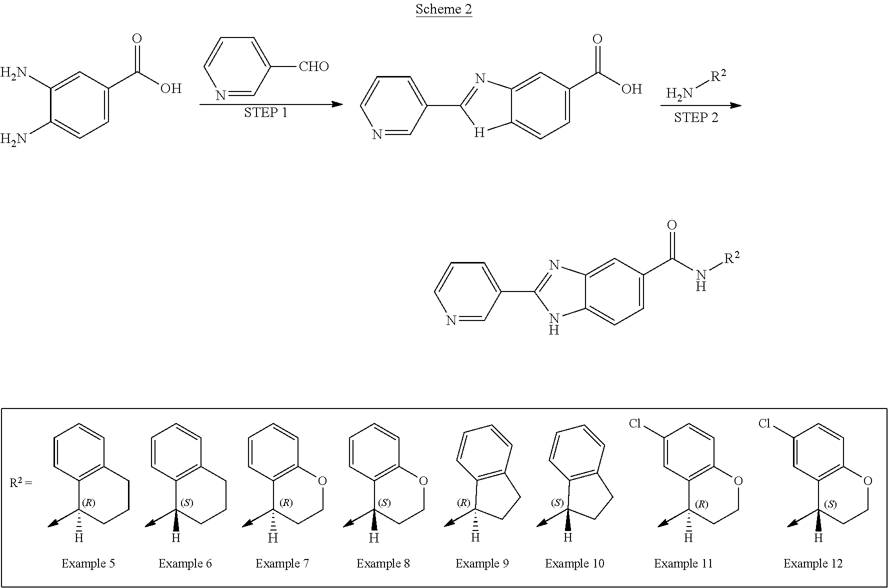

### Step 1 (Scheme 2): Synthesis of 2-(pyridin-3-yl)-1H-benzo[d]imidazole-5-carboxylic acid

To a solution of 3,4-diaminobenzoic acid (1.5 g, 9.85 mmol), in DMF (20.0 mL) were added sodium thiosulphate (2.33 g, 14.8 mmol) and nicotinaldehyde (1.25 g, 11.8 mmol) and the mixture was stirred at 100° C. for 16 h. The mixture was diluted with ice-cold water, the solid obtained was filtered off and washed with water (30 mL). The filterate was dried under vacuum to afford 2-(pyridin-3-yl)-1H-benzo[d]imidazole-5-carboxylic acid (1.2 g, 51%) as a brown solid.

### Step 2 (Scheme 2): Synthesis of Examples 5 to 12

To a solution of 2-(pyridin-3-yl)-1H-benzo[d]imidazole-5-carboxylic acid (1.0 eq.) in DMF (12 vol.) were added DIPEA (3.0 eq.), HOBt (2.0 eq.), and EDC.HCl (2.0 eq.) and the mixture was stirred at r.t. for 15 min. The amine (1.1 eq.) was added and the reaction mixture stirred at r.t. for 16 h. The reaction was quenched with ice water and stirred for 10 min. The solid precipitate was collected by filtration and purified by flash chromatography, eluting with 5% to 15% methanol in DCM to afford Examples 5 to 12 as off-white solids.

### Example 5: (R)-2-(pyridin-3-yl)-N-(1,2,3,4-tetrahvdronaphthalen-1-yl)-1H-benzo[d]imidazole-5-carboxamide

Prepared according to Scheme 2 using (1R)-1,2,3,4-tetrahydronaphthalen-1-amine as the amine in Step 2.

¹H NMR: δ_{H} (400 MHz, DMSO-d₆) 13.34 (1H, s), 9.37 (1H, s), 8.81 (1H, s), 8.71 (1H, d, J 4.0), 8.52 (1H, d, J 8.0), 8.32 (1H, br s), 7.86 (1H, s), 7.63-7.60 (2H, m), 7.25-7.16 (4H, m), 5.30 (1H, t, J 8.0), 2.79-2.67 (2H, m), 1.88-1.79 (4H, m).

### Example 6: (S)-2-(pyridin-3-yl)-N-(1,2,3,4-tetrahvdronaphthalen-1-yl)-1H-benzo[d]imidazole-5-carboxamide

Prepared according to Scheme 2 using (1S)-1,2,3,4-tetrahydronaphthalen-1-amine as the amine in Step 2.

¹H NMR: δ_{H} (400 MHz, DMSO-d₆) 13.34 (1H, s), 9.36 (1H, d, J 1.6), 8.80 (1H, d, J 8.8), 8.71 (1H, d, J 1.6), 8.53-8.50 (1H, m), 8.24 (1H, s), 7.86 (1H, d, J 8.4), 7.68-7.60 (2H, m), 7.24-7.23 (4H, m), 5.29 (1H, d, J 3.6), 2.79-2.51 (2H, m), 2.02-2.00 (2H, m), 1.88-1.79 (2H, m).

### Example 7: (R)—N-(chroman-4-yl)-2-(pyridin-3-yl)-1H-benzo[d]imidazole-5-carboxamide Prepared according to Scheme 2 using (R)-chroman-4-amine as the amine in Step 2

¹H NMR: δ_{H} (400 MHz, DMSO-d₆) 13.29 (1H, s), 9.36 (1H, s), 8.89 (1H, d, J5.6), 8.71 (1H, t, J 1.2), 8.51 (1H, d, J8.0), 8.34 (1H, br s), 7.86 (1H, d, J7.2), 7.63-7.60 (2H, m), 7.22-7.14 (2H, m), 6.88 (1H, t, J 7.2), 6.81 (1H, d, J 8.4), 5.34 (1H, q, J 6.4), 4.36-4.32 (1H, m), 4.27-4.23 (1H, m), 2.13-2.09 (2H, m).

### Example 8: (S)—N-(chroman-4-yl)-2-(pyridin-3-yl)-1H-benzo[d]imidazole-5-carboxamide

Prepared according to Scheme 2 using (S)-chroman-4-amine as the amine in Step 2.

¹H NMR: δ_{H} (400 MHz, DMSO-d₆) 13.34 (1H, s), 9.37 (1H, d, J 1.6), 8.90 (1H, d, J 8.4), 8.71 (1H, dd, J 4.8, 1.2), 8.52 (1H, d, J 8.0), 8.24 (1H, s), 7.87 (1H, d, J 8.0), 7.64-7.60 (2H, m), 7.23-7.15 (2H, m), 6.91-6.87 (2H, m), 5.34 (1H, q, J 6.8), 4.38-4.23 (2H, m), 2.14-2.10 (2H, m).

### Example 9: (R)—N-(2,3-dihydro-1H-inden-1-yl)-2-(pyridin-3-yl)-1H-benzo[d]imidazole-5-carboxamide

Prepared according to Scheme 2 using (R)-1-aminoindane as the amine in Step 2.

¹H NMR: δ_{H} (400 MHz, DMSO-d₆) 13.33 (1H, s), 9.37 (1H, d, J 1.2), 8.80 (1H, s), 8.71 (1H, d, J 3.6), 8.52 (1H, d, J 8.0), 8.29-8.22 (1H, m), 7.87 (1H, d, J 8.0), 7.71-7.60 (2H, m), 7.30-7.18 (4H, m), 5.64 (1H, d, J 8.0), 3.05-2.99 (1H, m), 2.91-2.83 (1H, m), 2.33-2.30 (1H, m), 2.07-2.02 (1H, m).

### Example 10: (S)—N-(2,3-dihydro-1H-inden-1-yl)-2-(pyridin-3-yl)-1H-benzo[d]imidazole-5-carboxamide

Prepared according to Scheme 2 using (S)-1-aminoindane as the amine in Step 2.

¹H NMR: δ_{H} (400 MHz, DMSO-d₆) 13.33 (1H, s), 9.37 (1H, s), 8.80 (1H, d, J5.6), 8.71 (1H, d, J 1.6), 8.52 (1H, dd, J 8.0, 1.6), 8.24 (1H, s), 7.86 (1H, d, J 8.0), 7.63-7.60 (2H, m), 7.27-7.18 (4H, m), 5.60 (1H, t, J 7.6), 3.33-3.32 (1H, m), 3.05-2.99 (1H, m), 2.91-2.85 (1H, m), 2.07-2.01 (1H, m).

### Example 11: (R)—N-(6-chlorochroman-4-yl)-2-(pyridin-3-yl)-1H-benzo[d]imidazole-5-carboxamide

Prepared according to Scheme 2 using (R)-6-chlorochroman-4-amine as the amine in Step 2.

¹H NMR: δ_{H} (400 MHz, DMSO-d₆) 13.32 (1H, s), 9.38 (1H, s), 8.93 (1H, d, J7.1), 8.72 (1H, d, J 4.6), 8.53 (1H, d, J 6.5), 8.26 (1H, s), 7.87 (1H, d, J 8.1), 7.64-7.61 (2H, m), 7.22 (2H, d, J 6.4), 6.86 (1H, d, J9.4), 5.34 (1H, d, J 6.9), 4.38-4.27 (2H, m), 2.15-2.14 (2H, m).

### Example 12: (S)—N-(6-chlorochroman-4-yl)-2-(pyridin-3-yl)-1H-benzo[d]imidazole-5-carboxamide

Prepared according to Scheme 2 using (S)-6-chlorochroman-4-amine as the amine in Step 2. ¹H NMR: δ_{H} (400 MHz, DMSO-d₆) 13.32 (1H, s), 9.38 (1H, s), 8.93 (1H, d, J7.1), 8.72 (1H, d, J 4.6), 8.53 (1H, d, J 6.5), 8.26 (1H, s), 7.87 (1H, d, J 8.1), 7.64-7.61 (2H, m), 7.22 (2H, d, J 6.4), 6.86 (1H, d, J9.4), 5.34 (1H, d, J 6.9), 4.38-4.27 (2H, m), 2.15-2.14 (2H, m).

### Examples 13 to 18

Examples 13 to 18 may be prepared according to the route shown in Scheme 3

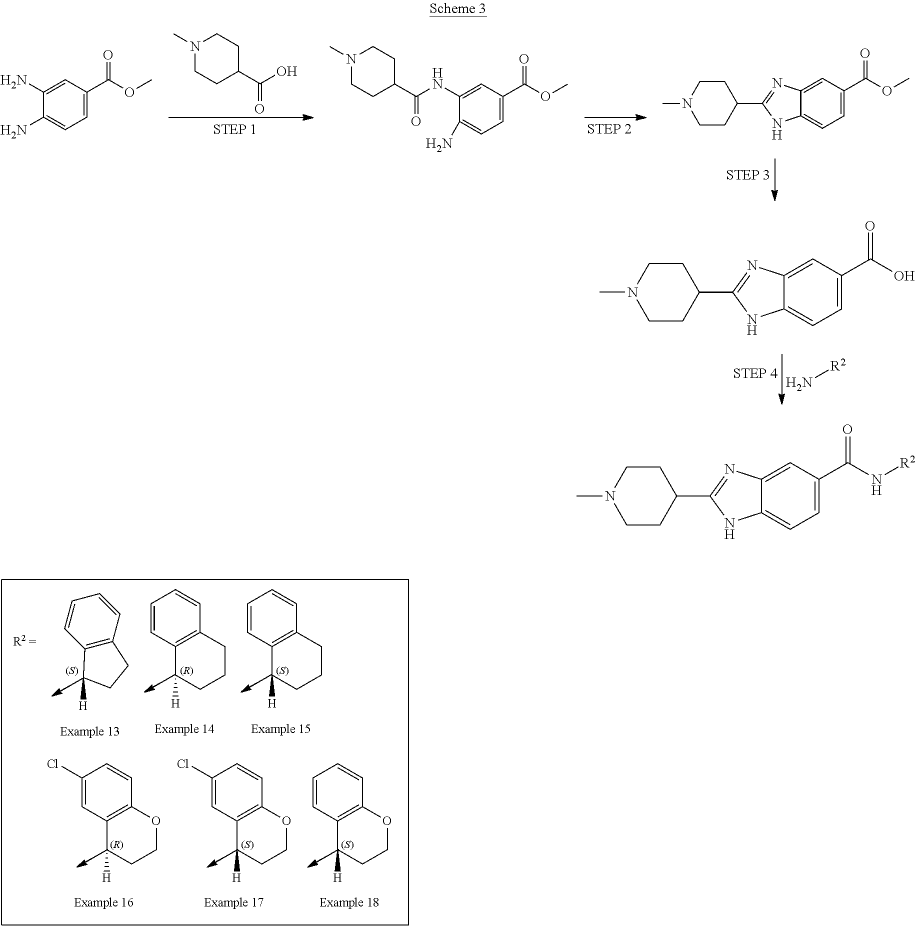

### Step 1 (Scheme 3): Synthesis of methyl 4-amino-3-(1-methylpiperidine-4-carboxamido)benzoate

To a solution of 1-methylpiperidine-4-carboxylic acid (1.0 g, 8.54 mmol) in DMF (1.0 mL) was added DIPEA (1.47 mL, 25.6 mmol), HOBt (1.73 g, 12.8 mmol), and EDC.HCl (2.44 g, 12.8 mmol). The mixture was stirred at r.t. for 15 min. Methyl 3,4-diaminobenzoate (1.41 g, 8.54 mmol) was added and the mixture stirred at r.t. for 16 h. The reaction mixture was diluted with water (10 mL) and the organic components were extracted with 10% MeOH in DCM (2×50 mL). The combined organic extract was washed with brine (10 mL), dried over anhydrous sodium sulfate, filtered and concentrated under reduced pressure. The crude product was purified by flash chromatography, eluting with 10% methanol in dichloromethane, to afford methyl 4-amino-3-(1-methylpiperidine-4-carboxamido)benzoate (0.9 g, 44%) as a brown solid.

### Step 2 (Scheme 3): Synthesis of methyl 2-(1-methylpiperidin-4-yl)-1H-benzo[d]imidazole-5-carboxylate

A solution of methyl 4-amino-3-(1-methylpiperidine-4-carboxamido)benzoate (0.90 g, 3.43 mmol) in acetic acid (15 ml) was stirred at 110° C. for 16 h. The reaction mixture was concentrated under reduced pressure to afford methyl 2-(1-methylpiperidin-4-yl)-1H-benzo[d]imidazole-5-carboxylate (0.8 g, 95%) as a brown solid.

### Step 3 (Scheme 3): Synthesis of 2-(1-methylpiperidin-4-yl)-1H-benzo[d]imidazole-5-carboxylic acid

To a solution of methyl 2-(1-methylpiperidin-4-yl)-1H-benzo[d]imidazole-5-carboxylate (0.70 g, 2.56 mmol) in THF:H₂O (3:1; 15 mL) was added LiOH·H₂O (0.21 g, 5.12 mmol) and the mixture was stirred at r.t. for 16 h. The reaction mixture was concentrated under reduced pressure to remove THF. The residue was diluted with water, acidified with 1.5 N aqueous HCl to pH 2 to 3 and concentrated under reduced pressure to afford 2-(1-methylpiperidin-4-yl)-1H-benzo[d]imidazole-5-carboxylic acid (0.6 g, 90%) as an off-white solid.

### General Procedure for Step 4 (Scheme 3): Amide Coupling

To a solution of 2-(1-methylpiperidin-4-yl)-1H-benzo[d]imidazole-5-carboxylic acid (1 eq.) in DMF (10 vol.) was added DIPEA (3 eq.), HOBt (1.5 eq.), and EDC.HCl (1.5 eq.). The mixture was stirred at r.t. for 15 min. The amine (1 eq.) was added and the reaction mixture stirred at r.t. for 16 h. The reaction was quenched with ice water and stirred for 10 min. The solid precipitate was collected by filtration and purified by preparative HPLC to afford Examples 13 to 18 as white solids.

### Example 13: (S)—N-(2,3-dihydro-1H-inden-1-yl)-2-(1-methylpiperidin-4-yl)-1H-benzo[d]imidazole-5-carboxamide

Prepared according to Scheme 3 using (S)-1-aminoindane as the amine in Step 4.

¹H NMR: δ_{H} (400 MHz, DMSO-d₆) 12.38 (1H, d, J 9.2), 8.73-8.65 (1H, m), 8.16-8.01 (1H, m), 7.78-7.72 (1H, m), 7.57-7.43 (1H, m), 7.28-7.17 (4H, m), 5.59 (1H, q, J 3.2), 3.04-2.97 (1H, m), 2.90-2.79 (4H, m), 2.20 (3H, s), 2.05-1.98 (6H, m), 1.88-1.82 (2H, m).

### Example 14: (R)-2-(1-methylpiperidin-4-yl)-N-(1,2,3,4-tetrahydronaphthalen-1-yl)-1H-benzo[d]imidazole-5-carboxamide

Prepared according to Scheme 3 using (1R)-1,2,3,4-tetrahydronaphthalen-1-amine as the amine in Step 4.

¹H NMR: δ_{H} (400 MHz, DMSO-d₆) 12.38 (1H, d, J 7.6), 8.73-8.65 (1H, m), 8.16 (1H, s), 7.79-7.72 (1H, m), 7.57-7.43 (1H, m), 7.22-7.13 (4H, m), 5.27 (1H, s), 2.86-2.78 (5H, m), 2.21 (3H, s), 2.05-1.99 (6H, m), 1.86-1.79 (4H, m).

### Example 15: (S)-2-(1-methylpiperidin-4-yl)-N-(1,2,3,4-tetrahvdronaphthalen-1-yl)-1H-benzo[d]imidazole-5-carboxamide

Prepared according to Scheme 3 using (1S)-1,2,3,4-tetrahydronaphthalen-1-amine as the amine in Step 4.

¹H NMR: δ_{H} (400 MHz, DMSO-d₆) 12.38 (1H, d, J 8.0), 8.72-8.01 (1H, m), 8.00 (1H, s), 7.79-7.72 (1H, m), 7.57-7.43 (1H, m), 7.22-7.13 (4H, m), 5.27 (1H, s), 2.86-2.78 (5H, m), 2.20 (3H, s), 2.04-1.98 (6H, m), 1.88-1.82 (4H, m).

### Example 16: (R)—N-(6-chlorochroman-4-yl)-2-(1-methylpiperidin-4-yl)-1H-benzo[d]imidazole-5-carboxamide

Prepared according to Scheme 3 using (R)-6-chlorochroman-4-amine as the amine in Step 4.

¹H NMR: δ_{H} (400 MHz, DMSO-d₆) 12.43 (1H, s), 8.86-8.79 (1H, m), 8.17-8.01 (1H, m), 7.78-7.73 (1H, m), 7.59-7.55 (1H, m), 7.22-7.17 (2H, m), 6.85 (1H, d, J 8.8), 5.31 (1H, q, J 7.2), 4.36-4.24 (2H, m), 2.92-2.83 (3H, m), 2.26 (3H, s), 2.12-2.09 (4H, m), 2.08-2.00 (2H, m), 1.91-1.85 (2H, m).

### Example 17: (S)—N-(6-chlorochroman-4-yl)-2-(1-methylpiperidin-4-yl)-1H-benzo[d]imidazole-5-carboxamide

Prepared according to Scheme 3 using (S)-6-chlorochroman-4-amine as the amine in Step 4.

¹H NMR: δ_{H} (400 MHz, DMSO-d₆) 12.42 (1H, d, J 4.8), 8.86-8.78 (1H, m), 8.18-8.01 (1H, m), 7.79-7.73 (1H, m), 7.59-7.45 (1H, m), 7.22-7.19 (2H, m), 7.17 (1H, d, J 2.0), 5.31 (1H, q, J 6.8), 4.37-4.24 (2H, m), 2.88 (3H, s), 2.24 (3H, s), 2.12-2.03 (6H, m), 2.00-1.84 (2H, m).

### Example 18: (S)—N-(chroman-4-yl)-2-(1-methylpiperidin-4-yl)-1H-benzo[d]imidazole-5-carboxamide

Prepared according to Scheme 3 using (S)-chroman-4-amine as the amine in Step 4.

¹H NMR: δ_{H} (400 MHz, DMSO-d₆) 12.39 (1H, d, J 6.0), 8.83-8.74 (1H, m), 8.16-8.01 (1H, m), 7.78-7.72 (1H, m), 7.57-7.43 (1H, m), 7.20-7.14 (2H, m), 6.87 (1H, t, J7.2), 6.80 (1H, d, J 8.0), 5.32 (1H, q, J 6.8), 4.35-4.31 (1H, m), 4.27-4.22 (1H, m), 2.86 (3H, s), 2.20 (3H, s), 2.13-2.08 (2H, m), 2.04-1.98 (4H, m), 1.88-1.82 (2H, m).

### Example 19 (S)—N-(chroman-4-yl)-2-(4-ethylpiperazin-1-yl)-1H-benzo[d]imidazole-5-carboxamide

Example 19 may be prepared according to the route shown in Scheme 4

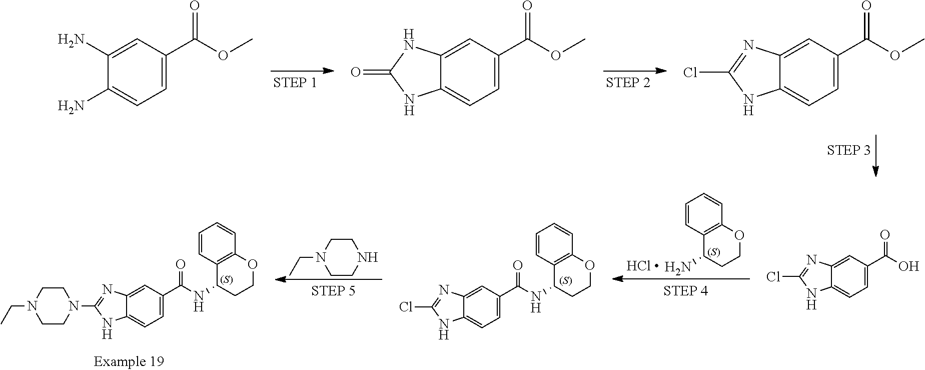

### Step 1 (Scheme 4): Synthesis of methyl 2-oxo-2,3-dihydro-1H-benzo[d]imidazole-5-carboxylate

To a solution of methyl 3,4-diaminobenzoate (25 g, 150 mmol, 1.0 eq.) in THF (250 mL), was added CDI (36.6 g, 225 mmol, 1.5 eq.). The reaction mixture was stirred at r.t. for 16 h, then quenched with ice water and the organic components were extracted twice with ethyl acetate. The combined organic layer was separated, dried over anhydrous sodium sulfate and concentrated under reduced pressure. The residue was purified by flash chromatography, eluting with 80% to 85% ethyl acetate in petroleum ether, to afford methyl 2-oxo-2,3-dihydro-1H-benzo[d]imidazole-5-carboxylate (28 g, 97%) as a solid.

### Step 2 (Scheme 4): Synthesis of methyl 2-chloro-1H-benzo[d]imidazole-5-carboxylate

POCl₃ (280 mL, 10 vol.) was added to methyl 2-oxo-2,3-dihydro-1H-benzo[d]imidazole-5-carboxylate (28 g, 192 mmol, 1.0 eq.) at 0° C. and the reaction mixture was warmed to 120° C. and stirred for 16 h, then concentrated under reduced pressure. The residue was basified with aq. NaHCO₃ solution (20 mL). The resulting solid precipitate was filtered off and dried under vacuum to afford methyl 2-chloro-1H-benzo[d]imidazole-5-carboxylate (13 g) as a brown solid.

### Step 3 (Scheme 4): Synthesis of 2-chloro-1H-benzo[d]imidazole-5-carboxylic acid

To the solution of methyl 2-chloro-1H-benzo[d]imidazole-5-carboxylate (6 g, 28.5 mmol, 1.0 eq.) in THF:water (3:1; 66 ml) was added lithium hydroxide hydrate (0.81 g, 34.2 mmol, 1.0 eq.). The reaction mixture was stirred at r.t. for 16 h, then concentrated under reduced pressure to remove THF. The residue was acidified with saturated aqueous citric acid solution to around pH 4. The resulting solid precipitate was filtered off and dried under vacuum, then triturated with ethyl acetate to afford crude 2-chloro-1H-benzo[d]imidazole-5-carboxylic acid (4.5 g), which was taken on to the next step without further purification.

### Step 4 (Scheme 4): Synthesis of (S)-2-chloro-N-(chroman-4-yl)-1H-benzo[d]imidazole-5-carboxamide

To a solution of 2-chloro-1H-benzo[d]imidazole-5-carboxylic acid (4.5 g, 22.9 mmol) in DMF (50 mL), were added DIPEA (11.8 g, 92 mmol, 4.0 eq.) and (S)-chroman-4-amine hydrochloride (3.76 g, 25.2 mmol, 1.1 eq.). The mixture was stirred for 10 min followed by the addition of n-propylphosphonic acid anhydride, cyclic trimer (14.57 g, 45.8 mmol, 2.0 eq.). The reaction mixture was stirred at r.t. for 16 h, then quenched with ice water (100 mL) and stirred for 10 min. The resulting solid precipitate was filtered off, dried under vacuum and purified by flash chromatography, eluting with 80% to 85% ethyl acetate in petroleum ether, to afford (S)-2-chloro-N-(chroman-4-yl)-1H-benzo[d]imidazole-5-carboxamide (0.25 g, 2.5%) as a pale brown solid.

### Step 5 (Scheme 4) leading to Example 19: (S)—N-(chroman-4-yl)-2-(4-ethylpiperazin-1-yl)-1H-benzo[d]imidazole-5-carboxamide

To a solution of (S)-2-chloro-N-(chroman-4-yl)-1H-benzo[d]imidazole-5-carboxamide (1 eq.) and DIPEA (4 eq.) in n-butanol (10 vol.), was added 1-ethylpiperazine (4 eq.) and the mixture was stirred at 120° C. in MW for 2 h. The reaction mixture was cooled and concentrated under reduced pressure. The residue was purified by preparative HPLC to afford the title compound as a white solid.

¹H NMR: δ_{H} (400 MHz, DMSO-d₆) 11.59 (1H, d, J 3.2), 8.67-8.62 (1H, m), 7.82 (1H, s), 7.75-7.63 (1H, m), 7.20-7.13 (3H, m), 6.88 (1H, t, J 1.2), 6.86 (1H, d, J 6.4), 5.31 (1H, d, J 7.2), 4.36-4.31 (1H, m), 4.26-4.21 (1H, m), 3.53 (4H, t, J 4.8), 2.52-2.50 (4H, m), 2.41-2.35 (2H, m), 2.12-2.08 (2H, m), 1.04 (3H, t, J 7.2).

### Examples 20 to 24

Examples 20 to 24 may be prepared according to the route shown in Scheme 5

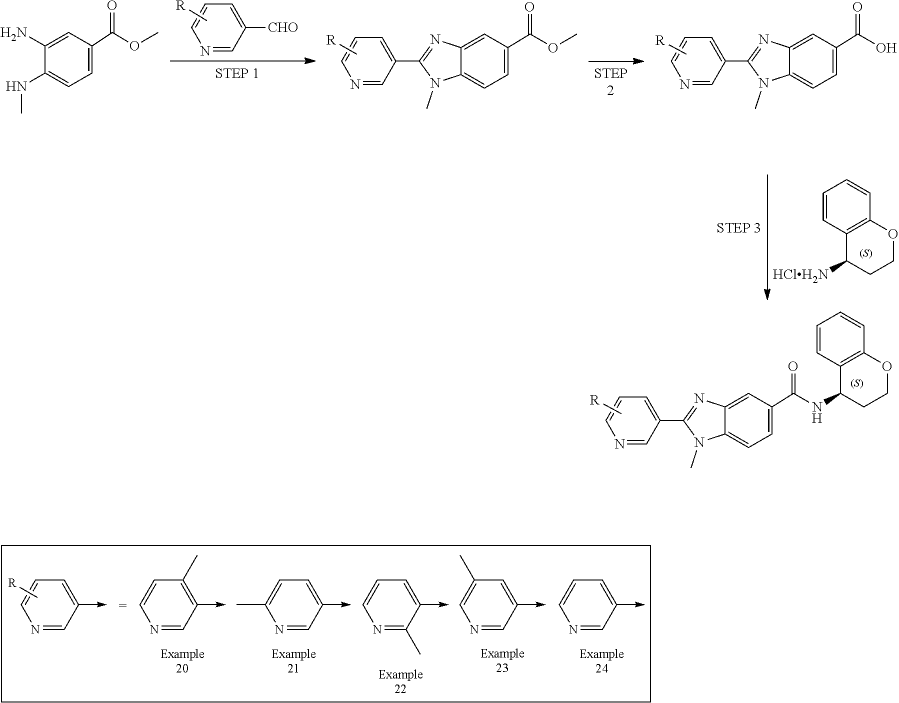

### General Procedure for Step 1 (Scheme 5): Benzimidazole Preparation

To a solution of methyl 3-amino-4-(methylamino)benzoate (1.0 eq.) and Na₂S₂O₅ (1.3 eq.) in DMF (16 vol.), was added the nicotinaldehyde derivative (1.0 eq.). The reaction was stirred at 100° C. for 16 h, then cooled to r.t. and quenched with ice water. The organic components were extracted twice with ethyl acetate. The combined organic layer was separated, dried over anhydrous Na₂SO₄ and concentrated under reduced pressure. The residue was triturated with n-pentane and dried under reduced pressure to afford the required benzimidazole ester as an off-white solid.

### General Procedure for Step 2 (Scheme 5): Ester Hydrolysis

To a THF:H₂O (3:1; 15 vol.) solution of the benzimidazole ester (1 eq.) was added LiOH·H₂O (2 eq.) and the mixture was stirred at r.t. for 16 h, then concentrated under reduced pressure to remove THF. The residue was diluted with water (5 vol.) and acidified with saturated aqueous citric acid solution to around pH 4. The resulting solid precipitate was filtered off, triturated with n-pentane and dried under vacuum to afford the required benzimidazole carboxylic acid as an off-white solid.

### General Procedure for Step 3 (Scheme 5): Amide Couplinq

To a solution of the benzimidazole carboxylic acid (1.0 eq.) in DMF (10 vol.) was added DIPEA (3.0 eq.), HOBt (2.0 eq.) and EDC.HCl (2.0 eq.). The mixture was stirred for 15 min. (S)-chroman-4-amine hydrochloride (1.1 eq.) was added and the reaction mixture was stirred at r.t. for 16 h, then quenched with ice water and the organic components were extracted twice with ethyl acetate. The combined organic layer was separated, dried over anhydrous Na₂SO₄ and concentrated under reduced pressure. The residue was purified by flash chromatography, eluting with 5% to 15% MeOH in DCM, to afford Examples 20 to 24 as off-white solids.

### Example 20: (S)—N-(chroman-4-yl)-1-methyl-2-(4-methylpyridin-3-yl)-1H-benzo[d]imidazole-5-carboxamide

Prepared according to Scheme 5 using 4-methylnicotinaldehyde as the nicotinaldehyde derivative in Step 1.

¹H NMR: δ_{H} (400 MHz, DMSO-d₆) 8.89 (1H, d, J 8.0), 8.67 (1H, s), 8.63 (1H, d, J 5.2), 8.35 (1H, d, J 1.2), 7.97 (1H, dd, J 8.4, 1.6), 7.73 (1H, d, J 8.4), 7.50 (1H, d, J 5.2), 7.20 (1H, d, J 8.0), 7.16 (1H, t, J 0.8), 6.89 (1H, t, J 6.4), 6.87 (1H, d, J 1.2), 5.35 (1H, q, J 6.8), 4.38-4.33 (1H, m), 4.29-4.24 (1H, m), 3.71 (3H, s), 2.34 (3H, s), 2.29-2.08 (2H, m).

### Example 21: (S)—N-(chroman-4-yl)-1-methyl-2-(6-methylpyridin-3-yl)-1H-benzo[d]imidazole-5-carboxamide

Prepared according to Scheme 5 using 6-methylnicotinaldehyde as the nicotinaldehyde derivative in Step 1.

¹H NMR: δ_{H} (400 MHz, DMSO-d₆) 8.94 (1H, d, J 2.0), 8.88 (1H, d, J 8.4), 8.33 (1H, d, J 1.2), 8.19 (1H, dd, J 8.0, 2.4), 7.95 (1H, dd, J 8.4, 1.6), 7.72 (1H, d, J 8.4), 7.49 (1H, d, J 8.0), 7.19 (1H, d, J 1.6), 7.16 (1H, t, J 7.2), 6.90 (1H, t, J 6.4), 6.87 (1H, d, J 1.2), 5.35 (1H, d, J 7.2), 4.37-4.28 (1H, m), 4.27-4.25 (1H, m), 3.94 (3H, s), 2.68 (3H, s), 2.16-2.10 (2H, m).

### Example 22: (S)—N-(chroman-4-yl)-1-methyl-2-(2-methylpyridin-3-yl)-1H-benzo[d]imidazole-5-carboxamide

Prepared according to Scheme 5 using 2-methylnicotinaldehyde as the nicotinaldehyde derivative in Step 1.

¹H NMR: δ_{H} (400 MHz, DMSO-d₆) 8.89 (1H, d, J 8.4), 8.67 (1H, dd, J 4.8, 1.6), 8.34 (1H, d, J 0.8), 7.98-7.94 (2H, m), 7.72 (1H, d, J 8.4), 7.46-7.43 (1H, m), 7.22 (1H, d, J 7.6), 7.18 (1H, t, J 1.2), 6.90 (1H, t, J 1.2), 6.88 (1H, d, J 6.0), 5.35 (1H, q, J 7.2), 4.38-4.33 (1H, m), 4.29-4.24 (1H, m), 3.69 (3H, s), 2.43 (3H, s), 2.16-2.10 (2H, m).

### Example 23: (S)—N-(chroman-4-yl)-1-methyl-2-(5-methylpyridin-3-yl)-1H-benzo[d]imidazole-5-carboxamide

Prepared according to Scheme 5 using 5-methylnicotinaldehyde as the nicotinaldehyde derivative in Step 1.

¹H NMR: δ_{H} (400 MHz, DMSO-d₆) 8.87 (2H, d, J 2.4), 8.62 (1H, d, J 1.2), 8.34 (1H, d, J 1.2), 8.13-8.12 (1H, m), 7.96 (1H, dd, J 8.4, 1.6), 7.75-7.73 (1H, m), 7.23-7.15 (2H, m), 6.90 (1H, t, J 6.0), 6.87 (1H, d, J 1.2), 5.35 (1H, q, J 6.4), 4.38-4.24 (2H, m), 3.95 (3H, s), 2.44 (3H, s), 2.18-2.10 (2H, m).

### Example 24: (S)—N-(chroman-4-yl)-1-methyl-2-(pyridin-3-yl)-1H-benzo[d]imidazole-5-carboxamide

Prepared according to Scheme 5 using nicotinaldehyde as the nicotinaldehyde derivative in Step 1.

¹H NMR: δ_{H} (400 MHz, DMSO-d₆) 9.08 (1H, d, J0.4), 8.89 (1H, d, J 8.0), 8.77 (1H, dd, J 4.8, 1.6), 8.35-8.30 (2H, m), 7.96 (1H, dd, J 8.8, 1.6), 7.74 (1H, d, J 8.4), 7.66-7.62 (1H, m), 7.23 (1H, d, J 7.6), 7.16 (1H, t, J 7.2), 6.90 (1H, t, J 6.4), 6.87 (1H, d, J 1.2), 5.35 (1H, q, J 6.8), 4.38-4.33 (1H, m), 4.29-4.24 (1H, m), 3.96 (3H, s), 2.19-2.08 (2H, m).

### Examples 25 to 29

Examples 25 to 29 may be prepared according to the route shown in Scheme 5, using methyl 4-amino-3-(methylamino)benzoate in Step 1 instead of methyl 3-amino-4-(methylamino)benzoate.

### Example 25: (S)—N-(chroman-4-yl)-1-methyl-2-(4-methylpyridin-3-yl)-1H-benzo[d]imidazole-6-carboxamide

Prepared according to Scheme 5 using methyl 4-amino-3-(methylamino)benzoate instead of methyl 3-amino-4-(methylamino)benzoate and using 4-methylnicotinaldehyde as the nicotinaldehyde derivative in Step 1.

¹H NMR: δ_{H} (400 MHz, DMSO-d₆) 8.88 (1H, d, J 8.4), 8.66 (1H, s), 8.64 (1H, d, J 5.2), 8.31 (1H, s), 7.92 (1H, dd, J 8.4, 1.2), 7.76 (1H, d, J 8.4), 7.50 (1H, d, J 5.2), 7.23-7.16 (2H, m), 6.90 (1H, t, J 7.2), 6.83 (1H, d, J 8.0), 5.37 (1H, q, J 6.4), 4.37-4.26 (2H, m), 3.71 (3H, s), 2.34 (3H, s), 2.18-2.08 (2H, m).

### Example 26: (S)—N-(chroman-4-yl)-1-methyl-2-(6-methylpyridin-3-yl)-1H-benzo[d]imidazole-6-carboxamide

Prepared according to Scheme 5 using methyl 4-amino-3-(methylamino)benzoate instead of methyl 3-amino-4-(methylamino)benzoate and using 6-methylnicotinaldehyde as the nicotinaldehyde derivative in Step 1.

¹H NMR: δ_{H} (400 MHz, DMSO-d₆) 8.97 (1H, d, J 1.6), 8.89 (1H, d, J 8.0), 8.31 (1H, d, J 1.2), 8.24 (1H, dd, J 8.0, 2.0), 7.92 (1H, dd, J 8.6, 1.2), 7.76 (1H, d, J 8.4), 7.53 (1H, d, J 8.4), 7.24-7.16 (2H, m), 6.91 (1H, t, J 6.4), 6.88 (1H, d, J 1.2), 5.36 (1H, q, J 6.4), 4.38-4.26 (2H, m), 3.96 (3H, s), 2.68 (3H, s), 2.20-2.16 (2H, m).

### Example 27: (S)—N-(chroman-4-yl)-1-methyl-2-(2-methylpyridin-3-yl)-1H-benzo[d]imidazole-6-carboxamide

Prepared according to Scheme 5 using methyl 4-amino-3-(methylamino)benzoate instead of methyl 3-amino-4-(methylamino)benzoate and using 2-methylnicotinaldehyde as the nicotinaldehyde derivative in Step 1.

¹H NMR: δ_{H} (400 MHz, DMSO-d₆) 8.88 (1H, d, J 8.0), 8.68-8.66 (1H, m), 8.30 (1H, s), 7.96-7.90 (2H, m), 7.75 (1H, d, J 8.8), 7.46-7.43 (1H, m), 7.23-7.16 (2H, m), 6.90 (1H, t, J 6.4), 6.88 (1H, d, J 0.8), 5.37 (1H, q, J 6.8), 4.38-4.25 (2H, m), 3.71 (3H, s), 2.43 (3H, s), 2.20-2.07 (2H, m).

### Example 28: (S)—N-(chroman-4-yl)-1-methyl-2-(5-methylpyridin-3-yl)-1H-benzo[d]imidazole-6-carboxamide

Prepared according to Scheme 5 using methyl 4-amino-3-(methylamino)benzoate instead of methyl 3-amino-4-(methylamino)benzoate and using 5-methylnicotinaldehyde as the nicotinaldehyde derivative in Step 1.

¹H NMR: δ_{H} (400 MHz, DMSO-d₆) 8.89-8.86 (2H, m), 8.62 (1H, d, J 1.2), 8.30 (1H, d, J 0.8), 8.14 (1H, t, J 0.8), 7.91 (1H, dd, J 8.4, 1.6), 7.75 (1H, d, J 8.4), 7.19 (1H, t, J 6.4), 7.16 (1H, d, J 1.2), 6.91 (1H, t, J 6.4), 6.88 (1H, d, J 0.8), 5.37 (1H, q, J 6.4), 4.38-4.26 (2H, m), 3.96 (3H, s), 2.44 (3H, s), 2.19-2.09 (2H, m).

### Example 29: (S)—N-(chroman-4-yl)-1-methyl-2-(pyridin-3-yl)-1H-benzo[d]imidazole-6-carboxamide

Prepared according to Scheme 5 using methyl 4-amino-3-(methylamino)benzoate instead of methyl 3-amino-4-(methylamino)benzoate and using nicotinaldehyde as the nicotinaldehyde derivative in Step 1.

¹H NMR: δ_{H} (400 MHz, DMSO-d₆) 9.09 (1H, d, J 1.6), 8.89 (1H, d, J 8.0), 8.78 (1H, dd, J 4.8, 1.6), 8.34-8.31 (2H, m), 7.91 (1H, dd, J 8.4, 1.6), 7.77 (1H, d, J 8.4), 7.66-7.63 (1H, m), 7.24-7.16 (2H, m), 6.91 (1H, t, J 0.8), 6.89 (1H, d, J6.4), 5.36 (1H, q, J 6.8), 4.37-4.26 (2H, m), 3.96 (3H, s), 2.18-2.09 (2H, m).

### Example 30: (S)—N-(chroman-4-yl)-2-(4-ethylpiperazin-1-yl)-1-methyl-1H-benzo[d]imidazole-6-carboxamide

Prepared according to Scheme 4 using methyl 4-amino-3-(methylamino)benzoate instead of methyl 3,4-diaminobenzoate in Step 1.

¹H NMR: δ_{H} (400 MHz, DMSO-d₆) 8.71 (1H, d, J 8.0), 8.01 (1H, s), 7.73 (1H, d, J 0.8), 7.40 (1H, d, J 8.4), 7.20-7.14 (2H, m), 6.86 (1H, t, J 6.8), 6.80 (1H, d, J 8.4), 5.31 (1H, q, J 6.4), 4.36-4.31 (1H, m), 4.27-4.22 (1H, m), 3.63 (3H, s), 3.34 (4H, s), 2.68 (4H, s), 2.41 (2H, q, J 7.2), 2.12-2.09 (2H, m), 1.05 (3H, t, J 7.2).

### Example 31: (S)—N-(chroman-4-yl)-1-methyl-2-(piperazin-1-yl)-1H-benzo[d]imidazole-6-carboxamide

Prepared according to Scheme 4 using methyl 4-amino-3-(methylamino)benzoate instead of methyl 3,4-diaminobenzoate in Step 1 and piperazine instead of 1-ethylpiperazine in Step 5.

¹H NMR: δ_{H} (400 MHz, DMSO-d₆) 8.72 (1H, d, J 8.4), 8.01 (1H, d, J 1.2), 7.74 (1H, dd, J 8.4, 1.6), 7.39 (1H, d, J 8.4), 7.20-7.13 (2H, m), 6.88 (1H, t, J 6.4), 6.85 (1H, d, J 0.8), 5.31 (1H, q, J 5.31), 4.36-4.22 (2H, m), 3.63 (3H, s), 3.18 (4H, t, J 4.4), 2.88 (4H, t, J 4.8), 2.15-2.05 (2H, m). Note: NH proton of piperazine not observed.

### Example 32: (S)—N-(chroman-4-yl)-2-(4-ethylpiperazin-1-yl)-1-methyl-1H-benzo[d]imidazole-5-carboxamide

Prepared according to Scheme 4 using methyl 3-amino-4-(methylamino)benzoate instead of methyl 3,4-diaminobenzoate in Step 1.

¹H NMR: δ_{H} (400 MHz, DMSO-d₆) 8.70 (1H, d, J 8.4), 8.02 (1H, d, J 1.2), 7.74 (1H, dd, J 8.4, 1.6), 7.40 (1H, d, J 8.4), 7.20-7.14 (2H, m), 6.89-6.79 (2H, m), 5.31 (1H, q, J 6.4), 4.32-4.31 (1H, m), 4.27-4.23 (1H, m), 3.63 (3H, s), 3.28 (4H, t, J 4.4), 2.68 (4H, s), 2.46-2.41 (2H, m), 2.12-2.07 (2H, m), 1.05 (3H, t, J 7.2).

### Example 33: (S)—N-(chroman-4-yl)-1-methyl-2-(piperazin-1-yl)-1H-benzo[d]imidazole-5-carboxamide

Prepared according to Scheme 4 using methyl 3-amino-4-(methylamino)benzoate instead of methyl 3,4-diaminobenzoate in Step 1 and piperazine instead of 1-ethylpiperazine in Step 5.

¹H NMR: δ_{H} (400 MHz, DMSO-d₆) 8.70 (1H, d, J 8.4), 8.01 (1H, d, J0.8), 7.74 (1H, dd, J 8.2, 1.2), 7.39 (1H, d, J 8.4), 7.20-7.13 (2H, m), 6.88 (1H, t, J 6.4), 6.85 (1H, d, J 1.2), 5.34-5.31 (1H, m), 4.36-4.23 (2H, m), 3.63 (3H, s), 3.17 (4H, t, J 4.4), 2.88 (4H, t, J 4.8), 2.13-2.08 (2H, m). Note: NH proton of piperazine not observed.

### Examples 34 to 39

Examples 34 to 39 may be prepared according to the route shown in Scheme 6

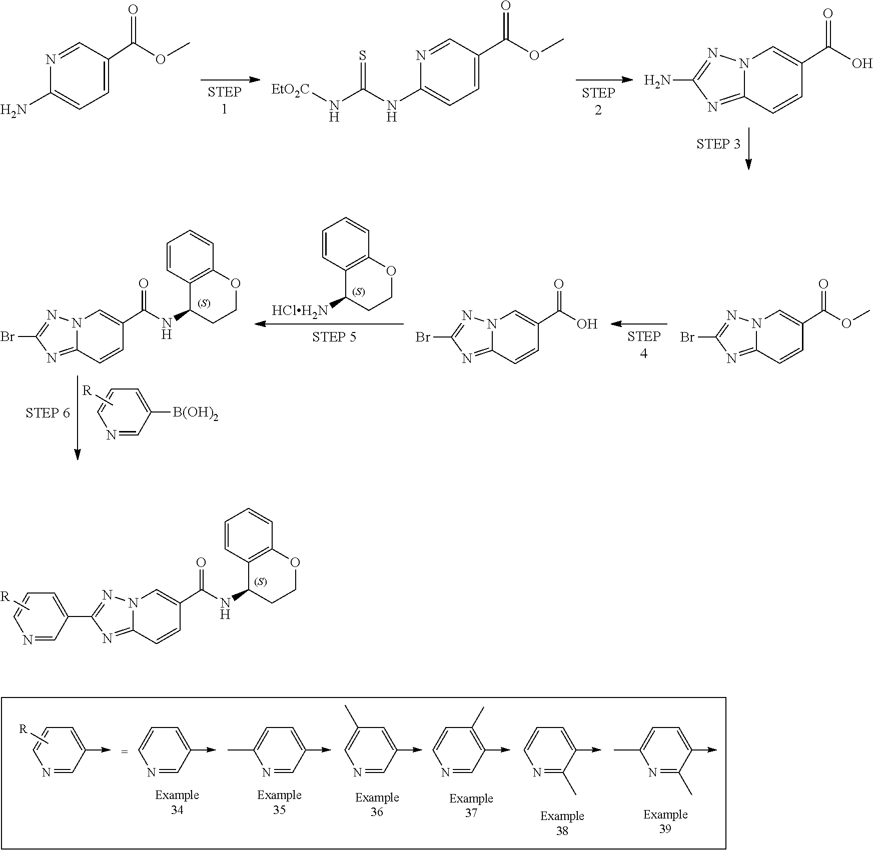

### Step 1 (Scheme 6): Synthesis of methyl 6-(3-(ethoxycarbonyl)thioureido)nicotinate

To a solution of methyl 6-aminonicotinate (10 g, 65.7 mmol) in 1,4 dioxane (100 mL) was added ethoxycarbonyl isothiocyanate (11.7 mL, 98.6 mmol). The reaction was stirred at r.t. for 16 h, then concentrated under reduced pressure. The residue was diluted with water and extracted with ethyl acetate (2×50 mL). The combined organic layer was separated, dried over anhydrous sodium sulfate and concentrated under reduced pressure to afford methyl 6-(3-(ethoxycarbonyl)thioureido)nicotinate (14 g, 77%) as a pale yellow solid.

### Step 2 (Scheme 6): Synthesis of methyl 2-amino-[1,2,4]triazolo[1,5-a]pyridine-6-carboxylate

To a stirred solution of methyl 6-(3-(ethoxycarbonyl)thioureido)nicotinate (15 g, 52.9 mmol, 1 eq.) and DIPEA (34.0 mL, 198 mmol, 4 eq.) in EtOH (150 mL), was added hydroxylamine.HCl (11.1 g, 158 mmol, 3.2 eq.). The reaction mixture was stirred at 60° C. for 16 h, then concentrated under reduced pressure. The residue was diluted with water and extracted with ethyl acetate (2×100 mL). The combined organic layer was separated, dried over anhydrous sodium sulfate and concentrated under reduced pressure to afford methyl 2-amino-[1,2,4]triazolo[1,5-a]pyridine-6-carboxylate (8.0 g, 79%) as an off-white solid.

### Step 3 (Scheme 6): Synthesis of methyl 2-bromo-[1,2,4]triazolo[1,5-a]pyridine-6-carboxylate

To a stirred solution of CuBr₂ (11.1 g, 50 mmol) and BuONO (8.58 mL, 94.4 mmol) in acetonitrile (80 mL) at 0° C. was added methyl 2-amino-[1,2,4]triazolo[1,5-a]pyridine-6-carboxylate (8.0 g, 41.7 mmol). The reaction mixture was stirred at r.t. for 2 h, then filtered through Celite®. The filtrate was diluted with water and the organic components were extracted with 5% MeOH in DCM (2×100 mL). The combined organic layer was washed with 1 N aq. HCl, dried over anhydrous sodium sulfate and concentrated under reduced pressure to afford methyl 2-bromo-[1,2,4]triazolo[1,5-a]pyridine-6-carboxylate (7.0 g, 65%).

### Step 4 (Scheme 6): Synthesis of 2-bromo-[1,2,4]triazolo[1,5-a]pyridine-6-carboxylic acid

To a solution of methyl 2-bromo-[1,2,4]triazolo[1,5-a]pyridine-7-carboxylate (2.0 g, 7.81 mmol) in THF:H₂O (3:1; 15 mL) was added LiOH·H₂O (0.98 g, 23.4 mmol). The mixture was stirred at r.t. for 16 h, then concentrated under reduced pressure to remove THF. The residue was diluted with water (5 mL) and acidified with saturated aqueous citric acid to around pH 4. The resulting solid precipitate was filtered off, triturated with n-pentane and dried under vacuum to afford 2-bromo-[1,2,4]triazolo[1,5-a]pyridine-6-carboxylic acid (1.6 g, 84%) as an off-white solid.

### Step 5 (Scheme 6): Synthesis of (S)-2-bromo-N-(chroman-4-yl)-[1,2,4]triazolo[1,5-a]pyridine-6-carboxamide

To a solution of 2-bromo-[1,2,4]triazolo[1,5-a]pyridine-6-carboxylic acid (1 eq) in DMF (5 vol.) was added (S)-chroman-4-amine (1.2 eq.) and DIPEA (4.0 eq.). The mixture was stirred for 15 min. T₃P (1.5 eq) was added and the reaction mixture was stirred at r.t. for 16 h, then quenched with ice water (10 vol.) and stirred for 10 min. The precipitate was filtered off and dried under reduced pressure to afford (S)-2-bromo-N-(chroman-4-yl)-[1,2,4]triazolo[1,5-a]pyridine-6-carboxamide as an off-white solid.

### General Procedure for Step 6 (Scheme 6): Suzuki Coupling Reaction

A solution of (S)-2-bromo-N-(chroman-4-yl)-[1,2,4]triazolo[1,5-a]pyridine-6-carboxamide (1 eq.) in THF:H₂O (9:1; 10 vol) was purged with nitrogen gas for 15 min, followed by addition of the (pyridin-3-yl)boronic acid derivative (1.2 eq.), Na₂CO₃ (3 eq.) and PdCl₂(dppf)·DCM (0.1 eq.). The reaction mixture was stirred under nitrogen at 70° C. for 16 h. The reaction mixture was cooled and filtered through Celite®. The filtrate was diluted with water and extracted twice with dichloromethane. The combined organic layer was separated, dried over anhydrous sodium sulfate and concentrated under reduced pressure. The residue was purified by preparative HPLC to afford Examples 34 to 39.

### Example 34: (S)—N-(chroman-4-yl)-2-(pyridin-3-yl)-[1,2,4]triazolo[1,5-a]pyridine-6-carboxamide

Prepared according to Scheme 6 using (pyridin-3-yl)boronic acid as the (pyridin-3-yl)boronic acid derivative in Step 6.

¹H NMR: δ_{H} (400 MHz, DMSO-d₆) 9.58 (1H, s), 9.37 (1H, s), 9.15 (1H, d, J 8.8), 8.74 (1H, d, J 6.0), 8.53 (1H, d, J 10.4), 8.19 (1H, d, J 12.0), 7.98 (1H, d, J 12.0), 7.63-7.59 (1H, m), 7.28-7.17 (2H, m), 6.93-6.82 (2H, m), 5.31 (1H, d, J 7.6), 4.30 (2H, s), 2.17 (2H, s).

### Example 35: (S)—N-(chroman-4-yl)-2-(6-methylpyridin-3-yl)-[1,2,4]triazolo[1,5-a]pyridine-6-carboxamide

Prepared according to Scheme 6 using (6-methylpyridin-3-yl)boronic acid as the (pyridin-3-yl)boronic acid derivative in Step 6.

¹H NMR: δ_{H} (400 MHz, DMSO-d₆) 9.55 (1H, s), 9.24 (1H, d, J2.4), 9.14 (1H, d, J 10.4), 8.40 (1H, dd, J 10.6, 3.2), 8.17 (1H, d, J 12.4), 7.94 (1H, d, J 12.4), 7.46 (1H, d, J 10.4), 7.27-7.17 (2H, m), 6.93-6.82 (2H, m), 5.30 (1H, q, J 8.0), 4.32-4.25 (2H, m), 2.63 (2H, s), 2.13 (3H, s).

### Example 36: (S)—N-(chroman-4-yl)-2-(5-methylpyridin-3-yl)-[1,2,4]triazolo[1,5-a]pyridine-6-carboxamide

Prepared according to Scheme 6 using (5-methylpyridin-3-yl)boronic acid as the (pyridin-3-yl)boronic acid derivative in Step 6.

¹H NMR: δ_{H} (400 MHz, DMSO-d₆) 9.56 (1H, s), 9.16 (2H, d, J 8.0), 8.58 (1H, s), 8.35 (1H, s), 8.19 (1H, d, J 8.4), 7.96 (1H, d, J9.2), 7.27-7.18 (2H, m), 6.91 (1H, t, J7.6), 6.84 (1H, d, J 8.4), 5.31 (1H, d, J 6.8), 4.30 (2H, d, J2.8), 2.33 (3H, s), 2.10-2.09 (2H, m).

### Example 37: (S)—N-(chroman-4-yl)-2-(4-methylpyridin-3-yl)-[1,2,4]triazolo[1,5-a]pyridine-6-carboxamide

Prepared according to Scheme 6 using (4-methylpyridin-3-yl)boronic acid as the (pyridin-3-yl)boronic acid derivative in Step 6.

¹H NMR: δ_{H} (400 MHz, DMSO-d₆) 9.62 (1H, s), 9.20 (1H, s), 9.17 (1H, d, J 8.0), 8.55 (1H, d, J 5.2), 8.19 (1H, dd, J9.2, 1.6), 7.99 (1H, d, J9.2), 7.44 (1H, d, J5.2), 7.26 (1H, d, J 7.6), 7.21 (1H, t, J 1.2), 6.89 (1H, t, J 6.4), 6.84 (1H, d, J 8.4), 5.30 (1H, q, J 6.0), 4.35-4.26 (2H, m), 2.72 (3H, s), 2.21-2.14 (1H, m), 2.10-2.06 (1H, m).

### Example 38: (S)—N-(chroman-4-yl)-2-(2-methylpyridin-3-yl)-[1,2,4]triazolo[1,5-a]pyridine-6-carboxamide

Prepared according to Scheme 6 using (2-methylpyridin-3-yl)boronic acid as the (pyridin-3-yl)boronic acid derivative in Step 6.

¹H NMR: δ_{H} (400 MHz, DMSO-d₆) 9.61 (1H, d, J0.4), 9.16 (1H, d, J7.6), 8.59 (1H, dd, J 4.8, 2.0), 8.45 (1H, dd, J 8.0, 1.6), 8.18 (1H, dd, J 9.2, 1.6), 7.99-7.97 (1H, m), 7.45-7.42 (1H, m), 7.22 (1H, d, J 1.6), 7.19 (1H, t, J 7.2), 6.92 (1H, t, J 0.8), 6.90 (1H, d, J 6.4), 5.30 (1H, q, J 6.0), 4.35-4.26 (2H, m), 2.90 (3H, s), 2.21-2.14 (1H, m), 2.12-2.05 (1H, m).

### Example 39: (S)—N-(chroman-4-yl)-2-(2,6-dimethylpyridin-3-yl)-[1,2,4]triazolo[1,5-a]pyridine-6-carboxamide

Prepared according to Scheme 6 using (2,6-dimethylpyridin-3-yl)boronic acid as the (pyridin-3-yl)boronic acid derivative in Step 6.

¹H NMR: δ_{H} (400 MHz, DMSO-d₆) 9.59 (1H, s), 9.15 (1H, d, J 8.0), 8.34 (1H, d, J 8.0), 8.17 (1H, dd, J9.4, 2.0), 7.95 (1H, d, J9.6), 7.29-7.18 (3H, m), 6.92-6.85 (2H, m), 5.30 (1H, q, J 6.0), 4.32-4.28 (2H, m), 3.51 (3H, s), 2.86 (3H, s), 2.21-2.14 (1H, m), 2.12-2.09 (1H, m).

### Examples 40 to 46

Examples 40 to 46 may be prepared according to the route shown in Scheme 7

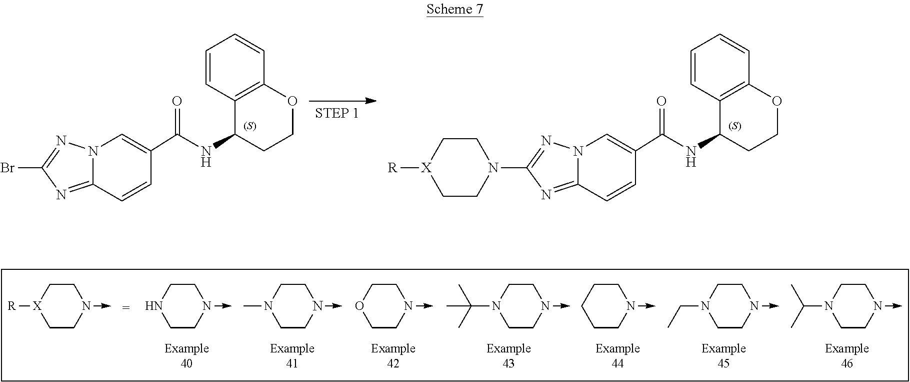

### Step 1 (Scheme 7): S_{N}Ar Reaction Leading to Examples 40 to 46

To a solution of (S)-2-bromo-N-(chroman-4-yl)-[1,2,4]triazolo[1,5-a]pyridine-6-carboxamide (1 eq.) and DIPEA (4 eq.) in n-butanol (10 vol.) was added the amine derivative (4 eq.). The mixture was stirred at 120° C. for 16 to 32 h, monitoring by TLC. Upon completion, the reaction mixture was cooled and concentrated under reduced pressure. The residue was purified by preparative HPLC to afford Examples 40 to 46 as white solids.

### Example 40: (S)—N-(chroman-4-yl)-2-(piperazin-1-yl)-[1,2,4]triazolo[1,5-a]pyridine-6-carboxamide

Prepared according to Scheme 7 using piperazine as the amine derivative.

¹H NMR: δ_{H} (400 MHz, DMSO-d₆) 9.20 (1H, d, J0.8), 8.95 (1H, d, J 8.0), 7.99 (1H, dd, J9.2, 1.6), 7.50 (1H, d, J9.2), 7.23-7.16 (2H, m), 6.89 (1H, t, J 6.4), 6.87 (1H, d, J 1.2), 5.27 (1H, q, J 6.0), 4.33-4.23 (2H, m), 3.46 (4H, t, J 4.8), 2.82 (4H, t, J 4.8), 2.18-2.03 (2H, m).

Note: NH proton of piperazine not observed.

### Example 41: (S)—N-(chroman-4-yl)-2-(4-methylpiperazin-1-yl)-[1,2,4]triazolo[1,5-a]pyridine-6-carboxamide

Prepared according to Scheme 7 using 1-methylpiperazine as the amine derivative.

¹H NMR: δ_{H} (400 MHz, DMSO-d₆) 9.20 (1H, d, J0.8), 8.95 (1H, d, J 8.0), 7.99 (1H, dd, J9.2, 2.0), 7.50 (1H, dd, J 9.2, 0.4), 7.23-7.16 (2H, m), 6.89 (1H, t, J 6.4), 6.87 (1H, d, J 1.2), 5.27 (1H, q, J 6.0), 4.30-4.26 (2H, m), 3.52 (4H, t, J 4.4), 2.34 (4H, t, J 1.6), 2.23 (3H, s), 2.15-2.03 (2H, m).

### Example 42: (S)—N-(chroman-4-yl)-2-morpholino-[1,2,4]triazolo[1,5-a]pyridine-6-carboxamide

Prepared according to Scheme 7 using morpholine as the amine derivative.

¹H NMR: δ_{H} (400 MHz, DMSO-d₆) 9.22 (1H, d, J 1.2), 8.96 (1H, d, J 8.0), 8.01 (1H, dd, J 9.2, 1.6), 7.53 (1H, dd, J 9.2, 0.4), 7.23-7.16 (2H, m), 6.89 (1H, t, J 6.4), 6.87 (1H, d, J 1.2), 5.27 (1H, q, J 6.0), 4.32-4.24 (2H, m), 3.71 (4H, t, J 4.4), 3.48 (4H, t, J 4.8), 2.16-2.02 (2H, m).

### Example 43: (S)-2-(4-(tert-butyl)piperazin-1-yl)-N-(chroman-4-yl)-[1,2,4]triazolo[1,5-a]pyridine-6-carboxamide

Prepared according to Scheme 7 using 1-tert-butylpiperazine as the amine derivative.

¹H NMR: δ_{H} (400 MHz, DMSO-d₆) 9.20 (1H, d, J0.8), 8.94 (1H, d, J 8.0), 7.98 (1H, dd, J9.2, 2.0), 7.49 (1H, dd, J 9.2, 0.8), 7.23-7.16 (2H, m), 6.89 (1H, t, J 6.4), 6.87 (1H, d, J 1.2), 5.26 (1H, t, J 6.8), 4.33-4.23 (2H, m), 3.48 (4H, t, J 4.8), 2.57 (4H, t, J5.2), 2.10 (2H, s), 1.04 (9H, s).

### Example 44: (S)—N-(chroman-4-yl)-2-(piperidin-1-yl)-[1,2,4]triazolo[1,5-a]pyridine-6-carboxamide

Prepared according to Scheme 7 using piperidine as the amine derivative.

¹H NMR: δ_{H} (400 MHz, DMSO-d₆) 9.19-9.18 (1H, m), 8.93 (1H, d, J 8.0), 7.97 (1H, dd, J9.2, 1.6), 7.47 (1H, dd, J 9.2, 0.4), 7.23-7.16 (2H, m), 6.89 (1H, t, J 6.4), 6.87 (1H, d, J 1.2), 5.27 (1H, q, J 6.0), 4.32-4.24 (2H, m), 3.52 (4H, d, J 5.6), 2.17-2.11 (1H, m), 2.08-2.01 (1H, m), 1.59 (6H, s).

### Example 45: (S)—N-(chroman-4-yl)-2-(4-ethylpiperazin-1-yl)-[1,2,4]triazolo[1,5-a]pyridine-6-carboxamide

Prepared according to Scheme 7 using 1-ethylpiperazine as the amine derivative.

¹H NMR: δ_{H} (400 MHz, DMSO-d₆) 9.20 (1H, t, J 0.8), 8.95 (1H, d, J 8.0), 7.99 (1H, dd, J 9.2, 1.6), 7.50 (1H, dd, J 9.2, 0.8), 7.23-7.16 (2H, m), 6.91-6.81 (2H, m), 5.26 (1H, t, J 7.2), 4.30-4.26 (2H, m), 3.51 (4H, t, J 4.8), 2.53-2.50 (4H, m), 2.47-2.45 (2H, m), 2.15-2.03 (2H, m), 1.03 (3H, t, J 7.2).

### Example 46: (S)—N-(chroman-4-yl)-2-(4-isopropylpiperazin-1-yl)-[1,2,4]triazolo[1,5-a]pyridine-6-carboxamide

Prepared according to Scheme 7 using 1-(propan-2-yl)piperazine as the amine derivative.

¹H NMR: δ_{H} (400 MHz, DMSO-d₆) 9.19 (1H, d, J0.8), 9.03 (1H, d, J 1.2), 8.01 (1H, dd, J9.2, 2.0), 7.45 (1H, dd, J 9.4, 0.4), 7.26 (1H, d, J 7.6), 7.20-7.16 (1H, m), 6.92 (1H, t, J 6.4), 6.90 (1H, d, J 1.2), 5.27 (1H, d, J 7.2), 4.30-4.25 (2H, m), 3.49 (4H, t, J 4.8), 2.82 (1H, s), 2.70 (4H, t, J 6.4), 2.02-2.00 (2H, m), 0.99 (6H, d, J 6.8).

### Example 47: (R)—N-(2,3-dihydro-1H-inden-1-yl)-2-(piperazin-1-yl)-[1,2,4]triazolo[1,5-a]pyridine-6-carboxamide

Prepared according to Scheme 7, using (R)-2-bromo-N-(2,3-dihydro-1H-inden-1-yl)-[1,2,4]triazolo[1,5-a]pyridine-6-carboxamide [prepared according to Scheme 6 using (R)-1-aminoindane instead of (S)-chroman-4-amine in Step 5] instead of (S)-2-bromo-N-(chroman-4-yl)-[1,2,4]triazolo[1,5-a]pyridine-6-carboxamide and piperazine as the amine derivative.

¹H NMR: δ_{H} (400 MHz, DMSO-d₆) 9.18 (1H, d, J0.8), 8.85 (1H, d, J 8.0), 7.99 (1H, dd, J9.2, 2.0), 7.49 (1H, dd, J 9.2, 0.4), 7.30-7.18 (4H, m), 5.56 (1H, d, J 8.0), 3.44 (4H, t, J 4.8), 3.02-3.00 (1H, m), 2.99-2.91 (1H, m), 2.87 (4H, t, J 8.0), 2.56-2.50 (1H, m), 2.01-1.96 (1H, m).

### Example 48: (S)—N-(2,3-dihydro-1H-inden-1-yl)-2-(piperazin-1-yl)-[1,2,4]triazolo[1,5-a]pyridine-6-carboxamide

Prepared according to Scheme 7, using (S)-2-bromo-N-(2,3-dihydro-1H-inden-1-yl)-[1,2,4]triazolo[1,5-a]pyridine-6-carboxamide [prepared according to Scheme 6 using (S)-1-aminoindane instead of (S)-chroman-4-amine in Step 5] instead of (S)-2-bromo-N-(chroman-4-yl)-[1,2,4]triazolo[1,5-a]pyridine-6-carboxamide and piperazine as the amine derivative. The title compound was isolated as a trifluoroacetate salt.

¹H NMR: δ_{H} (400 MHz, DMSO-d₆) 9.32 (2H, s), 9.25 (1H, s), 8.94 (1H, d, J 8.0), 8.05 (1H, d, J9.2), 7.57 (1H, d, J9.2), 7.28-7.18 (4H, m), 5.56 (1H, q, J 8.0), 3.76 (4H, t, J 4.4), 3.22 (4H, s), 3.05-3.01 (1H, m), 2.99-2.85 (1H, m), 2.51-2.51 (1H, m), 2.04-1.98 (1H, m).

### Example 49: (S)—N-(chroman-4-yl)-2-(piperidin-4-yl)-[1,2,4]triazolo[1,5-a]pyridine-6-carboxamide

Example 49 may be prepared according to the route shown in Scheme 8

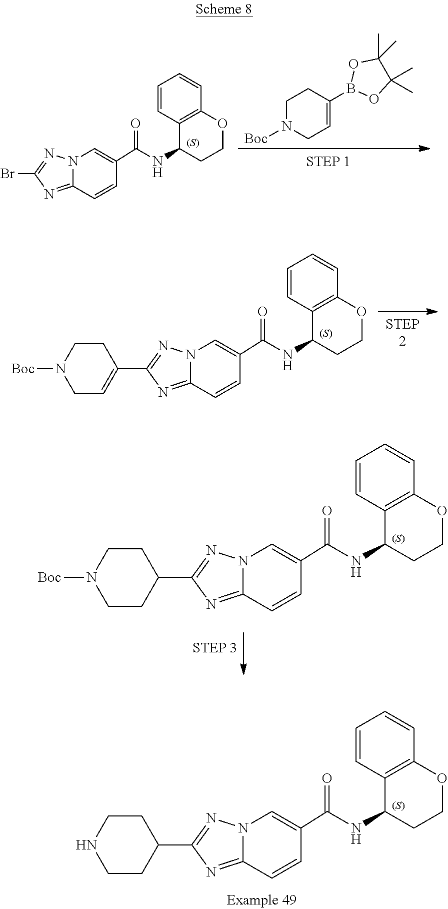

### Step 1 (Scheme 8): Synthesis of tert-butyl (S)-4-(6-(chroman-4-ylcarbamoyl)-[1,2,4]triazolo[1,5-a]pyridin-2-yl)-3,6-dihydropyridine-1 (2H)-carboxylate

To a de-gassed solution of (S)-2-bromo-N-(chroman-4-yl)-[1,2,4]triazolo[1,5-a]pyridine-6-carboxamide (1.0 g, 2.6 mmol, 1 eq.) in dioxane (27 mL) and H₂O (3 mL) under argon were added tert-butyl 4-(4,4,5,5-tetramethyl-1,3,2-dioxaborolan-2-yl)-3,6-dihydropyridine-1 (2H)-carboxylate (1.24 g, 4.01 mmol, 1.5 eq.), K₂CO₃ (1.10 g, 8.01 mol, 3 eq.) and Pd(PPh₃)₄ (0.3 g, 0.26 mol, 0.1 eq.). The mixture was heated to reflux under argon for 16 h, then cooled to r.t. and filtered through Celite®, washing with 5% MeOH in DCM. The filtrate was concentrated under reduced pressure. The residue was dissolved in 5% MeOH in DCM and washed with water followed by brine. The organic layer was dried over anhydrous Na₂SO₄ and concentrated under reduced pressure. The crude product was purified by flash chromatography, eluting with 70% EtOAc in hexanes to obtain tert-butyl (S)-4-(6-(chroman-4-ylcarbamoyl)-[1,2,4]triazolo[1,5-a]pyridin-2-yl)-3,6-dihydropyridine-1 (2H)-carboxylate (1.1 g, 86%) as a light brown solid.

### Step 2 (Scheme 8): Synthesis of tert-butyl (S)-4-(6-(chroman-4-ylcarbamoyl)-[1,2,4]triazolo[1,5-a]pyridin-2-yl)piperidine-1-carboxylate

To a de-gassed solution of tert-butyl (S)-4-(6-(chroman-4-ylcarbamoyl)-[1,2,4]triazolo[1,5-a]pyridin-2-yl)-3,6-dihydropyridine-1 (2H)-carboxylate (1.2 g, 2.5 mmol, 1 eq.) in ethanol (12 mL) under argon was added Pd/C (10%, dry; 120 mg). The mixture was stirred at ambient temperature under H₂ bladder pressure for 16 h. The reaction mixture was filtered through Celite®, washing with MeOH and the filtrate was concentrated under reduced pressure. The solid residue was triturated with hexanes and dried under reduced pressure to obtain tert-butyl (S)-4-(6-(chroman-4-ylcarbamoyl)-[1,2,4]triazolo[1,5-a]pyridin-2-yl)piperidine-1-carboxylate (1.1 g, 92%) as a brown solid.

### Step 3 (Scheme 8) leading to Example 49: Synthesis of (S)—N-(chroman-4-yl)-2-(piperidin-4-yl)-[1,2,4]triazolo[1,5-a]pyridine-6-carboxamide

To a solution of tert-butyl (S)-4-(6-(chroman-4-ylcarbamoyl)-[1,2,4]triazolo[1,5-a]pyridin-2-yl)piperidine-1-carboxylate (0.13 g, 0.27 mmol) in DCM (0.2 M in substrate) at 0° C. was added HCl in 1,4-dioxane (4 M; 8.0 equiv. HCl). The reaction was stirred at r.t. for 16 h, then concentrated under reduced pressure. The residue was purified by preparative HPLC to afford (S)—N-(chroman-4-yl)-2-(piperidin-4-yl)-[1,2,4]triazolo[1,5-a]pyridine-6-carboxamide hydrochloride salt (0.05 g, 45%) as an off-white solid.

¹H NMR: δ_{H} (400 MHz, DMSO-d₆) 9.46 (1H, d, J 0.4), 9.11 (1H, d, J 8.0), 8.98 (1H, s), 8.81 (1H, d, J 7.6), 8.12 (1H, dd, J9.2, 1.6), 7.84 (1H, d, J9.2), 7.24-7.17 (2H, m), 6.90 (1H, t, J 6.4), 6.87 (1H, d, J 1.2), 5.29 (1H, d, J 7.2), 4.31-4.27 (2H, m), 3.34-3.25 (3H, m), 3.12-3.06 (2H, m), 2.22-2.15 (3H, m), 2.09-2.06 (3H, m).

### Example 50: (S)—N-(chroman-4-yl)-2-(1-methylpiperidin-4-yl)-[1,2,4]triazolo[1,5-a]pyridine-6-carboxamide

Example 50 may be prepared according to the route shown in Scheme 9

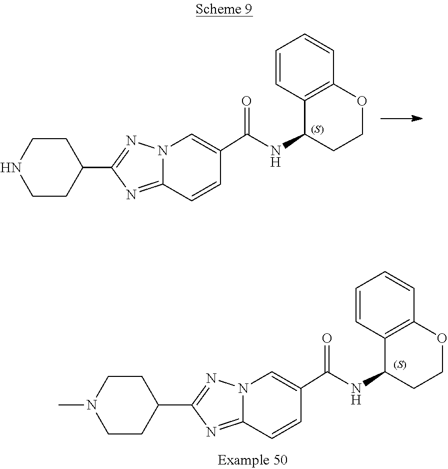

To a solution of (S)—N-(chroman-4-yl)-2-(piperidin-4-yl)-[1,2,4]triazolo[1,5-a]pyridine-6-carboxamide hydrochloride (0.15 g, 0.39 mmol) and acetic acid (0.1 mL) in methanol (5.0 mL) was added formaldehyde (37% in H₂O; 0.053 g, 0.59 mmol). The mixture was stirred at r.t. for 5 h, then NaCNBH₃ (0.049 g, 0.79 mmol) was added and stirring continued for 10 h at r.t. The reaction mixture was concentrated under reduced pressure, water was added and the organic components were extracted with EtOAc (2×10 mL). The combined organic layer was separated, dried over anhydrous Na₂SO₄ and concentrated under reduced pressure. The crude product was purified by preparative HPLC using NH₄HCO₃ as buffer to afford (S)—N-(chroman-4-yl)-2-(1-methylpiperidin-4-yl)-[1,2,4]triazolo[1,5-a]pyridine-6-carboxamide (0.05 g, 33%) as an off-white solid.

¹H NMR: δ_{H} (400 MHz, DMSO-d₆) 9.42 (1H, d, J0.4), 9.07 (1H, d, J 8.0), 8.08 (1H, dd, J9.2, 1.6), 7.80 (1H, dd, J 9.2, 0.8), 7.25-7.17 (2H, m), 6.90 (1H, t, J 6.4), 6.87 (1H, d, J 1.2), 5.28 (1H, q, J 6.0), 4.33-4.26 (2H, m), 2.83-2.80 (3H, m), 2.19-2.12 (4H, m), 2.10-2.00 (5H, m), 1.99-1.84 (2H, m).

### Example 51: (S)—N-(chroman-4-yl)-2-(1-ethylpiperidin-4-yl)-[1,2,4]triazolo[1,5-a]pyridine-6-carboxamide

Example 51 may be prepared according to the route shown in Scheme 10

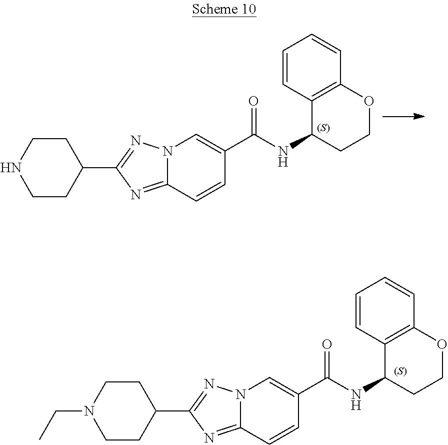

To a solution of (S)—N-(chroman-4-yl)-2-(piperidin-4-yl)-[1,2,4]triazolo[1,5-a]pyridine-6-carboxamide hydrochloride (0.15 g, 0.39 mmol) and acetic acid (0.1 mL) in methanol (5.0 mL) was added acetaldehyde (20-30% in H₂O; 0.078 g, 0.59 mmol). The mixture was stirred at r.t. for 5 h, then NaCNBH₃ (0.049 g, 0.79 mmol) was added and stirring continued for 10 h at r.t. The reaction mixture was concentrated under reduced pressure, water was added and the organic components were extracted with EtOAc (2×10 mL). The combined organic layer was separated, dried over anhydrous Na₂SO₄ and concentrated under reduced pressure. The crude product was purified by prepatative HPLC using NH₄HCO₃ as buffer to afford (S)—N-(chroman-4-yl)-2-(1-ethylpiperidin-4-yl)-[1,2,4]triazolo[1,5-a]pyridine-6-carboxamide (0.05 g, 31%) as an off-white solid.

¹H NMR: δ_{H} (400 MHz, DMSO-d₆) 9.42 (1H, s), 9.06 (1H, d, J 8.0), 8.08 (1H, dd, J9.4, 1.2), 7.80 (1H, d, J9.2), 7.25-7.17 (2H, m), 6.89 (1H, t, J 7.2), 6.83 (1H, d, J 8.0), 5.28 (1H, q, J 6.0), 4.33-4.25 (2H, m), 2.93-2.83 (3H, m), 2.37-2.32 (2H, m), 2.19-2.13 (1H, m), 2.10-2.01 (5H, m), 1.85-1.76 (2H, m), 1.02 (3H, t, J 7.2).

### Examples 52 and 53

Examples 52 and 53 may be prepared according to the route shown in Scheme 11.

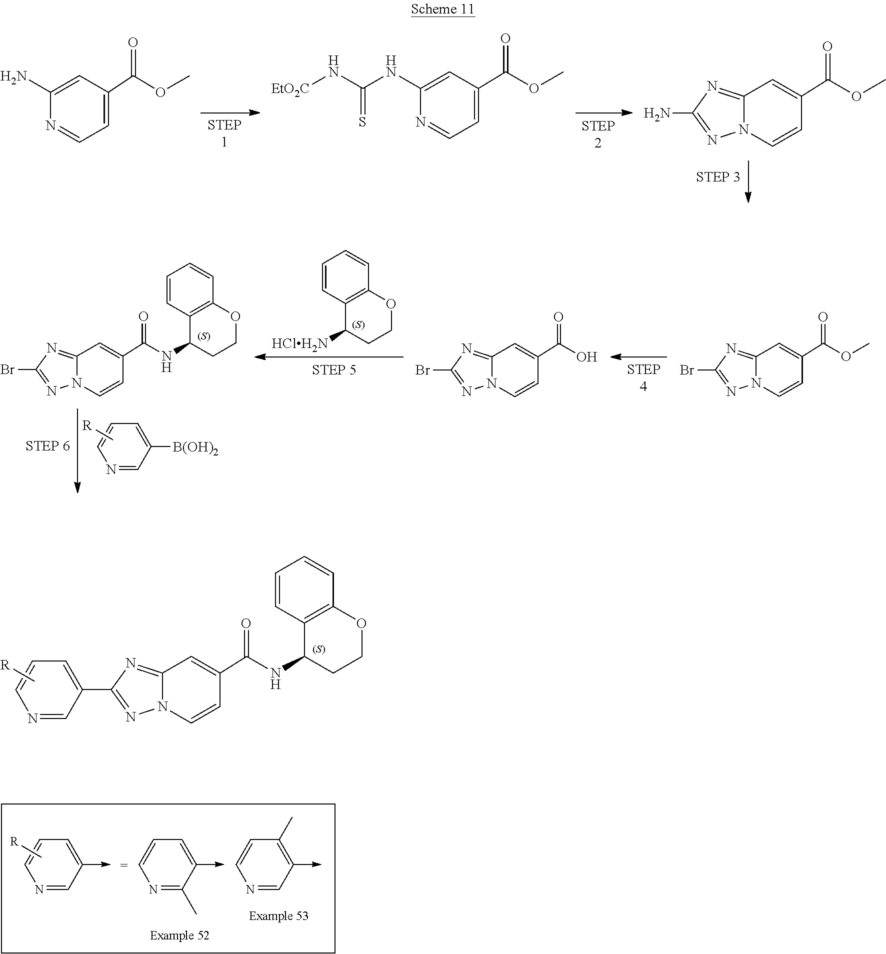

### Step 1 (Scheme 11): Synthesis of methyl 2-(3-(ethoxycarbonyl)thioureido)isonicotinate

To the solution of methyl 2-aminoisonicotinate (10 g, 65.7 mmol) in 1,4 dioxane (100 mL) was added ethoxycarbonyl isothiocyanate (11.7 ml, 98.6 mmol). The reaction was stirred at r.t. for 16 h, then concentrated under reduced pressure. The residue was diluted with water and extracted with ethyl acetate (2×50 mL). The combined organic layer was separated, dried over anhydrous sodium sulfate and concentrated under reduced pressure to afford methyl 2-(3-(ethoxycarbonyl)thioureido)isonicotinate (14 g, 77%) as a pale yellow solid.

### Step 2 (Scheme 11): Synthesis of methyl 2-amino-[1,2,4]triazolo[1,5-a]pyridine-7-carboxylate

To a stirred solution of methyl 2-(3-(ethoxycarbonyl)thioureido)isonicotinate (14 g, 49.4 mmol, 1 eq.) and DIPEA (34.0 ml, 198 mmol, 4 eq.) in EtOH (150 mL), was added hydroxylamine·HCl (11.1 g, 158 mmol, 3.2 eq.). The reaction mixture was stirred at 60° C. for 16 h, then concentrated under reduced pressure. The residue was diluted with water and extracted with ethyl acetate (2×100 mL). The combined organic layer was separated, dried over anhydrous sodium sulfate and concentrated under reduced pressure to afford methyl 2-amino-[1,2,4]triazolo[1,5-a]pyridine-7-carboxylate (8.0 g, 84%) as an off-white solid.

### Step 3 (Scheme 11): Synthesis of methyl 2-bromo-[1,2,4]triazolo[1,5-a]pyridine-7-carboxylate

To a stirred solution of CuBr₂ (11.1 g, 50 mmol) and BuONO (8.58 mL, 94.4 mmol) in acetonitrile (80 mL) at 0° C. was added methyl 2-amino-[1,2,4]triazolo[1,5-a]pyridine-7-carboxylate (8.0 g, 41.7 mmol). The reaction mixture was stirred at r.t. for 2 h, then filtered through Celite®. The filtrate was diluted with water and the organic components were extracted with 5% MeOH in DCM (2×100 mL). The combined organic layer was washed with 1 N aq. HCl, dried over anhydrous sodium sulfate and concentrated under reduced pressure to afford methyl 2-bromo-[1,2,4]triazolo[1,5-a]pyridine-7-carboxylate (5.5 g, 51%) as an off-white solid.

Step 4 (Scheme 11): Synthesis of 2-bromo-[1,2,4]triazolo[1,5-a]pyridine-7-carboxylic acid To a solution of methyl 2-bromo-[1,2,4]triazolo[1,5-a]pyridine-7-carboxylate (2.0 g, 7.81 mmol in THF:H₂O (3:1; 15 mL) was added LiOH·H₂O (0.98 g, 23.4 mmol). The mixture was stirred at r.t. for 16 h, then concentrated under reduced pressure to remove THF. The residue was diluted with water (5 mL) and acidified with saturated aqueous citric acid to around pH 4. The resulting solid precipitate was filtered off, triturated with n-pentane and dried under vacuum to afford 2-bromo-[1,2,4]triazolo[1,5-a]pyridine-7-carboxylic acid (1.6 g, 84%) as an off-white solid.

### Step 5 (Scheme 11): Synthesis of (S)-2-bromo-N-(chroman-4-yl)-[1,2,4]triazolo[1,5-a]pyridine-7-carboxamide

To a solution of 2-bromo-[1,2,4]triazolo[1,5-a]pyridine-7-carboxylic acid (1.6 g, 6.61 mmol) in DMF (6.0 mL) was added (S)-chroman-4-amine hydrochloride (1.46 g, 7.93 mmol) and DIPEA (4.5 ml, 26.4 mmol). The mixture was stirred for 15 min. T₃P (6.30 g, 9.91 mmol) was added and the reaction mixture was stirred at r.t. for 16 h, then quenched with ice water (20 mL) and stirred for 10 min. The precipitate was filtered off and dried under reduced pressure to afford (S)-2-bromo-N-(chroman-4-yl)-[1,2,4]triazolo[1,5-a]pyridine-7-carboxamide (1.7 g, 69%) as an off-white solid.

### General Procedure for Step 6 (Scheme 11): Suzuki Coupling Reaction

A solution of (S)-2-bromo-N-(chroman-4-yl)-[1,2,4]triazolo[1,5-a]pyridine-7-carboxamide (1 eq.) in THF:H₂O (9:1; 10 vol) was purged with nitrogen gas for 15 min, followed by addition of the (pyridin-3-yl)boronic acid derivative (1.2 eq.), Na₂CO₃ (3 eq.) and PdCl₂(dppf)·DCM (0.1 eq.). The reaction mixture was stirred under nitrogen at 70° C. for 16 h. The reaction mixture was cooled and filtered through Celite®. The filtrate was diluted with water and extracted twice with dichloromethane. The combined organic layer was separated, dried over anhydrous sodium sulfate and concentrated under reduced pressure. The residue was purified by preparative HPLC to afford Examples 52 and 53.

### Example 52: (S)—N-(chroman-4-yl)-2-(2-methylpyridin-3-yl)-[1,2,4]triazolo[1,5-a]pyridine-7-carboxamide

Prepared according to Scheme 11 using (2-methylpyridin-3-yl)boronic acid as the (pyridin-3-yl)boronic acid derivative in Step 6.

¹H NMR: δ_{H} (400 MHz, DMSO-d₆) 9.27 (1H, d, J 8.0), 9.13 (1H, dd, J 7.2, 0.8), 8.59 (1H, dd, J 4.6, 2.0), 8.46-8.41 (2H, m), 7.69 (1H, dd, J7.2, 1.6), 7.44-7.41 (1H, m), 7.26-7.17 (1H, m), 6.92 (1H, t, J 1.2), 6.90 (1H, d, J 6.0), 5.32 (1H, q, J 6.4), 4.36-4.26 (2H, m), 2.90 (3H, s), 2.21-2.08 (2H, m). Note: Amide NH proton was not observed.

### Example 53: (S)—N-(chroman-4-yl)-2-(4-methylpyridin-3-yl)-[1,2,4]triazolo[1,5-a]pyridine-7-carboxamide

Prepared according to Scheme 11 using (4-methylpyridin-3-yl)boronic acid as the (pyridin-3-yl)boronic acid derivative in Step 6.

¹H NMR: δ_{H} (400 MHz, DMSO-d₆) 9.26 (1H, d, J 8.0), 9.18 (1H, s), 9.13 (1H, d, J 7.2), 8.55 (1H, d, J 5.2), 8.47 (1H, s), 7.70 (1H, dd, J 7.2, 2.0), 7.44 (1H, d, J 5.2), 7.25 (1H, d, J 7.2), 7.20 (1H, t, J 8.8), 6.91 (1H, t, J 7.6), 6.84 (1H, d, J 8.0), 5.32 (1H, q, J 6.8), 4.36-4.26 (2H, m), 2.72 (3H, s), 2.20-2.10 (2H, m).

### Example 54: (S)—N-(chroman-4-yl)-2-(4-hydroxypiperidin-1-yl)-[1,2,4]triazolo[1,5-a]pyridine-7-carboxamide

Example 51 may be prepared according to the route shown in Scheme 12.

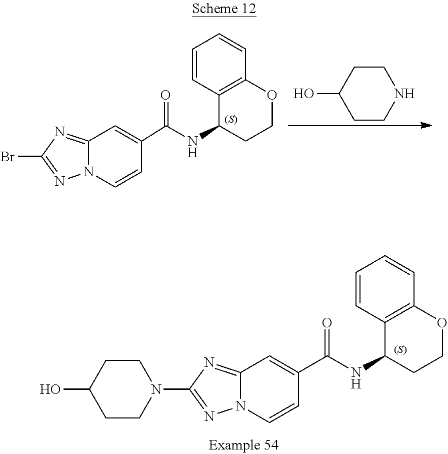

To a de-gassed solution of (S)-2-bromo-N-(chroman-4-yl)-[1,2,4]triazolo[1,5-a]pyridine-7-carboxamide (0.1 g, 0.26 mmol) in toluene (10 mL) under nitrogen were added 4-hydroxypiperidine (0.032 g, 0.32 mmol), Cs₂CO₃ (0.17 g, 0.32 mmol), BINAP (0.031 g, 0.052 mmol) and Pd(OAc)₂ (0.006 g, 0.026 mmol). The reaction was stirred at 110° C. for 16 h. The mixture was cooled to r.t., filtered through Celite®, washing with 10% MeOH/DCM and the filtrate was concentrated under reduced pressure. The residue was diluted with water (10 mL) and extracted with DCM (2×10 mL). The combined organic layer was washed with brine, dried over anhydrous Na₂SO₄ and concentrated under reduced pressure. The residue was purified by preparative HPLC using NH₄HCO₃ as buffer to afford (S)—N-(chroman-4-yl)-2-(4-hydroxypiperidin-1-yl)-[1,2,4]triazolo[1,5-a]pyridine-7-carboxamide (0.03 g, 30%).

¹H NMR: δ_{H} (400 MHz, DMSO-d₆) 9.12 (1H, d, J 8.0), 8.72 (1H, d, J 7.2), 7.96 (1H, s), 7.40 (1H, dd, J7.2, 1.6), 7.21-7.16 (2H, m), 6.89 (1H, t, J7.2), 6.82 (1H, d, J8.0), 5.28 (1H, d, J 6.8), 5.10-4.51 (1H, m), 4.31-4.25 (2H, m), 3.95-3.90 (2H, m), 3.72-3.67 (1H, m), 3.20-3.13 (2H, m), 2.15-2.06 (2H, m), 1.83-1.79 (2H, m), 1.45-1.37 (2H, m).

### Examples 55 to 61

Examples 55 to 61 may be prepared according to the route shown in Scheme 13.

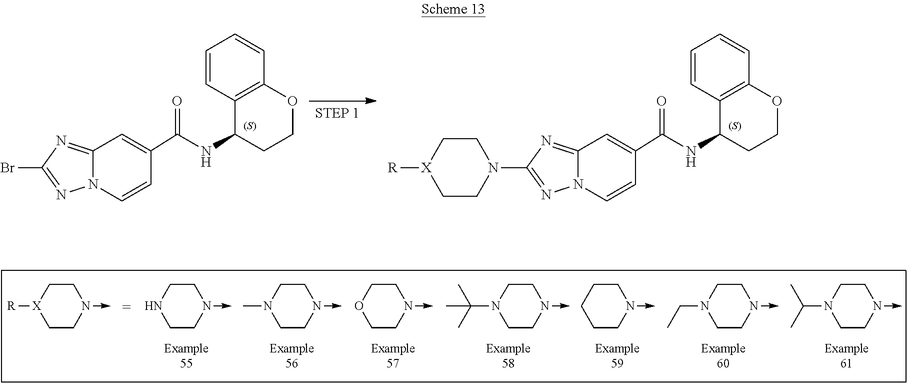

### Step 1 (Scheme 13): S_{N}Ar Reaction Leading to Examples 55 to 61

To a solution of (S)-2-bromo-N-(chroman-4-yl)-[1,2,4]triazolo[1,5-a]pyridine-7-carboxamide (1 eq.) and DIPEA (4 eq.) in n-butanol (10 vol.) was added the amine derivative (4 eq.). The mixture was stirred at 120° C. for 16 to 32 h, monitoring by TLC. Upon completion, the reaction mixture was cooled and concentrated under reduced pressure. The residue was purified by preparative HPLC to afford Examples 55 to 61 as white solids.

### Example 55: (S)—N-(chroman-4-yl)-2-(piperazin-1-yl)-[1,2,4]triazolo[1,5-a]pyridine-7-carboxamide

Prepared according to Scheme 13 using piperazine as the amine derivative.

¹H NMR: δ_{H} (400 MHz, DMSO-d₆) 9.11 (1H, d, J 8.0), 8.73 (1H, d, J 7.2), 7.97 (1H, d, J 1.2), 7.40 (1H, dd, J 7.0, 2.0), 7.21-7.16 (2H, m), 6.90 (1H, t, J 6.4), 6.87 (1H, d, J 0.8), 5.29 (1H, d, J 7.2), 4.31-4.26 (2H, m), 3.43 (4H, t, J 4.4), 2.79 (4H, t, J 5.2), 2.14-2.07 (m, 2H). Note: NH proton of piperazine not observed.

### Example 56: (S)—N-(chroman-4-yl)-2-(4-methylpiperazin-1-yl)-[1,2,4]triazolo[1,5-a]pyridine-7-carboxamide

Prepared according to Scheme 13 using 1-methylpiperazine as the amine derivative.

¹H NMR: δ_{H} (400 MHz, methanol-d₄) 8.58 (1H, dd, J 6.8, 0.8), 7.91-7.90 (1H, m), 7.43 (1H, dd, J 7.0, 1.6), 7.26 (1H, d, J 7.6), 7.21-7.16 (1H, m), 6.93 (1H, t, J 6.4), 6.90 (1H, d, J 1.2), 5.37 (1H, t, J6.0), 4.32-4.29 (2H, m), 3.68 (4H, t, J 4.4), 2.72 (4H, t, J 4.8), 2.46 (3H, s), 2.30-2.20 (2H, m). Note: Amide NH proton was not observed.

### Example 57: (S)—N-(chroman-4-yl)-2-morpholino-[1,2,4]triazolo[1,5-a]pyridine-7-carboxamide

Prepared according to Scheme 13 using morpholine as the amine derivative.

¹H NMR: δ_{H} (400 MHz, DMSO-d₆) 9.14 (1H, d, J 8.0), 8.76 (1H, dd, J 6.8, 0.8), 8.01-8.01 (1H, m), 7.44 (1H, dd, J 6.8, 2.0), 7.21-7.16 (2H, m), 6.90 (1H, t, J 6.4), 6.87 (1H, d, J 1.2), 5.29 (1H, q, J 6.8), 4.33-4.24 (2H, m), 3.72 (4H, t, J 4.8), 3.48 (4H, t, J 4.8), 2.16-2.07 (2H, m).

### Example 58: (S)-2-(4-(tert-butyl)piperazin-1-yl)-N-(chroman-4-yl)-[1,2,4]triazolo[1,5-a]pyridine-7-carboxamide

Prepared according to Scheme 13 using 1-tert-butylpiperazine as the amine derivative.

¹H NMR: δ_{H} (400 MHz, DMSO-d₆) 9.12 (1H, d, J 8.0), 8.74 (1H, d, J 6.8), 7.98 (1H, s), 7.41 (1H, d, J 5.6), 7.21-7.16 (2H, m), 6.91-6.81 (2H, m), 5.29 (1H, q, J 6.4), 4.34-4.23 (2H, m), 3.48 (4H, s), 2.68 (4H, s), 2.18-2.05 (2H, m), 1.04 (9H, s).

### Example 59: (S)—N-(chroman-4-yl)-2-(piperidin-1-yl)-[1,2,4]triazolo[1,5-a]pyridine-7-carboxamide

Prepared according to Scheme 13 using piperidine as the amine derivative.

¹H NMR: δ_{H} (400 MHz, DMSO-d₆) 9.11 (1H, d, J 8.4), 8.72 (1H, dd, J 6.8, 0.8), 7.96-7.96 (1H, m), 7.40-7.38 (1H, m), 7.21-7.16 (2H, m), 6.90 (1H, t, J 6.4), 6.87 (1H, d, J 1.2), 5.29 (1H, q, J 6.8), 4.33-4.24 (2H, m), 3.52 (4H, d, J 5.2), 2.19-2.02 (2H, m), 1.59 (6H, s).

### Example 60: (S)—N-(chroman-4-yl)-2-(4-ethylpiperazin-1-yl)-[1,2,4]triazolo[1,5-a]pyridine-7-carboxamide

Prepared according to Scheme 13 using 1-ethylpiperazine as the amine derivative.

¹H NMR: δ_{H} (400 MHz, DMSO-d₆) 9.18 (1H, d, J 8.0), 8.81 (1H, d, J 7.2), 8.06 (1H, s), 7.49 (1H, d, J 6.4), 7.21-7.17 (2H, m), 6.89 (1H, t, J 7.2), 6.82 (1H, d, J 8.0), 5.30 (1H, t, J 5.6), 4.33-4.22 (4H, m), 3.56 (2H, d, J 11.6), 3.45-3.42 (2H, m), 3.16-3.07 (4H, m), 2.14-2.08 (2H, m), 1.27 (3H, t, J 7.2).

### Example 61: (S)—N-(chroman-4-yl)-2-(4-isopropylpiperazin-1-yl)-[1,2,4]triazolo[1,5-a]pyridine-7-carboxamide

Prepared according to Scheme 13 using 1-(propan-2-yl)piperazine as the amine derivative.

¹H NMR: δ_{H} (400 MHz, DMSO-d₆) 9.13 (1H, d, J 8.0), 8.73 (1H, dd, J 7.0, 0.8), 7.98-7.98 (1H, m), 7.42-7.40 (1H, m), 7.21-7.16 (2H, m), 6.90 (1H, t, J 6.4), 6.87 (1H, d, J 1.2), 5.29 (1H, q, J 6.8), 4.34-4.23 (2H, m), 3.49 (4H, t, J 4.4), 2.71-2.67 (1H, m), 2.55-2.53 (4H, m), 2.15-2.06 (2H, m), 1.00 (6H, d, J 6.4).

### Example 62: (S)—N-(2,3-dihydro-1H-inden-1-yl)-2-(piperazin-1-yl)-[1,2,4]triazolo[1,5-a]pyridine-7-carboxamide

Prepared according to Scheme 13, using (S)-2-bromo-N-(2,3-dihydro-1H-inden-1-yl)-[1,2,4]triazolo[1,5-a]pyridine-7-carboxamide [prepared according to Scheme 11 using (S)-1-aminoindane instead of (S)-chroman-4-amine in Step 5] instead of (S)-2-bromo-N-(chroman-4-yl)-[1,2,4]triazolo[1,5-a]pyridine-7-carboxamide and piperazine as the amine derivative.

¹H NMR: δ_{H} (400 MHz, DMSO-d₆) 9.05 (1H, d, J 8.0), 8.75 (1H, d, J 7.2), 7.99 (1H, s), 7.44-7.42 (1H, m), 7.30-7.18 (4H, m), 5.57 (1H, q, J7.6), 3.51 (4H, brs), 3.05-3.03 (1H, m), 3.03-2.99 (5H, m), 2.91-2.83 (1H, m), 2.51-2.51 (1H, m), 2.04-1.98 (1H, m).

### Example 63: (R)—N-(2,3-dihydro-1H-inden-1-yl)-2-(piperazin-1-yl)-[1,2,4]triazolo[1,5-a]pyridine-7-carboxamide

Prepared according to Scheme 13, using (R)-2-bromo-N-(2,3-dihydro-1H-inden-1-yl)-[1,2,4]triazolo[1,5-a]pyridine-7-carboxamide [prepared according to Scheme 11 using (R)-1-aminoindane instead of (S)-chroman-4-amine in Step 5] instead of (S)-2-bromo-N-(chroman-4-yl)-[1,2,4]triazolo[1,5-a]pyridine-7-carboxamide and piperazine as the amine derivative.

¹H NMR: δ_{H} (400 MHz, DMSO-d₆) 9.02 (1H, d, J 8.4), 8.73 (1H, d, J 6.8), 7.97 (1H, s), 7.41 (1H, dd, J 7.0, 2.0), 7.30-7.18 (4H, m), 5.57 (1H, q, J 7.6), 3.44 (4H, t, J 4.8), 3.05-3.01 (1H, m), 2.99-2.91 (1H, m), 2.85 (4H, t, J 8.0), 2.68-2.67 (1H, m), 2.51-2.50 (1H, m), 2.03-1.98 (1H, m).

### Example 64: (S)—N-(2,3-dihydro-1H-inden-1-yl)-2-(4-methylpiperazin-1-yl)-[1,2,4]triazolo[1,5-a]pyridine-7-carboxamide

Prepared according to Scheme 13, using (S)-2-bromo-N-(2,3-dihydro-1H-inden-1-yl)-[1,2,4]triazolo[1,5-a]pyridine-7-carboxamide [prepared according to Scheme 11 using (S)-1-aminoindane instead of (S)-chroman-4-amine in Step 5] instead of (S)-2-bromo-N-(chroman-4-yl)-[1,2,4]triazolo[1,5-a]pyridine-7-carboxamide and 1-methylpiperazine as the amine derivative.

¹H NMR: δ_{H} (400 MHz, DMSO-d₆) 9.04 (1H, d, J 8.0), 8.74 (1H, d, J 7.2), 7.98 (1H, s), 7.42 (1H, d, J 7.2), 7.30-7.18 (4H, m), 5.57 (1H, q, J 8.0), 3.51 (4H, t, J 4.4), 3.05-3.01 (1H, m), 2.99-2.83 (1H, m), 2.51-2.51 (1H, m), 2.46 (4H, t, J 9.6), 2.34 (3H, s), 2.06-2.00 (1H, m).

### Example 65: (S)—N-(2,3-dihydro-1H-inden-1-yl)-2-(4-ethylpiperazin-1-yl)-[1,2,4]triazolo[1,5-a]pyridine-7-carboxamide

Prepared according to Scheme 13, using (S)-2-bromo-N-(2,3-dihydro-1H-inden-1-yl)-[1,2,4]triazolo[1,5-a]pyridine-7-carboxamide [prepared according to Scheme 11 using (S)-1-aminoindane instead of (S)-chroman-4-amine in Step 5] instead of (S)-2-bromo-N-(chroman-4-yl)-[1,2,4]triazolo[1,5-a]pyridine-7-carboxamide and 1-ethylpiperazine as the amine derivative.

¹H NMR: δ_{H} (400 MHz, DMSO-d₆) 9.03 (1H, d, J 8.4), 8.74 (1H, d, J 6.8), 7.98 (1H, d, J 1.2), 7.43-7.41 (1H, m), 7.30-7.18 (4H, m), 5.57 (1H, q, J 8.0), 3.51 (4H, t, J 4.8), 3.05-3.01 (1H, m), 2.99-2.83 (1H, m), 2.48-2.45 (7H, m), 2.16-2.00 (1H, m), 1.04 (3H, t, J 7.2).

### Example 66: 2-(3,8-diazabicyclo[3.2.1]octan-8-yl)-N-(4,4-difluorocyclohexyl)imidazo[1,2-a]pyridine-6-carboxamide

Example 66 may be prepared according to the route shown in Scheme 14.

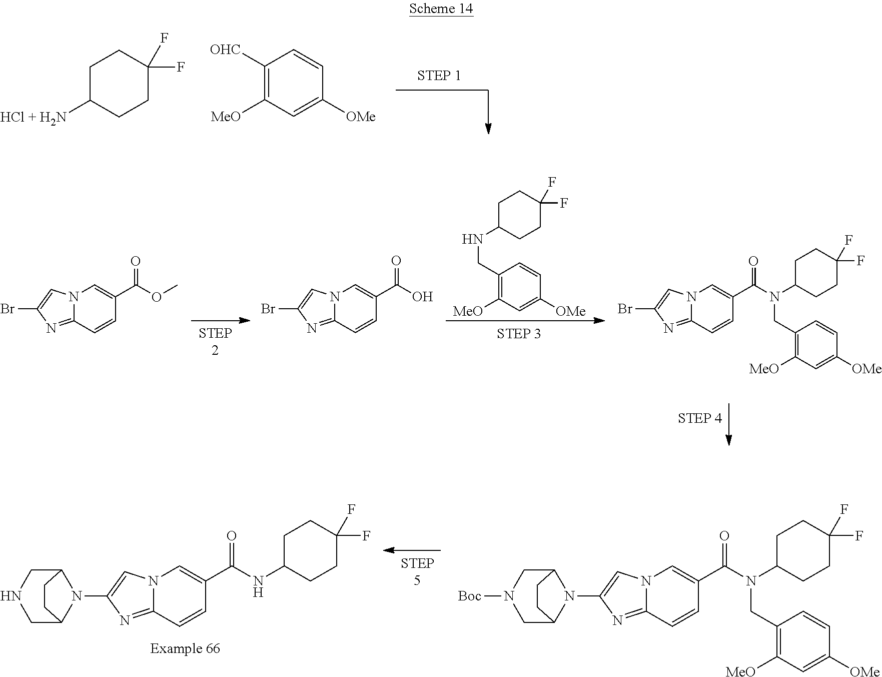

### Step 1 (Scheme 14): Synthesis of N-(2,4-dimethoxybenzyl)-4,4-difluorocyclohexan-1-amine

To a solution of 2,4-dimethoxybenzaldehyde (502 mg, 3.02 mmol) in dichloromethane (15 mL) were added N,N-diisopropylethylamine (560 μL, 3.21 mmol) and 4,4-difluorocyclohexylamine hydrochloride (525 mg, 3.06 mmol). The reaction mixture was stirred at room temperature under nitrogen for 2 h, after which sodium triacetoxyborohydride (1.2 g, 5.66 mmol) was added and stirring was continued overnight. The reaction mixture was partitioned between saturated aqueous NaHCO₃ and dichloromethane, the layers were separated, and the aqueous layer was extracted twice with dichloromethane. The combined organic layers were dried over Na₂SO₄ and concentrated to afford N-(2,4-dimethoxybenzyl)-4,4-difluorocyclohexan-1-amine (905 mg, quant.) as a cloudy gel.

### Step 2 (Scheme 14): Synthesis of 2-bromoimidazo[1,2-a]pyridine-6-carboxylic acid

To a suspension of methyl 2-bromoimidazo[1,2-a]pyridine-6-carboxylate (440 mg, 1.73 mmol) in tetrahydrofuran (10 mL) was added aqueous lithium hydroxide, 0.5 N (8 mL, 4.00 mmol) and the reaction was stirred at room temperature for 2 h. The mixture was partitioned between water and dichloromethane; the layers were separated and the basic water layer was neutralized to pH 5-6 with 2 N aqueous HCl. The resulting milky suspension was extracted 6× with dichloromethane, and the combined milky organic layers were concentrated, yielding crude 2-bromoimidazo[1,2-a]pyridine-6-carboxylic acid as a yellow solid, which was used as such without further purification.

### Step 3 (Scheme 14): Synthesis of 2-bromo-N-(4,4-difluorocyclohexyl)-N-(2,4-dimethoxybenzyl)imidazo[1,2-a]pyridine-6-carboxamide

To a solution of crude 2-bromoimidazo[1,2-a]pyridine-6-carboxylic acid (303 mg, 1.26 mmol) and N-(2,4-dimethoxybenzyl)-4,4-difluorocyclohexan-1-amine (247 mg, 0.87 mmol) in N,N-dimethylformamide (10 mL) were added EDCI.HCl (180 mg, 0.94 mmol) and Oxyma Pure (19 mg, 0.13 mmol) and the reaction was stirred at room temperature overnight. The reaction mixture was partitioned between water and EtOAc; the layers were separated and the aqueous layer was extracted with EtOAc. The combined organic layers were washed with brine, dried over Na₂SO₄ and concentrated. The residue was purified by silica gel flash chromatography, eluting with 0% to 75% ethyl acetate in heptane, to afford 2-bromo-N-(4,4-difluorocyclohexyl)-N-(2,4-dimethoxybenzyl)imidazo[1,2-a]pyridine-6-carboxamide (301 mg, 68% over 2 steps) as a white solid.

### Step 4 (Scheme 14): Synthesis of tert-butyl 8-(6-((4,4-difluorocyclohexyl)(2,4-dimethoxybenzyl)carbamoyl)imidazo[1,2-a]pyridin-2-yl)-3,8-diazabicyclo[3.2.1]octane-3-carboxylate

A suspension of 2-bromo-N-(4,4-difluorocyclohexyl)-N-(2,4-dimethoxybenzyl)imidazo[1,2-a]pyridine-6-carboxamide (153 mg, 0.30 mmol), 3-Boc-3,8-diazabicyclo[3.2.1]octane (135 mg, 0.64 mmol), (t-Bu)PhCPhos (21 mg, 0.052 mmol) and NaOtBu (75 mg, 0.78 mmol) in 1,4-dioxane (5 mL) was flushed with argon for 5 min, after which Pd₂dba₃ (41 mg, 0.045 mmol) was added and the reaction was heated in a sealed vial to 100° C. for 4 h. The reaction mixture was cooled to room temperature, diluted with water (25 mL) and extracted with ethyl acetate (2×20 mL). The combined organic layers were washed with brine (25 mL), dried over sodium sulfate and concentrated. The residue was purified by flash chromatography, eluting with 30% to 100% ethyl acetate in heptane, to afford tert-butyl 8-(6-((4,4-difluorocyclohexyl)(2,4-dimethoxybenzyl)carbamoyl)imidazo[1,2-a]pyridin-2-yl)-3,8-diazabicyclo[3.2.1]octane-3-carboxylate (103 mg, 54%) as a brown solid.

### Step 5 (Scheme 14) Leading to Example 66

To a solution of tert-butyl 8-(6-((4,4-difluorocyclohexyl)(2,4-dimethoxybenzyl) carbamoyl)imidazo[1,2-a]pyridin-2-yl)-3,8-diazabicyclo[3.2.1]octane-3-carboxylate (101 mg, 0.16 mmol) in dichloromethane (3 mL) was added trifluoroacetic acid (350 μL, 4.57 mmol) and the reaction was stirred at room temperature overnight. The reaction mixture was concentrated, the residue was transferred onto an SCX-cartridge eluted with 1N NH₃ in MeOH and concentrated. The resulting material was purified twice by preparative HPLC and the product was lyophilized from acetonitrile/water, yielding 2-(3,8-diazabicyclo[3.2.1]octan-8-yl)-N-(4,4-difluorocyclohexyl)imidazo[1,2-a]pyridine-6-carboxamide (12 mg, 20%) as a light yellow solid.

¹H NMR δ_{H} (400 MHz, CDCl₃) 8.63 (1H, t, J 1.3), 7.35 (1H, d, J9.2), 7.23 (1H, dd, J9.3, 1.8), 6.82 (1H, s), 5.90 (1H, d, J 7.8), 4.11 (1H, d, J 10.4), 3.65 (2H, s), 3.55 (2H, dd, J 11.3, 2.5), 3.03 (2H, d, J 11.1), 2.13 (4H, d, J12.6), 1.99-1.83 (6H, m), 1.68-1.57 (3H, m).

### Example 67: 2-(3,8-diazabicyclo[3.2.1]octan-8-yl)-N-cyclopentylimidazo[1,2-a]pyridine-6-carboxamide

Prepared according to Scheme 14, using cyclopentylamine instead of 4,4-difluorocyclohexylamine in Step 1.

¹H NMR δ_{H} (400 MHz, DMSO-d₆) 8.81 (1H, d, J 1.7), 8.25 (1H, d, J7.2), 7.51 (1H, dd, J9.1, 1.8), 7.34-7.24 (2H, m), 4.28-4.15 (1H, m), 4.02 (2H, dd, J 4.3, 2.3), 2.95 (2H, dd, J 12.2, 1.7), 1.96-1.81 (4H, m), 1.81-1.74 (2H, m), 1.74-1.64 (2H, m, J 4.9, 4.4), 1.61-1.46 (4H, m). Note: One signal (2H) coincides with the DMSO signal.

### Example 68: (S)—N-(chroman-4-yl)-2-(piperazin-1-yl)pyrazolo[1,5-a]pyrimidine-6-carboxamide

Example 68 may be prepared according to the route shown in Scheme 15.

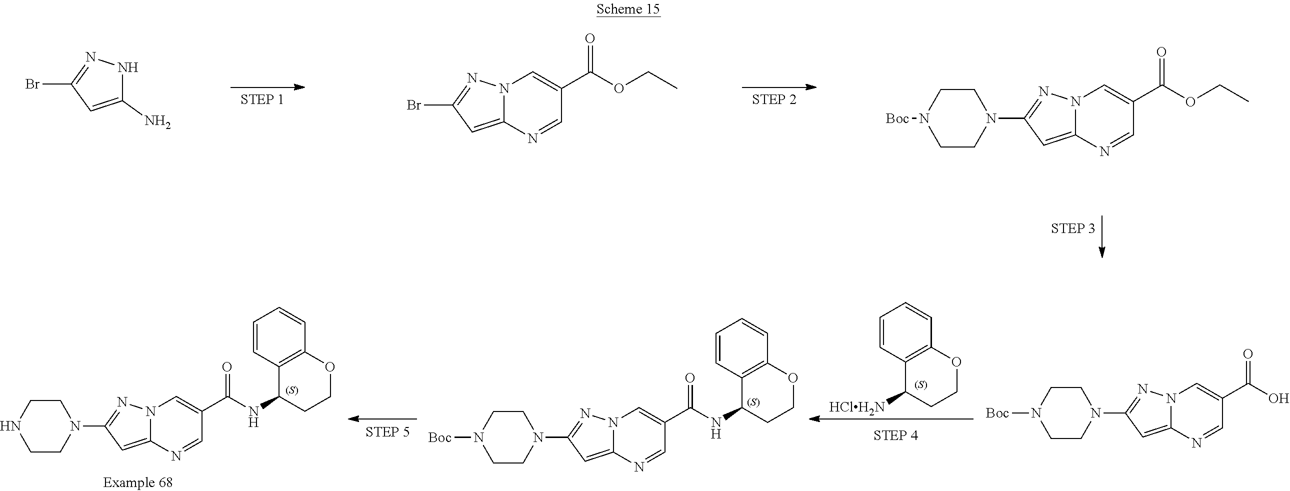

Step 1 (Scheme 15): Synthesis of ethyl 2-bromopyrazolo[1,5-a]pyrimidine-6-carboxylate

To a suspension of 3-bromo-1H-pyrazol-5-amine (3.0 g, 18.5 mmol) in EtOH (45 mL) was added ethyl 2-formyl-3-oxopropanoate (2.66 g, 18.5 mmol) and the reaction mixture was stirred at 70° C. for 16 h. The reaction mixture was cooled to r.t., concentrated under reduced pressure and triturated with n-pentane to afford ethyl 2-bromopyrazolo[1,5-a]pyrimidine-6-carboxylate (4.8 g, 95%) as a pale yellow solid.

### Step 2 (Scheme 15): Synthesis of ethyl 2-(4-(tert-butoxycarbonyl)piperazin-1-yl)pyrazolo[1,5-a]pyrimidine-6-carboxylate

To a degassed mixture of ethyl 2-bromopyrazolo[1,5-a]pyrimidine-6-carboxylate (3 g, 11.1 mmol), tert-butyl piperazine-1-carboxylate (4.63 g, 22.2 mmol), Cs₂CO₃ (7.22 g, 22.2 mmol) and BINAP (1.38 g, 2.22 mmol) in toluene (40 mL) under nitrogen was added Pd(OAc)₂ (0.24 g, 1.11 mmol) and the reaction was stirred at 110° C. for 16 h. The reaction mixture was cooled and filtered through Celite®, washing with dichloromethane. The filtrate was concentrated under reduced pressure and purified by flash chromatography, eluting with 5% methanol in dichloromethane to afford ethyl 2-(4-(tert-butoxycarbonyl)piperazin-1-yl)pyrazolo[1,5-a]pyrimidine-6-carboxylate (0.9 g, 21%) as a pale yellow solid.

### Step 3 (Scheme 15): Synthesis of 2-(4-(tert-butoxycarbonyl)piperazin-1-yl)pyrazolo[1,5-a]pyrimidine-6-carboxylic acid

To a solution of ethyl 2-(4-(tert-butoxycarbonyl)piperazin-1-yl)pyrazolo[1,5-a]pyrimidine-6-carboxylate (0.8 g, 2.4 mmol) in THF:H2 O (3:1; 15 mL) was added LiOH·H₂O (0.15 g, 3.6 mmol) and the mixture was stirred at r.t. for 16 h. The reaction mixture was concentrated under reduced pressure to remove THF. The residue was diluted with water and acidified with citric acid. The resulting solid precipitate was filtered off and dried under reduced pressure, then triturated with n-pentane to afford 2-(4-(tert-butoxycarbonyl)piperazin-1-yl)pyrazolo[1,5-a]pyrimidine-6-carboxylic acid (0.64 g, 86%) as a pale yellow solid.

### Step 4 (Scheme 15): Synthesis of tert-butyl (S)-4-(6-(chroman-4-ylcarbamoyl)pyrazolo[1,5-a]pyrimidin-2-yl)piperazine-1-carboxylate

To a solution of 2-(4-(tert-butoxycarbonyl)piperazin-1-yl)pyrazolo[1,5-a]pyrimidine-6-carboxylic acid (0.63 g, 2.21 mmol) in DMF (6 mL) were added (S)-chroman-4-amine hydrochloride (0.61 g, 3.32 mmol) and DIPEA (1.14 ml, 6.63 mmol). The mixture was stirred for 15 min followed by the addition of n-propylphosphonic acid anhydride, cyclic trimer (2.8 g, 4.42 mmol). The reaction mixture was stirred at r.t. for 16 h, then quenched with ice water (20 mL) and stirred for 10 min. The resulting solid precipitate was filtered off and dried under reduced pressure to afford tert-butyl (S)-4-(6-(chroman-4-ylcarbamoyl)pyrazolo[1,5-a]pyrimidin-2-yl)piperazine-1-carboxylate (0.64 g, 74%) as a pale yellow solid.

### Step 5 (Scheme 15) leading to Example 68: Synthesis of (S)—N-(chroman-4-yl)-2-(piperazin-1-yl)pyrazolo[1,5-a]pyrimidine-6-carboxamide

To a stirred solution of tert-butyl(S)-4-(6-(chroman-4-ylcarbamoyl)pyrazolo[1,5-a]pyrimidin-2-yl)piperazine-1-carboxylate (0.12 g, 0.25 mmol) in dichloromethane (5.0 mL) was added HCl in 1,4-dioxane (4M; 2.0 mL). The mixture was stirred at r.t. for 16 h, then concentrated under reduced pressure. The residue was basified with aqueous NaHCO₃ solution (8 mL) and the organic components were extracted with dichloromethane (2×10 mL). The combined organic layer was separated, dried over anhydrous Na₂SO₄ and concentrated under reduced pressure. The crude product was purified by preparative HPLC using NH₄HCO₃ as buffer to afford the title compound (0.05 g, 53%) as an off-white solid.

¹H NMR: δ_{H} (400 MHz, DMSO-d₆) 9.26 (1H, d, J 1.6), 8.88 (1H, d, J 7.6), 8.75 (1H, d, J 2.0), 7.21 (1H, d, J 10.0), 7.18 (1H, t, J 7.2), 6.90 (1H, t, J 6.4), 6.87 (1H, d, J 1.2), 6.22 (1H, s), 5.26 (1H, d, J 6.8), 4.30-4.26 (2H, m), 3.30-3.27 (5H, m), 2.80 (4H, t, J 4.8), 2.14-2.04 (2H, m).

### Example 69: (S)—N-(chroman-4-yl)-2-(4-methylpiperazin-1-yl)pyrazolo[1,5-a]pyrimidine-6-carboxamide

Example 69 may be prepared by reductive alkylation of Example 68 using formaldehyde, according to the method of Scheme 9.

¹H NMR: δ_{H} (400 MHz, DMSO-d₆) 9.26 (1H, d, J 1.6), 8.88 (1H, d, J 8.0), 8.76 (1H, d, J 2.4), 7.20 (1H, d, J 1.2), 7.17 (1H, t, J 6.8), 6.90 (1H, t, J 0.8), 6.87 (1H, d, J 1.2), 6.25 (1H, s), 5.26 (1H, q, J 6.0), 4.30-4.26 (2H, m), 3.34 (4H, s), 2.42 (4H, t, J 4.8), 2.22 (3H, s), 2.17-2.13 (2H, m).

### Example 70: (S)—N-(chroman-4-yl)-2-(4-ethylpiperazin-1-y)pyrazolo[1,5-a]pyrimidine-6-carboxamide

Example 70 may be prepared by reductive alkylation of Example 68 using acetaldehyde, according to the method of Scheme 10.

¹H NMR: δ_{H} (400 MHz, DMSO-d₆) 9.27 (1H, d, J 1.6), 8.87 (1H, d, J 8.0), 8.76 (1H, d, J 2.4), 7.25-7.16 (2H, m), 6.90 (1H, t, J 6.4), 6.87 (1H, d, J 1.2), 6.25 (1H, s), 5.26 (1H, d, J 7.2), 4.30-4.26 (2H, m), 3.35 (4H, s), 2.55 (4H, s), 2.48-2.33 (2H, m), 2.22-2.03 (2H, m), 1.04 (3H, t, J 7.2).

### Example 71: (S)—N-(2,3-dihydro-1H-inden-1-yl)-2-(piperazin-1-yl)pyrazolo[1,5-a]pyrimidine-6-carboxamide

Example 71 may be prepared according to Scheme 15, using (S)-1-aminoindane hydrochloride in Step 4 instead of (S)-chroman-4-amine hydrochloride.

¹H NMR: δ_{H} (400 MHz, DMSO-d₆) 9.25 (1H, d, J 1.6), 8.76 (2H, d, J2.4), 7.32-7.26 (2H, m), 7.24-7.20 (2H, m), 6.22 (1H, s), 5.55 (1H, q, J 8.0), 3.32-3.29 (4H, m), 3.04-3.00 (2H, m), 2.99-2.81 (5H, m), 2.53-2.53 (1H, m), 2.00-1.94 (1H, m).

### Examples 72 to 75

Examples 72 to 75 may be prepared according to the route shown in Scheme 16.

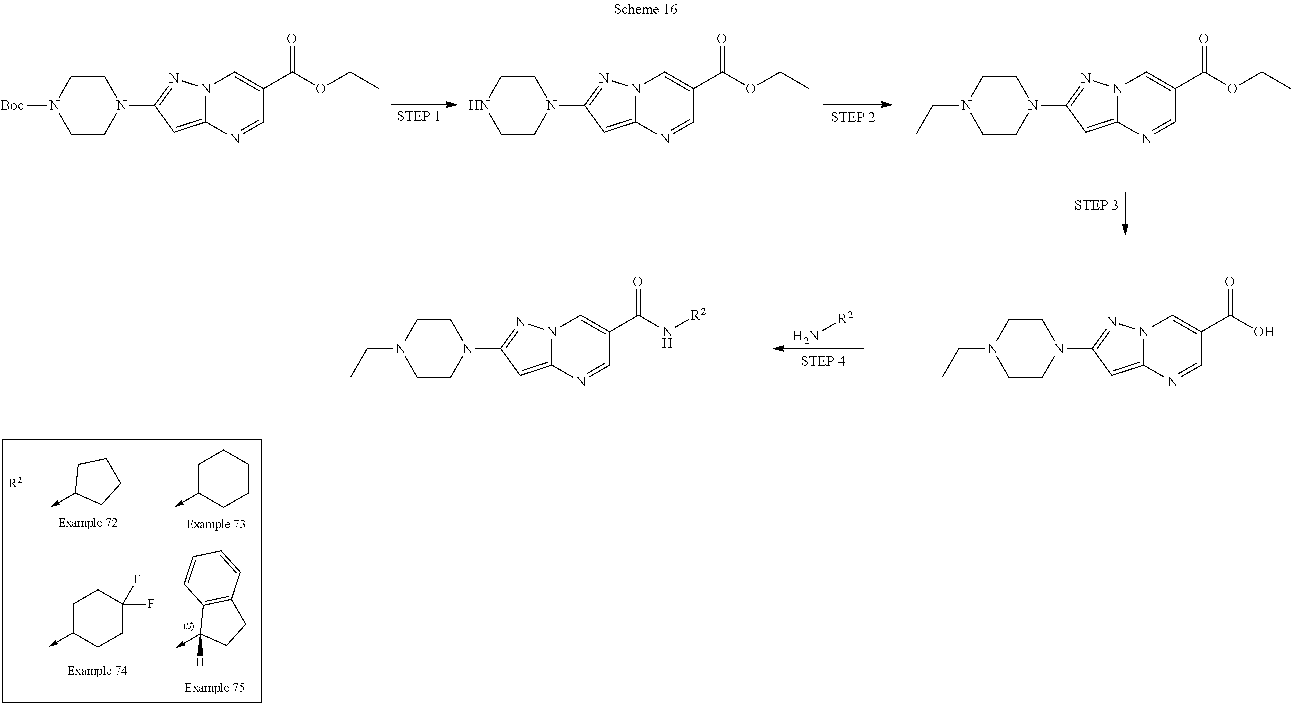

### Step 1 (Scheme 16): Synthesis of ethyl 2-(piperazin-1-yl)pyrazolo[1,5-a]pyrimidine-6-carboxylate

To a stirred solution of ethyl 2-(4-(tert-butoxycarbonyl)piperazin-1-yl)pyrazolo[1,5-a]pyrimidine-6-carboxylate (700 mg, 1.87 mmol) in DCM (15 mL) at 0° C. was added TFA (425 mg, 3.73 mmol). After 15 min the reaction mixture was raised to ambient temperature and maintained at 25° C. for 16 h. The reaction mixture was concentrated under reduced pressure, diluted with water and adjusted to approx. pH 7 using saturated aqueous NaHCO₃ solution. The organic components were extracted with 10% MeOH/DCM (2×25 mL). The combined organic extract was washed with brine (20 mL), dried over anhydrous sodium sulfate, filtered and concentrated under reduced pressure. The crude product was purified by flash chromatography, eluting with 5% to 10% methanol in DCM to obtain ethyl 2-(piperazin-1-yl)pyrazolo[1,5-a]pyrimidine-6-carboxylate (200 mg, 38%) as a pale yellow solid.

### Step 2 (Scheme 16): Synthesis of ethyl 2-(4-ethylpiperazin-1-yl)pyrazolo[1,5-a]pyrimidine-6-carboxylate

To a stirred solution of ethyl 2-(piperazin-1-yl)pyrazolo[1,5-a]pyrimidine-6-carboxylate (700 mg, 2.54 mmol) in acetone (5 mL) at 0° C. were added ethyl iodide (476 mg, 3.05 mmol) and K₂CO₃ (1050 mg, 7.63 mmol). After 15 min, the reaction mixture was heated to 55° C. for 16 h, then concentrated under reduced pressure. The residue was diluted with water (20 mL) and the organic components were extracted with 10% methanol in dichloromethane (2×25 mL). The combined organic extract was washed with brine (20 mL), dried over anhydrous sodium sulfate, filtered and concentrated under reduced pressure. The crude product (800 mg) was triturated with 10% ethyl acetate in hexanes to afford ethyl 2-(4-ethylpiperazin-1-yl)pyrazolo[1,5-a]pyrimidine-6-carboxylate (700 mg, 89%) as pale brown solid.

### Step 3 (Scheme 16): Synthesis of 2-(4-ethylpiperazin-1-yl)pyrazolo[1,5-a]pyrimidine-6-carboxylic acid

To a solution of ethyl 2-(4-ethylpiperazin-1-yl)pyrazolo[1,5-a]pyrimidine-6-carboxylate (650 mg, 2.14 mmol) in THF:H₂O (3:1; 5 mL) at 0° C. was added LiOH·H₂O (180 mg, 4.29 mmol). The reaction mixture was allowed to warm to r.t. and stirred at r.t. for 16 h. The reaction mixture was concentrated to dryness and the product as a lithium salt was used for the next step without purification.

### General Procedure for Step 4 (Scheme 16): Amide Coupling Leading to Examples 72 to 75

A mixture of 2-(4-ethylpiperazin-1-yl)pyrazolo[1,5-a]pyrimidine-6-carboxylic acid, lithium salt (1 eq.), amine (1.2 eq.) and DIPEA (3 eq). in DCM (10 vol.) was stirred for 15 min followed by the addition of n-propylphosphonic acid anhydride, cyclic trimer (50% in ethyl acetate; 2 eq.). The reaction mixture was stirred at r.t. for 16 h, then quenched with ice water. The organic components were extracted twice with 10% MeOH in DCM. The combined organic layer was separated, dried over anhydrous Na₂SO₄ and concentrated under reduced pressure. The crude product was purified by preparative HPLC to afford Examples 72 to 75 as off-white solids.

### Example 72: N-cyclopentyl-2-(4-ethylpiperazin-1-yl)pyrazolo[1,5-a]pyrimidine-6-carboxamide

Prepared according to Scheme 16 using cyclopentylamine as the amine in Step 4.

¹H NMR: δ_{H} (400 MHz, DMSO-d₆) 9.23 (1H, d, J 7.6), 8.70 (1H, d, J 2.0), 8.27 (1H, d, J 7.2), 6.23 (1H, d, J0.4), 4.23 (1H, d, J 6.4), 3.37 (4H, t, J 4.8), 2.48-2.41 (4H, m), 2.39-2.34 (2H, m), 1.92-1.89 (2H, m), 1.72-1.71 (2H, m), 1.57-1.51 (4H, m), 1.04 (3H, t, J 7.2).

### Example 73: N-cyclohexyl-2-(4-ethylpiperazin-1-yl)pyrazolo[1,5-a]pyrimidine-6-carboxamide

Prepared according to Scheme 16 using cyclohexylamine as the amine in Step 4.

¹H NMR: δ_{H} (400 MHz, DMSO-d₆) 9.22 (1H, t, J 1.2), 8.70 (1H, d, J 2.4), 8.21 (1H, d, J 7.6), 6.23 (1H, s), 3.77-3.75 (1H, m), 3.37 (4H, t, J 4.4), 2.56-2.50 (4H, m), 2.48-2.47 (2H, m), 1.86-1.85 (2H, m), 1.84-1.74 (2H, m), 1.63-1.60 (1H, m), 1.30-1.17 (4H, m), 1.16-1.11 (1H, m), 1.04 (3H, t, J 7.2).

### Example 74: N-(4,4-difluorocyclohexyl)-2-(4-ethylpiperazin-1-yl)pyrazolo[1,5-a]pyrimidine-6-carboxamide

Prepared according to Scheme 16 using 4,4-difluorocyclohexylamine as the amine in Step 4.

¹H NMR: δ_{H} (400 MHz, DMSO-d₆) 9.25 (1H, d, J2.0), 8.70 (1H, d, J2.0), 8.30-8.25 (1H, m), 6.25 (1H, s), 4.06-3.94 (1H, m), 3.43-3.33 (4H, m), 2.62-2.50 (4H, m), 2.44-2.32 (2H, m), 2.14-1.92 (6H, m), 1.68-1.62 (2H, m), 1.05 (3H, t, J 6.4).

### Example 75: (S)—N-(2,3-dihydro-1H-inden-1-yl)-2-(4-ethylpiperazin-1-yl)pyrazolo[1,5-a]pyrimidine-6-carboxamide

Prepared according to Scheme 16 using (S)-1-aminoindane as the amine in Step 4.

¹H NMR: δ_{H} (400 MHz, DMSO-d₆) 9.25 (1H, s), 8.77 (2H, d, J 2.0), 7.31-7.20 (4H, m), 6.25 (1H, s), 5.55 (1H, d, J 7.2), 3.37 (4H, s), 3.04-2.98 (1H, m), 2.91-2.85 (1H, m), 2.51 (4H, s), 2.40-2.33 (3H, m), 2.01-1.92 (1H, m), 1.04 (3H, t, J 7.2).

### Examples 76 to 79

Examples 76 to 79 may be prepared according to the route shown in Scheme 17.

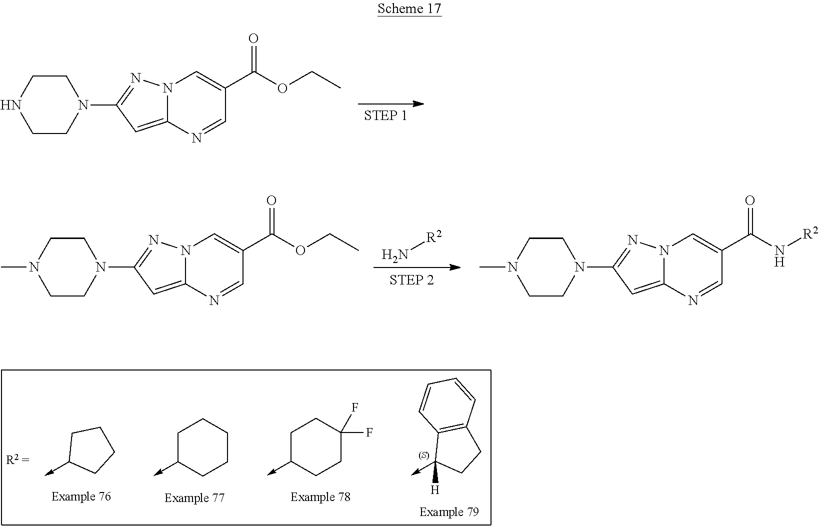

### Step 1 (Scheme 17): Synthesis of ethyl 2-(4-methylpiperazin-1-yl)pyrazolo[1,5-a]pyrimidine-6-carboxylate

To a stirred solution of ethyl 2-(piperazin-1-yl)pyrazolo[1,5-a]pyrimidine-6-carboxylate (130 mg, 0.47 mmol, 1 eq.) in a acetone (30 mL) were added K₂CO₃ (98 mg, 0.71 mmol, 1.5 eq.) and methyl iodide (53.6 mg, 0.38 mmol, 0.8 eq). The reaction mixture was stirred for 2 h at r.t., then quenched with ice-cold water and concentrated under reduced pressure. The residue was diluted with brine (10 mL) and the organic components were extracted with 10% MeOH in DCM (2×20 mL). The combined organic extract was dried over anhydrous sodium sulfate, filtered and concentrated under reduced pressure. The crude product was purified by flash chromatography, eluting with 0% to 10% methanol in dichloromethane to obtain ethyl 2-(4-methylpiperazin-1-yl)pyrazolo[1,5-a]pyrimidine-6-carboxylate (55 mg, 97%) as a pale brown solid.

### General Procedure for Step 2 (Scheme 17): Amide Coupling Leading to Examples 76 to 79

To a stirred solution of ethyl 2-(4-methylpiperazin-1-yl)pyrazolo[1,5-a]pyrimidine-6-carboxylate (1 eq.) in toluene (10 vol.) were added the amine (2 eq.), DIPEA (3 eq.) and triethylenediaminine bis(trimethylaluminum) (2 eq.). The mixture was heated at 110° C. for 16 h, then cooled to r.t., quenched with ice-cold water and concentrated under reduced pressure. The residue was diluted with water (5 vol.) and the organic components were extracted with 5% MeOH in DCM (2×10 vol.). The combined organic extract was washed with brine (5 vol.), dried over anhydrous sodium sulfate, filtered and concentrated under reduced pressure. The crude product was purified by preparative HPLC and the purified fractions were lyophilized to afford Examples 76 to 79 as off-white solids.

### Example 76: N-cyclopentyl-2-(4-methylpiperazin-1-yl)pyrazolo[1,5-a]pyrimidine-6-carboxamide

Prepared according to Scheme 17 using cyclopentylamine as the amine in Step 2.

¹H NMR: δ_{H} (400 MHz, DMSO-d₆) 9.23 (1H, d, J 1.6), 8.70 (1H, d, J 2.4), 8.29 (1H, d, J 7.2), 6.23 (1H, s), 4.25-4.20 (1H, m), 3.37 (4H, t, J 4.8), 2.43 (4H, t, J 4.8), 2.23 (3H, s), 1.91-1.87 (2H, m), 1.71-1.68 (2H, m), 1.57-1.54 (m, 4H).

### Example 77: N-cyclohexyl-2-(4-methylpiperazin-1-yl)pyrazolo[1,5-a]pyrimidine-6-carboxamide

Prepared according to Scheme 17 using cyclohexylamine as the amine in Step 2.

¹H NMR: δ_{H} (400 MHz, DMSO-d₆) 9.22 (1H, d, J 1.6), 8.70 (1H, d, J 2.0), 8.22 (1H, d, J 7.6), 6.23 (1H, s), 3.77-3.75 (1H, m), 3.37 (4H, t, J 4.8), 2.43 (4H, t, J 4.8), 2.23 (3H, s), 1.86-1.84 (2H, m), 1.76-1.74 (2H, m), 1.63-1.60 (1H, m), 1.30-1.24 (4H, m), 1.16-1.13 (1H, m).

### Example 78: N-(4,4-difluorocyclohexyl)-2-(4-methylpiperazin-1-yl)pyrazolo[1,5-a]pyrimidine-6-carboxamide

Prepared according to Scheme 17 using 4,4-difluorocyclohexylamine as the amine in Step 2.

¹H NMR: δ_{H} (400 MHz, DMSO-d₆) 9.24 (1H, d, J 1.6), 8.69 (1H, d, J 2.0), 8.29 (1H, d, J 7.6), 6.25 (1H, d, J0.4), 4.00 (1H, d, J 6.4), 3.37 (4H, t, J4.8), 2.43 (4H, t, J 4.8), 2.23 (3H, s), 2.08-1.89 (6H, m), 1.65-1.62 (2H, m).

### Example 79: (S)—N-(2,3-dihydro-1H-inden-1-yl)-2-(4-methylpiperazin-1-yl)pyrazolo[1,5-a]pyrimidine-6-carboxamide

Prepared according to Scheme 17 using (S)-1-aminoindane as the amine in Step 2.

¹H NMR: δ_{H} (400 MHz, DMSO-d₆) 9.25 (1H, t, J 1.6), 8.81-8.77 (2H, m), 7.31-7.24 (2H, m), 7.23-7.19 (2H, m), 6.25 (1H, s), 5.5 (1H, d, J 8.0), 3.39-3.37 (4H, m), 3.05-2.98 (1H, m), 2.91-2.83 (1H, m), 2.52-2.50 (1H, m), 2.50-2.45 (4H, m), 2.25 (3H, s), 1.99-1.92 (1H, m).

### Example 80: 2-(3,8-diazabicyclo[3.2.1]octan-8-yl)-N-cyclopentylpyrazolo[1,5-a]pyrimidine-6-carboxamide

Example 80 may be prepared according to the method of Scheme 15, using tert-butyl 3,8-diazabicyclo[3.2.1]octane-3-carboxylate instead of tert-butyl piperazine-1-carboxylate in Step 2 and cyclopentylamine instead of (S)-chroman-4-amine hydrochloride in Step 4.

¹H NMR: δ_{H} (400 MHz, DMSO-d₆) 9.23 (1H, d, J 1.6), 8.67 (1H, d, J2.4), 8.22-8.28 (1H, m), 6.17 (1H, s), 4.25-4.21 (1H, m), 4.15-4.10 (2H, m), 2.97-2.94 (2H, m), 1.93-1.84 (6H, m), 1.68-1.70 (2H, m), 1.56-1.50 (4H, m). Note: One signal (2H) coincides with the DMSO signal.

### Example 81: N-cyclopentyl-2-(3-methyl-3,8-diazabicyclo[3.2.1]octan-8-yl)pvrazolo[1,5-a]pyrimidine-6-carboxamide

Example 81 may be prepared by reductive alkylation of Example 80 using formaldehyde, according to the method of Scheme 9.

¹H NMR: δ_{H} (400 MHz, DMSO-d₆) 9.23 (1H, s), 8.68 (1H, d J2.0), 8.22-8.28 (1H, m), 6.21 (1H, s), 4.25-4.22 (3H, m), 2.68-2.51 (2H, m), 2.33-2.28 (2H, m), 2.10-2.08 (3H, m), 1.88-1.82 (6H, m), 1.84-1.71 (2H, m), 1.51-1.54 (4H, m).

### Example 82: (S)—N-(chroman-4-yl)-2-(piperidin-4-yl)pyrazolo[1,5-a]pyrimidine-6-carboxamide

Example 82 may be prepared according to the route shown in Scheme 18.

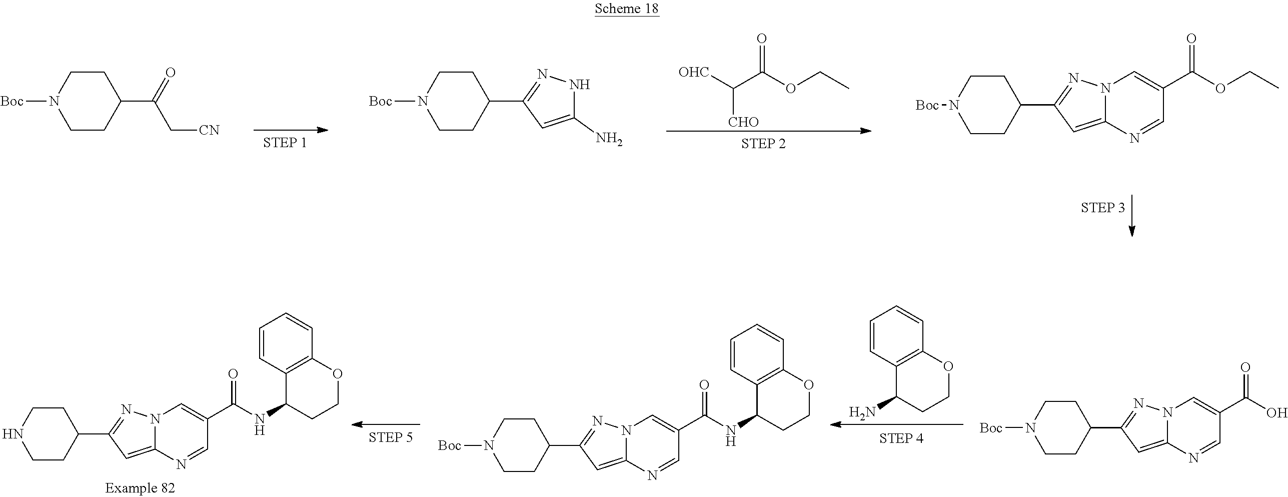

### Step 1 (Scheme 18): Synthesis of tert-butyl 4-(5-amino-1H-pyrazol-3-yl)piperidine-1-carboxylate

To a stirred solution of tert-butyl 4-(2-cyanoacetyl)piperidine-1-carboxylate (200 mg, 0.79 mmol) in ethanol (5 mL) at r.t. was added N₂H₄·H₂O (0.078 mL, 1.59 mmol). The reaction mixture was heated under reflux with stirring for 16 h, then concentrated under reduced pressure. The residue was diluted with water (5 mL) and extracted with 5% MeOH/DCM (2×10 mL). The combined organic extract was washed with brine (10 mL), dried over anhydrous sodium sulfate, filtered and concentrated under reduced pressure to afford a crude product (180 mg, 84%), which was used for the next step without further purification.

### Step 2 (Scheme 18): Synthesis of ethyl 2-(1-(tert-butoxycarbonyl)piperidin-4-yl)pyrazolo[1,5-a]pyrimidine-6-carboxylate

To a stirred solution of tert-butyl 4-(5-amino-1H-pyrazol-3-yl)piperidine-1-carboxylate (200 mg, 0.75 mmol) in ethanol (5 mL) at r.t. was added ethyl 2-formyl-3-oxopropanoate (108 mg, 0.751 mmol). The reaction mixture was heated under reflux with stirring for 16 h, then concentrated under reduced pressure. The residue was diluted with water (30 mL) and extracted with 5% MeOH in DCM (2×50 mL). The combined organic extract was washed with brine (25 mL), dried over anhydrous sodium sulfate, filtered and concentrated under reduced pressure. The crude product was purified by flash chromatography, eluting with 20% to 40% EtOAc in hexanes, to obtain the desired product (170 mg, 63%) as an off-white solid.

### Step 3 (Scheme 18): Synthesis of 2-(1-(tert-butoxycarbonyl)piperidin-4-yl)pyrazolo[1,5-a]pyrimidine-6-carboxylic acid

To a mixture of ethyl 2-(1-(tert-butoxycarbonyl)piperidin-4-yl)pyrazolo[1,5-a]pyrimidine-6-carboxylate (150 mg, 0.40 mmol) in THF:H2 O (3:1; 5 mL) was added LiOH·H₂O (19.2 mg, 0.80 mmol) and the mixture was stirred at r.t. for 16 h. The reaction mixture was concentrated under reduced pressure to remove THF. The residue was diluted with water (10 mL) and the pH adjusted to ˜4 using citric acid, followed by extraction with 5% MeOH/DCM (2×25 mL). The combined organic extract was washed with brine (10 mL), dried over anhydrous sodium sulfate, filtered and concentrated under reduced pressure. The crude product was triturated with 10% ethyl acetate in hexanes to afford the desired product (120 mg, 85%) as a pale yellow solid.

### General Procedure for Step 4 (Scheme 18): Amide Coupling

To a solution of 2-(1-(tert-butoxycarbonyl)piperidin-4-yl)pyrazolo[1,5-a]pyrimidine-6-carboxylic acid (1 eq.) in DCM (10 vol.) at 0° C. was added n-propylphosphonic acid anhydride, cyclic trimer (50% in ethyl acetate; 2 eq.) and DIPEA (4 eq.). The mixture was stirred for 15 min, followed by addition of (S)-chroman-4-amine (1.3 eq). The reaction mixture was warmed to r.t. and stirred for 16 h, then diluted with water (20 vol.) and extracted with 5% MeOH in DCM (2×15 vol.). The combined organic extract was washed with brine (10 vol.), dried over anhydrous sodium sulfate, filtered and concentrated under reduced pressure. The crude product was purified by preparative HPLC to afford tert-butyl (S)-4-(6-(chroman-4-ylcarbamoyl)pyrazolo[1,5-a]pyrimidin-2-yl)piperidine-1-carboxylate as an off-white solid.

### General Procedure for Step 5 (Scheme 18) Leading to Example 82: Boc Deprotection

To a stirred solution of tert-butyl (S)-4-(6-(chroman-4-ylcarbamoyl)pyrazolo[1,5-a]pyrimidin-2-yl)piperidine-1-carboxylate (1 eq.) in DCM (10 vol.) at 0° C. was added HCl in dioxane (4 M; 3 vol.). The reaction mixture was allowed to warm to r.t. and stirred for 16 h, then concentrated under reduced pressure. The residue was washed with acetonitrile and dried under reduced pressure to obtain (S)—N-(chroman-4-yl)-2-(piperidin-4-yl)pyrazolo[1,5-a]pyrimidine-6-carboxamide (Example 82, isolated as its hydrochloride salt) as an off-white solid.

¹H NMR: δ_{H} (400 MHz, DMSO-d₆) 9.54 (1H, d, J 1.6), 9.08 (1H, d, J 8.0), 8.94 (1H, d, J 2.0), 8.85 (1H, s), 8.63 (1H, d, J 10.8), 7.26 (1H, d, J 7.6), 7.19 (1H, t, J 8.4), 6.90 (1H, t, J 7.2), 6.83 (1H, d, J 8.4), 6.74 (1H, s), 5.28 (1H, q, J 6.0), 4.33-4.28 (2H, m), 3.35-3.17 (1H, m), 3.11-3.02 (2H, m), 2.21-2.17 (3H, m), 2.15-2.05 (1H, m), 1.98-1.91 (2H, m). Note: One signal (2H) coincides with the DMSO signal.

### Example 83: (S)—N-(2,3-dihydro-1H-inden-1-yl)-2-(piperidin-4-yl)pyrazolo[1,5-a]pyrimidine-6-carboxamide

Prepared according to the method of Scheme 18, using (S)-1-aminoindane instead of (S)-chroman-4-amine in Step 4. The title compound was isolated as the free base.

¹H NMR: δ_{H} (400 MHz, DMSO-d₆) 9.49 (1H, d, J 1.6), 8.95 (1H, d, J 8.0), 8.92 (1H, d, J 2.4), 7.34-7.25 (2H, m), 7.23-7.19 (2H, m), 6.68 (1H, s), 5.57 (1H, q, J 8.0), 3.07-2.86 (6H, m), 2.73-2.67 (2H, m), 2.01-1.95 (4H, m), 1.69-1.65 (2H, m).

### Example 84: (S)—N-(chroman-4-yl)-2-(1-ethylpiperidin-4-yl)pyrazolo[1,5-a]pyrimidine-6-carboxamide

Example 84 may be prepared according to the method of Scheme 19.

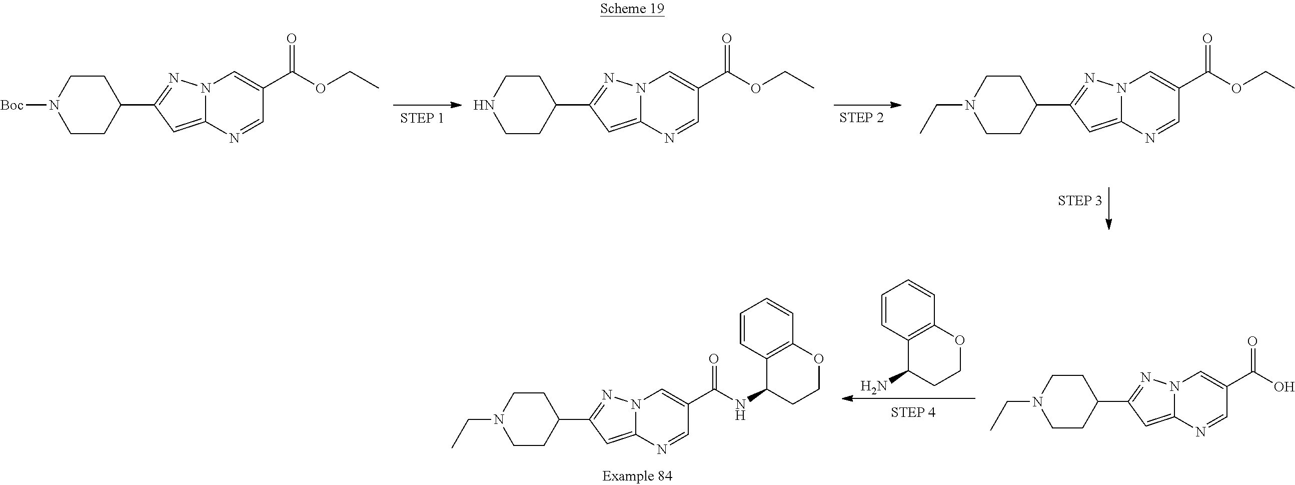 [SMILES: CCOC(=O)c1cnc2cc(C3CCN(C(=O)OC(C)(C)C)CC3)nn2c1 ; CCOC(=O)c1cnc2cc(C3CCNCC3)nn2c1 ; CCOC(=O)c1cnc2cc(C3CCN(CC)CC3)nn2c1 ; CCN1CCC(c2cc3ncc(C(=O)N[C@@H]4CCOc5ccccc54)cn3n2)CC1 ; N[C@@H]1CCOc2ccccc21 ; CCN1CCC(c2cc3ncc(C(=O)O)cn3n2)CC1]

### Step 1 (Scheme 19): Synthesis of ethyl 2-(piperidin-4-yl)pyrazolo[1,5-a]pyrimidine-6-carboxylate

To a stirred solution of ethyl 2-(1-(tert-butoxycarbonyl)piperidin-4-yl)pyrazolo[1,5-a]pyrimidine-6-carboxylate (1.8 g, 4.81 mmol, 1 eq.) in DCM (5 mL) at 0° C. was added TFA (1.11 mL, 14.4 mmol, 3 eq.). The reaction mixture was allowed to warm to r.t. and stirred for 16 h, then concentrated under reduced pressure. The residue was diluted with 10% MeOH in DCM (15 mL) and washed with aqueous sodium bicarbonate (5 mL). The organic layer was separated, dried over anhydrous sodium sulfate, filtered and concentrated under reduced pressure to obtain ethyl 2-(piperidin-4-yl)pyrazolo[1,5-a]pyrimidine-6-carboxylate (500 mg, 36%) as an off-white solid.

### Step 2 (Scheme 19): Synthesis of ethyl 2-(1-ethylpiperidin-4-yl)pyrazolo[1,5-a]pyrimidine-6-carboxylate

To a solution of ethyl 2-(piperidin-4-yl)pyrazolo[1,5-a]pyrimidine-6-carboxylate (100 mg, 0.37 mmol, 1 eq.) in acetone (10 mL) under nitrogen was added K₂CO₃ (101 mg, 0.73 mmol, 2 eq.), and ethyl iodide (68.2 mg, 0.44 mmol, 1.2 eq.). The reaction mixture was heated to reflux for 16 h under nitrogen, then concentrated under reduced pressure. The residue was dissolved in 10% MeOH in DCM (10 mL) and washed with brine (25 mL). The organic extract was dried over anhydrous sodium sulfate, filtered, and concentrated under reduced pressure to afford crude ethyl 2-(1-ethylpiperidin-4-yl)pyrazolo[1,5-a]pyrimidine-6-carboxylate (80 mg, 57%), which was used in the next step without further purification.

### Step 3 (Scheme 19): Synthesis of 2-(1-ethylpiperidin-4-yl)pyrazolo[1,5-a]pyrimidine-6-carboxylic acid, lithium salt

To a mixture of ethyl 2-(1-ethylpiperidin-4-yl)pyrazolo[1,5-a]pyrimidine-6-carboxylate (120 mg, 0.40 mmol, 1 eq.) in THF:water (9:1; 10 mL) was added lithium hydroxide (14.3 mg, 0.60 mmol, 1.5 eq.) at 0° C. The reaction mixture was stirred at rt for 16 h, then concentrated under reduced pressure to obtain crude 2-(1-ethylpiperidin-4-yl)pyrazolo[1,5-a]pyrimidine-6-carboxylic acid lithium salt, which was used for the subsequent step without further purification.

### Step 4 (Scheme 19) Leading to Example 84

To a stirred solution of 2-(1-ethylpiperidin-4-yl)pyrazolo[1,5-a]pyrimidine-6-carboxylic acid (190 mg, 0.69 mmol) in DCM (5 mL) at 0° C. were added n-propylphosphonic acid anhydride, cyclic trimer (0.63 mL, 1.39 mmol) and DIPEA (358 mg, 2.77 mmol). The mixture was stirred for 15 min, followed by addition of (S)-chroman-4-amine (134 mg, 0.90 mmol). The mixture was allowed to warm to r.t. and stirred for 16 h, then diluted with water (10 mL) and extracted with 5% MeOH in DCM (2×25 mL). The combined organic extract was washed with brine (10 mL), dried over anhydrous sodium sulfate, filtered and concentrated under reduced pressure. The crude product was purified by preparative HPLC to afford (S)—N-(chroman-4-yl)-2-(1-ethylpiperidin-4-yl)pyrazolo[1,5-a]pyrimidine-6-carboxamide (20 mg, 99%) as an off-white solid.

¹H NMR: δ_{H} (400 MHz, DMSO-d₆) 9.51 (1H, d, J1.2), 9.06-9.02 (1H, m), 8.93 (1H, d, J2.4), 7.27-7.17 (2H, m), 6.91 (1H, t, J 1.2), 6.88 (1H, d, J 1.2), 6.73 (1H, s), 5.28 (1H, q, J 6.8), 4.31-4.27 (2H, m), 3.17-3.07 (2H, m), 2.19-2.01 (5H, m), 1.87-1.84 (2H, m), 1.24-1.13 (3H, m). Note: One signal (4H) coincides with the DMSO signal.

### Examples 85 to 87

Examples 85 to 87 may be prepared according to the method of Scheme 20.

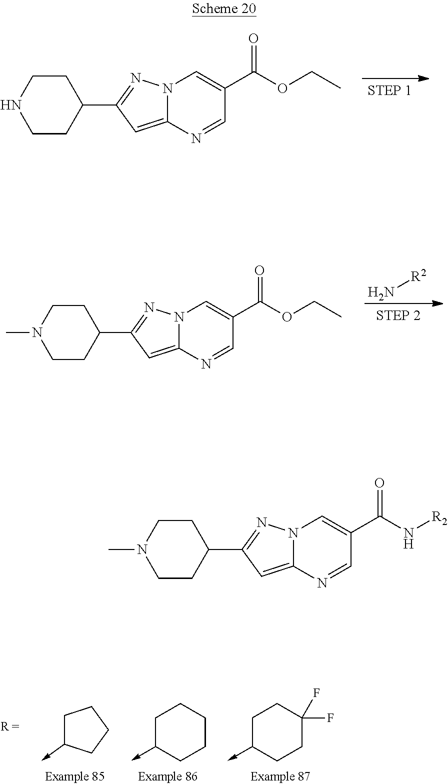

### Step 1 (Scheme 20): Synthesis of ethyl 2-(1-methylpiperidin-4-yl)pyrazolo[1,5-a]pyrimidine-6-carboxylate

To a stirred solution of ethyl 2-(piperidin-4-yl)pyrazolo[1,5-a]pyrimidine-6-carboxylate (1.13 g, 4.12 mmol, 1.0 eq.) in THF (50 mL) was added iodomethane (0.283 mL, 4.53 mmol, 1.1 eq.) followed by NaH (60% in mineral oil; 0.247 g, 6.18 mmol, 1.5 eq.) at 0° C. The mixture was stirred at r.t. for 2 h, then quenched with ice cold water and the organic components were extracted with 10% MeOH/DCM (2×50 mL). The combined organic extract was washed with brine (50 mL), dried over anhydrous sodium sulfate, filtered and concentrated under reduced pressure. The residue was purified by flash chromatography, eluting with 0% to 10% MeOH in DCM to obtain ethyl 2-(1-methylpiperidine-4-yl)pyrazolo[1,5-a]pyrimidine-6-carboxylate (320 mg, 25%) as a yellow solid.

### General Procedure for Step 2 (Scheme 20): Amide Coupling

To a stirred solution of ethyl 2-(1-methylpiperidin-4-yl)pyrazolo[1,5-a]pyrimidine-6-carboxylate (1 eq.) in toluene (10 vol.) were added the required amine (2 eq.), DIPEA (3 eq.) and triethylenediaminine bis(trimethylaluminum) (2 eq.). The reaction mixture was stirred at 110° C. for 16 h, then cooled to r.t., quenched with ice-cold water and concentrated under reduced pressure. The residue was diluted with water (5 vol.) and the organic components were extracted with 5% MeOH in DCM (2×10 vol.). The combined organic extract was washed with brine (5 vol.), dried over anhydrous sodium sulfate, filtered and concentrated under reduced pressure. The crude product was purified by preparative HPLC to afford Examples 85 to 87 as off-white solids.

### Example 85: N-cyclopentyl-2-(1-methylpiperidin-4-yl)pyrazolo[1,5-a]pyrimidine-6-carboxamide

Prepared according to Scheme 20 using cyclopentylamine as the amine in Step 2.

¹H NMR: δ_{H} (400 MHz, DMSO-d₆): 9.46 (1H, s), 8.85 (1H, d, J 1.6), 8.45 (1H, d, J 6.8), 6.69 (1H, s), 4.27-4.24 (1H, m), 2.86-2.77 (3H, m), 2.20 (3H, s), 2.09-1.91 (5H, m), 1.77-1.64 (5H, m), 1.56-1.36 (4H, m).

### Example 86: N-cyclohexyl-2-(1-methylpiperidin-4-yl)pyrazolo[1,5-a]pyrimidine-6-carboxamide

Prepared according to Scheme 20 using cyclohexylamine as the amine in Step 2.

¹H NMR: δ_{H} (400 MHz, DMSO-d₆) 9.46-9.45 (1H, m), 8.84 (1H, d, J 2.0), 8.38 (1H, d, J 7.6), 6.69 (1H, s), 3.89-3.70 (1H, m), 2.83-2.73 (2H, m), 2.71-2.65 (2H, m), 2.20 (3H, s), 2.04-1.95 (3H, m), 1.87-1.86 (2H, m), 1.77-1.74 (4H, m), 1.63-1.60 (1H, m), 1.36-1.34 (4H, m), 1.32-1.29 (1H, m).

### Example 87: N-(4,4-difluorocyclohexyl)-2-(1-methylpiperidin-4-yl)pyrazolo[1,5-a]pyrimidine-6-carboxamide

Prepared according to Scheme 20 using 4,4-difluorocyclohexylamine as the amine in Step 2.

¹H NMR: δ_{H} (400 MHz, DMSO-d₆) 9.48 (1H, d, J 1.6), 8.84 (1H, d, J 2.0), 8.44 (1H, d, J 7.6), 6.69 (1H, s), 3.94-3.92 (1H, m), 2.83-2.78 (3H, m), 2.46 (3H, s), 2.34-2.33 (4H, m), 2.08-2.00 (6H, m), 1.96-1.89 (2H, m), 1.78-1.75 (2H, m).

### Example 88: (S)—N-(chroman-4-yl)-2-(1-methylpiperidin-4-yl)pyrazolo[1,5-a]pyrimidine-6-carboxamide

Prepared according to Scheme 19 using ethyl 2-(1-methylpiperidin-4-yl)pyrazolo[1,5-a]pyrimidine-6-carboxylate in place of ethyl 2-(1-ethylpiperidin-4-yl)pyrazolo[1,5-a]pyrimidine-6-carboxylate in Step 3.

¹H NMR: δ_{H} (400 MHz, DMSO-d₆): 9.51 (1H, dd, J 2.2 and 0.8), 9.05 (1H, br. d, J 7.8), 8.90 (1H, d, J 2.2), 7.27-7.23 (1H, m), 7.20-7.16 (1H, m), 6.89 (1H, td, J 7.5 and 1.2), 6.82 (1H, dd, J 8.2 and 1.0), 6.69 (1H, app. s), 5.29-5.24 (1H, m), 4.33-4.22 (2H, m), 2.86-2.80 (2H, m), 2.75 (1H, tt, J 11.5 and 3.8), 2.18 (3H, s), 2.18-2.11 (1H, m), 2.09-1.91 (5H, m), 1.80-1.69 (2H, m).

### Example 89: N-(4-fluorobenzyl)-2-(piperidin-4-yl)pyrazolo[1,5-a]pyrimidine-6-carboxamide

Example 89 may be prepared according to the method of Scheme 18, using (4-fluorophenyl)methanamine instead of (S)-chroman-4-amine in Step 4.

¹H NMR: δ_{H} (400 MHz, DMSO-d₆): 9.47 (1H, d, J 1.6), 9.23-9.20 (1H, m), 8.88 (1H, d, J 2.4), 7.43-7.40 (2H, m), 7.19-7.15 (2H, m), 6.67 (1H, s), 4.50 (2H, d, J 6.0), 3.03-2.86 (2H, m), 2.68-2.58 (1H, m), 2.61-2.53 (2H, m), 1.90 (2H, d, J 12.8), 1.66-1.57 (2H, m).

### Example 90: (S)—N-(chroman-4-yl)-2-(pyridin-3-yl)pyrazolo[1,5-a]pyrimidine-6-carboxamide

Example 90 may be prepared according to the method of Scheme 21.

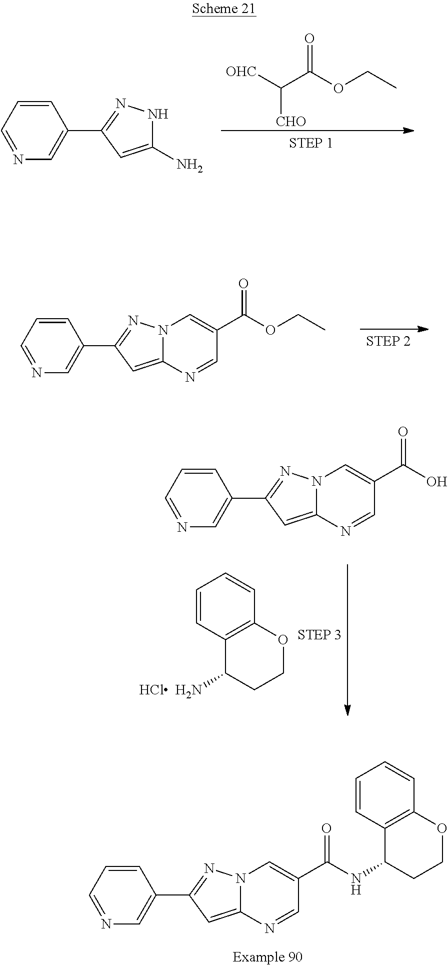

### Step 1 (Scheme 21): Synthesis of ethyl 2-(pyridin-3-yl)pyrazolo[1,5-a]pyrimidine-6-carboxylate

To a stirred suspension of 3-(pyridin-3-yl)-1H-pyrazol-5-amine (500 mg, 3.12 mmol) in IPA (5 mL) was added ethyl 2-formyl-3-oxopropanoate (495 mg, 3.43 mmol, 1.1 eq.). The reaction mixture was stirred at r.t. for 16 h, then concentrated under reduced pressure, diluted with DCM (20 mL) and washed with water and brine. The organic layer was dried over anhydrous Na₂SO₄ and concentrated under reduced pressure. The residue was triturated with n-pentane to afford ethyl 2-(pyridin-3-yl)pyrazolo[1,5-a]pyrimidine-6-carboxylate (400 mg, 48%) as a pale yellow solid.

### Step 2 (Scheme 21): Synthesis of 2-(pyridin-3-yl)pyrazolo[1,5-a]pyrimidine-6-carboxylic acid

To a stirred solution of ethyl 2-(pyridin-3-yl)pyrazolo[1,5-a]pyrimidine-6-carboxylate (0.4 g, 1.49 mmol) in THF:H₂O (3:1; 10 mL), was added LiOH·H₂O (0.125 g, 3.0 mmol). The mixture was stirred at r.t. for 16 h, then concentrated under reduced pressure to remove THF. The residue was diluted with water (3.5 mL) and acidified with saturated aqueous citric acid solution to around pH 4. The resulting solid precipitate was filtered off and triturated with n-pentane to afford 2-(pyridin-3-yl)pyrazolo[1,5-a]pyrimidine-6-carboxylic acid (0.25 g, 70%) as an off-white solid.

### Step 3 (Scheme 21) Leading to Example 90

To a solution of 2-(pyridin-3-yl)pyrazolo[1,5-a]pyrimidine-6-carboxylic acid (200 mg, 0.83 mmol, 1 eq.) and (S)-chroman-4-amine hydrochloride (185 mg, 0.1 mmol, 1.2 eq.) in DMF (10 vol.) was added DIPEA (430 mg, 3.33 mmol, 3 eq.). The mixture was stirred for 15 min, after which T₃P (50% in Ethyl acetate; 1.06 g, 1.66 mmol, 2 eq.) was added and the reaction mixture was stirred at r.t. for 16 h. After the completion of reaction, ice water was added to the reaction mixture and the organic components were extracted with DCM (2×20 mL). The combined organic layer was separated, dried over anhydrous Na₂SO₄ and concentrated under reduced pressure. The crude product was purified by preparative HPLC to afford (S)—N-(chroman-4-yl)-2-(pyridin-3-yl)pyrazolo[1,5-a]pyrimidine-6-carboxamide (11 mg) as an off-white solid.

¹H NMR: δ_{H} (400 MHz, DMSO-d₆) 9.66 (1H, d, J 1.6), 9.28 (1H, d, J 1.6), 9.14 (1H, d, J 7.6), 9.00 (1H, d, J 2.4), 8.67-8.65 (1H, m), 8.44-8.41 (1H, m), 7.58-7.55 (1H, m), 7.50 (1H, s), 7.28 (1H, d, J 8.0), 7.21 (1H, t, J 1.6), 6.92 (1H, t, J0.8), 6.90 (1H, d, J 6.4), 5.30 (1H, q, J 6.0), 4.32-4.29 (2H, m), 2.21-2.15 (1H, m), 2.11-2.05 (1H, m).

### Examples 91 and 92

Examples 91 and 92 may be prepared according to the method of Scheme 22.

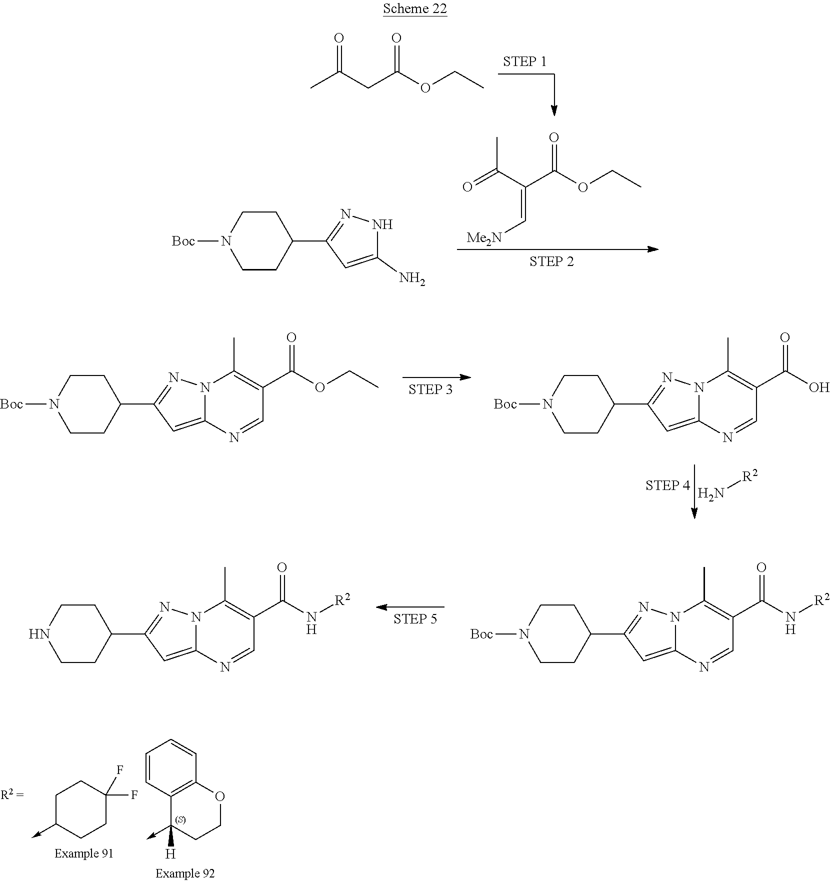

### Step 1 (Scheme 22): Synthesis of ethyl 2-((dimethylamino)methylene)-3-oxobutanoate

To a stirred solution of ethyl 3-oxobutanoate (10 g, 77 mmol) in ethanol (50 mL) was added DMF·DMA (11.3 mL, 85 mmol). The reaction mixture was stirred at r.t. for 16 h, then concentrated under reduced pressure to obtain crude ethyl 2-((dimethylamino)methylene)-3-oxobutanoate (12.3 g, 59%) as a yellow liquid, which was taken forward to the next step without further purification.

### Step 2 (Scheme 22): Synthesis of ethyl 2-(1-(tert-butoxycarbonyl)piperidin-4-yl)-7-methylpyrazolo[1,5-a]pyrimidine-6-carboxylate

To a stirred solution of tert-butyl 4-(5-amino-1H-pyrazol-3-yl)piperidine-1-carboxylate (5.1 g, 19.2 mmol) in ethanol (50 mL) was added ethyl 2-((dimethylamino)methylene)-3-oxobutanoate (10.6 g, 57.4 mmol). The resulting mixture was stirred at 80° C. for 16 h, then concentrated under reduced pressure. The residue was diluted with water and extracted twice with 5% MeOH in DCM. The combined organic extract was washed with brine (20 mL), dried over anhydrous sodium sulfate, filtered and concentrated under reduced pressure. The residue was purified by flash chromatography, eluting with 0% to 15% ethyl acetate in petroleum ether, to afford ethyl 2-(1-(tert-butoxycarbonyl)piperidin-4-yl)-7-methylpyrazolo[1,5-a]pyrimidine-6-carboxylate (6.7 g, 88%).

### Step 3 (Scheme 22): Synthesis of 2-(1-(tert-butoxycarbonyl)piperidin-4-yl)-7-methylpyrazolo[1,5-a]pyrimidine-6-carboxylic acid

To a stirred solution of ethyl 2-(1-(tert-butoxycarbonyl)piperidin-4-yl)-7-methylpyrazolo[1,5-a]pyrimidine-6-carboxylate (1.0 g, 2.57 mmol) in THF:H₂O (7:3; 20 mL) at 0° C. was added lithium hydroxide (0.123 g, 5.15 mmol). The reaction mixture was allowed to warm to r.t. and stirred for 16 h, then concentrated under reduced pressure, diluted with ice water and acidified with the saturated citric acid solution to approx. pH 4. The solid precipitate was filtered off, washed with water (2×10 mL) and dried under vacuum to afford 2-(1-(tert-butoxycarbonyl)piperidin-4-yl)-7-methylpyrazolo[1,5-a]pyrimidine-6-carboxylic acid (0.89 g, 92%) as an off-white solid.

### General Procedure for Step 4 (Scheme 22): Amide Coupling

To a stirred solution of 2-(1-(tert-butoxycarbonyl)piperidin-4-yl)-7-methylpyrazolo[1,5-a]pyrimidine-6-carboxylic acid (1 eq.) in DCM (10 vol.) at 0° C. was added n-propylphosphonic acid anhydride, cyclic trimer (50% in ethyl acetate; 2 eq.) and DIPEA (5 eq.). The mixture was stirred for 15 min, followed by addition of the amine (2 eq.). The reaction mixture was warmed to r.t. and stirred for 16 h, then diluted with water (20 vol.) and extracted with 5% MeOH in DCM (2×15 vol.). The combined organic extract was washed with brine (10 vol.), dried over anhydrous sodium sulfate, filtered and concentrated under reduced pressure. The crude product was purified by preparative HPLC with ammonium bicarbonate as buffer to afford the required Boc-protected amide as an off-white solid.

### General Procedure for Step 5 (Scheme 22) Leading to Examples 91 and 92: Boc Deprotection

To a stirred solution of the Boc-protected amide (1 eq.) in DCM (10 vol.) at 0° C. was added HCl in dioxane (4M; 3 vol.). The reaction mixture was allowed to warm to r.t. and stirred for 2 h, then concentrated under reduced pressure. The residue was washed with 5% ethyl acetate in petroleum ether and dried under vacuum to obtain Examples 91 and 92 as off-white solids.

### Example 91: N-(4,4-difluorocyclohexyl)-7-methyl-2-(piperidin-4-yl)pyrazolo[1,5-a]pyrimidine-6-carboxamide

Prepared according to the method of Scheme 22, using 4,4-difluorocyclohexylamine as the amine in Step 4. The title compound was isolated as a hydrochloride salt.

¹H NMR: δ_{H} (400 MHz, DMSO-d₆) 9.12-9.09 (1H, m), 8.84-8.82 (1H, m), 8.59 (1H, d, J 7.60), 8.52 (1H, s), 6.70 (1H, s), 4.02-4.00 (1H, m), 3.34 (2H, d, J 12.8), 3.22-3.18 (1H, m), 3.10-3.04 (2H, m), 2.83 (3H, s), 2.19 (2H, d, J 11.6), 1.98-1.92 (8H, m), 1.67-1.62 (2H, m).

### Example 92: (S)—N-(chroman-4-yl)-7-methyl-2-(piperidin-4-yl)pyrazolo[1,5-a]pyrimidine-6-carboxamide

Prepared according to the method of Scheme 22, using (S)-chroman-4-amine as the amine in Step 4. The title compound was isolated as a hydrochloride salt.

¹H NMR: δ_{H} (400 MHz, DMSO-d₆) 9.21-9.19 (1H, m), 9.11 (1H, d, J 8.0), 8.92-8.90 (1H, m), 8.56 (1H, s), 3.83 (1H, m), 7.18 (1H, t, J 1.6), 6.92 (1H, t, J0.8), 6.81 (1H, d, J 8.0), 6.70 (1H, s), 5.28-5.23 (1H, m), 4.27-4.23 (2H, m), 3.35-3.32 (2H, m), 3.22-3.18 (1H, m), 3.10-3.04 (2H, m), 2.87 (3H, s), 2.19 (3H, s), 2.07-1.95 (3H, m).

### Example 93: (S)—N-(chroman-4-yl)-2-(1-ethylpiperidin-4-yl)-7-methylpyrazolo[1,5-a]pyrimidine-6-carboxamide

Example 93 may be prepared according to the method of Scheme 19, using ethyl 2-(1-(tert-butoxycarbonyl)piperidin-4-yl)-7-methylpyrazolo[1,5-a]pyrimidine-6-carboxylate instead of ethyl 2-(1-(tert-butoxycarbonyl)piperidin-4-yl)pyrazolo[1,5-a]pyrimidine-6-carboxylate in Step 1.

¹H NMR: δ_{H} (400 MHz, DMSO-d₆) 9.04 (1H, d, J 8.0), 8.53 (1H, s), 7.32 (1H, d, J 7.6), 7.18 (1H, m), 6.93 (1H, td, J 7.5 and 1.2), 6.81 (1H, dd, J 8.2 and 1.2), 6.67 (1H, s), 5.27-5.25 (1H, m), 4.30-4.20 (2H, m), 3.00-2.90 (2H, m), 2.86-2.78 (4H, m), 2.37-2.33 (2H, m), 2.19-2.18 (1H, m), 2.07-1.94 (5H, m), 1.79-1.73 (2H, m), 1.03 (3H, t, J7.2).

### Example 94: N-(4,4-difluorocyclohexyl)-2-(1-ethylpiperidin-4-yl)-7-methylpyrazolo[1,5-a]pyrimidine-6-carboxamide

Example 94 may be prepared according to the method of Scheme 19, using ethyl 2-(1-(tert-butoxycarbonyl)piperidin-4-yl)-7-methylpyrazolo[1,5-a]pyrimidine-6-carboxylate instead of ethyl 2-(1-(tert-butoxycarbonyl)piperidin-4-yl)pyrazolo[1,5-a]pyrimidine-6-carboxylate in Step 1 and 4,4-difluorocyclohexylamine instead of (S)-chroman-4-amine in Step 4.

¹H NMR: δ_{H} (400 MHz, DMSO-d₆) 8.53-8.48 (2H, m), 6.68 (1H, s), 4.05-3.95 (1H, m), 3.00-2.90 (2H, m), 2.82-2.78 (4H, m), 2.38-2.33 (2H, m), 2.02-1.94 (10H, m), 1.79-1.73 (2H, m), 1.63-1.61 (2H, m), 1.04-1.01 (3H, m).

### Example 95: N-[(4S)-3,4-dihydro-2H-1-benzopyran-4-yl]-7-methyl-2-(1-methylpiperidin-4-yl)pyrazolo[1,5-a]pyrimidine-6-carboxamide

Example 95 may be prepared according to the method of Scheme 20, using ethyl 7-methyl-2-(piperidin-4-yl)pyrazolo[1,5-a]pyrimidine-6-carboxylate instead of ethyl 2-(piperidin-4-yl)pyrazolo[1,5-a]pyrimidine-6-carboxylate in Step 1 and (S)-chroman-4-amine as the amine in Step 2.

¹H NMR: δ_{H} (400 MHz, DMSO-d₆) 9.06 (1H, d, J 8.0), 8.53 (1H, s), 7.32 (1H, d, J 7.2), 7.20-7.16 (1H, m), 6.94-6.90 (1H, m), 6.82-6.80 (1H, m), 6.67 (1H, s), 5.24 (1H, m), 4.28-4.24 (2H, m), 2.88-2.67 (5H, m), 2.34-2.20 (1H, m), 2.19-2.10 (3H, m), 2.09-2.07 (1H, m), 2.06-2.04 (3H, m), 1.99-1.83 (2H, m), 1.80-1.77 (2H, m).

### Example 96: N-(4,4-difluorocyclohexyl)-7-methyl-2-(1-methylpiperidin-4-yl)pyrazolo[1,5-a]pyrimidine-6-carboxamide

Example 96 may be prepared according to the method of Scheme 20, using ethyl 7-methyl-2-(piperidin-4-yl)pyrazolo[1,5-a]pyrimidine-6-carboxylate instead of ethyl 2-(piperidin-4-yl)pyrazolo[1,5-a]pyrimidine-6-carboxylate in Step 1 and 4,4-difluorocyclohexylamine as the amine in Step 2.

¹H NMR: δ_{H} (400 MHz, DMSO-d₆) 8.53-8.48 (2H, m), 6.67 (1H, s), 4.05-3.95 (1H, m), 2.87-2.75 (6H, m), 2.21 (3H, s), 2.08-1.91 (10H, m), 1.82-1.70 (2H, m), 1.70-1.55 (2H, m).

### Example 97: N-cyclopentyl-2-(piperidin-4-yl)-benzo[d]oxazole-5-carboxamide

Example 97 may be prepared according to the method of Scheme 23.

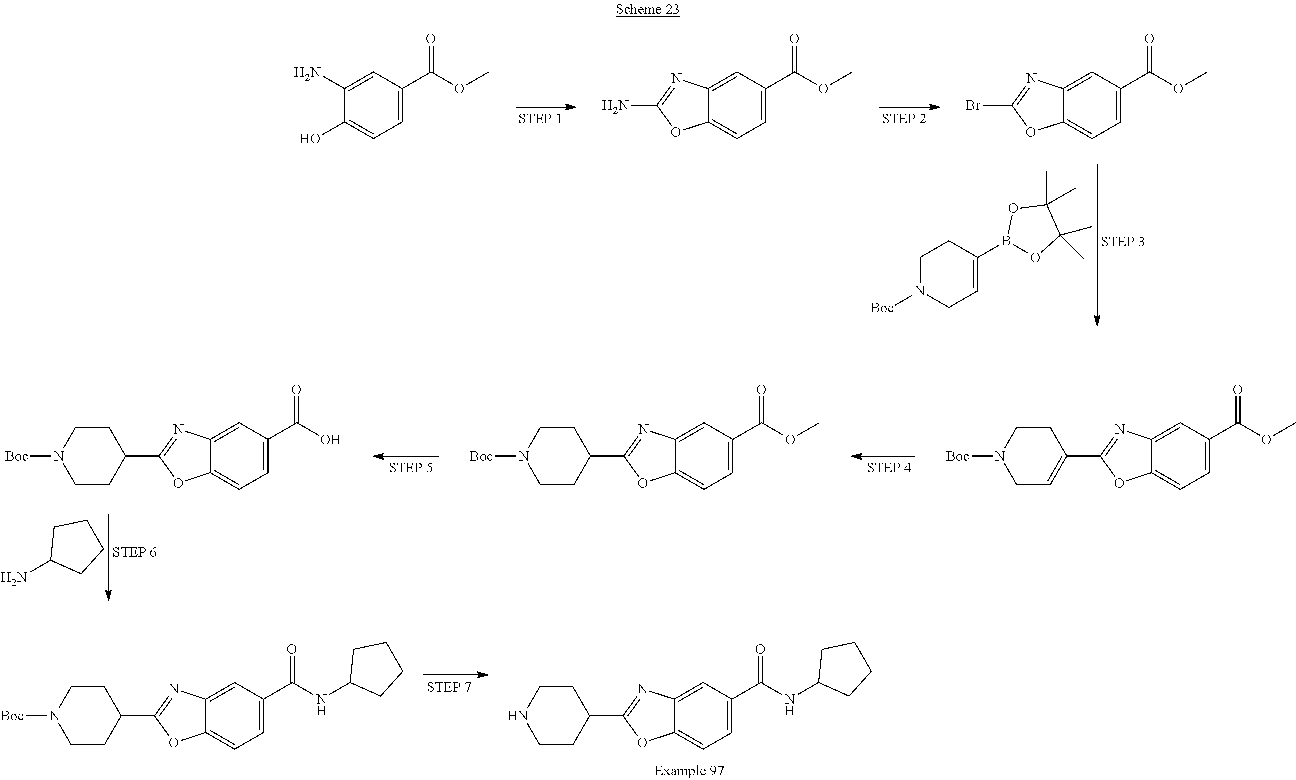

### Step 1 (Scheme 23): Synthesis of methyl 2-aminobenzo[d]oxazole-5-carboxylate

To a stirred solution of methyl 3-amino-4-hydroxybenzoate (10 g, 59.8 mmol) in methanol (10 mL) at 0° C. was added cyanogen bromide (7.60 g, 71.8 mmol). The reaction mixture was warmed to r.t. and stirred for 16 h, then concentrated under reduced pressure. Saturated sodium bicarbonate solution (50 mL) was added and the resulting solid was filtered off and dried. The crude product was purified by flash chromatography, eluting with 80 to 100% ethyl acetate in petroleum ether, to obtain methyl 2-aminobenzo[d]oxazole-5-carboxylate (7.8 g, 58%) as an off-white solid.

### Step 2 (Scheme 23): Synthesis of methyl 2-bromobenzo[d]oxazole-5-carboxylate

To a stirred solution of copper(II) bromide (12.4 g, 55.4 mmol) in acetonitrile (40 mL) at 0° C. was added tert-butyl nitrite (5.71 g, 55.4 mmol) dropwise. The mixture was stirred at 0° C. for 20 minutes, then methyl 2-aminobenzo[d]oxazole-5-carboxylate (5.6 g, 29.1 mmol) was added portionwise. The reaction mixture was allowed to warm to r.t. and stirred for 16 h, then diluted with EtOAc (50 mL), filtered through Celite®, washing with ethyl acetate (10 mL), and concentrated under reduced pressure. The residue was purified by flash chromatography, eluting with 0% to 15% ethyl acetate in petroleum ether to obtain methyl 2-bromobenzo[d]oxazole-5-carboxylate (4.0 g, 54%) as an off-white solid.

### Step 3 (Scheme 23): Synthesis of methyl 2-(1-(tert-butoxycarbonyl)-1,2,3,6-tetrahydropyridin-4-yl)benzo[d]oxazole-5-carboxylate

To a mixture of methyl 2-bromobenzo[d]oxazole-5-carboxylate (300 mg, 1.17 mmol) and tert-butyl 4-(4,4,5,5-tetramethyl-1,3,2-dioxaborolan-2-yl)-3,6-dihydropyridine-1 (2H)-carboxylate (435 mg, 1.41 mmol) in toluene (2.0 mL) were added potassium phosphate, anhydrous (612 mg, 3.51 mmol), ethanol (1 mL) and Pd(Ph₃P)₄ (135 mg, 0.117 mmol) under continuous bubbling of nitrogen. The reaction mixture was stirred at 100° C. for 1 h, then concentrated under reduced pressure. The residue was purified by flash chromatography, eluting with 20% to 40% ethyl acetate in petroleum ether, to afford methyl 2-(1-(tert-butoxycarbonyl)-1,2,3,6-tetrahydropyridin-4-yl)benzo[d]oxazole-5-carboxylate (85 mg, 20%) as an off-white solid.

### Step 4 (Scheme 23): Synthesis of methyl 2-(1-(tert-butoxycarbonyl)piperidin-4-yl)benzo[d]oxazole-5-carboxylate

To a stirred solution of methyl 2-(1-(tert-butoxycarbonyl)-1,2,3,6-tetrahydropyridin-4-yl)benzo[d]oxazole-5-carboxylate (0.28 g, 0.78 mmol) in ethanol (40 mL) was added Pd/C (10%, dry; 0.056 g). The reaction mixture was stirred at r.t. for 6 h under H₂ bladder pressure, then filtered through Celite®, washing with ethanol (10 mL) and concentrated under reduced pressure to obtain crude methyl 2-(1-(tert-butoxycarbonyl)piperidin-4-yl)benzo[d]oxazole-5-carboxylate (250 mg), which was taken forward to the next step without further purification.

### Step 5 (Scheme 23): Synthesis of 2-(1-(tert-butoxycarbonyl)piperidin-4-yl)benzo[d]oxazole-5-carboxylic acid

To a stirred solution of methyl 2-(1-(tert-butoxycarbonyl)piperidin-4-yl)benzo[d]oxazole-5-carboxylate (250 mg, 0.694 mmol) in THF:water (7:3; 10 mL) at 0° C. was added LiOH·H₂O (49.8 mg, 2.08 mmol). The reaction mixture was allowed to warm to r.t. and stirred for 5 h, then concentrated under reduced pressure. The residue was dissolved in water (2 mL), acidified with saturated citric acid solution to around pH 4 and extracted with 10% MeOH in DCM (2×10 mL). The combined organic extract was washed with brine (10 mL), dried over anhydrous sodium sulfate, filtered and concentrated under reduced pressure to afford 2-(1-(tert-butoxycarbonyl)piperidin-4-yl)benzo[d]oxazole-5-carboxylic acid (180 mg, 75%) as a solid.

### Step 6 (Scheme 23): Synthesis of tert-butyl 4-(5-(cyclopentylcarbamoyl)benzo[d]oxazol-2-yl)piperidine-1-carboxylate

To a stirred solution of 2-(1-(tert-butoxycarbonyl)piperidin-4-yl)benzo[d]oxazole-5-carboxylic acid (180 mg, 0.52 mmol) in DCM (10 mL) at 0° C. was added DIPEA (201 mg, 1.56 mmol) and n-propylphosphonic acid anhydride, cyclic trimer (50% in ethyl acetate; 661 mg, 1.04 mmol). The mixture was stirred at 0° C. for 15 minutes, after which cyclopentanamine (53.1 mg, 0.624 mmol) was added. The mixture was allowed to warm to r.t. and stirred for 16 h, then concentrated under reduced pressure, diluted with water (10 mL) and extracted with 10% MeOH in DCM (2×10 mL). The combined organic extract was washed with brine (10 mL), dried over anhydrous sodium sulfate, filtered and concentrated under reduced pressure. The residue was purified by flash chromatography, eluting with 20% to 40% ethyl acetate in petroleum ether, to obtain tert-butyl 4-(5-(cyclopentylcarbamoyl)benzo[d]oxazol-2-yl)piperidine-1-carboxylate (70 mg, 32%) as an off-white solid.

### Step 7 (Scheme 23) leading to Example 97: Synthesis of N-cyclopentyl-2-(piperidin-4-yl)benzo[d]oxazole-5-carboxamide

To a stirred solution of tert-butyl 4-(5-(cyclopentylcarbamoyl)benzo[d]oxazol-2-yl)piperidine-1-carboxylate (110 mg, 0.266 mmol) in DCM (10 mL) at 0° C. was added HCl in 1,4-dioxane (4 M, 0.067 mL, 0.266 mmol). The reaction was stirred at r.t. for 16 h, then concentrated under reduced pressure. The residue was diluted with brine (10 mL), basified with saturated sodium bicarbonate solution (5 mL) and extracted with 10% MeOH in DCM (2×10 mL). The combined organic extract was dried over anhydrous sodium sulfate, filtered and concentrated under reduced pressure. The residue was purified by preparative HPLC to afford N-cyclopentyl-2-(piperidin-4-yl)benzo[d]oxazole-5-carboxamide (15.0 mg, 18%) as an off-white solid.

¹H NMR: δ_{H} (400 MHz, DMSO-d₆) 8.36 (1H, d, J7.2), 8.22 (1H, d, J 1.2), 7.91 (1H, dd, J 1.6, 8.4), 7.76 (1H, d, J 8.4), 4.28-4.24 (1H, m), 3.33-3.03 (3H, m), 3.06-3.03 (2H, m), 2.34-2.22 (2H, m), 1.96-1.90 (4H, m), 1.90-1.71 (2H, m), 1.71-1.60 (4H, m).

### Example 98: N-cyclopentyl-2-(1-methylpiperidin-4-yl)benzo[d]oxazole-5-carboxamide

Example 98 may be prepared by reductive alkylation with formaldehyde according to the route shown in Scheme 9, using N-cyclopentyl-2-(piperidin-4-yl)benzo[d]oxazole-5-carboxamide instead of (S)—N-(chroman-4-yl)-2-(piperidin-4-yl)-[1,2,4]triazolo[1,5-a]pyridine-6-carboxamide hydrochloride.

¹H NMR: δ_{H} (400 MHz, DMSO-d₆) 8.30-8.36 (1H, m), 8.19 (1H, s), 7.87 (1H, dd, J 8.4, 2.0), 7.73 (1H, d, J 8.4), 4.24-4.21 (1H, m), 2.99-2.96 (1H, m), 2.81-2.78 (2H, m), 2.18 (3H, s), 2.08-2.03 (4H, m), 1.90-1.87 (4H, m), 1.82-1.70 (2H, m), 1.57-1.54 (4H, m).

### Example 99: N-cyclopentyl-2-(1-ethylpiperidin-4-yl)benzo[d]oxazole-5-carboxamide

Example 99 may be prepared by reductive alkylation with acetaldehyde according to the route shown in Scheme 10, using N-cyclopentyl-2-(piperidin-4-yl)benzo[d]oxazole-5-carboxamide instead of (S)—N-(chroman-4-yl)-2-(piperidin-4-yl)-[1,2,4]triazolo[1,5-a]pyridine-6-carboxamide hydrochloride.

¹H NMR: δ_{H} (400 MHz, DMSO-d₆) 8.30-8.36 (1H, m), 8.20 (1H, d, J 1.6), 7.88 (1H, dd, J 8.4, 2.0), 7.73 (1H, d, J 8.4), 4.25-4.24 (1H, m), 3.00-2.89 (3H, m), 2.37-2.33 (2H, m), 2.11-2.05 (4H, m), 1.89-1.81 (4H, m), 1.71-1.69 (2H, m), 1.60-1.55 (4H, m), 1.02 (3H, t, J 7.2).

### Example 100: (S)—N-(chroman-4-yl)-2-(piperazin-1-yl)benzo[d]oxazole-5-carboxamide

Example 100 may be prepared according to the method of Scheme 24.

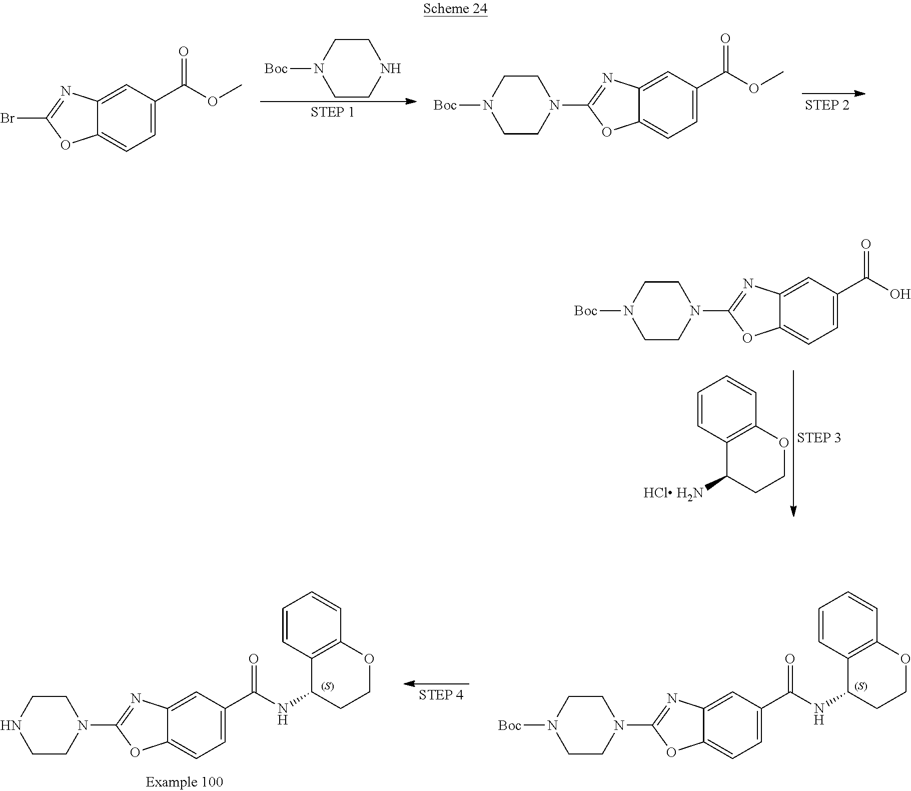

### Step 1 (Scheme 24): Synthesis of methyl 2-(4-(tert-butoxycarbonyl)piperazin-1-yl)benzo[d]oxazole-5-carboxylate

To a stirred solution of methyl 2-bromobenzo[d]oxazole-5-carboxylate (500 mg, 1.95 mmol) in acetonitrile (30 mL) at r.t. was added tert-butyl piperazine-1-carboxylate (436 mg, 2.34 mmol) and K₂CO₃ (540 mg, 3.91 mmol). The reaction was heated to 80° C. for 2 h, then diluted with water (10 mL) and extracted with 10% MeOH in DCM (2×20 mL). The combined organic extract was washed with brine (10 mL), dried over anhydrous sodium sulfate, filtered and concentrated under reduced pressure. The residue was purified by flash chromatography, eluting with 50% to 60% ethyl acetate in petroleum ether, to afford methyl 2-(4-(tert-butoxycarbonyl)piperazin-1-yl)benzo[d]oxazole-5-carboxylate (690 mg, 97%) as an off-white solid.

### Step 2 (Scheme 24): Synthesis of 2-(4-(tert-butoxycarbonyl)piperazin-1-yl)benzo[d]oxazole-5-carboxylic acid

To a stirred solution of methyl 2-(4-(tert-butoxycarbonyl)piperazin-1-yl)benzo[d]oxazole-5-carboxylate (620 mg, 1.72 mmol) in THF:water (7:3; 10 mL) at 0° C. was added LiOH·H₂O (82 mg, 3.43 mmol). The mixture was allowed to warm to r.t. and stirred for 16 h, then concentrated under reduced pressure, diluted with water (10 mL), and acidified with saturated aqueous citric acid to around pH 4. The solid precipitate was filtered off and dried under vacuum to afford 2-(4-(tert-butoxycarbonyl)piperazin-1-yl)benzo[d]oxazole-5-carboxylic acid (600 mg) as an off-white solid.

### Step 3 (Scheme 24): Synthesis of tert-butyl (S)-4-(5-(chroman-4-ylcarbamoyl)benzo[d]oxazol-2-yl)piperazine-1-carboxylate

To a stirred solution of 2-(4-(tert-butoxycarbonyl)piperazin-1-yl)benzo[d]oxazole-5-carboxylic acid (500 mg, 1.41 mmol) in DCM (10 mL) at 0° C. was added DIPEA (730 mg, 5.64 mmol) and n-propylphosphonic acid anhydride, cyclic trimer (50% in ethyl acetate; 1.8 g, 2.82 mmol). The mixture was stirred for 15 minutes, after which (S)-chroman-4-amine hydrochloride salt (314 mg, 1.69 mmol) was added and the mixture was allowed to warm to r.t. and stirred for 16 h. The reaction mixture was diluted with water (10 mL) and extracted with 10% MeOH in DCM (2×10 mL). The combined organic extract was washed with brine (10 mL), dried over anhydrous sodium sulfate, filtered and concentrated under reduced pressure. The residue was purified by flash chromatography, eluting with 50% to 80% ethyl acetate in petroleum ether, to afford tert-butyl (S)-4-(5-(chroman-4-ylcarbamoyl)benzo[d]oxazol-2-yl)piperazine-1-carboxylate (400 mg, 59%) as an off-white solid.

### Step 4 (Scheme 24) leading to Example 100: Synthesis of (S)—N-(chroman-4-yl)-2-(piperazin-1-yl)benzo[d]oxazole-5-carboxamide

To a stirred solution of tert-butyl (S)-4-(5-(chroman-4-ylcarbamoyl)benzo[d]oxazol-2-yl)piperazine-1-carboxylate (0.4 g, 0.84 mmol) in DCM (10 mL) at 0° C. was added HCl in dioxane (4M; 0.21 mL, 0.84 mmol). The reaction mixture was allowed to warm to r.t. and stirred for 16 h, then concentrated under reduced pressure, washed with acetonitrile (10 mL) and dried under lyophilization to obtain the title compound as a hydrochloride salt (40 mg, 12%).

¹H NMR: δ_{H} (400 MHz, DMSO-d₆) 9.20 (2H, br s), 8.84 (1H, d, J8.4), 7.89 (1H, d, J 1.6), 7.71 (1H, dd, J 1.6, 8.4), 7.52 (1H, d, J 8.4), 7.19-7.15 (2H, m), 6.89-6.80 (2H, m), 5.32-5.27 (1H, m), 4.26-4.22 (2H, m), 3.87-3.58 (4H, m), 3.21-3.27 (4H, m), 2.09-2.04 (2H, m).

### Example 101: N-cyclopentyl-2-(piperazin-1-yl)benzo[d]oxazole-5-carboxamide

Example 101 may be prepared according to the method of Scheme 24, using cyclopentylamine instead of (S)-chroman-4-amine hydrochloride salt in Step 3. The title compound was isolated as a hydrochloride salt.

¹H NMR: δ_{H} (400 MHz, DMSO-d₆) 9.26 (2H, s), 8.24 (1H, d, J7.2), 7.83 (1H, d, J 1.2), 7.62 (1H, dd, J 2.0, 8.4), 7.50 (1H, d, J 8.4), 4.23-4.22 (1H, m), 3.87-3.85 (4H, m), 3.27 (4H, m), 1.90-1.87 (2H, m), 1.70-1.67 (2H, m), 1.59-1.51 (4H, m).

### Example 102: (S)—N-(chroman-4-yl)-2-(4-ethylpiperazin-1-yl)benzo[d]oxazole-5-carboxamide

Example 102 may be prepared according to the method of Scheme 25.

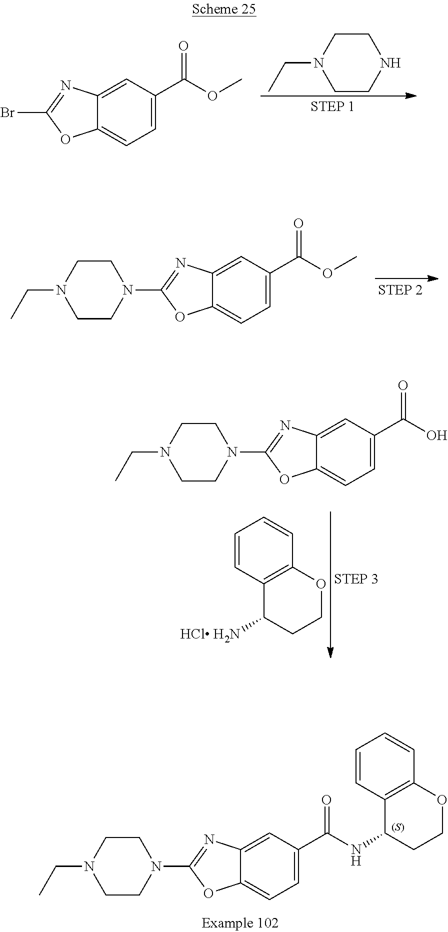

### Step 1 (Scheme 25): Synthesis of methyl 2-(4-ethylpiperazin-1-yl)benzo[d]oxazole-5-carboxylate

To a stirred solution of methyl 2-bromobenzo[d]oxazole-5-carboxylate (300 mg, 1.17 mmol) in acetonitrile (20 mL) was added K₂CO₃ (324 mg, 2.34 mmol) and 1-ethylpiperazine (161 mg, 1.41 mmol). The mixture was warmed to 80° C. and stirred for 2 h, then concentrated under reduced pressure. The residue was diluted with water (5 mL) and extracted with 10% MeOH in DCM (3×10 mL). The combined organic extract was washed with brine (10 mL), dried over anhydrous sodium sulfate, filtered and concentrated under reduced pressure to afford methyl 2-(4-ethylpiperazin-1-yl)benzo[d]oxazole-5-carboxylate as a solid (350 mg).

### Step 2 (Scheme 25): Synthesis of 2-(4-ethylpiperazin-1-yl)benzo[d]oxazole-5-carboxylic acid

To a stirred solution of methyl 2-(4-ethylpiperazin-1-yl)benzo[d]oxazole-5-carboxylate (350 mg, 1.21 mmol) in THF (7 mL) and water (3 mL) was added lithium hydroxide (57.9 mg, 2.42 mmol). The mixture was and stirred at r.t. for 16 h, then concentrated under reduced pressure. The residue was washed with acetone and 5% ethyl acetate in petroleum ether to obtain the lithium salt of the title compound (320 mg) as a yellow solid.

### Step 3 (Scheme 25) leading to Example 102: Synthesis of (S)—N-(chroman-4-yl)-2-(4-ethylpiperazin-1-yl)benzo[d]oxazole-5-carboxamide

To a stirred solution of 2-(4-ethylpiperazin-1-yl)benzo[d]oxazole-5-carboxylic acid, lithium salt (200 mg, 0.71 mmol) in DCM (10 mL) at 0° C. was added DIPEA (0.523 mL, 2.83 mmol) and n-propylphosphonic acid anhydride, cyclic trimer (50% in ethyl acetate; 0.902 mL, 1.42 mmol). The mixture was allowed to warm to r.t. and stirred for 15 minutes, followed by addition of (S)-chroman-4-amine, hydrochloride salt (158 mg, 0.85 mmol). The mixture was stirred at r.t. for 16 h, then diluted with water (5 mL) and extracted with 10% MeOH in DCM (3×10 mL). The combined organic extract was washed with brine (10 mL), dried over anhydrous sodium sulfate, filtered and concentrated under reduced pressure. The residue was purified by preparative HPLC to afford (S)—N-(chroman-4-yl)-2-(4-ethylpiperazin-1-yl)benzo[d]oxazole-5-carboxamide (70 mg, 24%) as an off-white solid.

¹H NMR: δ_{H} (400 MHz, DMSO-d₆) 8.79 (1H, d, J 8.4), 7.84 (1H, d, J 1.6), 7.65 (1H, dd, J 1.6, 8.2), 7.46 (1H, d, J 8.4), 7.19-7.14 (2H, m), 6.89-6.76 (2H, m), 5.32-5.27 (1H, m), 4.30-4.21 (2H, m), 3.63-3.61 (4H, m), 2.42-2.37 (2H, m), 2.10-2.05 (2H, m), 1.06-1.04 (3H, m). Note: One signal (4H) coincides with the DMSO signal.

### Example 103: N-cyclopentyl-2-(4-ethylpiperazin-1-yl)benzo[d]oxazole-5-carboxamide

Example 103 may be prepared according to the method of Scheme 25, using cyclopentylamine instead of (S)-chroman-4-amine hydrochloride salt in Step 3.

¹H NMR: δ_{H} (400 MHz, DMSO-d₆) 8.20 (1H, d, J 7.2), 7.77 (1H, s), 7.56 (1H, dd, J 1.6), 7.44 (1H, d, J 8.4), 4.27-4.18 (1H, m), 3.64-3.60 (4H, m), 2.50-2.42 (4H, m), 2.42-2.37 (2H, m), 1.92-1.86 (2H, m), 1.89-1.86 (2H, m), 1.58-1.51 (4H, m), 1.04 (3H, t, J 7.2).

### Example 104: (S)—N-(chroman-4-yl)-2-(4-methylpiperazin-1-yl)benzo[d]oxazole-5-carboxamide

Example 104 may be prepared according to the method of Scheme 25, using 1-methylpiperazine instead of 1-ethylpiperazine in Step 1.

¹H NMR: δ_{H} (400 MHz, DMSO-d₆) 8.79 (1H, d, J 8.0), 7.84 (1H, d, J 1.2), 7.65 (1H, dd, J 1.6, 8.4), 7.46 (1H, d, J 8.4), 7.18-7.14 (2H, m), 6.89-6.86 (1H, m), 6.81-6.79 (1H, m), 5.30-5.29 (1H, m), 4.32-4.30 (1H, m), 4.27-4.24 (1H, m), 3.63-3.61 (4H, m), 2.42-2.33 (4H, m), 2.24 (3H, s), 2.13-2.10 (2H, m).

### Example 105: N-cyclopentyl-2-(4-methylpiperazin-1-yl)benzo[d]oxazole-5-carboxamide

Example 105 may be prepared according to the method of Scheme 25, using 1-methylpiperazine instead of 1-ethylpiperazine in Step 1 and using cyclopentylamine instead of (S)-chroman-4-amine hydrochloride salt in Step 3.

¹H NMR: δ_{H} (400 MHz, DMSO-d₆) 8.22-8.18 (1H, m), 7.78-7.77 (1H, m), 7.57 (1H, dd, J 2.0, 8.4), 7.44 (1H, d, J 8.4), 4.25-4.20 (1H, m), 3.63-3.61 (4H, m), 2.45-2.42 (4H, m), 2.24 (3H, s), 1.92-1.86 (2H, m), 1.70-1.67 (2H, m), 1.58-1.51 (4H, m).

### Example 106: (S)—N-(chroman-4-yl)-2-(4-(2-hydroxyethyl)piperazin-1-yl)benzo[d]oxazole-5-carboxamide

Example 106 may be prepared according to the method of Scheme 26.

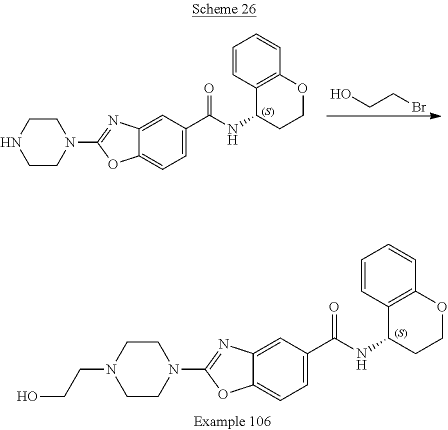

To a stirred solution of (S)—N-(chroman-4-yl)-2-(piperazin-1-yl)benzo[d]oxazole-5-carboxamide (100 mg, 0.264 mmol) in acetonitrile (10 mL) under nitrogen were added potassium carbonate (110 mg, 0.793 mmol) and 2-bromoethan-1-ol (100 mg, 0.793 mmol). The reaction mixture was stirred at 80° C. for 16 h, then concentrated under reduced pressure. The residue was diluted with ice cold water (15 mL) and extracted with 5% MeOH in DCM (3×20 mL). The combined organic extract was washed with brine (5 mL), dried over anhydrous sodium sulfate, filtered and concentrated under reduced pressure. The crude product was purified by preparative HPLC using NH₄HCO₃ as buffer to afford the title compound (96 mg, 86%) as an off-white solid.

¹H NMR: δ_{H} (400 MHz, DMSO-d₆) 8.79 (1H, d, J 8.4), 7.84 (1H, d, J 1.6), 7.65 (1H, dd, J 2.0, 8.4), 7.46 (1H, d, J 8.4), 7.19-7.14 (2H, m), 6.88-6.86 (1H, m), 6.81-6.79 (1H, m), 5.32-5.27 (1H, m), 4.25-4.21 (3H, m), 3.63-3.60 (4H, m), 3.55-3.52 (2H, m), 2.55-2.51 (4H, m), 2.51-2.50 (2H, m), 2.11-2.08 (2H, m).

### Example 107: N-cyclopentyl-2-(4-(2-hydroxyethyl)piperazin-1-yl)benzo[d]oxazole-5-carboxamide

Example 107 may be prepared according to the method of Scheme 26, using N-cyclopentyl-2-(piperazin-1-yl)benzo[d]oxazole-5-carboxamide instead of (S)—N-(chroman-4-yl)-2-(piperazin-1-yl)benzo[d]oxazole-5-carboxamide.

¹H NMR: δ_{H} (400 MHz, DMSO-d₆) 8.21 (1H, d, J7.2), 7.77 (1H, d, J 1.6), 7.56 (1H, dd, J 1.6, 8.2), 7.44 (1H, d, J 8.4), 4.49-4.25 (1H, m), 4.23-4.19 (1H, m), 3.70-3.56 (4H, m), 3.54-3.53 (2H, m), 2.54-2.51 (4H, m), 2.47-2.44 (2H, m), 1.89-1.86 (2H, m), 1.70-1.67 (2H, m), 1.67-1.56 (4H, m).

### Example 108: N-cyclopentyl-2-(4-ethylpiperazin-1-yl)benzo[d]oxazole-6-carboxamide

Example 108 may be prepared according to the method of Scheme 27:

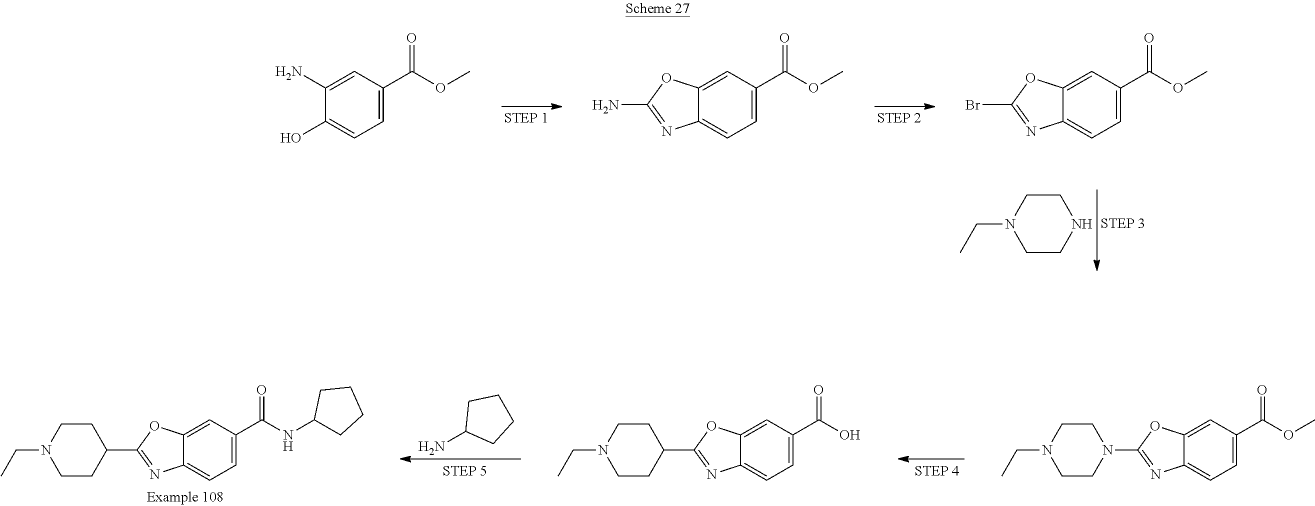

### Step 1 (Scheme 27): Synthesis of methyl 2-aminobenzo[d]oxazole-6-carboxylate

To a stirred solution of methyl 4-amino-3-hydroxybenzoate (10 g, 29.9 mmol) in methanol (100 mL) at 0° C. was added cyanogen bromide (7.60 g, 35.9 mmol). The reaction mixture was stirred at r.t. for 16 h, then concentrated under reduced pressure. The residue was diluted with water (10 mL) and basified with a saturated sodium bicarbonate solution. The solid precipitate was filtered off, washing with water and dried under vacuum to afford crude methyl 2-aminobenzo[d]oxazole-6-carboxylate (9.50 g, 81%) as a grey solid, which was taken forward to the next step without further purification.

### Step 2 (Scheme 27): Synthesis of methyl 2-bromobenzo[d]oxazole-6-carboxylate

To a stirred solution of copper(II) bromide (11.0 g, 49.4 mmol) in acetonitrile (40 mL) at 0° C. was added tert-butyl nitrite (5.10 g, 49.4 mmol) dropwise. The mixture was stirred at 0° C. for 40 minutes, then methyl 2-aminobenzo[d]oxazole-6-carboxylate (5.0 g, 26.0 mmol) was added portionwise. The reaction mixture was was allowed to warm to r.t. and stirred for 16 h, then diluted with EtOAc (50 mL), filtered through Celite®, washing with ethyl acetate (10 mL), and concentrated under reduced pressure. The residue was purified by flash chromatography, eluting with 0% to 20% ethyl acetate in petroleum ether to obtain methyl 2-bromobenzo[d]oxazole-6-carboxylate (1.7 g, 26%) as an off-white solid.

### Step 3 (Scheme 27): Synthesis of methyl 2-(4-ethylpiperazin-1-yl)benzo[d]oxazole-6-carboxylate

To a stirred solution of methyl 2-bromobenzo[d]oxazole-6-carboxylate (0.35 g, 1.37 mmol) in acetonitrile (10 mL) at r.t. were added potassium carbonate (0.38 g, 2.73 mmol) and 1-ethylpiperazine (0.187 g, 1.64 mmol). The reaction mixture was stirred at 80° C. for 2 h, then concentrated under reduced pressure. The residue was diluted with water (25 mL) and extracted with 10% MeOH in DCM (3×25 mL). The combined organic extract was washed with brine (30 mL), dried over anhydrous sodium sulfate, filtered and concentrated under reduced pressure to afford methyl 2-(4-ethylpiperazin-1-yl)benzo[d]oxazole-6-carboxylate (0.34 g) as a yellow solid.

### Step 4 (Scheme 27): Synthesis of 2-(4-ethylpiperazin-1-yl)benzo[d]oxazole-6-carboxylic acid

To a stirred solution of methyl 2-(4-ethylpiperazin-1-yl)benzo[d]oxazole-6-carboxylate (0.33 g, 1.14 mmol) in THF (2 mL) and water (1 mL) was added lithium hydroxide (0.144 g, 3.42 mmol) at 0° C. The reaction mixture was stirred at r.t. for 16 h, then concentrated under reduced pressure. The residue was triturated with pentane and dried over vaccum to afford 2-(4-ethylpiperazin-1-yl)benzo[d]oxazole-6-carboxylic acid, lithium salt (0.4 g) as a white solid.

### Step 5 (Scheme 27) leading to Example 108: Synthesis of N-cyclopentyl-2-(4-ethylpiperazin-1-yl)benzo[d]oxazole-6-carboxamide

To a stirred solution of 2-(4-ethylpiperazin-1-yl)benzo[d]oxazole-6-carboxylic acid, lithium salt (0.30 g, 1.09 mmol) in DCM (8 mL) at 0° C. was added n-propylphosphonic acid anhydride, cyclic trimer (1.39 g, 2.18 mmol) and DIPEA (0.563 g, 4.36 mmol). The mixture was stirred at 0° C. for 15 minutes, followed by addition of cyclopentanamine (50% in ethyl acetate; 0.111 g, 1.31 mmol). The mixture was stirred at r.t. for 16 h, then diluted with water (30 mL) and extracted with 15% MeOH in DCM (3×20 mL). The combined organic extract was washed with brine (40 mL), dried over anhydrous sodium sulfate, filtered and concentrated under reduced pressure. The residue was purified by preparative HPLC to afford N-cyclopentyl-2-(4-ethylpiperazin-1-yl)benzo[d]oxazole-6-carboxamide (0.08 g, 21%) as an off-white solid.

¹H NMR: δ_{H} (400 MHz, DMSO-d₆) 8.15 (1H, d, J 7.2), 7.89-7.88 (1H, m), 7.75 (1H, dd, J 1.6), 7.29 (1H, d, J 8.4), 4.25-4.20 (1H, m), 3.65-3.64 (4H, m), 2.68-2.67 (4H, m), 2.56-2.52 (2H, m), 2.51-2.50 (2H, m), 2.48 (2H, m), 2.42-2.37 (4H, m), 2.34-2.33 (3H, m).

### Example 109: N-cyclopentyl-2-(piperazin-1-yl)benzo[d]oxazole-6-carboxamide

The title compound may be prepared according to the method of Scheme 24, using methyl 2-bromobenzo[d]oxazole-6-carboxylate instead of methyl 2-bromobenzo[d]oxazole-5-carboxylate in Step 1 and cyclopentylamine instead of (S)-chroman-4-amine hydrochloride in Step 3.

¹H NMR: δ_{H} (400 MHz, DMSO-d₆) 8.15 (1H, d, J7.2), 7.88 (1H, d, J 1.2), 7.73 (1H, dd, J 1.2, 8.2), 7.28 (1H, d, J 8.0), 4.25-4.20 (1H, m), 3.57-3.54 (4H, m), 2.82-2.80 (4H, m), 1.92-1.87 (2H, m), 1.70 (2H, m), 1.56-1.54 (4H, m).

### Example 110: (S)—N-(chroman-4-yl)-2-(1-methyl-1H-pyrazol-4-yl)benzo[d]oxazole-6-carboxamide

The title compound may be prepared according to the method of Scheme 28.

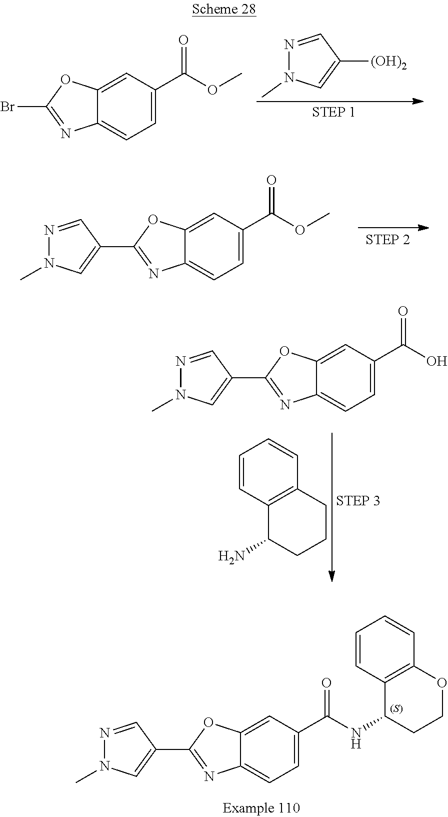

### Step 1 (Scheme 28): Synthesis of methyl 2-(1-methyl-1H-pyrazol-4-yl)benzo[d]oxazole-6-carboxylate

To a de-gassed solution of methyl 2-bromobenzo[d]oxazole-5-carboxylate (0.30 g, 1.17 mmol) in toluene (9 mL) and ethanol (3 mL) under nitrogen was added (1-methyl-1H-pyrazol-4-yl)boronic acid (0.177 g, 1.41 mmol), K₂CO₃ (0.486 g, 3.51 mmol) and Pd(Ph₃P)₄ (0.135 g, 0.117 mmol) under continuous bubbling of nitrogen. The mixture was stirred at 100° C. for 1 h, then cooled to r.t. and filtered through Celite®, washing with ethyl acetate (50 mL). The filtrate was concentrated under reduced pressure. The residue was purified by flash chromatography, eluting with 0% to 30% ethyl acetate in petroleum ether, to obtain methyl 2-(1-methyl-1H-pyrazol-4-yl)benzo[d]oxazole-6-carboxylate (0.20 g, 53%) as an off-white solid.

### Step 2 (Scheme 28): Synthesis of 2-(1-methyl-1H-pyrazol-4-yl)benzo[d]oxazole-6-carboxylic acid

To a stirred solution of methyl 2-(1-methyl-1H-pyrazol-4-yl)benzo[d]oxazole-6-carboxylate (0.20 g, 0.78 mmol) in THF (7 mL) and water (3 mL) at 0° C. was added lithium hydroxide (0.056 g, 2.33 mmol). The mixture was stirred at r.t. for 16 h, then concentrated under reduced pressure. The residue was diluted with water (10 mL) and washed with diethyl ether (20 mL). The aqueous layer was separated, acidified with saturated citric acid solution to around pH 4, then extracted with 10% MeOH in DCM (3×20 mL). The combined organic extract was washed with brine (10 mL), dried over anhydrous sodium sulfate, filtered and concentrated under reduced pressure to afford 2-(1-methyl-1H-pyrazol-4-yl)benzo[d]oxazole-6-carboxylic acid (0.10 g) as an off-white solid.

### Step 3 (Scheme 28) leading to Example 110: Synthesis of (S)—N-(chroman-4-yl)-2-(1-methyl-1H-pyrazol-4-yl)benzo[d]oxazole-6-carboxamide

To a stirred solution of 2-(1-methyl-1H-pyrazol-4-yl)benzo[d]oxazole-6-carboxylic acid (0.05 g, 0.21 mmol) in DCM (5 mL) at 0° C. was added n-propylphosphonic acid anhydride, cyclic trimer (0.13 g, 0.41 mmol) and DIPEA (0.11 g, 0.82 mmol). The mixture was stirred at 0° C. for 15 minutes, followed by addition of (S)-chroman-4-amine (0.046 g, 0.31 mmol). The mixture was stirred at r.t. for 16 h, then diluted with water (15 mL) and extracted with 10% MeOH in DCM (3×20 mL). The combined organic extract was washed with brine (30 mL), dried over anhydrous sodium sulfate, filtered and concentrated under reduced pressure. The residue was purified by preparative HPLC to afford (S)—N-(chroman-4-yl)-2-(1-methyl-1H-pyrazol-4-yl)benzo[d]oxazole-6-carboxamide (35 mg, 44%) as an off-white solid.

¹H NMR: δ_{H} (400 MHz, DMSO-d₆) 8.96 (1H, d, J 8.4), 8.62 (1H, s), 8.25 (1H, d, J 1.2), 8.16 (1H, m), 7.99 (1H, dd, J 1.6, 8.0), 7.75 (1H, d, J 8.4), 7.22-7.15 (2H, m), 6.91-6.87 (1H, m), 6.83-6.81 (1H, m), 5.35-5.30 (1H, m), 4.27-4.24 (2H, m), 3.97 (3H, s), 2.11-2.07 (2H, m).

### Examples 111 and 112

Examples 111 and 112 may be prepared according to the method of Scheme 29.

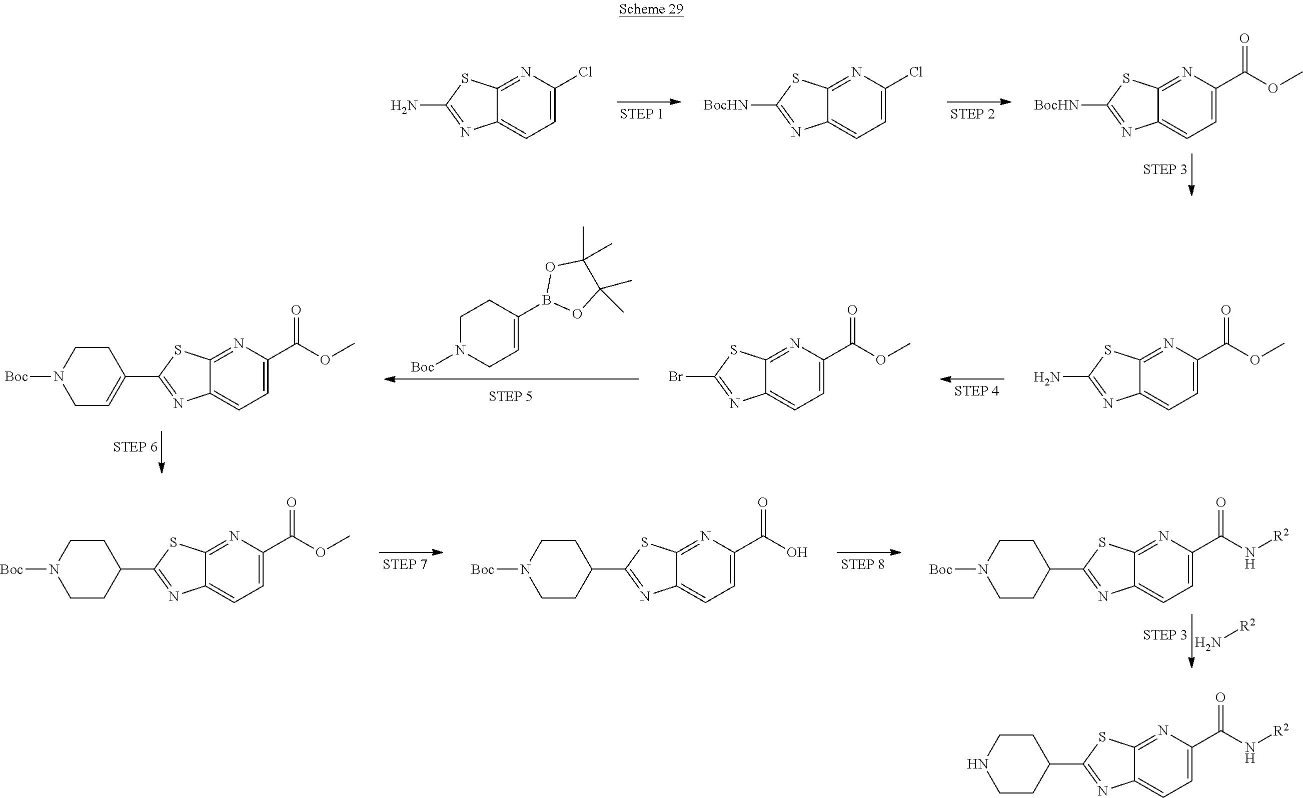

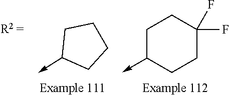

### Step 1 (Scheme 29): Synthesis of tert-butyl (5-chlorothiazolo[5,4-b]pyridin-2-yl)carbamate

To a stirred solution of 5-chlorothiazolo[5,4-b]pyridin-2-amine (500 mg, 2.69 mmol) in DCM (10 mL) at 0° C. were added DMAP (65.8 mg, 0.539 mmol) and Boc anhydride (0.938 mL, 4.04 mmol). The mixture was stirred at r.t. for 16 h, then diluted with water (10 mL) and extracted with DCM (2×50 mL). The combined organic extract was washed with brine (20 mL), dried over anhydrous sodium sulfate, filtered and concentrated under reduced pressure. The residue was triturated with 10% EtOAc in petroleum ether to afford tert-butyl (5-chlorothiazolo[5,4-b]pyridin-2-yl)carbamate (400 mg, 47%).

### Step 2 (Scheme 29): Synthesis of methyl 2-((tert-butoxycarbonyl)amino)thiazolo[5,4-b]pyridine-5-carboxylate

To a stirred, degassed solution of tert-butyl (5-chlorothiazolo[5,4-b]pyridin-2-yl)carbamate (3.50 g, 12.3 mmol) in methanol (5 mL) and DMF (2.5 mL) in a Tinyclave steel pressure reactor under nitrogen was added potassium carbonate (2.88 g, 20.8 mmol) and palladium(II) acetate (0.357 g, 1.59 mmol) followed by 1,3-bis(diphenylphosphino)propane (0.657 g, 1.59 mmol). The reaction mixture was placed under CO pressure (48 psi) at 85° C. for 16 h. The resulting mixture was filtered through Celite®, washing with ethyl acetate (50 mL). The filtrate was washed with water (50 mL), brine (30 mL), dried over anhydrous sodium sulfate, filtered and concentrated under reduced pressure to afford methyl 2-((tert-butoxycarbonyl)amino)thiazolo[5,4-b]pyridine-5-carboxylate as an off-white solid (2.20 g, 57%).

### Step 3 (Scheme 29): Synthesis of methyl 2-aminothiazolo[5,4-b]pyridine-5-carboxylate

To a stirred solution of methyl 2-((tert-butoxycarbonyl)amino)thiazolo[5,4-b]pyridine-5-carboxylate (2.20 g, 7.11 mmol) in DCM (25 mL) at 0° C. was added TFA (52.1 mL, 676 mmol). The mixture was stirred at r.t. for 16 h, then concentrated under reduced pressure. The residue was dissolved in 10% MeOH in DCM, basified with triethylamine to around pH9, filtered through Celite®, washing with 10% MeOH in DCM and concentrated under reduced pressure to afford methyl 2-aminothiazolo[5,4-b]pyridine-5-carboxylate (1.10 g, 66%).

### Step 4 (Scheme 29): Synthesis of methyl 2-bromothiazolo[5,4-b]pyridine-5-carboxylate

To a stirred solution of copper(II) bromide (801 mg, 3.58 mmol) in acetonitrile (30 mL) under nitrogen at 0° C. was added tert-butyl nitrite (798 mg, 7.74 mmol) dropwise. After 20 minutes, methyl 2-aminothiazolo[5,4-b]pyridine-5-carboxylate (300 mg, 1.43 mmol) was added. The reaction mixture was stirred at r.t. for 16 h, then diluted with DCM (50 mL) and washed with aqueous HCl (1.5N; 10 mL). The organic layer was separated and washed with water (20 mL) and brine (20 mL) dried over anhydrous sodium sulfate, filtered and concentrated under reduced pressure to afford methyl 2-bromothiazolo[5,4-b]pyridine-5-carboxylate (200 mg, 77%) as a yellow solid.

### Step 5 (Scheme 29): Synthesis of methyl 2-(1-(tert-butoxycarbonyl)-1,2,3,6-tetrahydropyridin-4-yl)thiazolo[5,4-b]pyridine-5-carboxylate

To a stirred solution of methyl 2-bromothiazolo[5,4-b]pyridine-5-carboxylate (1.05 g, 3.84 mmol) in dioxane (90 mL) and water (10 mL) at r.t. under continuous bubbling of nitrogen was added tert-butyl 4-(4,4,5,5-tetramethyl-1,3,2-dioxaborolan-2-yl)-3,6-dihydropyridine-1 (2H)-carboxylate (1.43 g, 4.61 mmol) followed by potassium carbonate (1.06 g, 7.69 mmol). The mixture was stirred for 10 min, then tetrakis(triphenylphosphine)palladium(0) (0.222 g, 0.192 mmol) was added and bubbling of nitrogen was continued for a further 5 min. The reaction mixture was heated at 100° C. with stirring for 3 h, then concentrated under reduced pressure. The residue was dissolved in DCM (100 mL), washed with water (2×50 mL) and brine (2×50 mL), dried over anhydrous sodium sulfate, filtered and concentrated under reduced pressure. The residue was purified by flash chromatography, eluting with 50% to 55% ethyl acetate in petroleum ether, to afford methyl 2-(1-(tert-butoxycarbonyl)-1,2,3,6-tetrahydropyridin-4-yl)thiazolo[5,4-b]pyridine-5-carboxylate (0.80 g, 55%) as a brown solid.

### Step 6 (Scheme 29): Synthesis of methyl 2-(1-(tert-butoxycarbonyl)piperidin-4-yl)thiazolo[5,4-b]pyridine-5-carboxylate

To a de-gassed, stirred solution of methyl 2-(1-(tert-butoxycarbonyl)-1,2,3,6-tetrahydropyridin-4-yl)thiazolo[5,4-b]pyridine-5-carboxylate (750 mg, 2.00 mmol) in ethanol (10 mL) under nitrogen was added platinum(IV) oxide (454 mg, 2.00 mmol) over 15 minutes. The reaction mixture was stirred under hydrogen bladder pressure at r.t. for 16 h, then filtered through Celite®, washing with methanol (10 mL). The filtrate was concentrated under reduced pressure to afford methyl 2-(1-(tert-butoxycarbonyl)piperidin-4-yl)thiazolo[5,4-b]pyridine-5-carboxylate (0.73 g, 69%) as a brown solid.

### Step 7 (Scheme 29): Synthesis of 2-(1-(tert-butoxycarbonyl)piperidin-4-yl)thiazolo[5,4-b]pyridine-5-carboxylic acid

To a stirred solution of methyl 2-(1-(tert-butoxycarbonyl)piperidin-4-yl)thiazolo[5,4-b]pyridine-5-carboxylate (700 mg, 1.86 mmol) in THF (10 mL) methanol (3 mL) and water (3 mL) at 0° C. was added lithium hydroxide monohydrate (78 mg, 1.86 mmol). The mixture was stirred at r.t. for 16 h, then concentrated under reduced pressure, diluted with water (10 mL), acidified with saturated aqueous citric acid solution to around pH4 and extracted with 10% methanol in DCM (2×50 mL). The combined organic extract was washed with brine (20 mL), dried over anhydrous sodium sulfate, filtered and concentrated under reduced pressure to afford 2-(1-(tert-butoxycarbonyl)piperidin-4-yl)thiazolo[5,4-b]pyridine-5-carboxylic acid (550 mg, 37%).

### General Procedure for Step 8 (Scheme 29): Amide Coupling

To a stirred solution of 2-(1-(tert-butoxycarbonyl)piperidin-4-yl)thiazolo[5,4-b]pyridine-5-carboxylic acid (1.0 eq.) in DCM (10 vol.) at 0° C. were added the required amine (1.1 eq.), n-propylphosphonic acid anhydride, cyclic trimer (2.0 eq.) and N,N-diisopropylethylamine (5.0 eq.). The reaction mixture was stirred at r.t. for 16 h, then diluted with DCM (10 vol.), washed with water (5 vol.), and brine (5 vol.), dried over anhydrous sodium sulfate and concentrated under reduced pressure. The residue was purified by flash chromatography, eluting with 20% to 40% ethyl acetate in hexane, to afford the required Boc-protected amide.

### General Procedure for Step 9 (Scheme 29) Leading to Examples 111 and 112: Boc Deprotection

To a stirred solution of the Boc-protected amide (1 eq.) in DCM (10 vol.) at 0° C. was added HCl in dioxane (4 M; 2 eq.). The reaction mixture was allowed to warm to r.t. and stirred for 16 h, then concentrated under reduced pressure. The residue was purified by preparative HPLC using sodium bicarbonate as buffer to obtain Examples 111 and 112 as off-white solids.

### Example 111: N-cyclopentyl-2-(piperidin-4-yl)thiazolo[5,4-b]pyridine-5-carboxamide

Prepared according to Scheme 29 using cyclopentylamine as the amine in Step 8.

¹H NMR: δ_{H} (400 MHz, DMSO-d₆) 8.74-8.70 (1H, m), 8.46 (1H, d, J 8.4), 8.16 (1H, d, J 8.4), 4.30-4.25 (1H, m), 3.28-3.24 (1H, m), 3.06-3.03 (2H, m), 2.68-2.63 (2H, m), 2.09-2.02 (2H, m), 1.92-1.89 (2H, m), 1.71-1.61 (8H, m).

### Example 112: N-(4,4-difluorocyclohexyl)-2-(piperidin-4-yl)thiazolo[5,4-b]pyridine-5-carboxamide

Prepared according to Scheme 29 using 4,4-difluorocyclohexylamine as the amine in Step 8.

¹H NMR: δ_{H} (400 MHz, DMSO-d₆) 8.89-8.85 (1H, m), 8.47 (1H, d, J 8.4), 8.17 (1H, d, J 8.4), 4.04-4.02 (1H, m), 3.28-3.24 (1H, m), 3.08-3.02 (2H, m), 2.68-2.60 (2H, m), 2.07-1.24 (12H, m).

### Example 113: N-cyclopentyl-2-(1-methylpiperidin-4-yl)thiazolo[5,4-b]pyridine-5-carboxamide

Example 113 may be prepared by reductive alkylation of Example 111 using formaldehyde, according to the method of Scheme 9.

¹H NMR: δ_{H} (400 MHz, DMSO-d₆) 8.73-8.69 (1H, m), 8.47 (1H, d, J 8.4), 8.16 (1H, d, J 8.4), 4.30-4.25 (1H, m), 3.18-3.13 (1H, m), 2.89-2.85 (2H, m), 2.21 (3H, s), 2.13-2.03 (4H, m), 1.92-1.81 (4H, m), 1.73-1.69 (2H, m), 1.60-1.54 (4H, m).

### Example 114: N-(4,4-difluorocyclohexyl)-2-(1-methylpiperidin-4-yl)thiazolo[5,4-b]pyridine-5-carboxamide

Example 114 may be prepared by reductive alkylation of Example 112 using formaldehyde, according to the method of Scheme 9.

¹H NMR: δ_{H} (400 MHz, DMSO-d₆) 8.92-8.88 (1H, m), 8.47 (1H, d, J 8.4), 8.17 (1H, d, J 8.4), 4.03 (1H, d, J 7.6), 3.19-3.12 (1H, m), 2.89-2.85 (2H, m), 2.21 (3H, s), 2.13-1.87 (14H, m).

### Examples 115 and 116

Examples 115 and 116 may be prepared according to the method of Scheme 30.

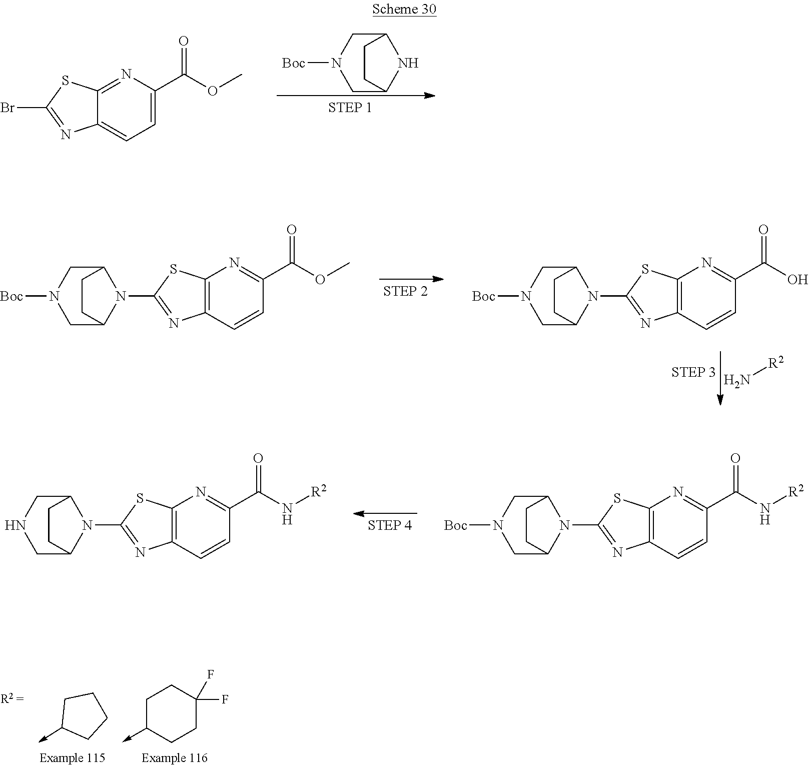

### Step 1 (Scheme 30): Synthesis of methyl 2-(3-(tert-butoxycarbonyl)-3,8-diazabicyclo[3.2.1]octan-8-yl)thiazolo[5,4-b]pyridine-5-carboxylate

To a stirred solution of methyl 2-bromothiazolo[5,4-b]pyridine-5-carboxylate (650 mg, 2.38 mmol) in acetonitrile (25 mL) was added potassium carbonate (658 mg, 4.76 mmol) and tert-butyl 3,8-diazabicyclo[3.2.1]octane-3-carboxylate (556 mg, 2.62 mmol). The reaction mixture was heated at 80° C. for 16 h, then concentrated under reduced pressure. The residue was partitioned between dichloromethane (30 mL) and water (10 mL). The organic layer was separated, washed with brine (10 mL), dried over anhydrous sodium sulfate, filtered and concentrated under reduced pressure. The residue was purified by flash chromatography, eluting with 30% ethyl acetate in hexane, to afford methyl 2-(3-(tert-butoxycarbonyl)-3,8-diazabicyclo[3.2.1]octan-8-yl)thiazolo[5,4-b]pyridine-5-carboxylate (550 mg, 53%) as an off-white solid.

### Step 2 (Scheme 30): Synthesis of 2-(3-(tert-butoxycarbonyl)-3,8-diazabicyclo[3.2.1]octan-8-yl)thiazolo[5,4-b]pyridine-5-carboxylic acid

To a stirred solution of methyl 2-(3-(tert-butoxycarbonyl)-3,8-diazabicyclo[3.2.1]octan-8-yl)thiazolo[5,4-b]pyridine-5-carboxylate (35 g, 84 mmol) in THF (300 mL), methanol (100 mL) and water (100 mL) at 0° C. was added lithium hydroxide (4.01 g, 167 mmol). The mixture was stirred at r.t. for 16 h, then concentrated under reduced pressure, diluted with water (200 mL), acidified with saturated aqueous citric acid solution to around pH4 and extracted with 10% MeOH in DCM (3×100 mL). The combined organic extract was washed with brine (100 mL), dried over anhydrous sodium sulfate, filtered and concentrated under reduced pressure to obtain 2-(3-(tert-butoxycarbonyl)-3,8-diazabicyclo[3.2.1]octan-8-yl)thiazolo[5,4-b]pyridine-5-carboxylic acid (32 g, 94%) as an off-white solid.

### General Procedure for Step 3 (Scheme 30): Amide Coupling

To a stirred solution of 2-(3-(tert-butoxycarbonyl)-3,8-diazabicyclo[3.2.1]octan-8-yl) thiazolo[5,4-b]pyridine-5-carboxylic acid (1.0 eq.) in DCM (10 vol.) at 0° C. were added the required amine (1.1 eq.), n-propylphosphonic acid anhydride cyclic trimer (2.0 eq.) and N,N-diisopropylethylamine (5.0 eq.). The reaction mixture was stirred at r.t. for 16 h, then diluted with DCM (10 vol.), washed with water (5 vol.) and brine (5 vol.), dried over anhydrous sodium sulfate and concentrated under reduced pressure. The residue was triturated with 10% ethyl acetate in hexane and dried to afford the required Boc-protected amide as an off-white solid.

### General Procedure for Step 4 (Scheme 30) Leading to Examples 115 and 116: Boc Deprotection

To a stirred solution of the Boc-protected amide (1 eq.) in DCM (10 vol.) at 0° C. was added HCl in dioxane (4 M; 2 eq.). The reaction mixture was allowed to warm to r.t. and stirred for 16 h, then concentrated under reduced pressure. The residue was purified by preparative HPLC using TFA as buffer. The purified fractions were collected and concentrated, dissolved in 10% methanol in DCM, washed with saturated sodium bicarbonate solution, dried over anhydrous sodium sulfate, filtered and concentrated under reduced pressure to obtain Examples 115 and 116 as off-white solids.

### Example 115: 2-(3,8-diazabicyclo[3.2.1]octan-8-yl)-N-cyclopentylthiazolo[5,4-b]pyridine-5-carboxamide

Prepared according to Scheme 30 using cyclopentylamine as the amine in Step 3.

¹H NMR: δ_{H} (400 MHz, DMSO-d₆) 8.45-8.41 (1H, m), 7.93 (1H, d, J 8.4), 7.79 (1H, d, J 8.4), 4.31-4.20 (3H, m), 2.98-2.94 (2H, m), 2.69-2.66 (2H, m), 2.02-2.00 (4H, m), 1.92-1.87 (2H, m), 1.72-1.69 (2H, m), 1.63-1.58 (4H, m).

### Example 116: 2-(3,8-diazabicyclo[3.2.1]octan-8-yl)-N-(4,4-difluorocyclohexyl)thiazolo[5,4-b]pyridine-5-carboxamide

Prepared according to Scheme 30 using 4,4-difluorocyclohexylamine as the amine in Step 3.

¹H NMR: δ_{H} (400 MHz, DMSO-d₆) 8.62-8.57 (1H, m), 7.95 (1H, d, J 8.4), 7.79 (1H, d, J 8.4), 4.37-4.27 (2H, m), 4.02-3.96 (2H, m), 2.99-2.95 (2H, m), 2.71-2.67 (2H, m), 2.03-1.73 (11H, m).

### Example 117: 2-(3,8-diazabicyclo[3.2.1]octan-8-yl)-N-(4,4-difluorocyclohexyl)thiazolo[4,5-b]pyridine-6-carboxamide

Example 117 may be prepared according to the method of Scheme 31.

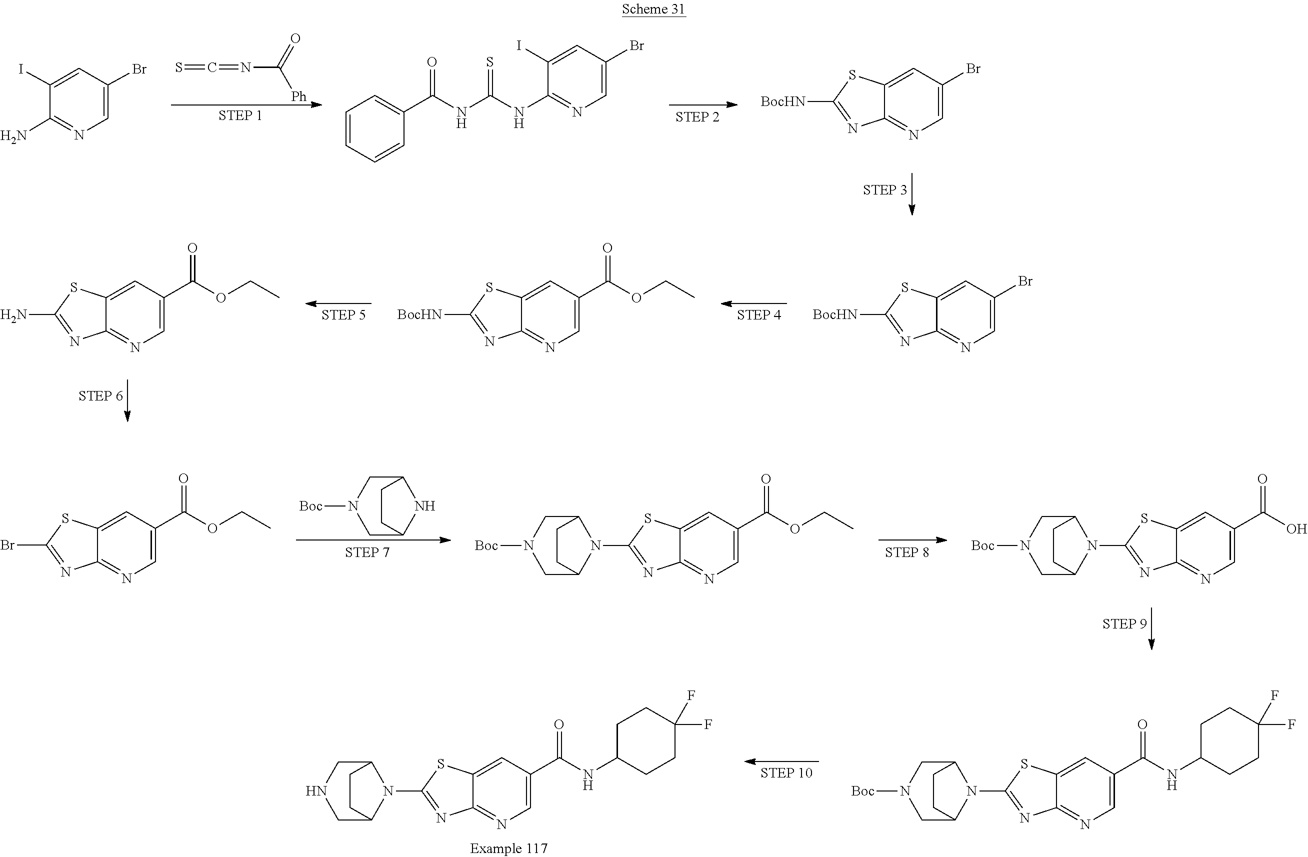

### Step 1 (Scheme 31): Synthesis of N-((5-bromo-3-iodopyridin-2-yl)carbamothioyl)benzamide

To a stirred solution of 5-bromo-3-iodopyridin-2-amine (8.00 g, 26.8 mmol) in acetone (24 mL) was added benzoyl isothiocyanate (5.28 mL, 38.8 mmol). The mixture was stirred at r.t. for 20 h, then filtered. The filtrate was triturated with hexane and dried to afford N-((5-bromo-3-iodopyridin-2-yl)carbamothioyl)benzamide (10.5 g, 83%) as an off-white solid.

### Step 2 (Scheme 31): Synthesis of 6-bromothiazolo[4,5-b]pyridin-2-amine

To a stirred solution of N-((5-bromo-3-iodopyridin-2-yl)carbamothioyl)benzamide (10.5 g, 22.7 mmol) in methanol (23 mL) was added aqueous sodium hydroxide (6.0 N; 133 mL, 795 mmol). The mixture was heated to reflux for 16 h, then allowed to cool and concentrated under reduced pressure to remove methanol. The residue was diluted with cold water (100 mL) and poured into a saturated solution of ammonium chloride (600 mL) with cooling. The mixture was stirred for 1 h at r.t. The solid precipitate was filtered off and dried under vacuum to afford 6-bromothiazolo[4,5-b]pyridin-2-amine (4.3 g, 76%) as an off-white solid.

### Step 3 (Scheme 31): Synthesis of tert-butyl (6-bromothiazolo[5,4-b]pyridin-2-yl)carbamate

To a stirred solution of 6-bromothiazolo[4,5-b]pyridin-2-amine (4.3 g, 18.7 mmol) in DCM (25 mL) and THF (60 mL) was added Boc anhydride (8.59 mL, 37.4 mmol) followed by 4-dimethylaminopyridine (0.457 g, 3.74 mmol). The mixture was stirred at r.t. for 4 h, then diluted with cold water (50 mL) and extracted with ethyl acetate (3×100 mL). The combined organic extract was washed with brine, dried over anhydrous sodium sulfate, filtered and concentrated under reduced pressure to afford tert-butyl (6-bromothiazolo[5,4-b]pyridin-2-yl)carbamate (5.0 g, 80%) as a yellow solid.

### Step 4 (Scheme 31): Synthesis of ethyl 2-((tert-butoxycarbonyl)amino)thiazolo[4,5-b]pyridine-6-carboxylate

To a stirred, degassed solution of tert-butyl (6-bromothiazolo[4,5-b]pyridin-2-yl)carbamate (5.50 g, 16.6 mmol) in ethanol (200 mL) in a Tinyclave steel pressure reactor under nitrogen was added N-ethyl-N-isopropylpropan-2-amine (11.64 mL, 66.6 mmol) and Pd(dppf)Cl₂·DCM complex (5.44 g, 6.66 mmol). The reaction mixture was placed under CO pressure (60 psi) at 85° C. for 16 h, then concentrated under reduced pressure. The residue was purified by flash chromatography, eluting with 10% to 12% ethyl acetate in petroleum ether, to afford ethyl 2-((tert-butoxycarbonyl)amino)thiazolo[4,5-b]pyridine-6-carboxylate (1.5 g, 25%) as an off-white solid.

### Step 5 (Scheme 31): Synthesis of ethyl 2-aminothiazolo[4,5-b]pyridine-6-carboxylate

To a stirred solution of ethyl 2-((tert-butoxycarbonyl)amino)thiazolo[4,5-b]pyridine-6-carboxylate (1.50 g, 4.64 mmol) in DCM (10 mL) was added HCl (4.0 M in dioxane; 10.0 mL, 40.0 mmol). The mixture was stirred at r.t. for 16 h, then concentrated under reduced pressure. The residue was dissolved in 20% MeOH in DCM (20 mL), then basified with triethylamine to around pH 9 with cooling, stirred for 5 min, then concentrated under reduced pressure to afford ethyl 2-aminothiazolo[4,5-b]pyridine-6-carboxylate (1.01 g, 98%) as a light yellow solid.

### Step 6 (Scheme 31): Synthesis of ethyl 2-bromothiazolo[4,5-b]pyridine-6-carboxylate

Ethyl 2-bromothiazolo[4,5-b]pyridine-6-carboxylate may be prepared following the method of Step 4 (Scheme 29), using ethyl 2-aminothiazolo[4,5-b]pyridine-6-carboxylate instead of methyl 2-aminothiazolo[5,4-b]pyridine-5-carboxylate.

### Steps 7 to 10 (Scheme 31) leading to Example 117: 2-(3,8-diazabicyclo[3.2.1]octan-8-yl)-N-(4,4-difluorocyclohexyl)thiazolo[4,5-b]pyridine-6-carboxamide

Steps 7 to 10 of Scheme 31 were carried out according to the method of Scheme 30, Steps 1 to 4, using ethyl 2-bromothiazolo[4,5-b]pyridine-6-carboxylate instead of methyl 2-bromothiazolo[5,4-b]pyridine-5-carboxylate in Step 1 and using 4,4-difluorocyclohexylamine as the amine in Step 3, to afford 2-(3,8-diazabicyclo[3.2.1]octan-8-yl)-N-(4,4-difluorocyclohexyl)thiazolo[4,5-b]pyridine-6-carboxamide as an off-white solid.

¹H NMR: δ_{H} (400 MHz, DMSO-d₆) 8.76 (1H, d, J2.0), 8.54 (1H, d, J2.0), 8.35-8.31 (1H, m), 4.39-4.21 (2H, m), 4.05-3.96 (1H, m), 2.97-2.91 (2H, m), 2.71-2.65 (2H, m), 2.10-1.89 (10H, m), 1.67-1.61 (2H, m).

### Example 118: N-cyclopentyl-2-(piperidin-4-yl)thiazolo[4,5-b]pyridine-6-carboxamide

Example 118 may be prepared according to the method of Scheme 32.

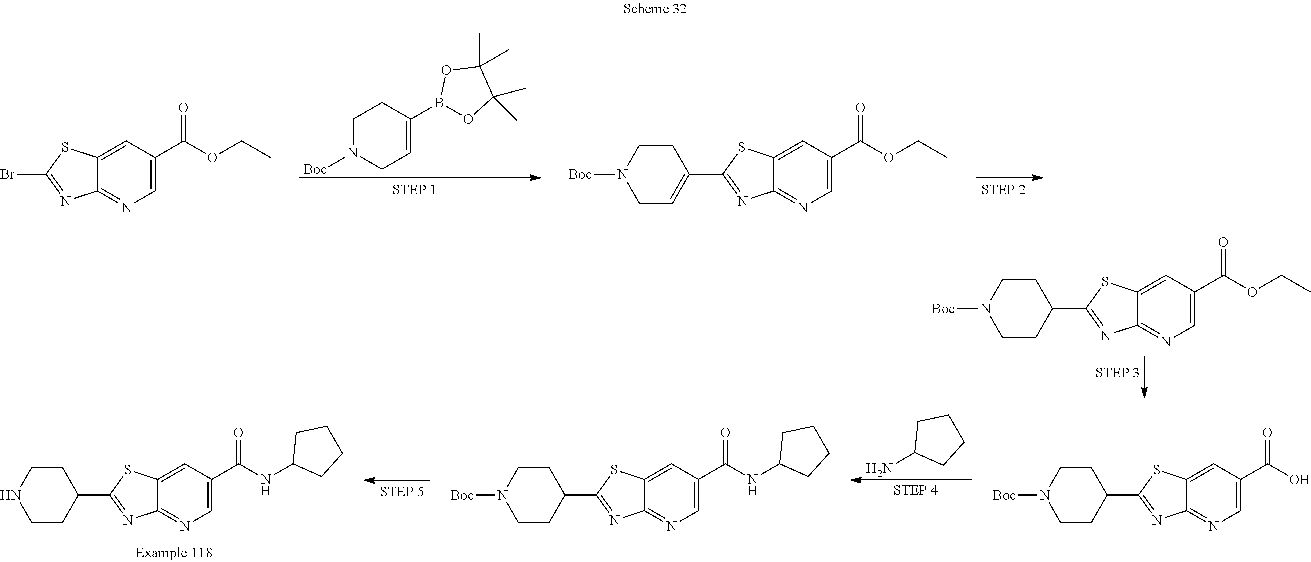

### Step 1 (Scheme 32): Synthesis of ethyl 2-(1-(tert-butoxycarbonyl)-1,2,3,6-tetrahydropyridin-4-yl)thiazolo[4,5-b]pyridine-6-carboxylate

To a stirred solution of ethyl 2-bromothiazolo[4,5-b]pyridine-6-carboxylate (600 mg, 2.09 mmol) in 1,4-dioxane (18 mL) and water (2 mL) was added tert-butyl 4-(4,4,5,5-tetramethyl-1,3,2-dioxaborolan-2-yl)-3,6-dihydropyridine-1 (2H)-carboxylate (775 mg, 2.51 mmol) followed by cesium carbonate (1.36 g, 4.18 mmol). The reaction mixture was purged with nitrogen for 15 min, then Pd(dppf)Cl₂·CH₂Cl₂ (171 mg, 0.209 mmol) was added. The mixture was stirred at 100° C. for 3 h, then cooled and partitioned between ethyl acetate and water. The aqueous layer was further extracted with ethyl acetate and the combined organic extract was dried over anhydrous sodium sulfate, filtered and concentrated under reduced pressure. The residue was purified by flash chromatography, eluting with 22% ethyl acetate in petroleum ether to afford ethyl 2-(1-(tert-butoxycarbonyl)-1,2,3,6-tetrahydropyridin-4-yl)thiazolo[4,5-b]pyridine-6-carboxylate (440 mg, 46%) as a brown solid.

### Steps 2 to 5 (Scheme 32) leading to Example 118: N-cyclopentyl-2-(piperidin-4-yl)thiazolo[4,5-b]pyridine-6-carboxamide

Steps 2 to 5 of Scheme 32 were carried out according to the method of Scheme 29, Steps 6 to 9, using ethyl 2-(1-(tert-butoxycarbonyl)-1,2,3,6-tetrahydropyridin-4-yl)thiazolo[4,5-b]pyridine-6-carboxylate instead of methyl 2-(1-(tert-butoxycarbonyl)-1,2,3,6-tetrahydropyridin-4-yl)thiazolo[5,4-b]pyridine-5-carboxylate in Step 6 and cyclopentylamine as the amine in Step 8, to afford N-cyclopentyl-2-(piperidin-4-yl)thiazolo[4,5-b]pyridine-6-carboxamide as an off-white solid.

¹H NMR: δ_{H} (400 MHz, DMSO-d₆) 9.06 (1H, d, J2.0), 8.96 (1H, d, J2.0), 8.60-8.56 (1H, m), 4.28-4.24 (1H, m), 3.47-3.35 (1H, m), 3.10-3.05 (2H, m), 2.63-2.51 (2H, m), 2.09-2.04 (2H, m), 1.94-1.90 (2H, m), 1.77-1.74 (4H, m), 1.72-1.56 (4H, m).

### Examples 119 and 120

Examples 119 and 120 may be prepared according to the method of Scheme 33.

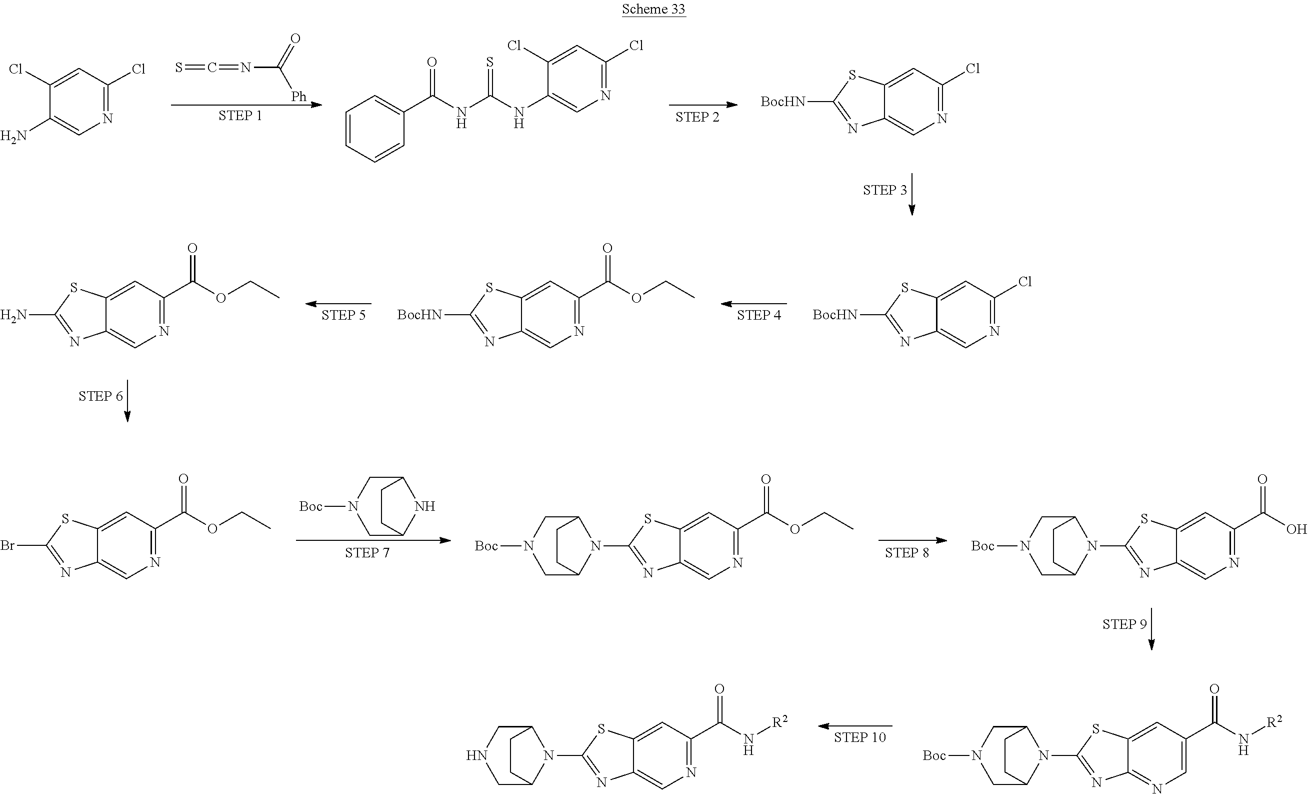

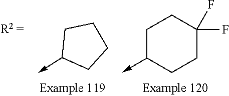

### Step 1 (Scheme 33): Synthesis of N-((4,6-dichloropyridin-3-yl)carbamothioyl)benzamide

To a stirred solution of 4,6-dichloropyridin-3-amine (1.00 g, 6.13 mmol) in acetone (10 mL) was added benzoyl isothiocyanate (1.00 mL, 7.36 mmol). The reaction mixture was stirred at r.t. for 16 h, then filtered, triturated with petroleum ether and dried under vacuum to afford N-((4,6-dichloropyridin-3-yl)carbamothioyl)benzamide (1.80 g, 87%) as a light brown solid.

### Step 2 (Scheme 33): Synthesis of 6-chlorothiazolo[4,5-c]pyridin-2-amine

To a stirred solution of N-((4,6-dichloropyridin-3-yl)carbamothioyl)benzamide (900 mg, 2.76 mmol) in methanol (10 mL) was added aqueous sodium hydroxide (6.0 N; 13.8 mL, 83 mmol). The mixture was heated to reflux for 16 h, then cooled to r.t., followed by addition of saturated aqueous ammonium chloride (200 mL). The mixture was stirred for 10 min at r.t. The resulting precipitate was filtered off and dried under vacuum to afford 6-chlorothiazolo[4,5-c]pyridin-2-amine (450 mg, 87%) as a light brown solid.

### Step 3 (Scheme 30): Synthesis of (tert-butyl (6-chlorothiazolo[4,5-c]pyridin-2-yl)carbamate

To a stirred solution of 6-chlorothiazolo[4,5-c]pyridin-2-amine (2.20 g, 11.9 mmol) in DCM (25 mL) and THF (25 mL) was added 4-(dimethylamino)pyridine (0.724 g, 5.93 mmol) followed by Boc anhydride (4.08 mL, 17.8 mmol). The mixture was stirred at r.t. for 16 h, then diluted with DCM (50 mL), washed with water (30 mL) and brine (30 mL), dried over anhydrous sodium sulfate, filtered and concentrated under reduced pressure. The residue was triturated with petroleum ether and dried under vacuum to afford tert-butyl (6-chlorothiazolo[4,5-c]pyridin-2-yl)carbamate (2.50 g, 69%) as an off-white solid.

### Step 4 (Scheme 33): Synthesis of ethyl 2-((tert-butoxycarbonyl)amino)thiazolo[4,5-c]pyridine-6-carboxylate

To a stirred, degassed solution of tert-butyl (6-chlorothiazolo[4,5-c]pyridin-2-yl)carbamate (1.2 g, 4.20 mmol) in ethanol (70 mL) in a Tinyclave steel pressure reactor under nitrogen was added N-ethyl-N-isopropylpropan-2-amine (5.87 mL, 33.6 mmol) and Pd(dppf)Cl₂·CH₂Cl₂ (2.74 g, 3.36 mmol). The reaction mixture was placed under CO pressure (60 psi) at 85° C. for 16 h, then filtered through Celite® and concentrated under reduced pressure. The residue was purified by HPLC using formic acid as a buffer to afford ethyl 2-((tert-butoxycarbonyl)amino)thiazolo[4,5-c]pyridine-6-carboxylate (400 mg, 77%) as a light yellow solid.

### Step 5 (Scheme 33): Synthesis of ethyl 2-aminothiazolo[4,5-c]pyridine-6-carboxylate

To a stirred solution of ethyl 2-((tert-butoxycarbonyl)amino)thiazolo[4,5-c]pyridine-6-carboxylate (800 mg, 2.47 mmol) in DCM (10 mL) was added HCl (4.0 M in dioxane; 1.86 mL, 7.44 mmol). The mixture was stirred at r.t. for 16 h, then concentrated under reduced pressure. The residue was diluted with 10% MeOH in DCM (10 mL) and basified with triethylamine to around pH 9 with cooling, stirred for 5 min, then concentrated under reduced pressure to afford ethyl 2-aminothiazolo[4,5-c]pyridine-6-carboxylate (550 mg, 91%) as a light brown solid.

### Step 6 (Scheme 33): Synthesis of ethyl 2-bromothiazolo[4,5-c]pyridine-6-carboxylate

To a stirred solution of copper(II) bromide (1.20 g, 5.38 mmol) in acetonitrile (20 mL) was added tert-butyl nitrite (0.645 mL, 5.38 mmol). The mixture was stirred at r.t. for 30 min, then cooled to 0° C. and ethyl 2-aminothiazolo[4,5-c]pyridine-6-carboxylate (600 mg, 2.69 mmol) was added. The mixture was allowed to warm to r.t. and stirred for 16 h, then diluted with cold water (20 mL) and extracted with ethyl acetate (2×20 mL). The combined organic extract was washed with brine (10 mL), dried over anhydrous sodium sulfate, filtered and concentrated under reduced pressure to afford ethyl 2-bromothiazolo[4,5-c]pyridine-6-carboxylate (600 mg, 67%) as a light yellow solid.

### Steps 7 to 10 (Scheme 33) Leading to Examples 119 and 120

Steps 7 to 10 of Scheme 33 were carried out according to the method of Scheme 30, Steps 1 to 4, using ethyl 2-bromothiazolo[4,5-c]pyridine-6-carboxylate instead of methyl 2-bromothiazolo[5,4-b]pyridine-5-carboxylate in Step 1.

### Example 119: 2-(3,8-diazabicyclo[3.2.1]octan-8-yl)-N-cyclopentylthiazolo[4,5-c]pyridine-6-carboxamide

Prepared according to Scheme 33 using cyclopentylamine as the amine in Step 9.

¹H NMR: δ_{H} (400 MHz, DMSO-d₆) 8.64 (1H, d, J0.6), 8.49 (1H, d, J0.6), 8.41-8.37 (1H, m), 4.27-4.24 (3H, m), 2.97-2.93 (2H, m), 2.68-2.64 (2H, m), 2.01-1.96 (4H, m), 1.92-1.86 (2H, m), 1.72-1.66 (2H, m), 1.62-1.54 (4H, m).

### Example 120: 2-(3,8-diazabicyclo[3.2.1]octan-8-yl)-N-(4,4-difluorocyclohexyl)thiazolo[4,5-c]pyridine-6-carboxamide

Prepared according to Scheme 33 using 4,4-difluorocyclohexylamine as the amine in Step 9.

¹H NMR: δ_{H} (400 MHz, DMSO-d₆) 8.65 (1 h, s), 8.57-8.53 (1H, m), 8.51 (1H, s), 4.35-4.20 (2H, br. m), 4.04-3.94 (1H, m), 2.97-2.93 (2H, m), 2.68-2.64 (2H, m), 2.08-1.92 (8H, m), 1.89-1.83 (2H, m), 1.80-1.71 (2H, m).

### Examples 121 and 122

Examples 121 and 122 may be prepared according to the method of Scheme 34.

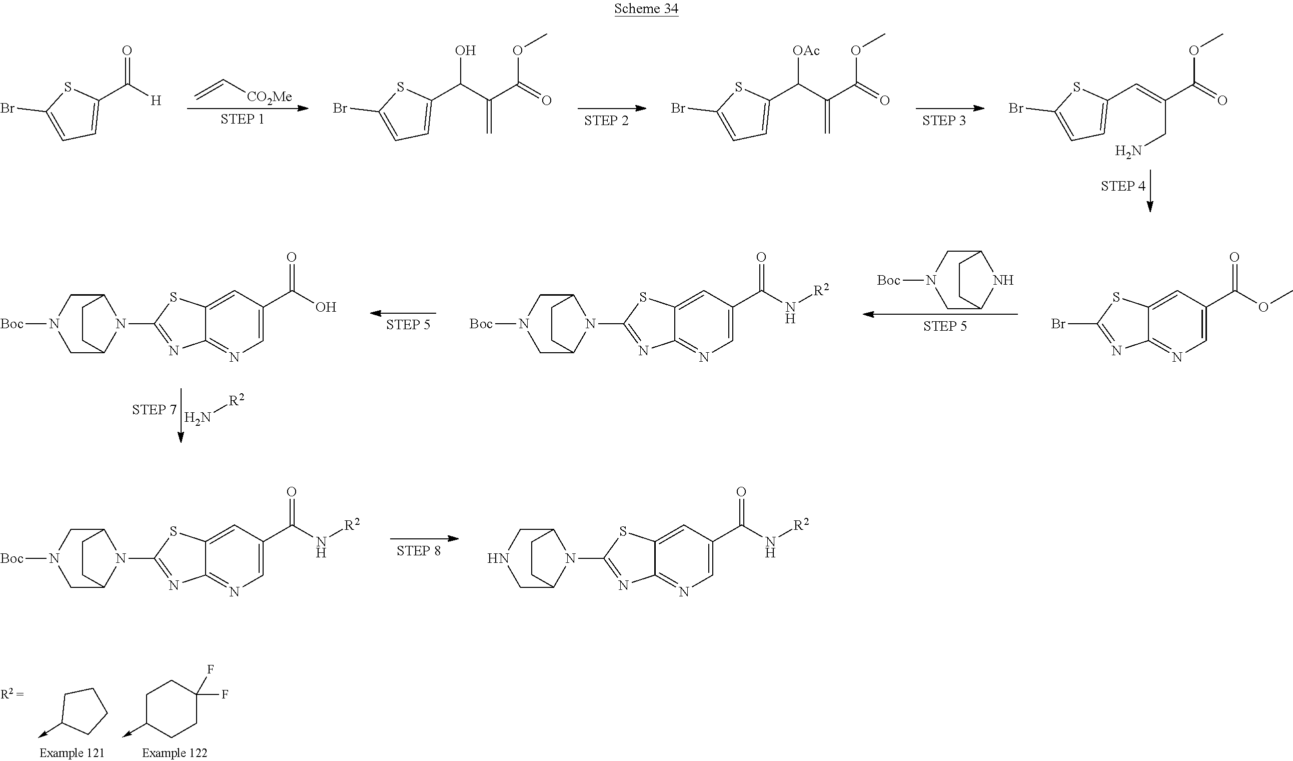

### Step 1 (Scheme 34): Synthesis of methyl 2-((5-bromothiophen-2-yl)(hydroxy)methyl)acrylate

To a mixture of 5-bromothiophene-2-carbaldehyde (2.00 g, 10.5 mmol) and methyl acrylate (2.85 mL, 31.4 mmol) at r.t. was added DABCO (1.17 g, 10.5 mmol). The mixture was stirred at r.t. for 16 h, then diluted with DCM (50 mL), washed with water (30 mL) and brine (10 mL), dried over anhydrous sodium sulfate, filtered and concentrated under reduced pressure to afford methyl 2-((5-bromothiophen-2-yl)(hydroxy)methyl)acrylate (2.90 g, 89%) as a yellow sticky gel.

### Step 2 (Scheme 34): Synthesis of methyl 2-(acetoxy(5-bromothiophen-2-yl)methyl)acrylate

To a stirred solution of methyl 2-((5-bromothiophen-2-yl)(hydroxy)methyl)acrylate (3.00 g, 10.8 mmol) in DCM (10 mL) at r.t. was added acetic anhydride (1.53 mL, 16.2 mmol) followed by 4-(dimethylamino)pyridine (0.265 g, 2.17 mmol) portionwise. The mixture was stirred at r.t. for 1 h, then diluted with DCM (60 mL), washed with water (20 mL) and brine (20 mL), dried over anhydrous sodium sulfate, filtered and concentrated under reduced pressure. The residue was purified by flash chromatography, eluting with 2% to 4% ethyl acetate in petroleum ether, to afford methyl 2-(acetoxy(5-bromothiophen-2-yl)methyl)acrylate (920 mg, 24%) as a colourless liquid.

### Step 3 (Scheme 34): Synthesis of methyl 2-(aminomethyl)-3-(5-bromothiophen-2-yl)acrylate

To a stirred solution of methyl 2-(acetoxy(5-bromothiophen-2-yl)methyl)acrylate (800 mg, 2.51 mmol) at 0° C. was added ammonia in methanol (7.0 M; 32 mL, 224 mmol). The mixture was stirred at 0° C. for 15 min, then at r.t. for 1 h. The reaction mixture was concentrated under reduced pressure. The residue was purified by flash chromatography, eluting with 10% to 20% methanol in DCM, to afford methyl 2-(aminomethyl)-3-(5-bromothiophen-2-yl)acrylate (270 mg, 31%) as a pale yellow solid.

### Step 4 (Scheme 34): Synthesis of methyl 2-bromothieno[3,2-b]pyridine-6-carboxylate

To a stirred solution of methyl 2-(aminomethyl)-3-(5-bromothiophen-2-yl)acrylate (140 mg, 0.507 mmol) in acetonitrile (10 mL) at r.t. was added potassium carbonate (596 mg, 4.31 mmol) followed by iodine (901 mg, 3.55 mmol). The reaction mixture was stirred at r.t. for 2 h, then diluted with saturated aqueous sodium thiosulfate solution (10 mL), stirred for 10 min and extracted with ethyl acetate (20 mL). The organic extract was washed with brine (10 mL), dried over anhydrous sodium sulfate, filtered and concentrated under reduced pressure. The residue was purified by flash chromatography, eluting with 15% to 20% ethyl acetate in petroleum ether, to afford methyl 2-bromothieno[3,2-b]pyridine-6-carboxylate (35 mg, 22%) as a pale yellow solid.

### Step 5 (Scheme 34): Synthesis of methyl 2-(3-(tert-butoxycarbonyl)-3,8-diazabicyclo[3.2.1]octan-8-yl)thieno[3,2-b]pyridine-6-carboxylate

To a de-gassed mixture of methyl 2-bromothieno[3,2-b]pyridine-6-carboxylate (740 mg, 2.72 mmol) and tert-butyl 3,8-diazabicyclo[3.2.1]octane-3-carboxylate (693 mg, 3.26 mmol) in toluene (20 mL) were added cesium carbonate (1.77 g, 5.44 mmol), palladium(II) acetate (244 mg, 1.09 mmol) and BINAP (339 mg, 0.544 mmol). The mixture was stirred at 110° C. for 16 h, then concentrated under reduced pressure. The residue was purified by flash chromatography, eluting with 40% ethyl acetate in petroleum ether, to afford methyl 2-(3-(tert-butoxycarbonyl)-3,8-diazabicyclo[3.2.1]octan-8-yl)thieno[3,2-b]pyridine-6-carboxylate (400 mg, 36%) as an off-white solid.

### Step 6 (Scheme 34): Synthesis of 2-(3-(tert-butoxycarbonyl)-3,8-diazabicyclo[3.2.1]octan-8-yl)thieno[3,2-b]pyridine-6-carboxylic acid

To a stirred solution of methyl 2-(3-(tert-butoxycarbonyl)-3,8-diazabicyclo[3.2.1]octan-8-yl)thieno[3,2-b]pyridine-6-carboxylate (400 mg, 0.991 mmol) in THF (8 mL), methanol (8 mL) and water (4 mL), was added lithium hydroxide monohydrate (81 mg, 2.0 mmol). The mixture was stirred at r.t. for 16 h, then concentrated under reduced pressure, washed with 10% ethyl acetate in petroleum ether and dried to afford 2-(3-(tert-butoxycarbonyl)-3,8-diazabicyclo[3.2.1]octan-8-yl)thieno[3,2-b]pyridine-6-carboxylic acid, lithium salt (250 mg, 65%).

### General Procedure for Step 7 (Scheme 34): Amide Coupling

To a stirred solution of 2-(3-(tert-butoxycarbonyl)-3,8-diazabicyclo[3.2.1]octan-8-yl)thieno[3,2-b]pyridine-6-carboxylic acid, lithium salt (1.0 eq.) in DCM (10 vol.) were added the required amine (2.0 eq.), N-ethyl-N-isopropylpropan-2-amine (5.0 eq.) and n-propylphosphonic acid anhydride cyclic trimer (50% in ethyl acetate; 2.0 eq.). The mixture was stirred at r.t. for 16 h, then diluted with ice-cold water (10 vol.) and extracted with DCM (2×10 vol.). The combined organic extract was washed with water (5 vol.) and brine (5 vol.), dried over anhydrous sodium sulfate and concentrated under reduced pressure. The residue was triturated with 10% ethyl acetate in hexane and dried to afford the required Boc-protected amide as an off-white solid

### General Procedure for Step 8 (Scheme 34) Leading to Examples 121 and 122: Boc Deprotection

To a stirred solution of the Boc-protected amide (1 eq.) in DCM (10 vol.) at 0° C. was added HCl in dioxane (4 M; 2 eq.). The reaction mixture was allowed to warm to r.t. and stirred for

16 h, then concentrated under reduced pressure. The residue was purified by preparative HPLC using ammonium bicarbonate as a buffer to afford Examples 121 and 122 as off-white solids.

### Example 121: 2-(3,8-diazabicyclo[3.2.1]octan-8-yl)-N-cyclopentylthieno[3,2-b]pyridine-6-carboxamide

Prepared according to Scheme 34 using cyclopentylamine as the amine in Step 7.

¹H NMR: δ_{H} (400 MHz, DMSO-d₆) 8.78 (1H, d, J 1.8), 8.40 (1H, d, J 1.8), 8.25-8.21 (1H, m), 6.37 (1H, s), 4.27-4.20 (1H, m), 4.04 (2H, s), 3.05-3.01 (2H, m), 2.67-2.51 (2H, m), 1.99-1.96 (4H, m), 1.94-1.88 (2H, m), 1.72-1.67 (2H, m), 1.59-1.53 (4H, m).

### Example 122: 2-(3,8-diazabicyclo[3.2.1]octan-8-yl)-N-(4,4-difluorocyclohexyl)thieno[3,2-b]pyridine-6-carboxamide

Prepared according to Scheme 34 using 4,4-difluorocyclohexylamine as the amine in Step 7.

¹H NMR: δ_{H} (400 MHz, DMSO-d₆) 8.78 (1H, d, J 1.8), 8.41 (1H, d, J 1.8), 8.27-8.23 (1H, m), 6.38 (1H, s), 4.06-3.96 (3H, m), 3.05-2.99 (2H, m), 2.68-2.52 (2H, m), 2.08-1.99 (2H, m), 1.98-1.86 (8H, m), 1.69-1.59 (2H, m).

### Biological Assays

### Cell Culture

HEK 293 and MDCK cells (Public Health England, Cell Culture Collections) were maintained in Dulbecco's Modified Eagle's Medium (DMEM) supplemented with 10% (v/v) fetal bovine serum (FBS) (Seralab), 2 mM L-glutamine and 100 U/ml penicillin streptomycin cocktail (ThermoFisher); termed complete media. Clonal HEK 293 cell lines were maintained in complete media supplemented with 0.6 mg/mL G418 (Enzo Life Sciences). Mouse inner medullary collecting duct (m-IMCD3) cells [American Type Culture Collection (ATCC)] were maintained in media consisting of DMEM/Hams F-12 50/50 Mix (DMEM F12; Corning) supplemented with penicillin streptomycin cocktail (100 U/ml) and FBS (10% v/v) (DMEM F12-10% FBS).

Experiment 1: Measurement of Modulation of Enzyme Activity by PDE4 Long Form Activators of the Present Invention Using Full-Length Human PDE4 Isoforms; Long Forms PDE4D5, PDE4C3, PDE4B1 and Short Form PDE4B2
(Marchmont, R. J. and Houslay, M. D. Biochem. J. 187: 381-92, 1980)

### Exogenous Expression of Long Form PDE4 Enzymes and Stable Cell Line Generation

For transient transfection of exogenous PDE4 long isoforms, HEK 293 cells were transfected with pcDNA3.1 or pDEST^{T}M PDE4 expression vectors using Lipofectamine LTX/Plus reagent (Invitrogen) as outlined by the manufacturer.

Where stable cell lines were generated the clonal isolates were expanded to obtain cell lines that stably expressed the full-length human PDE4 long isoforms and the full length human PDE4B2 short isoform. These were called the HEK-PDE4D5, HEK-PDE4B1 and HEK-PDE4B2 cell lines, respectively.

### Lysate Preparation (Using PDE4D5 as a Typical Example)

HEK-PDE4D5 cells were seeded out in 100 mm plates and incubated at 37° C. in an atmosphere of 5% CO₂, 95% air. Cell lysates were prepared using KHEM buffer [50 mM KCl, 10 mM EGTA, 50 mM HEPES (pH 7.2), 1.92 mM MgCl₂].

To prepare the cell lysates, the 100 mm plates containing the cells were placed on ice and washed with ice-cold PBS (phosphate buffered saline, pH 7.4). KHEM buffer (500 μL) was added to the cells. Cells were then scraped off the plate and triturated using a needle (BD Microlance™ 0.8, 40 mm). The lysed cells were then centrifuged at 2000 rpm for 10 minutes to remove cell debris and the supernatant (cell lysate containing recombinant PDE4D5) was transferred to a fresh tube and kept on ice.

### Cytosol Fraction Preparation (Using PDE4D5 as a Typical Example)

The cell lysate containing recombinant PDE4D5 was transferred into a centrifuge tube and placed into an ultracentrifuge (BECKMAN COULTER) and spun at high speed (100,000 g) for 30 minutes at 4° C. The cytosol fraction was then collected and its protein amount determined using a BCA protein assay.

### PDE Assay —(Using PDE4D5 as a Typical Example)

PDE assays were performed in thin walled V-bottomed 96-well plates. The assays were performed at a final concentration of 10 mM Tris/5 mM MgCl₂ plus PDE4D5 cell lysate cytosol fraction, containing over-expressed PDE4D5, with and without test compound. The lysate/compound mix were incubated together for 15 min at room temperature on an orbital shaker prior to addition of [³H] cAMP (final concentration 1 μM [³H] cAMP; Perkin Elmer) to a final volume of 50 μL per reaction. The reactions were then incubated for 10 minutes at 30° C., terminated by heating for 2 min at 95° C. and allowed to cool. Snake venom (12.5 μL of 1 mg/mL; Crotalus atrox, Sigma) was then added and the plates were agitated and incubated for a further 15 minutes at 30° C. Dowex ion exchange resin (Sigma, chloride form, 200-400 mesh; 200 μL; prepared as a 1:1 Dowex:water stock, thoroughly re-suspended and diluted 2:1 with ethanol) was then added to each well and the plates incubated for 15 min at room temperature on an orbital shaker ensuring sufficient agitation for resin suspension (550 RPM). The reaction mixture was then transferred to a 96 well filter plate (Millipore; 0.45 μm pore size) and filtered into a receiving 96-well plate to remove the dowex suspension. 30 μL of the filtered solution was then transferred to the wells of an Opti-plate (Perkin Elmer) 96 well assay plate and 120 μL of Microscint 40 scintillation fluid was added. The plate was then placed on an orbital shaker for 10 min at high speed (900 rpm) to mix the sample with scintillation fluid prior to quantitation using a plate based scintillation counter (Top-Count).

The % increase in counts in the presence of test compound at a particular concentration indicates the % increase in enzyme activity at that concentration.

Data are shown in FIG. 1 and Tables 2 and 3.

### Experiment 2: Reduction of Intracellular cAMP Levels in MDCK Cells by PDE4 Long Form Activators

MDCK cells were seeded at 100,000 cells per well, and left to adhere overnight. The cells were then treated with the test compound for 40 minutes, prior to stimulation with forskolin (1 μM, Sigma) for 20 minutes. Media was aspirated, and hydrochloric acid (0.1M) was added to lyse the cells. The cAMP assay (Enzo Life Sciences) was performed according to the manufacturer's instructions.

PDE4 long form activators reduced intracellular cAMP levels in forskolin-stimulated MDCK cells.

### Experiment 3: Inhibition of In Vitro Cyst Formation in MDCK Cells Treated with PDE4 Long Form Activators

In this study, the well-established three-dimensional (3D) MDCK cell model is used to investigate the effects of PDE4 long form activators on the formation of kidney cysts and evaluate their potential in the treatment of polycystic kidney diseases. 3D cysts are generated based on the method of Mao et al. (Mao, Z., Streets, A. J., Ong, A. C. M. Am. J. Physiol. Renal Physiol. 300 (6): F1375-F1384, 2011), with some modifications.

The assay is conducted in the wells of a 96-well plate culture dish using a total volume of 130 μL of collagen suspension in growth media for each matrix plug per individual well. Rat Collagen I (Fisher Scientific) is prepared on ice by neutralising with 1 M NaOH and diluting with a 2× volume of DMEM-2% FBS. 30 μl of collagen/Media mix is added into the well of a 96 well plate and the collagen is set to a gel by incubating at 37° C. for at least 15 minutes. A second layer of 100 μL collagen/Matrigel suspension containing MDCK cells (1.0×10⁴ MDCK cells per well) is then layered over the first layer and the collagen/cell mix again set to a gel by incubating at 37° C. 100 μL DMEM growth media is added and cells are incubated at 37° C. for 24 hours. 24 hours after cell seeding, DMEM-2% FBS is added along with the test compound indicated in the presence of 300 nM prostaglandin E2 (PGE2) (Sigma Aldrich) in quadruplicate wells per condition. Media, together with the test compound and PGE2 are replenished every 3 days for 10-15 days.

After 10-15 days of culture z stack images of the wells are captured using the Nikon Eclipse Ti2-E microscope. Nikon General Analysis software is used to measure the following parameters in each well: the mean cyst area, the number of cysts, and the total cyst area.

PDE4 long form activators inhibited in vitro cyst formation in MDCK cells.

### Experiment 4: Inhibition of In Vitro Cyst Formation in m-IMCD3 Cells Treated with PDE4 Long Form Activators

The mouse Inner Medullary Collecting Duct cell line (m-IMCD3) spontaneously forms cystic spheroids in 3D culture with a Type 1 collagen/Matrigel extracellular matrix. This process can be stimulated with agents which raise intracellular cAMP and is used as an in vitro model for the formation of cystic structures in the kidneys of patients with ADPKD.

Rat Collagen I (Fisher Scientific) is prepared on ice by neutralising with 1 M NaOH and diluting with a 2×volume of DMEM/F12+10% FBS. This is mixed 1:1.1 with ice cold Matrigel (Corning) for coating plates (coating mix), and 1:0.93 with Matrigel for cell plating (plating mix).

The assay is conducted in the wells of a 96-well plate culture dish using a total volume of 130 μL of collagen/Matrigel/DMEM F12-10% FBS suspension in growth media for each matrix plug per individual well. Initially, 30 μL of collagen/Matrigel/DMEM F12-10% FBS (coating mix) is added into the well of a 96 well plate and the collagen is set to a gel by incubating at 37° C. for at least 15 minutes. A second layer of 100 μL collagen/Matrigel suspension (plating mix) containing m-IMCD3 cells (2.75×106 m-IMCD3 cells per 96-well plate) is layered over the coating mix and the collagen/Matrigel/cell mix again set to a gel by incubating at 37° C. Cell cultures are maintained at 37° C. in an atmosphere of 5% CO₂, 95% air.

Between 18 and 24 h after plating, test compound(s) as DMSO stock solutions [0.1% (v/v) final DMSO concentration] and PGE2 (100 nM final concentration) in DMEM F12-10% FBS are added in quadruplicate wells per condition. Media, together with the test compound and PGE2 are replenished after 2 or 3 days. After 6 days of culture z stack images of the wells are captured using the Nikon Eclipse Ti2-E microscope. Nikon General Analysis software is used to measure the following parameters in each well: the mean cyst area, the number of cysts, and the total cyst area.

PDE4 long form activators inhibited in vitro cyst formation in m-IMCD3 cells. Data are shown in FIG. 2 and Table 4 as mean cyst area (%), compared to 100% for (DMSO+PGE2) and 0% for DMSO control.

### Experiment 5: Inhibition of Proliferation of LNCaP Human Prostate Cancer Cells

In this study, the potential utility of PDE4 long form activators in the treatment of prostate cancer is studied using the LNCaP human prostate cancer cell line. The experiments are carried out according to the method described by Henderson et al. (Henderson, D. J. P., Byrne, A., Dulla, K., Jenster, G., Hoffmann, R., Baillie, G. S., Houslay, M. D. Br. J. Cancer 110: 1278-1287, 2014).

### LNCaP Cell Culture

Androgen-sensitive (AS) LNCaP cells are maintained in RPM11640 supplemented with 10% FBS (Seralabs), 2 mM L-glutamine and 1,000 U penicillin-streptomycin. LNCaP androgen-insensitive (Al) cells are generated by culturing the LNCaP-AS cells in RPM11640 supplemented with 10% charcoal stripped FBS, 2 mM L-glutamine and 1,000 U penicillin-streptomycin for a minimum of four weeks. All tissue culture reagents are from Life Technologies.

### Xcelligence (Roche) Proliferation Assay

Cell proliferation is measured as a function of changing electrical impedance. Values are represented by cell index number, a dimensionless unit of measurement representing the cell status, which increases as cells adhere to 96-well electrode plates and divide.

LNCaP AI/AS cells are plated at a density of 25,000 cells per well in a 96-well electrode plate (in triplicate), in the presence/absence of various concentrations of test compound.

Cell indices are measured every 10 minutes for up to 100 hours, analysed using RTCA software and normalised to the cell index of vehicle-treated cells (n=3). PDE4 long form activators inhibited the proliferation of AS and AI LNCaP human prostate cancer cells.

### Experiment 6: In Vivo Preclinical Model of Hyperparathyroidism: Inhibition of PTH-Induced cAMP Elevation in Urine in the Anaesthetised Rat

Within the kidney, the binding of parathyroid hormone (PTH) to PTH receptors results in the Gₐₛ-mediated elevation of intracellular cAMP. This increase of intracellular cAMP results in extrusion of cAMP to the urine (Yates et al., J Clin Invest 81: 932-938, 1988). This experiment is based upon a modified Ellsworth-Howard assay (Kruse, K. and Kracht, U., European Journal of Pediatrics 146: 373-377, 1987) and conducted in anaesthetized rats. In this experiment, rats were anaesthetized using isoflurane and catheterised to allow the collection of urine from the bladder. After an initial stabilization period, test compounds were administered by i.v. infusion (modelled to steady state) from time 0-120 min. PTH challenge infusion (33 μg/kg/hour) was started after 60 min of test compound infusion and sustained for one hour (60-120 min). Urine collection was conducted in 30-minute periods. Urine cAMP levels were assessed by ELISA (R&D systems). Urine samples were prepared for analysis as per the manufacturer's instructions. A standard curve for cAMP was assayed for each experiment and samples were assessed using a standard dilution range of 1:2, 1:4, 1:8 and 1:16, ensuring that the resulting data remained on the linear portion of the standard curve. Control animals treated with vehicle alone (no PTH) showed no increase from baseline in urinary cAMP concentrations over the course of the experiment. Urine cAMP concentration remained below 50,000 pmol/mL. Control animals treated with PTH infusion plus vehicle (PTH challenge) showed an increase from baseline in cAMP concentration in urine collected from 90 to 120 min.

Treatment with PDE4 long form activators suppressed the elevation in cAMP concentration in urine in response to PTH challenge.

### Table 1: Small Molecule PDE4 Long Form Activators (Examples 1 to 122), According to the Present Invention

| | | Molecular | |
|---|---|---|---|
| | | weight | |
| Compound | Chemical name | (Daltons) | Chemical structure |
| Example 1 | (S)-N-(chroman-4-yl)-2-(4- methylpyridin-3-yl)-1H- benzo[d]imidazole-5-carboxamide | 384 | 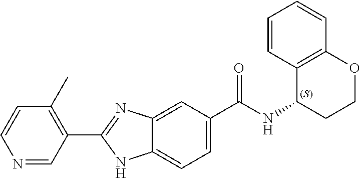 |
| Example 2 | (S)-N-(chroman-4-yl)-2-(6- methylpyridin-3-yl)-1H- benzo[d]imidazole-5-carboxamide | 384 | 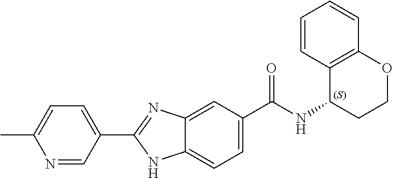 |
| Example 3 | (S)-N-(chroman-4-yl)-2-(2- methylpyridin-3-yl)-1H- benzo[d]imidazole-5-carboxamide | 384 | 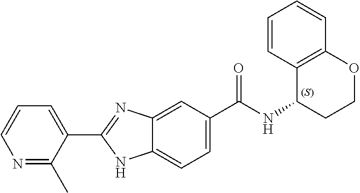 |
| Example 4 | (S)-N-(chroman-4-yl)-2-(5- methylpyridin-3-yl)-1H- benzo[d]imidazole-5-carboxamide | 384 | 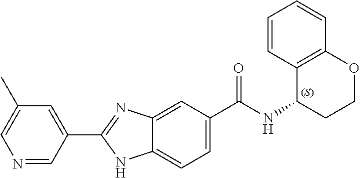 |
| Example 5 | (R)-2-(pyridin-3-yl)-N-(1,2,3,4- tetrahydronaphthalen-1-yl)-1H- benzo[d]imidazole-5-carboxamide | 368 | 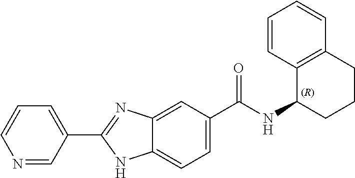 |
| Example 6 | (S)-2-(pyridin-3-yl)-N-(1,2,3,4- tetrahydronaphthalen-1-yl)-1H- benzo[d]imidazole-5-carboxamide | 368 | 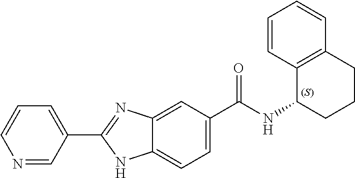 |
| Example 7 | (R)-N-(chroman-4-yl)-2-(pyridin-3- yl)-1H-benzo[d]imidazole-5-carboxamide | 370 | 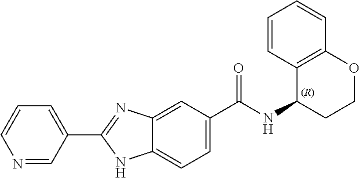 |
| Example 8 | (S)-N-(chroman-4-yl)-2-(pyridin-3- yl)-1H-benzo[d]imidazole-5-carboxamide | 370 | 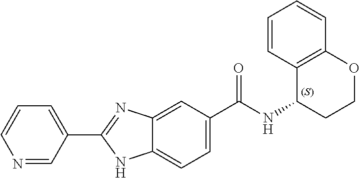 |
| Example 9 | (R)-N-(2,3-dihydro-1H-inden-1-yl)- 2-(pyridin-3-yl)-1H- benzo[d]imidazole-5-carboxamide | 354 | 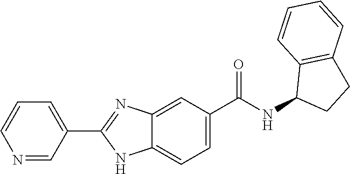 [SMILES: O=C(N[C@@H]1CCc2ccccc21)c1ccc2[nH]c(-c3cccnc3)nc2c1] |
| Example 10 | (S)-N-(2,3-dihydro-1H-inden-1-yl)- 2-(pyridin-3-yl)-1H- benzo[d]imidazole-5-carboxamide | 354 | 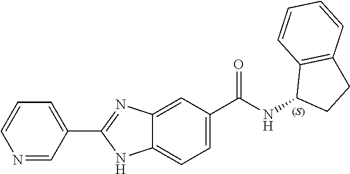 |
| Example 11 | (R)-N-(6-chlorochroman-4-yl)-2- (pyridin-3-yl)-1H- benzo[d]imidazole-5-carboxamide | 405 | 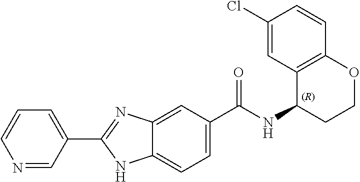 |
| Example 12 | (S)-N-(6-chlorochroman-4-yl)-2- (pyridin-3-yl)-1H- benzo[d]imidazole-5-carboxamide | 405 | 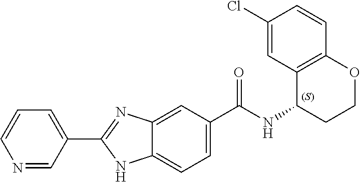 |
| Example 13 | (S)-N-(2,3-dihydro-1H-inden-1-yl)- 2-(1-methylpiperidin-4-yl)-1H- benzo[d]imidazole-5-carboxamide | 374 | 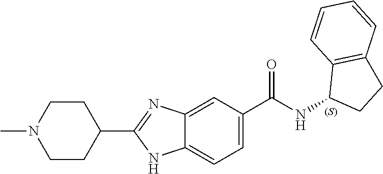 |
| Example 14 | (R)-2-(1-methylpiperidin-4-yl)-N- (1,2,3,4-tetrahydronaphthalen-1-yl)- 1H-benzo[d]imidazole-5-carboxamide | 389 | 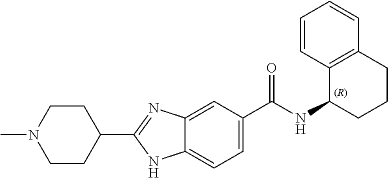 |
| Example 15 | (S)-2-(1-methylpiperidin-4-yl)-N- (1,2,3,4-tetrahydronaphthalen-1-yl)- 1H-benzo[d]imidazole-5-carboxamide | 389 | 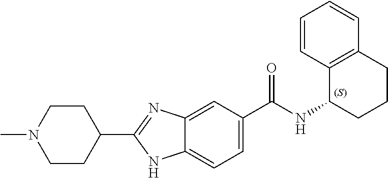 |
| Example 16 | (R)-N-(6-chlorochroman-4-yl)-2-(1- methylpiperidin-4-yl)-1H- benzo[d]imidazole-5-carboxamide | 425 | 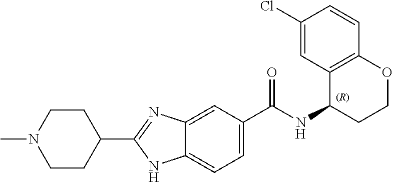 |
| Example 17 | (S)-N-(6-chlorochroman-4-yl)-2-(1- methylpiperidin-4-yl)-1H- benzo[d]imidazole-5-carboxamide | 425 | 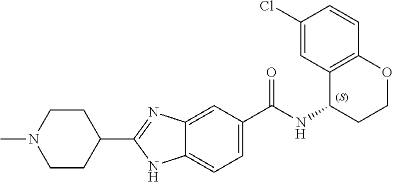 |
| Example 18 | (S)-N-(chroman-4-yl)-2-(1- methylpiperidin-4-yl)-1H- benzo[d]imidazole-5-carboxamide | 390 | 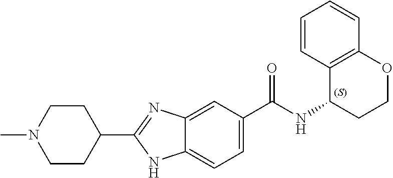 |
| Example 19 | (S)-N-(chroman-4-yl)-2-(4- ethylpiperazin-1-yl)-1H- benzo[d]imidazole-5-carboxamide | 406 | 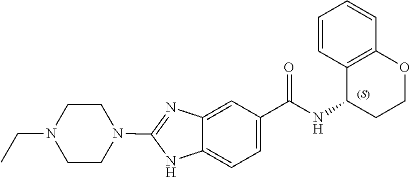 |
| Example 20 | (S)-N-(chroman-4-yl)-1-methyl-2-(4- methylpyridin-3-yl)-1H- benzo[d]imidazole-5-carboxamide | 398 | 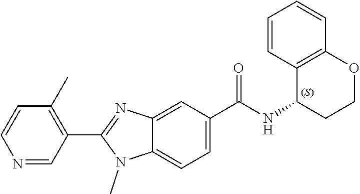 |
| Example 21 | (S)-N-(chroman-4-yl)-1-methyl-2-(6- methylpyridin-3-yl)-1H- benzo[d]imidazole-5-carboxamide | 398 | 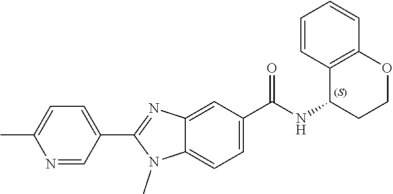 |
| Example 22 | (S)-N-(chroman-4-yl)-1-methyl-2-(2- methylpyridin-3-yl)-1H- benzo[d]imidazole-5-carboxamide | 398 | 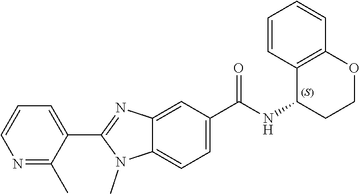 |
| Example 23 | (S)-N-(chroman-4-yl)-1-methyl-2-(5- methylpyridin-3-yl)-1H- benzo[d]imidazole-5-carboxamide | 398 | 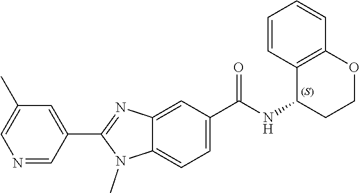 |
| Example 24 | (S)-N-(chroman-4-yl)-1-methyl-2-(5- (pyridin-3-yl)-1H-benzo[d]imidazole- 5-carboxamide | 384 | 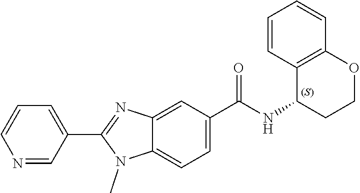 |
| Example 25 | (S)-N-(chroman-4-yl)-1-methyl-2-(4- methylpyridin-3-yl)-1H- benzo[d]imidazole-6-carboxamide | 398 | 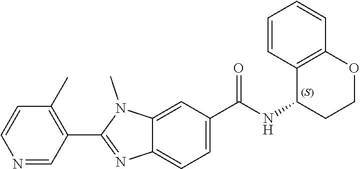 |
| Example 26 | (S)-N-(chroman-4-yl)-1-methyl-2-(6- methylpyridin-3-yl)-1H- benzo[d]imidazole-6-carboxamide | 398 | 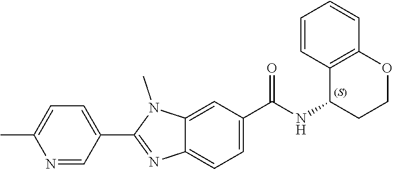 |
| Example 27 | (S)-N-(chroman-4-yl)-1-methyl-2-(2- methylpyridin-3-yl)-1H- benzo[d]imidazole-6-carboxamide | 398 | 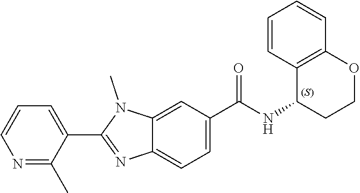 |
| Example 28 | (S)-N-(chroman-4-yl)-1-methyl-2-(5- methylpyridin-3-yl)-1H- benzo[d]imidazole-6-carboxamide | 398 | 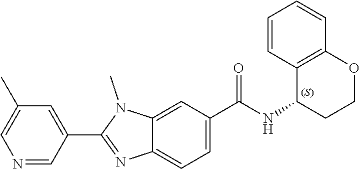 |
| Example 29 | (S)-N-(chroman-4-yl)-1-methyl-2- (pyridin-3-yl)-1H- benzo[d]imidazole-6-carboxamide | 384 | 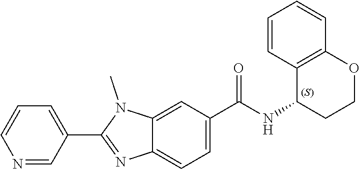 |
| Example 30 | (S)-N-(chroman-4-yl)-2-(4- ethylpiperazin-1-yl)-1-methyl-1H- benzo[d]imidazole-6-carboxamide | 420 | 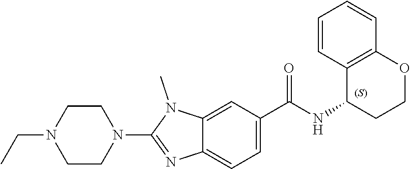 |
| Example 31 | (S)-N-(chroman-4-yl)-1-methyl-2- (piperazin-1-yl)-1H- benzo[d]imidazole-6-carboxamide | 391 | 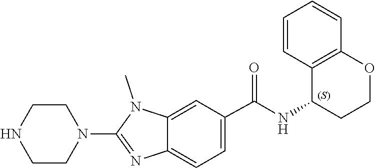 |
| Example 32 | (S)-N-(chroman-4-yl)-2-(4- ethylpiperazin-1-yl)-1-methyl-1H- benzo[d]imidazole-5-carboxamide | 420 | 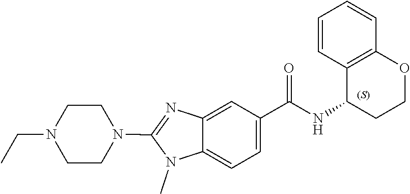 |
| Example 33 | (S)-N-(chroman-4-yl)-1-methyl-2- (piperazin-1-yl)-1H- benzo[d]imidazole-5-carboxamide | 391 | 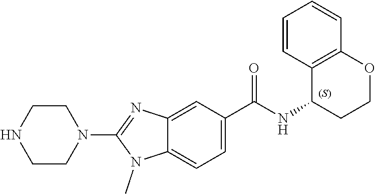 |
| Example 34 | (S)-N-(chroman-4-yl)-2-(pyridin-3- yl)-[1,2,4]triazolo[1,5-a]pyridine-6- carboxamide | 371 | 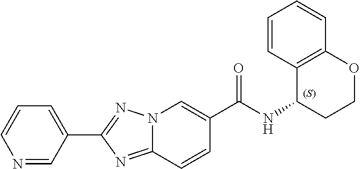 |
| Example 35 | (S)-N-(chroman-4-yl)-2-(6- methylpyridin-3-yl)- [1,2,4]triazolo[1,5-a]pyridine-6- carboxamide | 385 | 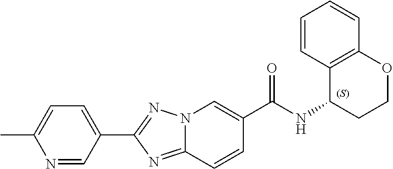 |
| Example 36 | (S)-N-(chroman-4-yl)-2-(5- methylpyridin-3-yl)- [1,2,4]triazolo[1,5-a]pyridine-6- carboxamide | 385 | 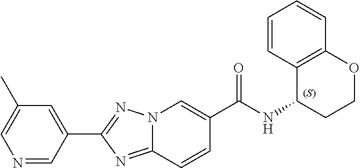 |
| Example 37 | (S)-N-(chroman-4-yl)-2-(4- methylpyridin-3-yl)- [1,2,4]triazolo[1,5-a]pyridine-6- carboxamide | 385 | 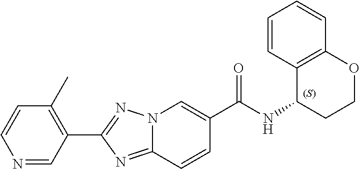 |
| Example 38 | (S)-N-(chroman-4-yl)-2-(2- methylpyridin-3-yl)- [1,2,4]triazolo[1,5-a]pyridine-6- carboxamide | 385 | 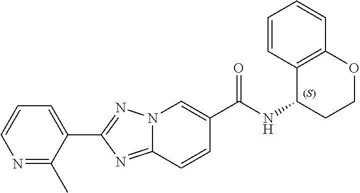 |
| Example 39 | (S)-N-(chroman-4-yl)-2-(2,6- dimethylpyridin-3-yl)- [1,2,4]triazolo[1,5-a]pyridine-6- carboxamide | 399 | 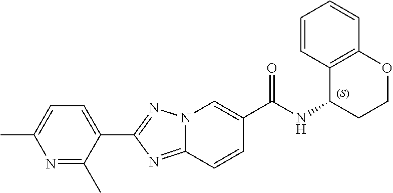 |
| Example 40 | (S)-N-(chroman-4-yl)-2-(piperazin- 1-yl)-[1,2,4]triazolo[1,5-a]pyridine-6- carboxamide | 378 | 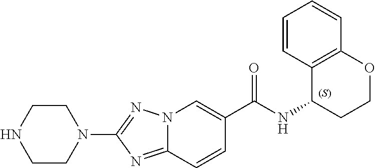 |
| Example 41 | (S)-N-(chroman-4-yl)-2-(4- methylpiperazin-1-yl)- [1,2,4]triazolo[1,5-a]pyridine-6- carboxamide | 392 | 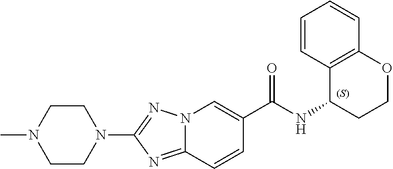 |
| Example 42 | (S)-N-(chroman-4-yl)-2-morpholino- [1,2,4]triazolo[1,5-a]pyridine-6- carboxamide | 379 | 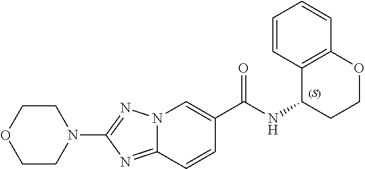 |
| Example 43 | (S)-2-(4-(tert-butyl)piperazin-1-yl)- N-(chroman-4-yl)- [1,2,4]triazolo[1,5-a]pyridine-6- carboxamide | 435 | 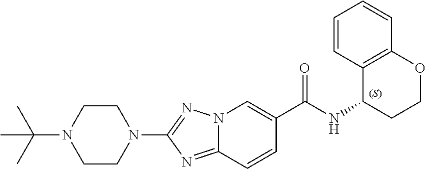 |
| Example 44 | (S)-N-(chroman-4-yl)-2-(piperidin-1- yl)-[1,2,4]triazolo[1,5-a]pyridine-6- carboxamide | 377 | 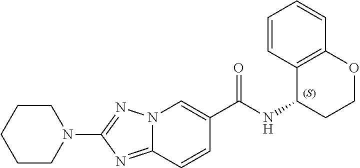 |
| Example 45 | (S)-N-(chroman-4-yl)-2-(4- ethylpiperazin-1-yl)- [1,2,4]triazolo[1,5-a]pyridine-6- carboxamide | 406 | 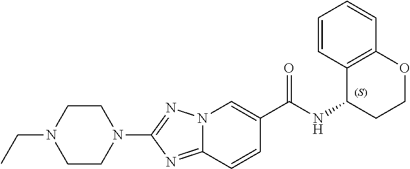 |
| Example 46 | (S)-N-(chroman-4-yl)-2-(4- isopropylpiperazin-1-yl)- [1,2,4]triazolo[1,5-a]pyridine-6- carboxamide | 421 | 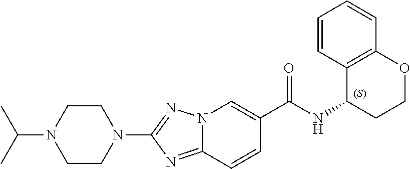 |
| Example 47 | (R)-N-(2,3-dihydro-1H-inden-1-yl)- 2-(piperazin-1-yl)- [1,2,4]triazolo[1,5-a]pyridine-6- carboxamide | 362 | 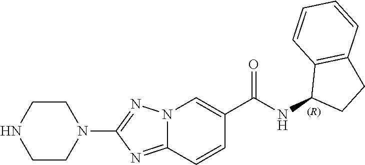 |
| Example 48 | (S)-N-(2,3-dihydro-1H-inden-1-yl)- 2-(piperazin-1-yl)- [1,2,4]triazolo[1,5-a]pyridine-6- carboxamide | 362 | 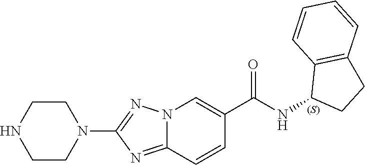 |
| Example 49 | (S)-N-(chroman-4-yl)-2-(piperidin-4- yl)-[1,2,4]triazolo[1,5-a]pyridine-6- carboxamide | 377 | 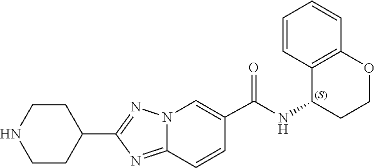 |
| Example 50 | (S)-N-(chroman-4-yl)-2-(1- methylpiperidin-4-yl)- [1,2,4]triazolo[1,5-a]pyridine-6- carboxamide | 391 | 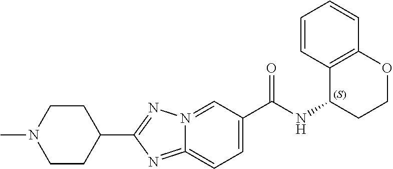 |
| Example 51 | (S)-N-(chroman-4-yl)-2-(1- ethylpiperidin-4-yl)- [1,2,4]triazolo[1,5-a]pyridine-6- carboxamide | 406 | 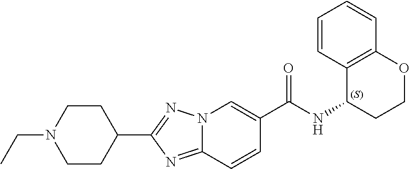 |
| Example 52 | (S)-N-(chroman-4-yl)-2-(2- methylpyridin-3-yl)- [1,2,4]triazolo[1,5-a]pyridine-7- carboxamide | 385 | 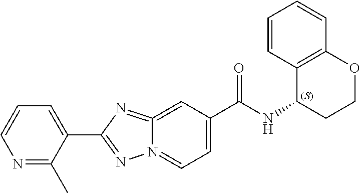 |
| Example 53 | (S)-N-(chroman-4-yl)-2-(4- methylpyridin-3-yl)- [1,2,4]triazolo[1,5-a]pyridine-7- carboxamide | 385 | 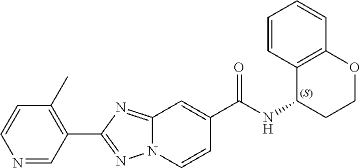 |
| Example 54 | (S)-N-(chroman-4-yl)-2-(4- hydroxypiperidin-1-yl)- [1,2,4]triazolo[1,5-a]pyridine-7- carboxamide | 393 | 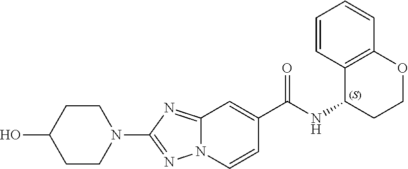 |
| Example 55 | (S)-N-(chroman-4-yl)-2-(piperazin- 1-yl)-[1,2,4]triazolo[1,5-a]pyridine-7- carboxamide | 378 | 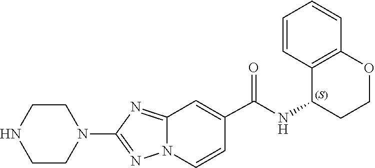 |
| Example 56 | (S)-N-(chroman-4-yl)-2-(4- methylpiperazin-1-yl)- [1,2,4]triazolo[1,5-a]pyridine-7- carboxamide | 392 | 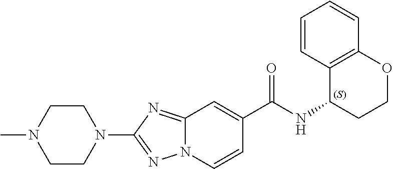 |
| Example 57 | (S)-N-(chroman-4-yl)-2-morpholino- [1,2,4]triazolo[1,5-a]pyridine-7- carboxamide | 379 | 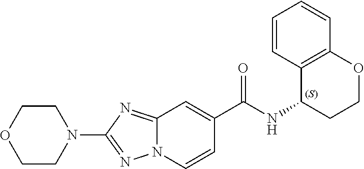 |
| Example 58 | (S)-2-(4-(tert-butyl)piperazin-1-yl)- N-(chroman-4-yl)- [1,2,4]triazolo[1,5-a]pyridine-7- carboxamide | 435 | 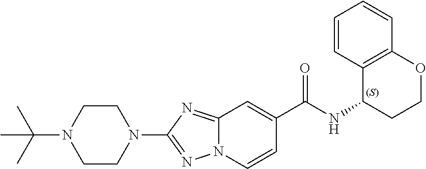 |
| Example 59 | (S)-N-(chroman-4-yl)-2-(piperidin-1- yl)-[1,2,4]triazolo[1,5-a]pyridine-7- carboxamide | 377 | 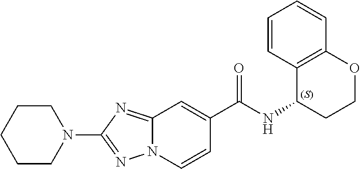 |
| Example 60 | (S)-N-(chroman-4-yl)-2-(4- ethylpiperazin-1-yl)- [1,2,4]triazolo[1,5-a]pyridine-7- carboxamide | 406 | 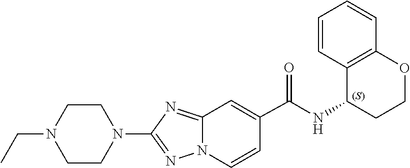 |
| Example 61 | (S)-N-(chroman-4-yl)-2-(4- isopropylpiperazin-1-yl)- [1,2,4]triazolo[1,5-a]pyridine-7- carboxamide | 421 | 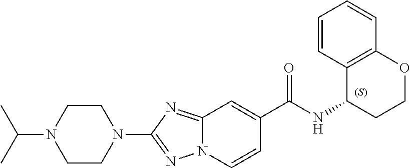 |
| Example 62 | (S)-N-(2,3-dihydro-1H-inden-1-yl)- 2-(piperazin-1-yl)- [1,2,4]triazolo[1,5-a]pyridine-7- carboxamide | 362 | 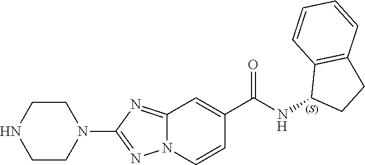 |
| Example 63 | (R)-N-(2,3-dihydro-1H-inden-1-yl)- 2-(piperazin-1-yl)- [1,2,4]triazolo[1,5-a]pyridine-7- carboxamide | 362 | 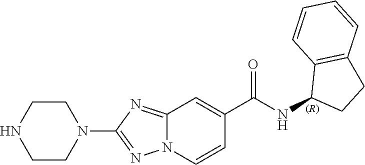 |
| Example 64 | (S)-N-(2,3-dihydro-1H-inden-1-yl)- 2-(4-methylpiperazin-1-yl)- [1,2,4]triazolo[1,5-a]pyridine-7- carboxamide | 376 | 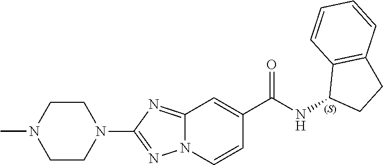 |
| Example 65 | (S)-N-(2,3-dihydro-1H-inden-1-yl)- 2-(4-ethylpiperazin-1-yl)- [1,2,4]triazolo[1,5-a]pyridine-7- carboxamide | 390 | 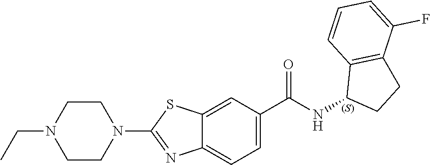 |
| Example 66 | 2-(3,8-diazabicyclo[3.2.1]octan-8- yl)-N-(4,4- difluorocyclohexyl)imidazo[1,2- a]pyridine-6-carboxamide | 389 | 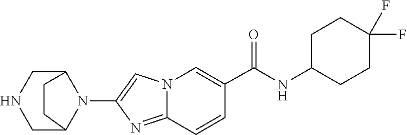 [SMILES: O=C(NC1CCC(F)(F)CC1)c1ccc2nc(N3C4CCC3CNC4)cn2c1] |
| Example 67 | 2-(3,8-diazabicyclo[3.2.1]octan-8- yl)-N-cyclopentylimidazo[1,2- a]pyridine-6-carboxamide | 339 | 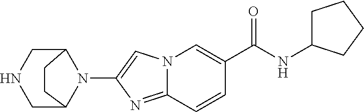 [SMILES: O=C(NC1CCCC1)c1ccc2nc(N3C4CCC3CNC4)cn2c1] |
| Example 68 | (S)-N-(chroman-4-yl)-2-(piperazin- 1-yl)pyrazolo[1,5-a]pyrimidine-6- carboxamide | 378 | 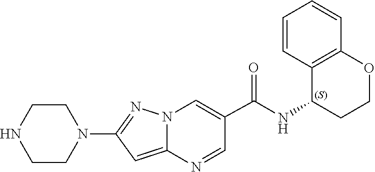 |
| Example 69 | (S)-N-(chroman-4-yl)-2-(4- methylpiperazin-1-yl)pyrazolo [1,5-a]pyrimidine-6-carboxamide | 392 | 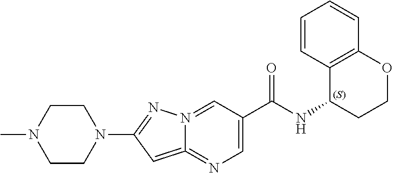 |
| Example 70 | (S)-N-(chroman-4-yl)-2-(4- ethylpiperazin-1-yl)pyrazolo [1,5-a]pyrimidine-6-carboxamide | 406 | 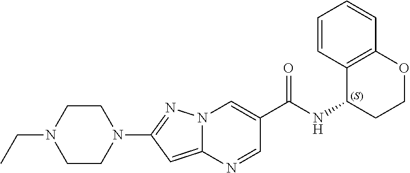 |
| Example 71 | (S)-N-(2,3-dihydro-1H-inden-1-yl)- 2-(piperazin-1-yl)pyrazolo[1,5- a]pyrimidine-6-carboxamide | 362 | 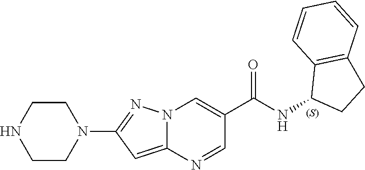 |
| Example 72 | N-cyclopentyl-2-(4-ethylpiperazin-1- yl)pyrazolo[1,5-a]pyrimidine-6- carboxamide | 342 | 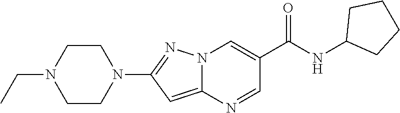 [SMILES: CCN1CCN(c2cc3ncc(C(=O)NC4CCCC4)cn3n2)CC1] |
| Example 73 | N-cyclohexyl-2-(4-ethylpiperazin-1- yl)pyrazolo[1,5-a]pyrimidine-6- carboxamide | 356 | 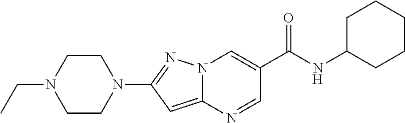 |
| Example 74 | N-(4,4-difluorocyclohexyl)-2-(4- ethylpiperazin-1-yl)pyrazolo[1,5-a] pyrimidine-6-carboxamide | 392 | 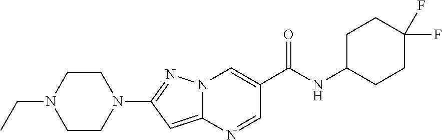 [SMILES: CCN1CCN(c2cc3ncc(C(=O)NC4CCC(F)(F)CC4)cn3n2)CC1] |
| Example 75 | (S)-N-(2,3-dihydro-1H-inden-1-yl)- 2-(4-ethylpiperazin-1- yl)pyrazolo[1,5-a]pyrimidine-6- carboxamide | 390 | 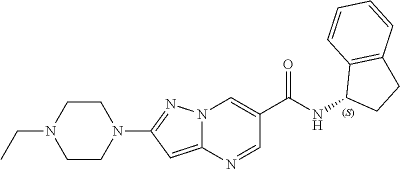 |
| Example 76 | N-cyclopentyl-2-(4-methylpiperazin- 1-yl)pyrazolo[1,5-a]pyrimidine-6- carboxamide | 328 | 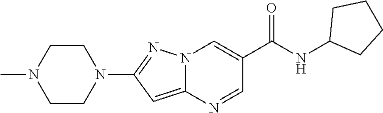 [SMILES: CN1CCN(c2cc3ncc(C(=O)NC4CCCC4)cn3n2)CC1] |
| Example 77 | N-cyclohexyl-2-(4-methylpiperazin- 1-yl)pyrazolo[1,5-a]pyrimidine-6- carboxamide | 342 | 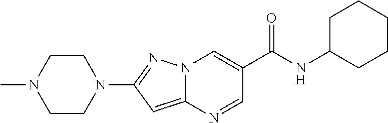 |
| Example 78 | N-(4,4-difluorocyclohexyl)-2-(4- methylpiperazin-1-yl)pyrazolo [1,5-a]pyrimidine-6-carboxamide | 378 | 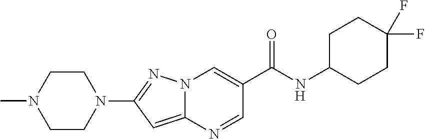 |
| Example 79 | (S)-N-(2,3-dihydro-1H-inden-1-yl)- 2-(4-methylpiperazin-1- yl)pyrazolo[1,5-a]pyrimidine-6- carboxamide | 376 | 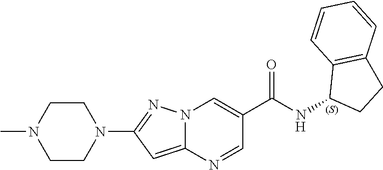 |
| Example 80 | 2-(3,8-diazabicyclo[3.2.1]octan-8-yl)- N-cyclopentylpyrazolo[1,5-a]pyrimidine-6- carboxamide | 340 | 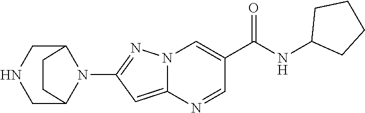 |
| Example 81 | N-cyclopentyl-2-(3-methyl-3,8- diazabicyclo[3.2.1]octan-8- yl)pyrazolo[1,5-a]pyrimidine-6- carboxamide | 354 | 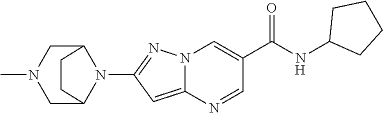 |
| Example 82 | (S)-N-(chroman-4-yl)-2-(piperidin-4- yl)pyrazolo[1,5-a]pyrimidine-6- carboxamide | 377 | 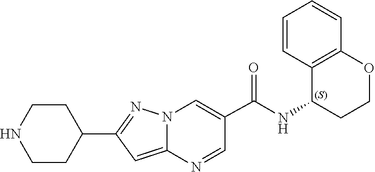 |
| Example 83 | (S)-N-(2,3-dihydro-1H-inden-1-yl)-2- (piperidin-4-yl)pyrazolo[1,5-a]pyrimidine-6- carboxamide | 361 | 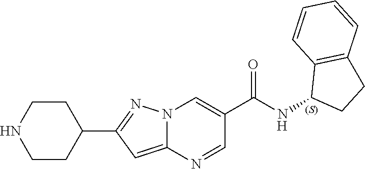 |
| Example 84 | (S)-N-(chroman-4-yl)-2-(1- ethylpiperidin-4-yl)pyrazolo [1,5-a]pyrimidine-6- carboxamide | 405 | 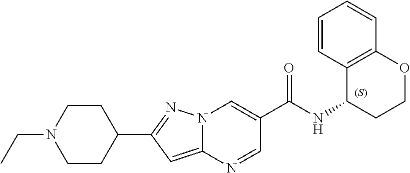 |
| Example 85 | N-cyclopentyl-2-(1-methylpiperidin-4- yl)pyrazolo[1,5-a]pyrimidine-6- carboxamide | 327 | 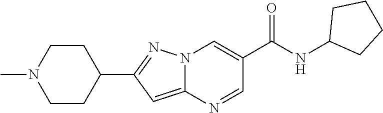 [SMILES: CN1CCC(c2cc3ncc(C(=O)NC4CCCC4)cn3n2)CC1] |
| Example 86 | N-cyclohexyl-2-(1-methylpiperidin-4- yl)pyrazolo[1,5-a]pyrimidine-6- carboxamide | 341 | 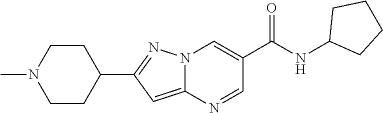 [SMILES: CN1CCC(c2cc3ncc(C(=O)NC4CCCC4)cn3n2)CC1] |
| Example 87 | N-(4,4-difluorocyclohexyl)-2-(1- methylpiperidin-4-yl)pyrazolo [1,5-a]pyrimidine-6- carboxamide | 377 | 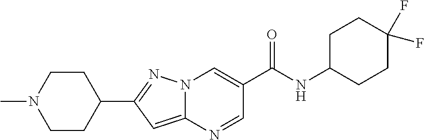 [SMILES: CN1CCC(c2cc3ncc(C(=O)NC4CCC(F)(F)CC4)cn3n2)CC1] |
| Example 88 | (S)-N-(chroman-4-yl)-2-(1- methylpiperidin-4-yl)pyrazolo[1,5-a] pyrimidine-6- carboxamide | 391 | 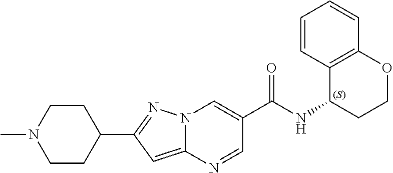 |
| Example 89 | N-(4-fluorobenzyl)-2-(piperidin-4- yl)pyrazolo[1,5-a]pyrimidine-6- carboxamide | 353 | 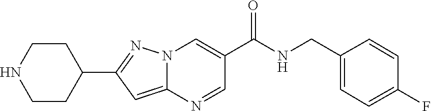 [SMILES: O=C(NCc1ccc(F)cc1)c1cnc2cc(C3CCNCC3)nn2c1] |
| Example 90 | (S)-N-(chroman-4-yl)-2-(pyridin-3- yl)pyrazolo[1,5-a]pyrimidine-6- carboxamide | 371 | 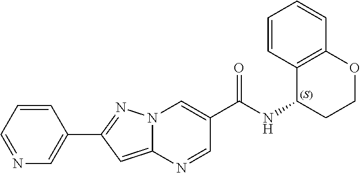 |
| Example 91 | N-(4,4-difluorocyclohexyl)-7-methyl- 2-(piperidin-4-yl)pyrazolo [1,5-a]pyrimidine-6-carboxamide | 377 | 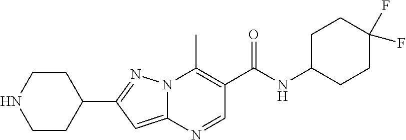 [SMILES: Cc1c(C(=O)NC2CCC(F)(F)CC2)cnc2cc(C3CCNCC3)nn12] |
| Example 92 | (S)-N-(chroman-4-yl)-7-methyl-2- (piperidin-4-yl)pyrazolo [1,5-a]pyrimidine-6- carboxamide | 391 | 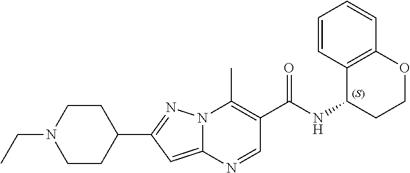 |
| Example 93 | (S)-N-(chroman-4-yl)-2-(1- ethylpiperidin-4-yl)-7- methylpyrazolo[1,5-a]pyrimidine-6- carboxamide | 419 | 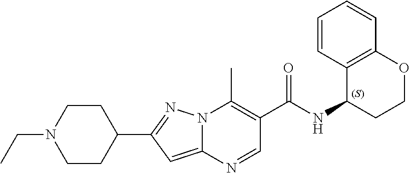 |
| Example 94 | N-(4,4-difluorocyclohexyl)-2-(1- ethylpiperidin-4-yl)-7- methylpyrazolo[1,5-a]pyrimidine-6- carboxamide | 405 | 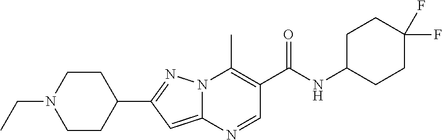 [SMILES: CCN1CCC(c2cc3ncc(C(=O)NC4CCC(F)(F)CC4)c(C)n3n2)CC1] |
| Example 95 | (S)-N-(chroman-4-yl)-7-methyl-2-(1- methylpiperidin-4-yl)pyrazolo [1,5-a]pyrimidine-6-carboxamide | 405 | 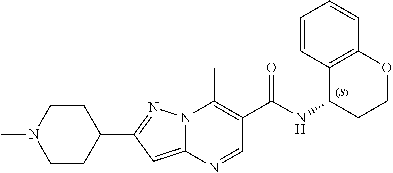 |
| Example 96 | N-(4,4-difluorocyclohexyl)-7-methyl- 2-(1-methylpiperidin-4- yl)pyrazolo[1,5-a]pyrimidine-6- carboxamide | 391 | 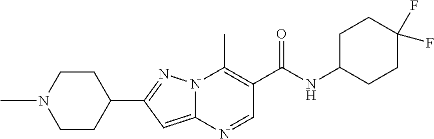 [SMILES: Cc1c(C(=O)NC2CCC(F)(F)CC2)cnc2cc(C3CCN(C)CC3)nn12] |
| Example 97 | N-cyclopentyl-2-(piperidin-4- yl)benzo[d]oxazole-5-carboxamide | 313 | 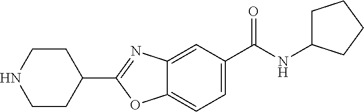 [SMILES: O=C(NC1CCCC1)c1ccc2oc(C3CCNCC3)nc2c1] |
| Example 98 | N-cyclopentyl-2-(1-methylpiperidin-4- yl)benzo[d]oxazole-5-carboxamide | 327 | 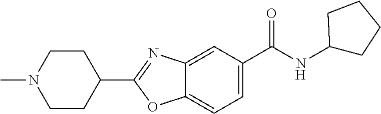 |
| Example 99 | N-cyclopentyl-2-(1-ethylpiperidin-4- yl)benzo[d]oxazole-5-carboxamide | 341 | 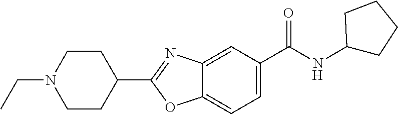 |
| Example 100 | (S)-N-(chroman-4-yl)-2-(piperazin-1- yl)benzo[d]oxazole-5-carboxamide | 378 | 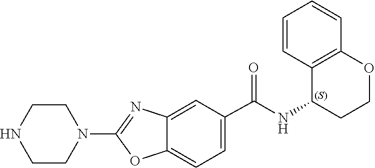 |
| Example 101 | N-cyclopentyl-2-(piperazin-1- yl)benzo[d]oxazole-5-carboxamide | 314 | 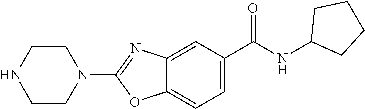 [SMILES: O=C(NC1CCCC1)c1ccc2oc(N3CCNCC3)nc2c1] |
| Example 102 | (S)-N-(chroman-4-yl)-2-(4- ethylpiperazin-1- yl)benzo[d]oxazole-5-carboxamide | 406 | 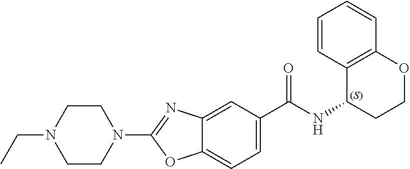 |
| Example 103 | N-cyclopentyl-2-(4-ethylpiperazin-1- yl)benzo[d]oxazole-5-carboxamide | 342 | 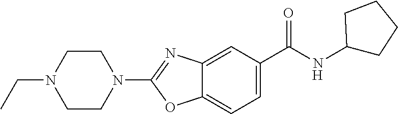 |
| Example 104 | (S)-N-(chroman-4-yl)-2-(4- methylpiperazin-1- yl)benzo[d]oxazole-5-carboxamide | 392 | 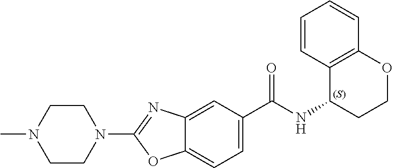 |
| Example 105 | N-cyclopentyl-2-(4-methylpiperazin- 1-yl)benzo[d]oxazole-5-carboxamide | 328 | 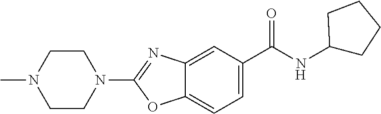 |
| Example 106 | (S)-N-(chroman-4-yl)-2-(4-(2- hydroxyethyl)piperazin-1- yl)benzo[d]oxazole-5-carboxamide | 422 | 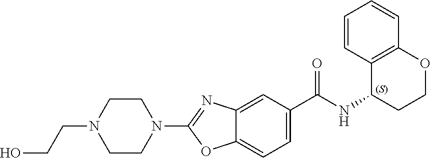 |
| Example 107 | N-cyclopentyl-2-(4-(2- hydroxyethyl)piperazin-1- yl)benzo[d]oxazole-5-carboxamide | 358 | 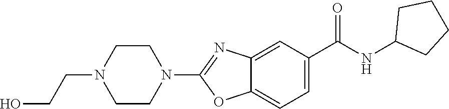 [SMILES: O=C(NC1CCCC1)c1ccc2oc(N3CCN(CCO)CC3)nc2c1] |
| Example 108 | N-cyclopentyl-2-(4-ethylpiperazin-1- yl)benzo[d]oxazole-6-carboxamide | 342 | 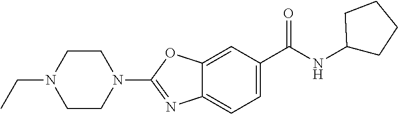 [SMILES: CCN1CCN(c2nc3ccc(C(=O)NC4CCCC4)cc3o2)CC1] |
| Example 109 | N-cyclopentyl-2-(piperazin-1- yl)benzo[d]oxazole-6-carboxamide | 314 | 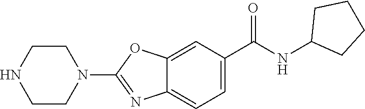 [SMILES: O=C(NC1CCCC1)c1ccc2nc(N3CCNCC3)oc2c1] |
| Example 110 | (S)-N-(chroman-4-yl)-2-(1-methyl- 1H-pyrazol-4- yl)benzo[d]oxazole-6-carboxamide | 374 | 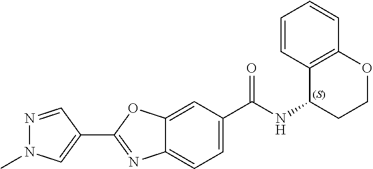 |
| Example 111 | N-cyclopentyl-2-(piperidin-4- yl)thiazolo[5,4-b]pyridine-5- carboxamide | 330 | 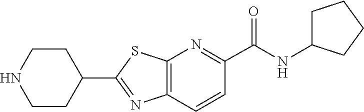 [SMILES: O=C(NC1CCCC1)c1ccc2nc(C3CCNCC3)sc2n1] |
| Example 112 | N-(4,4-difluorocyclohexyl)-2- (piperidin-4-yl)thiazolo[5,4- b]pyridine-5-carboxamide | 380 | 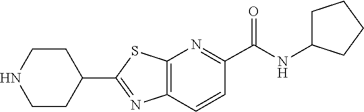 [SMILES: O=C(NC1CCCC1)c1ccc2nc(C3CCNCC3)sc2n1] |
| Example 113 | N-cyclopentyl-2-(1-methylpiperidin-4- yl)thiazolo[5,4- b]pyridine-5-carboxamide | 344 | 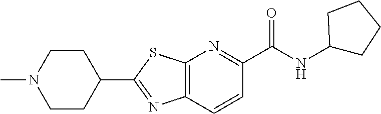 [SMILES: CN1CCC(c2nc3ccc(C(=O)NC4CCCC4)nc3s2)CC1] |
| Example 114 | N-(4,4-difluorocyclohexyl)-2-(1- methylpiperidin-4-yl)thiazolo[5,4- b]pyridine-5-carboxamide | 394 | 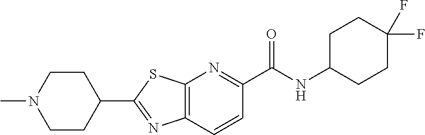 [SMILES: CN1CCC(c2nc3ccc(C(=O)NC4CCC(F)(F)CC4)nc3s2)CC1] |
| Example 115 | 2-(3,8-diazabicyclo[3.2.1]octan-8-yl)- N-cyclopentylthiazolo[5,4- b]pyridine-5-carboxamide | 357 | 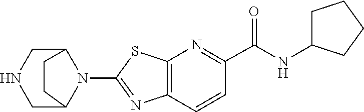 [SMILES: O=C(NC1CCCC1)c1ccc2nc(N3C4CCC3CNC4)sc2n1] |
| Example 116 | 2-(3,8-diazabicyclo[3.2.1]octan-8-yl)- N-(4,4- difluorohexyl)thiazolo[5,4- b]pyridine-5-carboxamide | 407 | 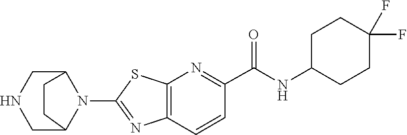 [SMILES: O=C(NC1CCC(F)(F)CC1)c1ccc2nc(N3C4CCC3CNC4)sc2n1] |
| Example 117 | 2-(3,8-diazabicyclo[3.2.1]octan-8-yl)- N-(4,4- difluorocyclohexyl)thiazolo[4,5- b]pyridine-6-carboxamide | 407 | 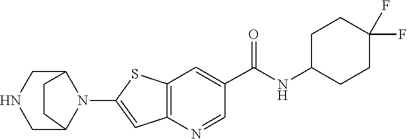 [SMILES: O=C(NC1CCC(F)(F)CC1)c1cnc2cc(N3C4CCC3CNC4)sc2c1] |
| Example 118 | N-cyclopentyl-2-(piperidin-4- yl)thiazolo[4,5- b]pyridine-6-carboxamide | 330 | 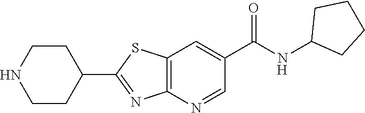 |
| Example 119 | 2-(3,8-diazabicyclo[3.2.1]octan-8-yl)- N-cyclopentylthiazolo[4,5- c]pyridine-6-carboxamide | 357 | 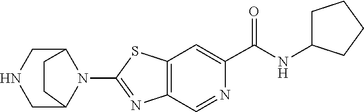 [SMILES: O=C(NC1CCCC1)c1cc2sc(N3C4CCC3CNC4)nc2cn1] |
| Example 120 | 2-(3,8-diazabicyclo[3.2.1]octan-8-yl)- N-(4,4- difluorocyclohexyl)thiazolo[4,5- c]pyridine-6-carboxamide | 407 | 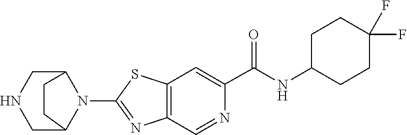 [SMILES: O=C(NC1CCC(F)(F)CC1)c1cc2sc(N3C4CCC3CNC4)nc2cn1] |
| Example 121 | 2-(3,8-diazabicyclo[3.2.1]octan-8-yl)- N-cyclopentylthieno[3,2-b]pyridine-6- carboxamide | 356 | 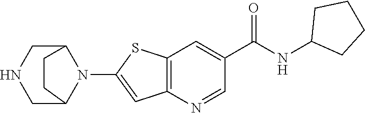 |
| Example 122 | 2-(3,8-diazabicyclo[3.2.1]octan-8-yl)- N-(4,4-difluorocyclohexyl)thieno[3,2- b]pyridine-6-carboxamide | 406 | 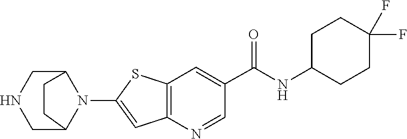 [SMILES: O=C(NC1CCC(F)(F)CC1)c1cnc2cc(N3C4CCC3CNC4)sc2c1] |

TABLE 2
Enzyme assay data for PDE4D5, a long form of PDE4
Using the method described in Experiment 1, the
following PDE4D5 activation data were obtained
for exemplary compounds of the present invention.

| Compound Example | Increase in PDE4D5 |
|---|---|
| Number | activity at 100 μM* (%) |
| Example 2 | 115 |
| Example 4 | 140 |
| Example 8 | 52 |
| Example 10 | 46 |
| Example 11 | 35 |
| Example 15 | 123 |
| Example 28 | 45 |
| Example 34 | 176 |
| Example 35 | 68 |
| Example 36 | 57 |
| Example 37 | 127 |
| Example 39 | 63 |
| Example 53 | 33 |
| Example 90 | 99 |

*Measured as mean % increase in counts over basal activity

TABLE 3
Enzyme assay data for PDE4B2, a short form of PDE4
Using the method described in Experiment 1, the
following PDE4B2 data were obtained for exemplary
compounds of the present invention.

| Compound Example | % increase in PDE4B2 |
|---|---|
| Number | activity at 100 μM* |
| Example 2 | −39 |
| Example 4 | −6 |
| Example 8 | −35 |
| Example 10 | −35 |
| Example 15 | 3 |
| Example 28 | 5 |
| Example 34 | 0 |
| Example 35 | −11 |
| Example 36 | 6 |
| Example 37 | −24 |
| Example 39 | −28 |
| Example 53 | −8 |
| Example 90 | −8 |

*Measured as mean % increase in counts over basal activity

TABLE 4
Inhibition of PGE2-stimulated in vitro cyst formation
in m-IMCD3 cells
Using the method described in Experiment 4, the following
m-IMCD3 kidney cell cyst suppression data were obtained
for exemplary compounds of the present invention.

| | % Cyst area* after |
|---|---|
| Compound Example | incubation with 10 μM |
| Number | test compound |
| Example 1 | 26 |
| Example 2 | 17 |
| Example 3 | 20 |
| Example 4 | 18 |
| Example 21 | 3 |
| Example 22 | 18 |
| Example 23 | 12 |
| Example 24 | 21 |
| Example 28 | 8 |
| Example 31 | 3 |
| Example 33 | 19 |
| Example 40 | 11 |
| Example 45 | 15 |
| Example 46 | 4 |
| Example 48 | 22 |
| Example 50 | 6 |
| Example 51 | 6 |
| Example 52 | 19 |
| Example 53 | 20 |
| Example 56 | 14 |
| Example 59 | 26 |
| Example 60 | 18 |
| Example 61 | 10 |
| Example 62 | 0 |
| Example 64 | 1 |
| Example 67 | 27 |
| Example 68 | 9 |
| Example 69 | 7 |
| Example 70 | 5 |
| Example 73 | −3 |
| Example 75 | 19 |
| Example 76 | 9 |
| Example 77 | 0 |
| Example 80 | 14 |
| Example 81 | −3 |
| Example 82 | 3 |
| Example 84 | 7 |
| Example 86 | 7 |
| Example 88 | 15 |
| Example 89 | −3 |
| Example 91 | 5 |
| Example 94 | 13 |
| Example 95 | 13 |
| Example 96 | 11 |
| Example 98 | −3 |
| Example 101 | 21 |
| Example 102 | 8 |
| Example 103 | −1 |
| Example 106 | −3 |
| Example 108 | 24 |
| Example 116 | −1 |
| Example 120 | 29 |
| Example 121 | 3 |

*Compared to 100% for (DMSO + PGE2) and 0% for DMSO control

35. It will be appreciated that the above description is made by way of example and not limitation of the scope of the appended claims, including any equivalents as included within the scope of the claims. Various modifications are possible and will be readily apparent to the skilled person in the art. Likewise, features of the described embodiments can be combined with any appropriate aspect described above and optional features of any one aspect can be combined with any other appropriate aspect.

1. A compound of Formula I 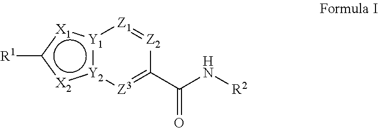
or a pharmaceutically acceptable salt or derivative thereof, wherein:
one of X₁ and X₂ is N and the other is N or CR^{3a}, one of Y₁ and Y₂ is N and the other is C, and one of Z₁, Z₂ and Z₃ is N or CR^{3b} and the others are each CR^{3b}; or
one of X₁ and X₂ is N and the other is NR^{3c} or O, Y₁ and Y₂ are each C, and one of Z₁, Z₂ and Z₃ is N or CR^{3b} and the others are each CR^{3b}; or
one of X₁ and X₂ is S and the other is N or CR^{3a}, Y₁ and Y₂ are each C, and one of Z₁, Z₂ and Z₃ is N and the others are each CR^{3b};
R¹ is a 4- to 10-membered monocyclic, bridged or bicyclic ring containing at least 1 ring N heteroatom and optionally a ring O heteroatom, and wherein R¹ is optionally substituted with 1 or more R⁴;
R² is
(v) (C5-7)cycloalkyl, fused to a 6-membered aromatic or heteroaromatic ring that contains 0, 1 or 2 ring N atoms;
(vi) a 5- to 7-membered non-aromatic heterocycle containing one ring O heteroatom, optionally fused to a 6-membered aromatic or heteroaromatic ring that contains 0, 1 or 2 ring N atoms;
(vii) CH₂Ar, where Ar is a 6-membered aromatic or heteroaromatic ring that contains 0, 1 or 2 ring N atoms; or
(viii) a (C3-8)alkyl group that may be straight chain, branched or cyclic, or a combination thereof;
and wherein R² is optionally substituted with 1 or more R⁵;
each R^{3a} is independently H or (C1-6)alkyl, the (C1-6)alkyl being optionally substituted by 1 or more halogen;
each R^{3b} is independently H, (C1-6)alkyl, (C1-6)alkoxy, CN or halogen, the (C1-6)alkyl and (C1-6)alkoxy being optionally substituted by 1 or more halogen;
each R^{3c} is independently H or (C1-6)alkyl, the (C1-6)alkyl being optionally substituted by 1 or more halogen;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy; and
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH;
for use in the treatment or prevention of a disease or disorder that can be ameliorated by activation of long isoforms of PDE4 or a disease or disorder mediated by excessive intracellular cyclic AMP signalling.

2. The compound, or a pharmaceutically acceptable salt or derivative thereof, for use of claim 1, wherein R¹ is a 5- to 6-membered saturated, monocyclic ring containing at least 1 ring N heteroatom and optionally a ring O heteroatom; a 5- to 6-membered aromatic, monocyclic ring containing 1 or 2 ring N heteroatoms; or a 7- to 8-membered saturated, bridged ring containing 1 or 2 ring N heteroatoms; a 9-membered saturated, bridged ring system containing 2 ring N heteroatoms and a ring O-heteroatom; or a 7- to 10-membered saturated, fused or spiro ring system containing 1 or 2 ring N heteroatoms; and wherein R¹ is optionally substituted with 1, 2 or 3 R⁴, optionally R¹ is a 7- to 8-membered saturated, bridged ring containing 1 or 2 ring N heteroatoms.

3. The compound, or a pharmaceutically acceptable salt or derivative thereof, for use of claim 1 or 2, wherein R¹ is a 7- to 8-membered saturated, bridged ring containing 1 or 2 ring N heteroatoms, wherein at least 1 ring N heteroatom is not at the point of attachment of R¹, and wherein R¹ is optionally substituted with 1 R⁴.

4. The compound, or a pharmaceutically acceptable salt or derivative thereof, for use of any preceding claim, wherein R² is:
(i) (C5-7)cycloalkyl, fused to a 6-membered aromatic or heteroaromatic ring that contains 0, 1 or 2 ring N atoms, wherein the (C5-7)cycloalkyl is optionally substituted with 1 to 3 substituents independently selected from OH, halogen, (C1-4)alkyl and (C1-4)alkoxy, the (C1-4)alkyl and (C1-4)alkoxy groups being optionally substituted with one or more fluoro, and the 6-membered aromatic or heteroaromatic ring is optionally substituted with 1 to 3 substituents independently selected from (C1-4)alkyl, (C1-4)alkoxy, CN and halogen, the (C1-4)alkyl and (C1-4)alkoxy groups being optionally substituted with one or more fluoro;
(ii) a 5- to 7-membered non-aromatic heterocycle containing one ring O heteroatom, optionally fused to a 6-membered aromatic or heteroaromatic ring that contains 0, 1 or 2 ring N atoms, wherein the 5- to 7-membered non-aromatic heterocycle is optionally substituted with 1 to 3 substituents on one or more ring carbon atoms independently selected from OH, halogen, (C1-4)alkyl and (C1-4)alkoxy, the (C1-4)alkyl and (C1-4)alkoxy groups being optionally substituted with one or more fluoro, and the 6-membered aromatic or heteroaromatic ring is optionally substituted with 1 to 3 substituents independently selected from (C1-4)alkyl, (C1-4)alkoxy, CN and halogen, the (C1-4)alkyl and (C1-4)alkoxy groups being optionally substituted with one or more fluoro;
(iii) CH₂Ar, wherein the Ar is optionally substituted with 1 to 3 substituents selected from halogen, CN, (C1-4)alkyl, (C1-4)alkoxy and the CH₂ is optionally substituted with (C1-4)alkyl the (C1-4)alkyl group being optionally substituted with OH or (C1-4)alkyloxy; or
(iv) a (C3-8)alkyl group that may be straight chain, branched or cyclic or a combination thereof, optionally substituted with 1 or more halogen, (C1-4)alkoxy or OH.

5. The compound, or a pharmaceutically acceptable salt or derivative thereof, for use of any preceding claim, wherein R² is:
(i) (C5-6)cycloalkyl, optionally fused to a phenyl ring;
(ii) a 5- to 6-membered non-aromatic heterocycle containing one ring O heteroatom, optionally fused to a phenyl ring;
wherein R² is optionally substituted with 1 or more R⁵.

6. The compound, or a pharmaceutically acceptable salt or derivative thereof, for use of any preceding claim, wherein R² is a group of formula 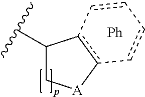
wherein A is O or CH₂; p is 1, 2 or 3; Ph is an optionally present, fused phenyl ring, and wherein R² is optionally substituted with 1 or more R⁵; optionally wherein A is O or C(R⁵)₂ (for example, CF₂).

7. The compound, or a pharmaceutically acceptable salt or derivative thereof, for use of any preceding claim, wherein:
a) each R^{3a} is independently —H or CH₃;
b) each R^{3b} is independently —H, —CH₃, —OCH₃, halo, CN or cyclopropyl; and/or
c) each R^{3c} is independently —H or CH₃.

8. The compound, or a pharmaceutically acceptable salt or derivative thereof, for use of any preceding claim, wherein 1 or 2 (preferably 1) of R^{3a}, R^{3b} and R^{3c} is as defined in any of claims 1 or 7 and the others where present are each —H.

9. The compound, or a pharmaceutically acceptable salt or derivative thereof, for use of any preceding claim, wherein the bicyclic heteroaromatic ring system containing X₁, X₂, Y₁, Y₂, Z₁, Z₂ and Z₃ is selected from:

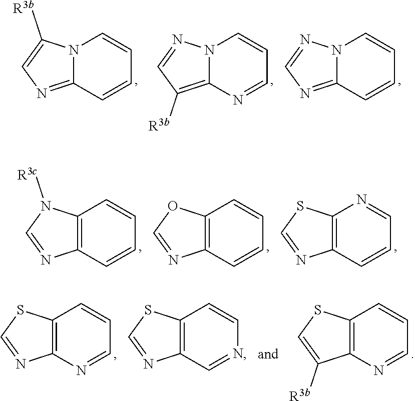

10. The compound, or a pharmaceutically acceptable salt or derivative thereof, for use of any preceding claim, wherein one of X₁ and X₂ is N and the other is N or CR^{3a}, one of Y₁ and Y₂ is N and the other is C, and one of Z₁, Z₂ and Z₃ is N or CR^{3b} and the others are each CR^{3b}.

11. The compound, or a pharmaceutically acceptable salt or derivative thereof, for use of any of claims 1-9, wherein one of X₁ and X₂ is S and the other is N or CR^{3a}, Y₁ and Y₂ are each C, and one of Z₁, Z₂ and Z₃ is N and the others are each CR^{3b}.

12. The compound, or a pharmaceutically acceptable salt or derivative thereof, for use of any preceding claim, wherein
R¹ is a 6-membered saturated
monocyclic ring containing 1 or 2 ring N heteroatoms, wherein at least 1 ring N heteroatom is not at the point of attachment of R¹, or a 7- to 8-membered saturated, bridged ring system containing 2 ring N heteroatoms, and wherein R¹ is optionally substituted with 1 R⁴;
R² is
(i) (C5-6)cycloalkyl, optionally fused to a phenyl ring; or
(ii) a 5- to 6-membered non-aromatic heterocycle containing one ring O heteroatom, optionally fused to a phenyl ring;
and wherein R² is optionally substituted with 1 or more R⁵;
R^{3a}, R^{3b} and R^{3c}, where present, are each independently H or methyl (optionally wherein 0 or 1 of R^{3a}, R^{3b} and R^{3c} is methyl and the others are H);
R⁴, where present, is (C1-6)alkyl optionally substituted with OH; and
R⁵, where present, is OH.

13. A compound of Formula II 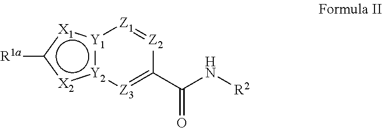
or a pharmaceutically acceptable salt or derivative thereof, wherein:
one of X₁ and X₂ is N and the other is N or CR^{3a}, one of Y₁ and Y₂ is N and the other is C, and one of Z₁, Z₂ and Z₃ is N or CR^{3b} and the others are each CR^{3b}; or
one of X₁ and X₂ is S and the other is N or CR^{3a}, Y₁ and Y₂ are each C, and one of Z₁, Z₂ and Z₃ is N and the others are each CR^{3b};
R^{1a} is a 4- to 10-membered non-aromatic ring that can be monocyclic, bridged or bicyclic containing at least 1 ring N heteroatom and optionally a ring O heteroatom, wherein at least 1 ring N heteroatom is not at the point of attachment of R^{1a}, and wherein R^{1a} is optionally substituted with 1 or more R⁴;
R² is
(v) (C5-7)cycloalkyl, fused to a 6-membered aromatic or heteroaromatic ring that contains 0, 1 or 2 ring N atoms;
(vi) a 5- to 7-membered non-aromatic heterocycle containing one ring O heteroatom, optionally fused to a 6-membered aromatic or heteroaromatic ring that contains 0, 1 or 2 ring N atoms;
(vii) CH₂Ar, where Ar is a 6-membered aromatic or heteroaromatic ring that contains 0, 1 or 2 ring N atoms; or
(viii) a (C3-8)alkyl group that may be straight chain, branched or cyclic, or a combination thereof;
and wherein R² is optionally substituted with 1 or more R⁵;
each R^{3a} is independently H or (C1-6)alkyl, the (C1-6)alkyl being optionally substituted by 1 or more halogen;
each R^{3b} is independently H, (C1-6)alkyl, (C1-6)alkoxy, CN or halogen, the (C1-6)alkyl and (C1-6)alkoxy being optionally substituted by 1 or more halogen;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy; and
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH.

14. The compound or a pharmaceutically acceptable salt or derivative thereof of claim 13, wherein:
a) each R⁴ is independently halogen, CN, OH, (C1-2)alkyl, (C1-6)alkoxy or —(C1-6)alkylene-(C1-6)alkoxy, the (C1-2)alkyl, (C1-6)alkoxy and —(C1-6)alkylene-(C1-6)alkoxy being optionally substituted with 1 or more substituents independently selected from halogen, OH and (C1-6)alkoxy; and/or
b) R² is (C5-7)cycloalkyl, fused to a 6-membered aromatic or heteroaromatic ring that contains 0, 1 or 2 ring N atoms; a 5- to 7-membered non-aromatic heterocycle containing one ring O heteroatom, optionally fused to a 6-membered aromatic or heteroaromatic ring that contains 0, 1 or 2 ring N atoms; or CH₂Ar, where Ar is a 6-membered aromatic or heteroaromatic ring that contains 0, 1 or 2 ring N atoms; and wherein R² is optionally substituted with 1 or more R⁵.

15. The compound or a pharmaceutically acceptable salt or derivative thereof of claim 13 or 14, wherein R^{1a} is a 5- to 6-membered saturated, monocyclic ring containing at least 1 ring N heteroatom and optionally a ring O heteroatom; or a 7- to 8-membered saturated, bridged ring containing 1 or 2 ring N heteroatoms; a 9-membered saturated, bridged ring system containing 2 ring N heteroatoms and a ring O-heteroatom; or a 7- to 10-membered saturated, fused or spiro ring system containing 1 or 2 ring N heteroatoms; and wherein R^{1a} is optionally substituted with 1, 2 or 3 R⁴.

16. The compound, or a pharmaceutically acceptable salt or derivative thereof, of any of claims 13-15, wherein R^{1a} is a 6-membered saturated monocyclic ring containing 1 or 2 ring N heteroatoms or a 7- to 8-membered saturated, bridged ring system containing 2 ring N heteroatoms, wherein R^{1a} is optionally substituted with 1 R⁴, optionally wherein R^{1a} is a 7- to 8-membered saturated, bridged ring system containing 2 ring N heteroatoms, wherein R^{1a} is optionally substituted with 1 R⁴.

17. The compound, or a pharmaceutically acceptable salt or derivative thereof, of any of claims 13-16, wherein R² is:
(i) (C5-7)cycloalkyl, fused to a 6-membered aromatic or heteroaromatic ring that contains 0, 1 or 2 ring N atoms, wherein the (C5-7)cycloalkyl is optionally substituted with 1 to 3 substituents independently selected from OH, halogen, (C1-4)alkyl, and (C1-4)alkoxy, the (C1-4)alkyl and (C1-4)alkoxy groups being optionally substituted with one or more fluoro, and the 6-membered aromatic or heteroaromatic ring is optionally substituted with 1 to 3 substituents independently selected from (C1-4)alkyl, (C1-4)alkoxy, CN and halogen, the (C1-4)alkyl and (C1-4)alkoxy groups being optionally substituted with one or more fluoro;
(ii) a 5- to 7-membered non-aromatic heterocycle containing one ring O heteroatom, optionally fused to a 6-membered aromatic or heteroaromatic ring that contains 0, 1 or 2 ring N atoms, wherein the 5- to 7-membered heterocycle is optionally substituted with 1 to 3 substituents on one or more ring carbon atoms independently selected from OH, halogen, (C1-4)alkyl, (C1-4)alkoxy, the (C1-4)alkyl and (C1-4)alkoxy groups being optionally substituted with one or more fluoro, and the 6-membered aromatic or heteroaromatic ring is optionally substituted with 1 to 3 substituents independently selected from (C1-4)alkyl, (C1-4)alkoxy, CN and halogen, the (C1-4)alkyl and (C1-4)alkoxy groups being optionally substituted with one or more fluoro;
(iii) CH₂Ar, wherein Ar is optionally substituted with 1 to 3 substituents selected from halogen, CN, (C1-4)alkyl, (C1-4)alkoxy and the CH₂ is optionally substituted with (C1-4)alkyl the (C1-4)alkyl group being optionally substituted with OH or (C1-4)alkyloxy; or
(iv) a (C3-8)alkyl group that may be straight chain, branched or cyclic or a combination thereof, optionally substituted with 1 or more halogen, OH or (C1-4)alkoxy.

18. The compound, or a pharmaceutically acceptable salt or derivative thereof, of any of claims 13-17, wherein R² is:
(i) (C5-6)cycloalkyl, optionally fused to a phenyl ring;
(ii) a 5- to 6-membered non-aromatic heterocycle containing one ring O heteroatom, optionally fused to a phenyl ring; and
wherein R² is optionally substituted with 1 or more R⁵.

19. The compound, or a pharmaceutically acceptable salt or derivative thereof, of any of claims 13-18, wherein R² is a group of formula 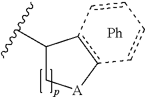
wherein A is O or CH₂; p is 1, 2 or 3; Ph is an optionally present, fused phenyl ring, and wherein R² is optionally substituted with 1 or more R⁵; optionally wherein A is O or C(R⁵)₂ (for example, CF₂).

20. The compound, or a pharmaceutically acceptable salt or derivative thereof, of any of claims 13-19, wherein
R^{1a} is a 6-membered saturated monocyclic ring containing 1 or 2 ring N heteroatoms, wherein at least 1 ring N heteroatom is not at the point of attachment of R^{1a}, or a 7- to 8-membered saturated, bridged ring system containing 2 ring N heteroatoms, and wherein R^{1a} is optionally substituted with 1 R⁴;
R² is
(i) (C5-6)cycloalkyl, optionally fused to a phenyl ring; or
(ii) a 5- to 6-membered non-aromatic heterocycle containing one ring O heteroatom, optionally fused to a phenyl ring;
and wherein R² is optionally substituted with 1 R⁵;
R^{3a} and R^{3b}, where present, are each independently H or methyl (optionally wherein 0 or 1 of R^{3a} and R^{3b} is methyl and the others are H);
R⁴, where present, is (C1-6)alkyl optionally substituted with OH; and
R⁵, where present, is OH.

21. A compound of Formula III 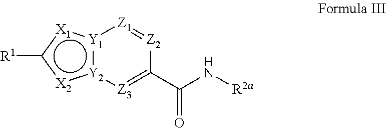
or a pharmaceutically acceptable salt or derivative thereof, wherein:
one of X₁ and X₂ is N and the other is N or CR^{3a}, one of Y₁ and Y₂ is N and the other is C, and one of Z₁, Z₂ and Z₃ is N or CR^{3b} and the others are each CR^{3b}; or
one of X₁ and X₂ is S and the other is N or CR^{3a}, Y₁ and Y₂ are each C, and one of Z₁, Z₂ and Z₃ is N and the others are each CR^{3b};
R¹ is a 4- to 10-membered monocyclic, bridged or bicyclic ring containing at least 1 ring N heteroatom and optionally a ring O heteroatom, and wherein R¹ is optionally substituted with 1 or more R⁴;
R^{2a} is
(iii) (C5-7)cycloalkyl fused to a 6-membered aromatic or heteroaromatic ring that contains 0, 1 or 2 ring N atoms;
(35) a 5- to 7-membered non-aromatic heterocycle containing one ring O heteroatom, optionally fused to a 6-membered aromatic or heteroaromatic ring that contains 0, 1 or 2 ring N atoms; or
(iv) a (C5-6)cycloalkyl group;
and wherein R^{2a} is optionally substituted with 1 or more R⁵;
each R^{3a} is independently H or (C1-6)alkyl, the (C1-6)alkyl being optionally substituted by 1 or more halogen;
each R^{3b} is independently —H, (C1-6)alkyl, (C1-6)alkoxy, CN or halogen, the (C1-6)alkyl and (C1-6)alkoxy being optionally substituted by 1 or more halogen;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy; and
each R⁵ is independently halogen, OH, CN, (C1-6)alkyl, (C1-6)alkoxy or —(C1-6)alkylene-(C1-6)alkoxy the (C1-6)alkyl and (C1-6)alkoxy being optionally substituted by 1 or more halogen or OH; and wherein when one of X₁ and X₂ is S and the other is N, Y₁ and Y₂ are each C, one of Z₁, Z₂ and Z₃ is N and R^{2a} is (iv) a (C5-6)cycloalkyl group, R^{2a} is substituted by at least 2 R⁵.

22. The compound, or a pharmaceutically acceptable salt or derivative thereof, of any of claims 13-21, wherein 1 or 2 (preferably 1) of R^{3a} and R^{3b} is a group other than H and the others, where present, are each H.

23. A compound of Formula IV 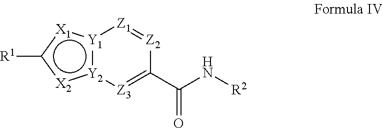
or a pharmaceutically acceptable salt or derivative thereof, wherein:
one of X₁ and X₂ is N and the other is N or CR^{3a}, one of Y₁ and Y₂ is N and the other is C, and one of Z₁, Z₂ and Z₃ is N or CR^{3b} and the others are each CR^{3b}; or
one of X₁ and X₂ is S and the other is N or CR^{3a}, Y₁ and Y₂ are each C, and one of Z₁, Z₂ and Z₃ is N and the others are each CR^{3b};
R¹ is a 4- to 10-membered monocyclic, bridged or bicyclic ring containing at least 1 ring N heteroatom and optionally a ring O heteroatom, and wherein R¹ is optionally substituted with 1 or more R⁴;
R² is
(v) (C5-7)cycloalkyl, fused to a 6-membered aromatic or heteroaromatic ring that contains 0, 1 or 2 ring N atoms;
(vi) a 5- to 7-membered non-aromatic heterocycle containing one ring O heteroatom, optionally fused to a 6-membered aromatic or heteroaromatic ring that contains 0, 1 or 2 ring N atoms;
(vii) CH₂Ar, where Ar is a 6-membered aromatic or heteroaromatic ring that contains 0, 1 or 2 ring N atoms; or
(viii) a (C3-8)alkyl group that may be straight chain, branched or cyclic, or a combination thereof;
and wherein R² is optionally substituted with 1 or more R⁵;
each R^{3a} is independently H or (C1-6)alkyl, the (C1-6)alkyl being optionally substituted by 1 or more halogen;
each R^{3b} is independently H, (C1-6)alkyl, (C1-6)alkoxy, CN or halogen, the (C1-6)alkyl and (C1-6)alkoxy being optionally substituted by 1 or more halogen, wherein at least 1 R^{3b} is other than H;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and (C1-6)alkylene-(C1-6)alkoxy being optionally substituted with 1 or more substituents independently selected from halogen, OH and (C1-6)alkoxy; and
each R⁵ is independently halogen, OH, CN, (C1-6)alkyl, (C1-6)alkoxy or —(C1-6)alkylene-(C1-6)alkoxy the (C1-6)alkyl and (C1-6)alkoxy being optionally substituted by 1 or more halogen or OH.

24. The compound, or a pharmaceutically acceptable salt or derivative thereof, of any of claims 13-23, wherein each R^{3a} is independently —H or CH₃, and/or each R^{3b} is independently —H, —CH₃, —OCH₃, halo, CN or cyclopropyl.

25. The compound, or a pharmaceutically acceptable salt or derivative thereof, of any of claims 13-24, wherein R² or R^{2a} is:
(i) (C5-7)cycloalkyl fused to a 6-membered aromatic or heteroaromatic ring that contains 0, 1 or 2 ring N atoms, wherein the (C5-7)cycloalkyl is optionally substituted with 1 to 3 substituents independently selected from OH, halogen, (C1-4)alkyl, (C1-4)alkoxy, the (C1-4)alkyl and (C1-4)alkoxy groups being optionally substituted with one or more fluoro, and the 6-membered aromatic or heteroaromatic ring is optionally substituted with 1 to 3 substituents independently selected from (C1-4)alkyl, (C1-4)alkoxy, CN and halogen, the (C1-4)alkyl and (C1-4)alkoxy groups being optionally substituted with one or more fluoro;
(ii) a 5- to 7-membered non-aromatic heterocycle containing one ring O heteroatom, optionally fused to a 6-membered aromatic or heteroaromatic ring that contains 0, 1 or 2 ring N atoms, wherein the 5- to 7-membered non-aromatic heterocycle is optionally substituted with 1 to 3 substituents on one or more ring carbon atoms independently selected from OH, halogen, (C1-4)alkyl, and (C1-4)alkoxy, the (C1-4)alkyl and (C1-4)alkoxy groups being optionally substituted with one or more fluoro, and the 6-membered aromatic or heteroaromatic ring is optionally substituted with 1 to 3 substituents independently selected from (C1-4)alkyl, (C1-4)alkoxy, CN and halogen, the (C1-4)alkyl and (C1-4)alkoxy groups being optionally substituted with one or more fluoro; or
(35) (iv) a (C5-6)cycloalkyl group, substituted by 2 or 3 substituents on one or more ring carbon atoms independently selected from OH, halogen, (C1-4)alkyl, (C1-4)alkoxy, the (C1-4)alkyl and (C1-4)alkoxy groups being optionally substituted with one or more fluoro, optionally wherein the (C5-6)cycloalkyl group is substituted by 2 halogen substituents (optionally on a single ring carbon atom).

26. The compound, or a pharmaceutically acceptable salt or derivative thereof, of any of claims 13-25, wherein R² or R^{2a} is:
(i) (C5-6)cycloalkyl, optionally fused to a phenyl ring;
(ii) a 5- to 6-membered non-aromatic heterocycle containing one ring O heteroatom, optionally fused to a phenyl ring; or
wherein R² or R^{2a} is optionally substituted, wherein when R^{2a} is a (C5-6)cycloalkyl group not fused to a phenyl ring it is substituted by at least 2 R⁵.

27. The compound, or a pharmaceutically acceptable salt or derivative thereof, of any of claims 13-26, wherein R² or R^{2a} is a group of formula 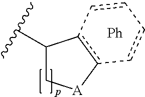
wherein A is O or CH₂; p is 1, 2 or 3; Ph is an optionally present, fused phenyl ring, and wherein R² or R^{2a} is optionally substituted with 1 or more R⁵; and wherein when A is CH₂, Ph is present or A is C(R⁵)₂ (for example, CF₂).

28. The compound, or a pharmaceutically acceptable salt or derivative thereof, of claim 21, wherein
R¹ is a 6-membered saturated monocyclic ring containing 1 or 2 ring N heteroatoms, wherein at least 1 ring N heteroatom is not at the point of attachment of R¹, or a 7- to 8-membered saturated, bridged ring system containing 2 ring N heteroatoms, and wherein R¹ is optionally substituted with 1 R⁴;
R^{2a} is
(i) (C5-6)cycloalkyl, optionally fused to a phenyl ring;
(ii) a 5- to 6-membered non-aromatic heterocycle containing one ring O heteroatom, optionally fused to a phenyl ring; or
and wherein R^{2a} is optionally substituted with 1 or 2 R⁵, wherein when R^{2a} is a (C5-6)cycloalkyl group, not fused to a phenyl ring, it is substituted by 2 R⁵;
R⁴, where present, is (C1-6)alkyl optionally substituted with OH;
R⁵, where present, is OH.

29. The compound or a pharmaceutically acceptable salt or derivative thereof, of any of claims 21 to 28, wherein R¹ is a 5- to 6-membered saturated, monocyclic ring containing at least 1 ring N heteroatom and optionally a ring O heteroatom; or a 7- to 8-membered saturated, bridged ring containing 1 or 2 ring N heteroatoms; a 9-membered saturated, bridged ring system containing 2 ring N heteroatoms and a ring O-heteroatom; or a 7- to 10-membered saturated, fused or spiro ring system containing 1 or 2 ring N heteroatoms; and wherein R¹ is optionally substituted with 1, 2 or 3 R⁴.

30. The compound, or a pharmaceutically acceptable salt or derivative thereof, of any of claims 21 to 29, wherein R¹ is a 6-membered saturated monocyclic ring containing 1 or 2 ring N heteroatoms, wherein at least 1 ring N heteroatom is not at the point of attachment of R¹, a 7- to 8-membered saturated, bridged ring system containing 2 ring N heteroatoms, and wherein R¹ is optionally substituted with 1 R⁴, optionally R¹ is a 7- to 8-membered saturated, bridged ring containing 2 ring N heteroatoms, wherein at least 1 ring N heteroatom is not at the point of attachment of R¹, and wherein R¹ is optionally substituted with 1 R⁴.

31. The compound, or a pharmaceutically acceptable salt or derivative thereof, of any of claims 13-30, wherein the bicyclic heteroaromatic ring system containing X₁, X₂, Y₁, Y₂, Z₁, Z₂ and Z₃ is selected from:

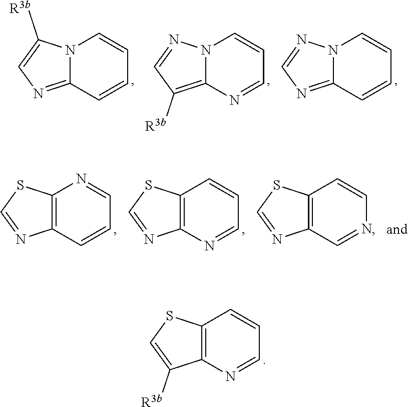

32. A compound selected from:
(S)—N-(chroman-4-yl)-2-(4-methylpyridin-3-yl)-1H-benzo[d]imidazole-5-carboxamide;
(S)—N-(chroman-4-yl)-2-(6-methylpyridin-3-yl)-1H-benzo[d]imidazole-5-carboxamide;
(S)—N-(chroman-4-yl)-2-(2-methylpyridin-3-yl)-1H-benzo[d]imidazole-5-carboxamide;
(S)—N-(chroman-4-yl)-2-(5-methylpyridin-3-yl)-1H-benzo[d]imidazole-5-carboxamide;
(R)-2-(pyridin-3-yl)-N-(1,2,3,4-tetrahydronaphthalen-1-yl)-1H-benzo[d]imidazole-5-carboxamide;
(S)-2-(pyridin-3-yl)-N-(1,2,3,4-tetrahydronaphthalen-1-yl)-1H-benzo[d]imidazole-5-carboxamide;
(R)—N-(chroman-4-yl)-2-(pyridin-3-yl)-1H-benzo[d]imidazole-5-carboxamide;
(S)—N-(chroman-4-yl)-2-(pyridin-3-yl)-1H-benzo[d]imidazole-5-carboxamide;
(R)—N-(2,3-dihydro-1H-inden-1-yl)-2-(pyridin-3-yl)-1H-benzo[d]imidazole-5-carboxamide;
(S)—N-(2,3-dihydro-1H-inden-1-yl)-2-(pyridin-3-yl)-1H-benzo[d]imidazole-5-carboxamide;
(R)—N-(6-chlorochroman-4-yl)-2-(pyridin-3-yl)-1H-benzo[d]imidazole-5-carboxamide;
(S)—N-(6-chlorochroman-4-yl)-2-(pyridin-3-yl)-1H-benzo[d]imidazole-5-carboxamide;
(S)—N-(2,3-dihydro-1H-inden-1-yl)-2-(1-methylpiperidin-4-yl)-1H-benzo[d]imidazole-5-carboxamide;
(R)-2-(1-methylpiperidin-4-yl)-N-(1,2,3,4-tetrahydronaphthalen-1-yl)-1H-benzo[d]imidazole-5-carboxamide;
(S)-2-(1-methylpiperidin-4-yl)-N-(1,2,3,4-tetrahydronaphthalen-1-yl)-1H-benzo[d]imidazole-5-carboxamide;
(R)—N-(6-chlorochroman-4-yl)-2-(1-methylpiperidin-4-yl)-1H-benzo[d]imidazole-5-carboxamide;
(S)—N-(6-chlorochroman-4-yl)-2-(1-methylpiperidin-4-yl)-1H-benzo[d]imidazole-5-carboxamide;
(S)—N-(chroman-4-yl)-2-(1-methylpiperidin-4-yl)-1H-benzo[d]imidazole-5-carboxamide;
(S)—N-(chroman-4-yl)-2-(4-ethylpiperazin-1-yl)-1H-benzo[d]imidazole-5-carboxamide;
(S)—N-(chroman-4-yl)-1-methyl-2-(4-methylpyridin-3-yl)-1H-benzo[d]imidazole-5-carboxamide;
(S)—N-(chroman-4-yl)-1-methyl-2-(6-methylpyridin-3-yl)-1H-benzo[d]imidazole-5-carboxamide;
(S)—N-(chroman-4-yl)-1-methyl-2-(2-methylpyridin-3-yl)-1H-benzo[d]imidazole-5-carboxamide;
(S)—N-(chroman-4-yl)-1-methyl-2-(5-methylpyridin-3-yl)-1H-benzo[d]imidazole-5-carboxamide;
(S)—N-(chroman-4-yl)-1-methyl-2-(pyridin-3-yl)-1H-benzo[d]imidazole-5-carboxamide;
(S)—N-(chroman-4-yl)-1-methyl-2-(4-methylpyridin-3-yl)-1H-benzo[d]imidazole-6-carboxamide;
(S)—N-(chroman-4-yl)-1-methyl-2-(6-methylpyridin-3-yl)-1H-benzo[d]imidazole-6-carboxamide;
(S)—N-(chroman-4-yl)-1-methyl-2-(2-methylpyridin-3-yl)-1H-benzo[d]imidazole-6-carboxamide;
(S)—N-(chroman-4-yl)-1-methyl-2-(5-methylpyridin-3-yl)-1H-benzo[d]imidazole-6-carboxamide;
(S)—N-(chroman-4-yl)-1-methyl-2-(pyridin-3-yl)-1H-benzo[d]imidazole-6-carboxamide;
(S)—N-(chroman-4-yl)-2-(4-ethylpiperazin-1-yl)-1-methyl-1H-benzo[d]imidazole-6-carboxamide;
(S)—N-(chroman-4-yl)-1-methyl-2-(piperazin-1-yl)-1H-benzo[d]imidazole-6-carboxamide;
(S)—N-(chroman-4-yl)-2-(4-ethylpiperazin-1-yl)-1-methyl-1H-benzo[d]imidazole-5-carboxamide;
(S)—N-(chroman-4-yl)-1-methyl-2-(piperazin-1-yl)-1H-benzo[d]imidazole-5-carboxamide;
(S)—N-(chroman-4-yl)-2-(pyridin-3-yl)-[1,2,4]triazolo[1,5-a]pyridine-6-carboxamide;
(S)—N-(chroman-4-yl)-2-(6-methylpyridin-3-yl)-[1,2,4]triazolo[1,5-a]pyridine-6-carboxamide;
(S)—N-(chroman-4-yl)-2-(5-methylpyridin-3-yl)-[1,2,4]triazolo[1,5-a]pyridine-6-carboxamide;
(S)—N-(chroman-4-yl)-2-(4-methylpyridin-3-yl)-[1,2,4]triazolo[1,5-a]pyridine-6-carboxamide;
(S)—N-(chroman-4-yl)-2-(2-methylpyridin-3-yl)-[1,2,4]triazolo[1,5-a]pyridine-6-carboxamide;
(S)—N-(chroman-4-yl)-2-(2,6-dimethylpyridin-3-yl)-[1,2,4]triazolo[1,5-a]pyridine-6-carboxamide;
(S)—N-(chroman-4-yl)-2-(piperazin-1-yl)-[1,2,4]triazolo[1,5-a]pyridine-6-carboxamide;
(S)—N-(chroman-4-yl)-2-(4-methylpiperazin-1-yl)-[1,2,4]triazolo[1,5-a]pyridine-6-carboxamide;
(S)—N-(chroman-4-yl)-2-morpholino-[1,2,4]triazolo[1,5-a]pyridine-6-carboxamide;
(S)-2-(4-(tert-butyl)piperazin-1-yl)-N-(chroman-4-yl)-[1,2,4]triazolo[1,5-a]pyridine-6-carboxamide;
(S)—N-(chroman-4-yl)-2-(piperidin-1-yl)-[1,2,4]triazolo[1,5-a]pyridine-6-carboxamide;
(S)—N-(chroman-4-yl)-2-(4-ethylpiperazin-1-yl)-[1,2,4]triazolo[1,5-a]pyridine-6-carboxamide;
(S)—N-(chroman-4-yl)-2-(4-isopropylpiperazin-1-yl)-[1,2,4]triazolo[1,5-a]pyridine-6-carboxamide;
(R)—N-(2,3-dihydro-1H-inden-1-yl)-2-(piperazin-1-yl)-[1,2,4]triazolo[1,5-a]pyridine-6-carboxamide;
(S)—N-(2,3-dihydro-1H-inden-1-yl)-2-(piperazin-1-yl)-[1,2,4]triazolo[1,5-a]pyridine-6-carboxamide;
(S)—N-(chroman-4-yl)-2-(piperidin-4-yl)-[1,2,4]triazolo[1,5-a]pyridine-6-carboxamide;
(S)—N-(chroman-4-yl)-2-(1-methylpiperidin-4-yl)-[1,2,4]triazolo[1,5-a]pyridine-6-carboxamide;
(S)—N-(chroman-4-yl)-2-(1-ethylpiperidin-4-yl)-[1,2,4]triazolo[1, 5-a]pyridine-6-carboxamide;
(S)—N-(chroman-4-yl)-2-(2-methylpyridin-3-yl)-[1,2,4]triazolo[1,5-a]pyridine-7-carboxamide;
(S)—N-(chroman-4-yl)-2-(4-methylpyridin-3-yl)-[1,2,4]triazolo[1,5-a]pyridine-7-carboxamide;
(S)—N-(chroman-4-yl)-2-(4-hydroxypiperidin-1-yl)-[1,2,4]triazolo[1,5-a]pyridine-7-carboxamide;
(S)—N-(chroman-4-yl)-2-(piperazin-1-yl)-[1,2,4]triazolo[1,5-a]pyridine-7-carboxamide;
(S)—N-(chroman-4-yl)-2-(4-methylpiperazin-1-yl)-[1,2,4]triazolo[1,5-a]pyridine-7-carboxamide;
(S)—N-(chroman-4-yl)-2-morpholino-[1,2,4]triazolo[1,5-a]pyridine-7-carboxamide;
(S)-2-(4-(tert-butyl)piperazin-1-yl)-N-(chroman-4-yl)-[1,2,4]triazolo[1,5-a]pyridine-7-carboxamide;
(S)—N-(chroman-4-yl)-2-(piperidin-1-yl)-[1,2,4]triazolo[1,5-a]pyridine-7-carboxamide;
(S)—N-(chroman-4-yl)-2-(4-ethylpiperazin-1-yl)-[1,2,4]triazolo[1,5-a]pyridine-7-carboxamide;
(S)—N-(chroman-4-yl)-2-(4-isopropylpiperazin-1-yl)-[1,2,4]triazolo[1,5-a]pyridine-7-carboxamide;
(S)—N-(2,3-dihydro-1H-inden-1-yl)-2-(piperazin-1-yl)-[1,2,4]triazolo[1,5-a]pyridine-7-carboxamide;
(R)—N-(2,3-dihydro-1H-inden-1-yl)-2-(piperazin-1-yl)-[1,2,4]triazolo[1,5-a]pyridine-7-carboxamide;
(S)—N-(2,3-dihydro-1H-inden-1-yl)-2-(4-methylpiperazin-1-yl)-[1,2,4]triazolo[1,5-a]pyridine-7-carboxamide;
(S)—N-(2,3-dihydro-1H-inden-1-yl)-2-(4-ethylpiperazin-1-yl)-[1,2,4]triazolo[1,5-a]pyridine-7-carboxamide;
2-(3,8-diazabicyclo[3.2.1]octan-8-yl)-N-(4,4-difluorocyclohexyl)imidazo[1,2-a]pyridine-6-carboxamide;
2-(3,8-diazabicyclo[3.2.1]octan-8-yl)-N-cyclopentylimidazo[1,2-a]pyridine-6-carboxamide;
(S)—N-(chroman-4-yl)-2-(piperazin-1-yl)pyrazolo[1,5-a]pyrimidine-6-carboxamide;
(S)—N-(chroman-4-yl)-2-(4-methylpiperazin-1-yl)pyrazolo[1,5-a]pyrimidine-6-carboxamide;
(S)—N-(chroman-4-yl)-2-(4-ethylpiperazin-1-yl)pyrazolo[1,5-a]pyrimidine-6-carboxamide;
(S)—N-(2,3-dihydro-1H-inden-1-yl)-2-(piperazin-1-yl)pyrazolo[1,5-a]pyrimidine-6-carboxamide;
N-cyclopentyl-2-(4-ethylpiperazin-1-yl)pyrazolo[1,5-a]pyrimidine-6-carboxamide;
N-cyclohexyl-2-(4-ethylpiperazin-1-yl)pyrazolo[1,5-a]pyrimidine-6-carboxamide;
N-(4,4-difluorocyclohexyl)-2-(4-ethylpiperazin-1-yl)pyrazolo[1,5-a]pyrimidine-6-carboxamide;
(S)—N-(2,3-dihydro-1H-inden-1-yl)-2-(4-ethylpiperazin-1-yl)pyrazolo[1,5-a]pyrimidine-6-carboxamide;
N-cyclopentyl-2-(4-methylpiperazin-1-yl)pyrazolo[1,5-a]pyrimidine-6-carboxamide;
N-cyclohexyl-2-(4-methylpiperazin-1-yl)pyrazolo[1,5-a]pyrimidine-6-carboxamide;
N-(4,4-difluorocyclohexyl)-2-(4-methylpiperazin-1-yl)pyrazolo[1,5-a]pyrimidine-6-carboxamide;
(S)—N-(2,3-dihydro-1H-inden-1-yl)-2-(4-methylpiperazin-1-yl)pyrazolo[1,5-a]pyrimidine-6-carboxamide;
2-(3,8-diazabicyclo[3.2.1]octan-8-yl)-N-cyclopentylpyrazolo[1,5-a]pyrimidine-6-carboxamide;
N-cyclopentyl-2-(3-methyl-3,8-diazabicyclo[3.2.1]octan-8-yl)pyrazolo[1,5-a]pyrimidine-6-carboxamide;
(S)—N-(chroman-4-yl)-2-(piperidin-4-yl)pyrazolo[1,5-a]pyrimidine-6-carboxamide;
(S)—N-(2,3-dihydro-1H-inden-1-yl)-2-(piperidin-4-yl)pyrazolo[1,5-a]pyrimidine-6-carboxamide;
(S)—N-(chroman-4-yl)-2-(1-ethylpiperidin-4-yl)pyrazolo[1,5-a]pyrimidine-6-carboxamide;
N-cyclopentyl-2-(1-methylpiperidin-4-yl)pyrazolo[1,5-a]pyrimidine-6-carboxamide;
N-cyclohexyl-2-(1-methylpiperidin-4-yl)pyrazolo[1,5-a]pyrimidine-6-carboxamide;
N-(4,4-difluorocyclohexyl)-2-(1-methylpiperidin-4-yl)pyrazolo[1,5-a]pyrimidine-6-carboxamide;
(S)—N-(chroman-4-yl)-2-(1-methylpiperidin-4-yl)pyrazolo[1,5-a]pyrimidine-6-carboxamide;
N-(4-fluorobenzyl)-2-(piperidin-4-yl)pyrazolo[1,5-a]pyrimidine-6-carboxamide;
(S)—N-(chroman-4-yl)-2-(pyridin-3-yl)pyrazolo[1,5-a]pyrimidine-6-carboxamide;
N-(4,4-difluorocyclohexyl)-7-methyl-2-(piperidin-4-yl)pyrazolo[1,5-a]pyrimidine-6-carboxamide;
(S)—N-(chroman-4-yl)-7-methyl-2-(piperidin-4-yl)pyrazolo[1,5-a]pyrimidine-6-carboxamide;
(S)—N-(chroman-4-yl)-2-(1-ethylpiperidin-4-yl)-7-methylpyrazolo[1,5-a]pyrimidine-6-carboxamide;
N-(4,4-difluorocyclohexyl)-2-(1-ethylpiperidin-4-yl)-7-methylpyrazolo[1,5-a]pyrimidine-6-carboxamide;
(S)—N-(chroman-4-yl)-7-methyl-2-(1-methylpiperidin-4-yl)pyrazolo[1,5-a]pyrimidine-6-carboxamide;
N-(4,4-difluorocyclohexyl)-7-methyl-2-(1-methylpiperidin-4-yl)pyrazolo[1,5-a]pyrimidine-6-carboxamide;
N-cyclopentyl-2-(piperidin-4-yl)benzo[d]oxazole-5-carboxamide;
N-cyclopentyl-2-(1-methylpiperidin-4-yl)benzo[d]oxazole-5-carboxamide;
N-cyclopentyl-2-(1-ethylpiperidin-4-yl)benzo[d]oxazole-5-carboxamide;
(S)—N-(chroman-4-yl)-2-(piperazin-1-yl)benzo[d]oxazole-5-carboxamide;
N-cyclopentyl-2-(piperazin-1-yl)benzo[d]oxazole-5-carboxamide;
(S)—N-(chroman-4-yl)-2-(4-ethylpiperazin-1-yl)benzo[d]oxazole-5-carboxamide;
N-cyclopentyl-2-(4-ethylpiperazin-1-yl)benzo[d]oxazole-5-carboxamide;
(S)—N-(chroman-4-yl)-2-(4-methylpiperazin-1-yl)benzo[d]oxazole-5-carboxamide;
N-cyclopentyl-2-(4-methylpiperazin-1-yl)benzo[d]oxazole-5-carboxamide;
(S)—N-(chroman-4-yl)-2-(4-(2-hydroxyethyl)piperazin-1-yl)benzo[d]oxazole-5-carboxamide;
N-cyclopentyl-2-(4-(2-hydroxyethyl)piperazin-1-yl)benzo[d]oxazole-5-carboxamide;
N-cyclopentyl-2-(4-ethylpiperazin-1-yl)benzo[d]oxazole-6-carboxamide;
N-cyclopentyl-2-(piperazin-1-yl)benzo[d]oxazole-6-carboxamide;
(S)—N-(chroman-4-yl)-2-(1-methyl-1H-pyrazol-4-yl)benzo[d]oxazole-6-carboxamide;
N-cyclopentyl-2-(piperidin-4-yl)thiazolo[5,4-b]pyridine-5-carboxamide;
N-(4,4-difluorocyclohexyl)-2-(piperidin-4-yl)thiazolo[5,4-b]pyridine-5-carboxamide;
N-cyclopentyl-2-(1-methylpiperidin-4-yl)thiazolo[5,4-b]pyridine-5-carboxamide;
N-(4,4-difluorocyclohexyl)-2-(1-methylpiperidin-4-yl)thiazolo[5,4-b]pyridine-5-carboxamide;
2-(3,8-diazabicyclo[3.2.1]octan-8-yl)-N-cyclopentylthiazolo[5,4-b]pyridine-5-carboxamide;
2-(3,8-diazabicyclo[3.2.1]octan-8-yl)-N-(4,4-difluorocyclohexyl)thiazolo[5,4-b]pyridine-5-carboxamide;
2-(3,8-diazabicyclo[3.2.1]octan-8-yl)-N-(4,4-difluorocyclohexyl)thiazolo[4,5-b]pyridine-6-carboxamide;
N-cyclopentyl-2-(piperidin-4-yl)thiazolo[4,5-b]pyridine-6-carboxamide;
2-(3,8-diazabicyclo[3.2.1]octan-8-yl)-N-cyclopentylthiazolo[4,5-c]pyridine-6-carboxamide;
2-(3,8-diazabicyclo[3.2.1]octan-8-yl)-N-(4,4-difluorocyclohexyl)thiazolo[4,5-c]pyridine-6-carboxamide;
2-(3,8-diazabicyclo[3.2.1]octan-8-yl)-N-cyclopentylthieno[3,2-b]pyridine-6-carboxamide;
2-(3,8-diazabicyclo[3.2.1]octan-8-yl)-N-(4,4-difluorocyclohexyl)thieno[3,2-b]pyridine-6-carboxamide;
or a pharmaceutically acceptable salt or derivative thereof.

33. A pharmaceutical composition comprising a compound or a pharmaceutically acceptable salt or derivative as defined in any of claims 1-32, and a pharmaceutically acceptable excipient.

34. A compound or pharmaceutically acceptable salt or derivative of any of claims 13-32 for use in therapy.

35. A compound or pharmaceutically acceptable salt or derivative of any of claims 13-32 or a pharmaceutical composition of claim 33 for use in the treatment or prevention of a disease or disorder that can be ameliorated by activation of long isoforms of PDE4 or a disease or disorder mediated by excessive intracellular cyclic AMP signalling.

36. The compound or pharmaceutically acceptable salt or derivative for use of any of claims 1 to 12 or the compound or pharmaceutically acceptable salt or derivative or pharmaceutical composition for use of claim 35 in the treatment or prevention of a disease or disorder mediated by excessive intracellular cyclic AMP signalling.

37. The compound or pharmaceutically acceptable salt or derivative or pharmaceutical composition for use of claim 36 wherein the excessive intracellular cyclic AMP signalling is caused by:
a. excessive hormone levels produced by an adenoma.
b. a gain-of-function gene mutation in a G-protein coupled receptor (GPCR);
c. an activating mutation in the GNAS1 gene, which encodes the α-subunit of the G-protein Gₛ; or
d. a bacterial toxin.

38. The compound or pharmaceutically acceptable salt or derivative or pharmaceutical composition for use of any of claims 1-12 or 34-37, wherein the disease is cancer.

39. The compound or pharmaceutically acceptable salt or derivative or pharmaceutical composition for use of claim 38, wherein the cancer is prostate cancer.

40. The compound or pharmaceutically acceptable salt or derivative or pharmaceutical composition for use of any of claims 1-12 or 34-37, wherein the disease is:
a. pituitary adenoma, Cushing's disease, polycystic kidney disease or polycystic liver disease;
b. hyperthyroidism, Jansens's metaphyseal chondrodysplasia, hyperparathyroidism, or familial male-limited precocious puberty;
c. McCune-Albright syndrome;
d. cholera, whooping cough, anthrax, or tuberculosis;
e. HIV, AIDS, or Common Variable Immunodeficiency (CVID);
f. melanoma, pancreatic cancer, leukaemia, prostate cancer, adrenocortical tumours, testicular cancer, primary pigmented nodular adrenocortical diseases (PPNAD), or Carney Complex;
g. autosomal dominant polycystic kidney disease (ADPKD) or autosomal recessive polycystic kidney disease (ARPKD); or
h. maturity onset diabetes of young type 5 (MODY5); or
i. cardiac hypertrophy.

41. The compound or pharmaceutically acceptable salt or derivative or pharmaceutical composition for use of claim 40, wherein the disease is:
a. autosomal dominant polycystic kidney disease (ADPKD); or
b. autosomal recessive polycystic kidney disease (ARPKD).

42. The compound or pharmaceutically acceptable salt or derivative or pharmaceutical composition for use of claim 40, wherein the disease is hyperparathyroidism.
